(19) United States
(12) Patent Application Publication
(10) Pub. No.: US 2013/0150325 A1
(43) Pub. Date: Jun. 13, 2013

(54) 3-(Fluorovinyl)pyrazoles and their use

(71) Applicants: Michael HÄRTER, Leverkusen (DE); Hartmut BECK, Wuppertal (DE); Karl-Heinz THIERAUCH, Berlin (DE); Peter ELLINGHAUS, Melle (DE); Susanne GRESCHAT, Wagenfeld (DE); Joachim SCHUHMACHER, Wuppertal (DE)

(72) Inventors: Michael HÄRTER, Leverkusen (DE); Hartmut BECK, Wuppertal (DE); Karl-Heinz THIERAUCH, Berlin (DE); Peter ELLINGHAUS, Melle (DE); Susanne GRESCHAT, Wagenfeld (DE); Joachim SCHUHMACHER, Wuppertal (DE)

(73) Assignee: BAYER INTELLECTUAL PROPERTY GMBH, Monheim (DE)

(21) Appl. No.: 13/551,175

(22) Filed: Jul. 17, 2012

(30) Foreign Application Priority Data
Jul. 21, 2011 (EP) 11174843.0

(51) Int. Cl. A61K 31/415; C07D 401/06; A61K 31/4439; C07D 413/14; A61K 31/5377; C07D 401/10; A61K 31/454; C07D 403/10; A61K 31/4155; C07D 413/10; A61K 31/496; C07F 7/10; A61K 31/695; A61K 45/06; C07D 231/12

(52) U.S. Cl. CPC A61K 31/415; C07D 231/12; C07D 401/06; A61K 31/4439; C07D 413/14; A61K 31/5377; C07D 401/10; A61K 31/454; C07D 403/10; A61K 31/4155; C07D 413/10; A61K 31/496; C07F 7/10; A61K 31/695; A61K 45/06
USPC 514/63; 548/373.1; 514/406; 546/275.4; 514/341; 544/124; 514/236.5; 546/211; 514/326; 548/364.1; 514/210.2; 544/140; 544/371; 514/254.05; 544/229; 544/364; 514/253.09

(57) ABSTRACT

The present application relates to novel 3-(fluorovinyl)pyrazole derivatives, to processes for their preparation, to their use for treatment and/or prevention of diseases and to their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

The present application relates to novel 3-(fluorovinyl)pyrazole derivatives, to processes for their preparation, to their use for treatment and/or prevention of diseases and to their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

Cancer diseases are the consequence of uncontrolled cell growth of the most diverse tissue. In many cases the new cells penetrate into existing tissue (invasive growth), or they metastase into remote organs. Cancer diseases occur in the most diverse organs and often have tissue-specific courses of the disease. The term cancer disease as a generic term therefore describes a large group of defined diseases of various organs, tissue and cell types.

In the year 2002 4.4 million people worldwide were diagnosed with tumour diseases of the breast, intestine, ovaries, lung or prostate. In the same year, approx. 2.5 million deaths were assumed to be a consequence of these diseases (Globocan 2002 Report). In the USA alone, for the year 2005 over 1.25 million new cases and over 500,000 deaths were predicted from cancer diseases. The majority of these new cases concern cancer diseases of the intestine (˜100,000), lung (˜170,000), breast (˜210,000) and prostate (˜230,000). A further increase in cancer diseases of approx. 15% over the next 10 years is assumed (American Cancer Society, Cancer Facts and Figures 2005).

Tumours in early stages can possibly be removed by surgical and radiotherapy measures. Metastased tumours as a rule can only be treated palliatively by chemotherapeutics. The aim here is to achieve the optimum combination of an improvement in the quality of life and prolonging of life.

Chemotherapies are often composed of combinations of cytotoxic medicaments. The majority of these substances have as their action mechanism bonding to tubulin, or they are compounds which interact with the formation and processing of nucleic acids. More recently these also include enzyme inhibitors, which interfere with epigenetic DNA modification or cell cycle progression (e.g. histone deacetylase inhibitors, aurora kinase inhibitors). Since such therapies are toxic, more recently the focus has increasingly been on targeted therapies in which specific processes in the cell are blocked without there being a high toxic load. These include in particular inhibitors of kinases which inhibit the phosphorylation of receptors and signal transmission molecules. An example of these is imatinib, which is employed very successfully for treatment of chronic myeloid leukaemia (CML) and gastrointestinal stromal tumours (GIST). Further examples are substances which block EGFR kinase and HER2, such as erlotinib, and VEGFR kinase inhibitors, such as sorafenib and sunitinib, which are employed on kidney cell carcinomas, liver carcinomas and advanced stages of GIST.

The life expectancy of colorectal carcinoma patients has been successfully prolonged with an antibody directed against VEGF. Bevacizumab inhibits growth of blood vessels, which obstructs rapid expansion of tumours since this requires connection to the blood vessel system for a continuously functioning supply and disposal.

One stimulus of angiogenesis is hypoxia, which occurs again and again with solid tumours since the blood supply is inadequate because of the unregulated growth. If there is a lack of oxygen, cells switch their metabolism from oxidative phosphorylation to glycolysis so that the ATP level in the cell is stabilized. This process is controlled by a transcription factor, which is regulated upwards depending on the oxygen content in the cell. This transcription factor, called “hypoxia-induced factor” (HIF), is normally removed posttranslationally by rapid degradation and prevented from transportation into the cell nucleus. This is effected by hydroxylation of two proline units in the oxygen degradable domain (ODD) and an asparagine unit in the vicinity of the C terminus by the enzymes prolyl dehydrogenase and FIH (“factor inhibiting HIF”). After the modification of the proline units, HIF can be degraded with mediation by the Hippel-Lindau protein (part of a ubiquitin-E3-ligase complex) via the proteasome apparatus (Maxwell, Wiesener et al., 1999). In the event of oxygen deficiency, the degradation does not take place and the protein is regulated upwards and leads to transcription or blockade of the transcription of numerous (more than 100) other proteins (Semenza and Wang, 1992; Wang and Semenza, 1995).

The transcription factor HIF is formed by the regulated α-subunit and a constitutively present β-subunit (ARNT, aryl hydrocarbon receptor nuclear translocator). There are three different species of the α-subunit, 1α, 2α and 3α, the last of these being rather to be assumed as a suppressor (Makino, Cao et al., 2001). The HIF subunits are bHLH (basic helix loop helix) proteins, which dimerize via their HLH and PAS (Per-Arnt-Sim) domain, which starts their transactivation activity (Jiang, Rue et al., 1996).

In the most important tumour entities, overexpression of the HIF1α protein is correlated with increasing density of blood vessels and enhanced VEGF expression (Hirota and Semenza, 2006). At the same time glucose metabolism is changed to glycolysis, and the Krebs cycle is reduced in favour of the production of cell units. This also implies a change in fat metabolism. Such changes appear to guarantee the survival of the tumours. On the other hand, if the activity of HIF is now inhibited, the development of tumours could consequently be suppressed. This has already been observed in various experimental models (Chen, Zhao et al., 2003; Stoeltzing, McCarty et al., 2004; Li, Lin et al., 2005; Mizukami, Jo et al., 2005; Li, Shi et al., 2006). Specific inhibitors of the metabolism controlled by HIF should therefore be suitable as tumour therapeutics.

The object of the present invention was therefore to provide novel compounds which act as inhibitors of the transactivating action of the transcription factor HIF and can be employed as such for treatment and/or prevention of diseases, in particular of hyperproliferative and angiogenic diseases, such as cancer diseases.

WO 2005/030121-A2 and WO 2007/065010-A2 describe the suitability of certain pyrazole derivatives for inhibiting the expression of HIF and HIF-regulated genes in tumour cells. WO 2008/141731-A2, WO 2010/054762-A1, WO 2010/054763-A1 and WO 2010/054764-A1 disclose certain heteroaryl-substituted pyrazole derivatives as inhibitors of the HIF regulation path for the treatment of cancer diseases.

EP 1 310 485-A1 describes disubstituted heteroaryl compounds as TGFβ inhibitors for the treatment of fibroses. WO 2008/097538-A1 discloses certain 2-phenylvinyl-substituted heterocyclic compounds for the treatment of Alzheimer's disease. WO 2009/121623-A2 claims the use of 1,3-disubstituted pyrroles and pyrazoles for the treatment of muscular dystrophies.

The present invention provides compounds of the general formula (I)

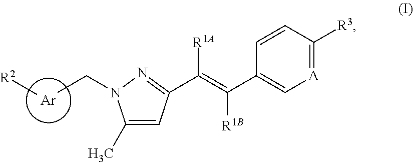

in which
one of the two radicals R^{1A} and R^{1B} represents fluorine and the other represents hydrogen,
Ar with the substituent R² represents a phenyl or pyridyl ring of the formula

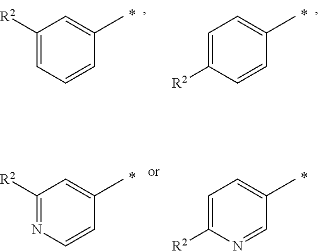

in which * denotes the point of attachment to the neighbouring CH₂ group,
R² represents hydrogen or a substituent selected from the group consisting of halogen, cyano, (C₁-C₆)-alkyl, (C₂-C₆)-alkenyl, (C₃-C₆)-cycloalkyl, (C₁-C₆)-alkoxy, (C₃-C₆)-cycloalkoxy, (C₁-C₄)-alkoxycarbonyl, (C₁-C₄)-alkylsulphonyl, —NR⁵R⁶ and —C(═O)—NR⁵R⁶, where (C₁-C₆)-alkyl for its part may be substituted up to three times by fluorine and up to two times by identical or different radicals selected from the group consisting of hydroxyl, (C₁-C₄)-alkoxy, (C₁-C₄)-alkylcarbonyloxy and (C₃-C₆)-cycloalkyl
   and
   the cycloalkyl groups mentioned for their part may be substituted up to two times by identical or different radicals selected from the group consisting of fluorine, (C₁-C₄)-alkyl, trifluoromethyl, hydroxyl, hydroxymethyl, (C₁-C₄)-alkoxy and (C₁-C₄)-alkylcarbonyloxy,
   and in which
   R⁵ and R⁶ independently of one another represent hydrogen, (C₁-C₆)-alkyl or (C₃-C₆)-cycloalkyl
   or
   R⁵ and R⁶ are attached to one another and together with the nitrogen atom to which they are attached form a 4- to 6-membered heterocycle which may contain a further heteroatom from the group consisting of N, O, S and S(O)₂ and which may be substituted up to two times by identical or different substituents selected from the group consisting of fluorine, cyano, hydroxyl, (C₁-C₄)-alkoxy, oxo, (C₁-C₄)-alkyl and (C₃-C₆)-cycloalkyl,
      where (C₁-C₄)-alkyl for its part may be substituted up to three times by fluorine,
R³ represents a substituent selected from the group consisting of halogen, cyano, pentafluorothio, tri-(C₁-C₄)-alkylsilyl, (C₁-C₆)-alkyl, —NR⁷R⁸, —OR⁸, —SR⁸, —S(O)₂—R⁸, (C₃-C₆)-cycloalkyl and 4- to 6-membered heterocyclyl,
   where (C₁-C₆)-alkyl for its part may be substituted by a radical selected from the group consisting of amino, —NR⁷R⁸, hydroxyl, —OR⁸, (C₃-C₆)-cycloalkyl and 4- to 6-membered heterocyclyl and also up to six times by fluorine
   and
   the cycloalkyl and heterocyclyl groups mentioned for their part may be substituted up to two times by identical or different radicals selected from the group consisting of fluorine, (C₁-C₄)-alkyl, trifluoromethyl, hydroxyl and (C₁-C₄)-alkoxy,
   and in which
   R⁷ represents hydrogen or (C₁-C₄)-alkyl
   and
   R⁸ represents (C₁-C₆)-alkyl or (C₃-C₆)-cycloalkyl,
      where (C₁-C₆)-alkyl for its part may be substituted by a radical selected from the group consisting of hydroxyl, (C₁-C₄)-alkoxy, —NR⁹R¹⁰ and —C(═O)—NR⁹R¹⁰ and also up to three times by fluorine, in which
      R⁹ and R¹⁰ independently of one another represent hydrogen or (C₁-C₄)-alkyl or are attached to one another and together with the nitrogen atom to which they are attached form a pyrrolidine, piperidine or morpholine ring,
         and
A represents N or C—R⁴, in which
   R⁴ represents hydrogen, fluorine, chlorine, cyano, methyl, trifluoromethyl or methoxy, and salts, solvates and solvates of the salts thereof.

Compounds according to the invention are the compounds of the formula (I) and their salts, solvates and solvates of the salts, the compounds included in the formula (I) of the formulae mentioned in the following and their salts, solvates and solvates of the salts, and the compounds included in the formula (I) and mentioned in the following as working examples and their salts, solvates and solvates of the salts, where the compounds included in the formula (I) and mentioned in the following are not already salts, solvates and solvates of the salts.

The compounds according to the invention can exist in different stereoisomeric forms depending on their structure, i.e. in the form of configuration isomers or optionally also as conformation isomers (enantiomers and/or diastereomers, including those in the case of atropisomers). The present invention therefore includes the enantiomers and diastereomers and their particular mixtures. The stereoisomerically uniform constituents can be isolated from such mixtures of enantiomers and/or diastereomers in a known manner; chromatography processes are preferably used for this, in particular HPLC chromatography on an achiral or chiral phase.

Where the compounds according to the invention can occur in tautomeric forms, the present invention includes all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds according to the invention can be prepared by generally used processes known to those skilled in the art, for example by the methods described below and the methods described in the working examples, by using corresponding isotopic modifications of the particular reagents and/or starting compounds therein.

Preferred salts in the context of the present invention are physiologically acceptable salts of the compounds according to the invention. Salts which are not themselves suitable for pharmaceutical uses but can be used, for example, for isolation or purification of the compounds according to the invention are also included.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, such as, by way of example and preferably, alkali metal salts (for example sodium and potassium salts), alkaline earth metal salts (for example calcium and magnesium salts) and ammonium salts derived from ammonia or organic amines having 1 to 16 carbon atoms, such as, by way of example and preferably, ethylamine, diethylamine, triethylamine, N,N-diisopropylethylamine, mono ethanolamine, diethanolamine, triethanolamine, dimethylaminoethanol, diethylaminoethanol, procaine, dicyclohexylamine, dibenzylamine, N-methylmorpholine, N-methylpiperidine, arginine, lysine and 1,2-ethylenediamine.

Solvates in the context of the invention are described as those forms of the compounds according to the invention which form a complex in the solid or liquid state by coordination with solvent molecules. Hydrates are a specific form of solvates, in which the coordination takes place with water. Hydrates are preferred solvates in the context of the present invention.

The N-oxides of pyridyl rings and tertiary cyclic amine groupings contained in compounds according to the invention are similarly included in the present invention.

The present invention moreover also includes prodrugs of the compounds according to the invention. The term “prodrugs” here designates compounds which themselves can be biologically active or inactive, but are converted (for example metabolically or hydrolytically) into compounds according to the invention during their dwell time in the body.

In the context of the present invention, the substituents have the following meaning, unless specified otherwise:

(C₁-C₆)-Alkyl and (C₁-C₄)-alkyl in the context of the invention represent a straight-chain or branched alkyl radical having 1 to 6 or, respectively, 1 to 4 carbon atoms. A straight-chain or branched alkyl radical having 1 to 4 carbon atoms is preferred. There may be mentioned by way of example and preferably: methyl, ethyl, n-propyl, isopropyl, n-butyl, iso-butyl, sec-butyl, tert-butyl, n-pentyl, 2-pentyl, 3-pentyl, neopentyl, n-hexyl, 2-hexyl and 3-hexyl.

Tri-(C₁-C₄)-alkylsilyl in the context of the invention represents a silyl group having three identical or different straight-chain or branched alkyl substituents, each of which contains 1 to 4 carbon atoms. There may be mentioned by way of example and preferably: trimethylsilyl, tert-butyldimethylsilyl and triisopropylsilyl.

(C₁-C₄)-Alkylsulphonyl in the context of the invention represents a straight-chain or branched alkyl radical having 1 to 4 carbon atoms which is attached via a sulphonyl group [—S(═O)₂—] to the remainder of the molecule. There may be mentioned by way of example and preferably: methylsulphonyl, ethylsulphonyl, n-propylsulphonyl, isopropylsulphonyl, n-butylsulphonyl and tert-butylsulphonyl.

(C₁-C₄)-Alkylcarbonyl in the context of the invention represents a straight-chain or branched alkyl radical having 1 to 4 carbon atoms which is attached via a carbonyl group [—C(═O)—] to the remainder of the molecule. There may be mentioned by way of example and preferably: acetyl, propionyl, n-butyryl, iso-butyryl, n-pentanoyl and pivaloyl.

(C₁-C₄)-Alkylcarbonyloxy in the context of the invention represents an oxy radical having a straight-chain or branched alkylcarbonyl substituent which has 1 to 4 carbon atoms in the alkyl radical and is attached via the carbonyl group to the oxygen atom. There may be mentioned by way of example and preferably: acetoxy, propionoxy, n-butyroxy, iso-butyroxy, n-pentanoyloxy and pivaloyloxy.

(C₂-C₆)-Alkenyl in the context of the invention represents a straight-chain or branched alkenyl radical having 2 to 6 carbon atoms and a double bond. A straight-chain or branched alkenyl radical having 2 to 4 carbon atoms is preferred. There may be mentioned by way of example and preferably: vinyl, n-prop-1-en-1-yl, allyl, isopropenyl, 2-methyl-2-propen-1-yl, n-but-1-en-1-yl, n-but-2-en-1-yl, n-but-3-en-1-yl, n-pent-2-en-1-yl, n-pent-3-en-1-yl, n-pent-4-en-1-yl, 3-methylbut-2-en-1-yl and 4-methylpent-3-en-1-yl.

(C₁-C₆)-Alkoxy and (C₁-C₄)-alkoxy in the context of the invention represent a straight-chain or branched alkoxy radical having 1 to 6 and 1 to 4 carbon atoms, respectively. A straight-chain or branched alkoxy radical having 1 to 4 carbon atoms is preferred. There may be mentioned by way of example and preferably: methoxy, ethoxy, n-propoxy, isopropoxy, n-butoxy, isobutoxy, sec-butoxy, tert-butoxy, n-pentoxy, 2-pentoxy, 3-pentoxy, neopentoxy, n-hexoxy, 2-hexoxy and 3-hexoxy.

(C₁-C₄)-Alkoxycarbonyl in the context of the invention represents a straight-chain alkoxy radical having 1 to 4 carbon atoms which is linked via a carbonyl group [—C(═O)—], attached to the oxygen atom, to the remainder of the molecule. There may be mentioned by way of example and preferably: methoxycarbonyl, ethoxycarbonyl, n-propoxycarbonyl, isopropoxycarbonyl, n-butoxycarbonyl and tert-butoxycarbonyl.

(C₃-C₆)-Cycloalkyl in the context of the invention represents a monocyclic saturated cycloalkyl group having 3 to 6 ring carbon atoms. There may be mentioned by way of example and preferably: cyclopropyl, cyclobutyl, cyclopentyl and cyclohexyl.

(C₃-C₆)-Cycloalkoxy in the context of the invention represents a monocyclic saturated cycloalkyloxy radical having 3 to 6 ring carbon atoms. There may be mentioned by way of example and preferably: cyclopropyloxy, cyclobutyloxy, cyclopentyloxy and cyclohexyloxy.

4- to 6-membered heterocyclyl in the context of the invention represents a monocyclic saturated heterocycle having a total of 4 to 6 ring atoms which contains one or two ring heteroatoms from the group consisting of N, O, S and S(O)₂ and is attached via a ring carbon atom or optionally a ring nitrogen atom. Preference is given to 4- or 5-membered heterocyclyl having a ring heteroatom from the group consisting of N and O and to 6-membered heterocyclyl having one or two ring heteroatoms from the group consisting of N and O. The following may be mentioned by way of example: azetidinyl, oxetanyl, thietanyl, pyrrolidinyl, pyrazolidinyl, tetrahydrofuranyl, thiolanyl, 1,1-dioxidothiolanyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, morpholinyl, thiomorpholinyl and 1,1-dioxidothiomorpholinyl. Preference is given to azetidinyl, oxetanyl, pyrrolidinyl, tetrahydrofuranyl, piperidinyl, piperazinyl, tetrahydropyranyl and morpholinyl.

Halogen in the context of the invention includes fluorine, chlorine, bromine and iodine. Chlorine, fluorine or bromine are preferred, and fluorine or chlorine are particularly preferred.

An oxo substituent in the context of the invention represents an oxygen atom, which is bonded to a carbon atom via a double bond.

In the context of the present invention, all radicals which occur more than once are defined independently of one another. If radicals in the compounds according to the invention are substituted, the radicals may be mono- or polysubstituted, unless specified otherwise. Substitution by one, two or three identical or different substituents is preferred. Particular preference is given to substitution by one or two identical or different substituents. Very particular preference is given to substitution by one substituent.

In the context of the present invention, preference is given to compounds of the formula (I) in which

one of the two radicals R^{1A} and R^{1B} represents fluorine and the other represents hydrogen,
Ar with the substituent R² represents a phenyl or pyridyl ring of the formula

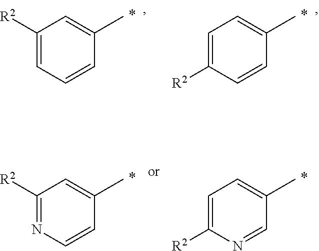

in which * denotes the point of attachment to the neighbouring CH₂ group,
R² represents a substituent selected from the group consisting of chlorine, (C₁-C₄)-alkyl, (C₃-C₆)-cycloalkyl, methoxy, ethoxy, methoxycarbonyl, ethoxycarbonyl, —NR⁵R⁶ and —C(═O)—NR⁵R⁶,
   where (C₁-C₄)-alkyl for its part may be substituted by a radical selected from the group consisting of hydroxyl, acetoxy, cyclopropyl and cyclobutyl and up to three times by fluorine
   and
   (C₃-C₆)-cycloalkyl and cyclopropyl and cyclobutyl for their part may be substituted up to two times by identical or different radicals selected from the group consisting of fluorine, methyl, trifluoromethyl, hydroxyl, hydroxymethyl, methoxy and acetoxy,
   and in which
   R⁵ represents hydrogen or methyl,
   R⁶ represents hydrogen or (C₁-C₄)-alkyl,
   or
   R⁵ and R⁶ are attached to one another and together with the nitrogen atom to which they are attached form a 4- to 6-membered heterocycle which may contain a further heteroatom from the group consisting of N, O and S and which may be substituted by a radical selected from the group consisting of cyano, hydroxyl, methoxy, ethoxy, (C₁-C₄)-alkyl, cyclopropyl and cyclobutyl,
      where (C₁-C₄)-alkyl for its part may be substituted up to three times by fluorine,
R³ represents a substituent selected from the group consisting of pentafluorothio, trimethylsilyl, (C₁-C₆)-alkyl, —OR⁸, —SR⁸, (C₃-C₆)-cycloalkyl and 4- to 6-membered heterocyclyl,
   where (C₁-C₆)-alkyl for its part may be substituted by hydroxyl or —OR⁸ and also up to six times by fluorine
   and
   (C₃-C₆)-cycloalkyl and 4- to 6-membered heterocyclyl for their part may be substituted up to two times by identical or different radicals selected from the group consisting of fluorine, methyl, trifluoromethyl, hydroxyl, methoxy and ethoxy,
   and in which
   R⁸ represents (C₁-C₄)-alkyl which may be substituted by a radical selected from the group consisting of hydroxyl, methoxy and ethoxy and also up to three times by fluorine,
      and
A represents N or C—R⁴, in which
   R⁴ represents hydrogen, fluorine or chlorine,
      and salts, solvates and solvates of the salts thereof.

A particular embodiment of the present invention comprises compounds of the formula (I) in which

R^{1A} represents fluorine
and
R^{1B} represents hydrogen,
and salts, solvates and solvates of the salts thereof.

A further particular embodiment of the present invention comprises compounds of the formula (I) in which

R^{1A} represents hydrogen and
R^{1B} represents fluorine,
and salts, solvates and solvates of the salts thereof.

A further particular embodiment of the present invention comprises compounds of the formula (I) in which

Ar with the substituent R² represents a phenyl or pyridyl ring of the formula

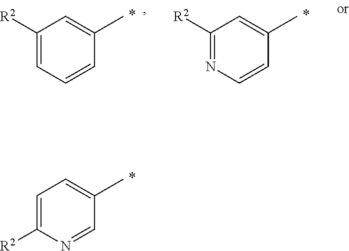

in which * denotes the point of attachment to the neighbouring CH₂ group,
   and salts, solvates and solvates of the salts thereof.

A further particular embodiment of the present invention comprises compounds of the formula (I) in which

A represents C—R⁴, in which
R⁴ represents hydrogen or fluorine,
   and salts, solvates and solvates of the salts thereof.

Particular preference in the context of the present invention is given to compounds of the formula (I) in which
R^{1B} represents hydrogen,
Ar with the substituent R² represents a phenyl or pyridyl ring of the formula

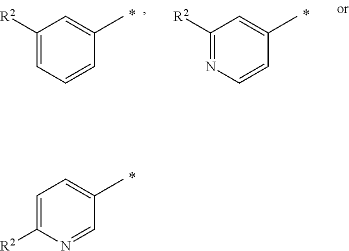

in which * denotes the point of attachment to the neighbouring CH₂ group,
R² represents a substituent selected from the group consisting of (C₁-C₄)-alkyl, cyclopropyl, cyclobutyl, —NR⁵R⁶ and —C(═O)—NR⁵R⁶,
   where (C₁-C₄)-alkyl for its part may be substituted by a radical selected from the group consisting of hydroxyl, acetoxy, cyclopropyl and cyclobutyl and also up to three times by fluorine
   and
   the cyclopropyl and cyclobutyl groups mentioned for their part may be substituted by a radical selected from the group consisting of hydroxyl, hydroxymethyl and acetoxy,
   and in which
   R⁵ represents hydrogen,
   R⁶ represents (C₁-C₄)-alkyl,
   or
   R⁵ and R⁶ are attached to one another and together with the nitrogen atom to which they are attached form a 4- to 6-membered heterocycle which may contain a further heteroatom from the group consisting of N and O and which may be substituted by a radical selected from the group consisting of cyano, hydroxyl, (C₁-C₄)-alkyl and cyclopropyl,
      where (C₁-C₄)-alkyl for its part may be substituted up to three times by fluorine,
R³ represents a substituent selected from the group consisting of trifluoromethoxy, trifluoromethylsulphanyl, pentafluorothio, trimethylsilyl, (C₁-C₄)-alkyl, cyclopropyl, cyclobutyl, cyclohexyl, oxetan-3-yl and tetrahydro-2H-pyran-4-yl,
   where (C₁-C₄)-alkyl for its part may be substituted by hydroxyl and also up to six times by fluorine
   and
   cyclopropyl, cyclobutyl, cyclohexyl, oxetanyl and tetrahydropyranyl for their part may be substituted by fluorine or trifluoromethyl,
      and
A represents C—R⁴, in which
   R⁴ represents hydrogen or fluorine,
      and salts, solvates and solvates of the salts thereof.

Particular preference in the context of the present invention is also given to compounds of the formula (I) in which
R^{1A} represents hydrogen,
R^{1B} represents fluorine,
Ar with the substituent R² represents a phenyl or pyridyl ring of the formula

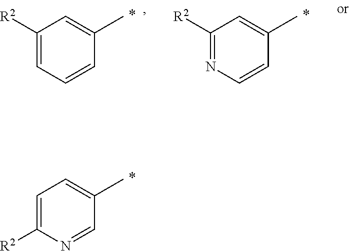

in which * denotes the point of attachment to the neighbouring CH₂ group,
R² represents a substituent selected from the group consisting of (C₁-C₄)-alkyl, cyclopropyl, cyclobutyl, —NR⁵R⁶ and —C(═O)—NR⁵R⁶,
   where (C₁-C₄)-alkyl for its part may be substituted by a radical selected from the group consisting of hydroxyl, acetoxy, cyclopropyl and cyclobutyl and also up to three times by fluorine
   and
   the cyclopropyl and cyclobutyl groups mentioned for their part may be substituted by a radical selected from the group consisting of hydroxyl, hydroxymethyl and acetoxy,
   and in which
   R⁵ represents hydrogen,
   R⁶ represents (C₁-C₄)-alkyl,
   or
   R⁵ and R⁶ are attached to one another and together with the nitrogen atom to which they are attached form a 4- to 6-membered heterocycle which may contain a further heteroatom from the group consisting of N and O and which may be substituted by a radical selected from the group consisting of cyano, hydroxyl, (C₁-C₄)-alkyl and cyclopropyl,
   where (C₁-C₄)-alkyl for its part may be substituted up to three times by fluorine,
R³ represents a substituent selected from the group consisting of trifluoromethoxy, trifluoromethylsulphanyl, pentafluorothio, trimethylsilyl, (C₁-C₄)-alkyl, cyclopropyl, cyclobutyl, cyclohexyl, oxetan-3-yl and tetrahydro-2H-pyran-4-yl,
   where (C₁-C₄)-alkyl for its part may be substituted by hydroxyl and also up to six times by fluorine
   and
   cyclopropyl, cyclobutyl, cyclohexyl, oxetanyl and tetrahydropyranyl for their part may be substituted by fluorine or trifluoromethyl,
      and
A represents C—R⁴, in which
   R⁴ represents hydrogen or fluorine,
      and salts, solvates and solvates of the salts thereof.

Very particular preference in the context of the present invention is given to compounds of the formula (I) in which
R^{1A} represents fluorine,
R^{1B} represents hydrogen,
Ar with the substituent R² represents a phenyl or pyridyl ring of the formula

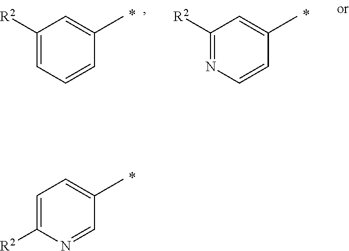

in which * denotes the point of attachment to the neighbouring CH₂ group,
R² represents the group —NR⁵R⁶, in which
   R⁵ represents hydrogen,
   R⁶ represents methyl or ethyl,
   or
   R⁵ and R⁶ are attached to one another and together with the nitrogen atom to which they are attached form a substituted heterocycle of the formula

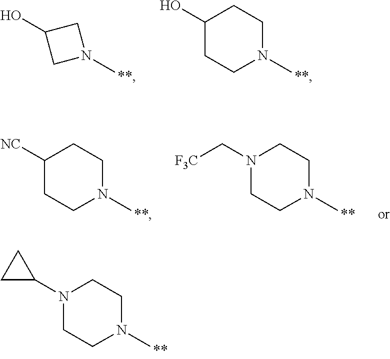

in which ** denotes the point of attachment to the ring Ar,
   or
R² represents a substituted isopropyl, isobutyl or cyclopropyl group of the formula

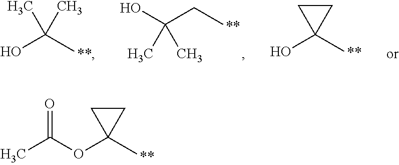

in which ** denotes the point of attachment to the ring Ar,
R³ represents trifluoromethyl, trifluoromethoxy, trifluoromethylsulphanyl, pentafluorothio, trimethylsilyl, tert-butyl or a group of the formula

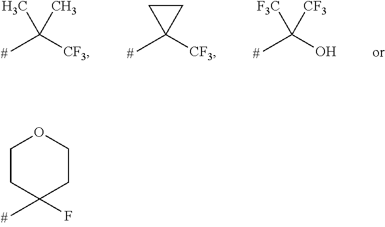

in which # denotes the point of attachment to the neighbouring ring,
   and
A represents C—R⁴, in which
   R⁴ represents hydrogen or fluorine,
      and salts, solvates and solvates of the salts thereof.

The definitions of radicals indicated specifically in the respective combinations or preferred combinations of radicals are replaced as desired irrespective of the particular combinations indicated for the radicals also by definitions of radicals of other combinations. Combinations of two or more of the abovementioned preferred ranges are very particularly preferred.

The present invention furthermore provides a process for preparing the compounds of the formula (I) according to the invention, characterized in that either

[A-1] a fluorinated pyrazolylmethylbenzothiazolylsulphone of the formula (II)

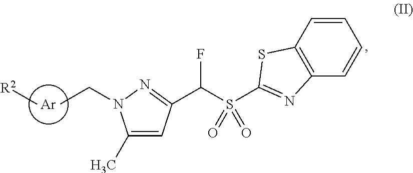

in which Ar and R² have the meanings given above,
is reacted in an inert solvent in the presence of a base with an aldehyde of the formula (III)

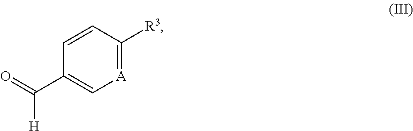

in which A and R³ have the meanings given above,
to give a compound of the formula (I-A) according to the invention

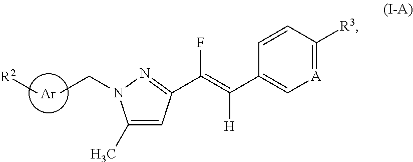

in which A, Ar, R² and R³ have the meanings given above,
   or
   [A-2] initially a fluorinated pyrazolylmethylbenzothiazolylsulphone of the formula (IV)

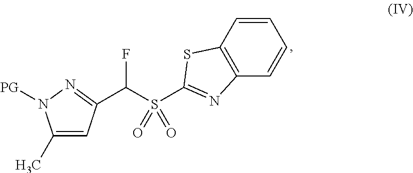

in which
PG represents a suitable protective group such as, for example, tetrahydro-2H-pyran-2-yl,
is reacted in an inert solvent in the presence of a base with an aldehyde of the formula (III)

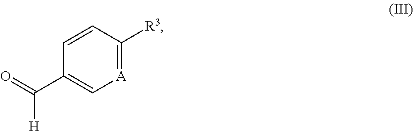

in which A and R³ have the meanings given above,
to give a compound of the formula (V)

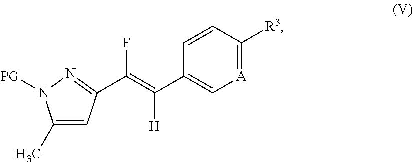

in which A, PG and R³ have the meanings given above,
the protective group PG is then removed by customary methods and the resulting pyrazole derivative of the formula (VI)

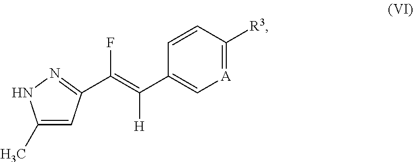

in which A and R³ have the meanings given above,
is then alkylated in an inert solvent in the presence of a base with a compound of the formula (VII)

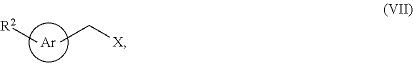

in which Ar and R² have the meanings given above
and
X represents a leaving group such as, for example, chlorine, bromine, iodine, mesylate, triflate or tosylate,
to give a compound of the formula (I-A) according to the invention

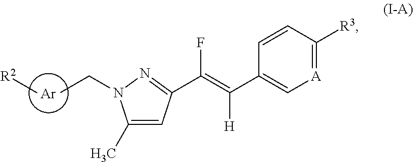

in which A, Ar, R² and R³ have the meanings given above,
   or
   [B-1] a fluorinated arylmethylbenzothiazolylsulphone of the formula (VIII)

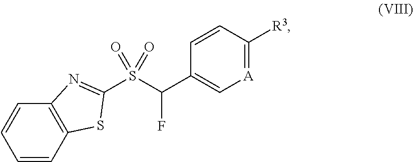

in which A and R³ have the meanings given above,
is reacted in an inert solvent in the presence of a base with a pyrazolecarbaldehyde of the formula (IX)

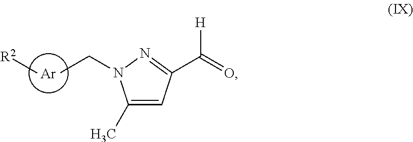

in which Ar and R² have the meanings given above,
to give a compound of the formula (I-B) according to the invention

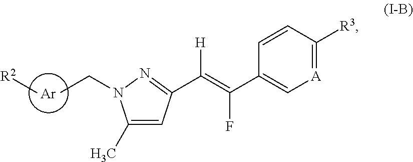

in which A, Ar, R² and R³ have the meanings given above,
   or
   [B-2] a fluorinated arylmethylbenzothiazolylsulphone of the formula (VIII)

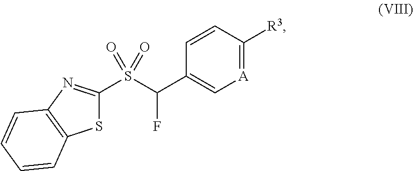

in which A and R³ have the meanings given above,
is reacted in an inert solvent in the presence of a base first with a protected pyrazolecarbaldehyde of the formula (X)

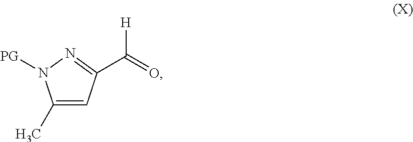

in which
PG represents a suitable protective group such as, for example, tetrahydro-2H-pyran-2-yl,
to give a compound of the formula (XI)

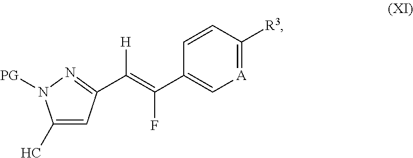

in which A, PG and R³ have the meanings given above,
the protective group PG is then removed by customary methods and the resulting pyrazole derivative of the formula (XII)

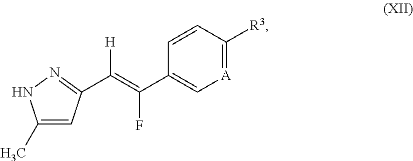

in which A and R³ have the meanings given above,
is then alkylated in an inert solvent in the presence of a base with a compound of the formula (VII)

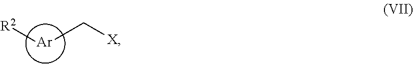

in which Ar and R² have the meanings given above
and
X represents a leaving group such as, for example, chlorine, bromine, iodine, mesylate, triflate or tosylate,
to give a compound of the formula (I-B) according to the invention

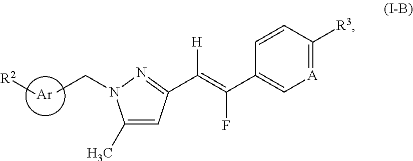

in which A, Ar, R² and R³ have the meanings given above,
   and the compounds of the formula (I-A) or (I-B) obtained in this manner are optionally separated into their enantiomers and/or diastereomers and/or converted with the appropriate (i) solvents and/or (ii) bases or acids into their solvates, salts and/or solvates of the salts.

The process steps (II)+(III)→(I-A), (IV)+(III)→(V), (VIII)+(IX)→(I-B) and (VIII)+(X)→(XI) are carried out using a method known from the literature in the sense of a “modified Julia olefination” [see P. R. Blakemore, J. Chem. Soc. Perkin Trans. 1, 2563-2585 (2002); E. Pfund et al., J. Org. Chem. 72, 7871-7877 (2007)]. Suitable inert solvents for these reactions are in particular ethers such as diethyl ether, diisopropyl ether, methyl tert-butyl ether, tetrahydrofuran, 1,4-dioxane, 1,2-dimethoxyethane or bis(2-methoxyethyl)ether. Preferred for use as base are non-nucleophilic alkali amides, such as lithium diisopropylamide (LDA) or lithium, sodium or potassium bis(trimethylsilyl)amide (Li-, Na-, K-HMDS), or strong tertiary amine bases, such as 1,8-diazabicyclo[5.4.0]undec-7-ene (DBU) or 1,5-diazabicyclo[4.3.0]non-5-ene (DBN); preference is given to lithium bis(trimethylsilyl)amide. The reactions are generally carried out in a temperature range of from −30° C. to +25° C., preferably at from 0° C. to +10° C.

Suitable temporary pyrazole protective groups PG in the compounds (IV) and (X) are, for example, groups such as tetrahydro-2H-pyran-2-yl (THP), phenylsulphonyl, p-tolylsulphonyl or tert-butoxycarbonyl (Boc). Introduction and removal of these protective groups is carried out by generally customary methods [see, for example, T. W. Greene and P. G. M. Wuts, Protective Groups in Organic Synthesis, Wiley, New York, 1999]. Preference is given to using the tetrahydropyranyl (THP) group. Its removal in process steps (V)→(VI) and (XI)→(XII) is preferably carried out with the aid of anhydrous hydrogen chloride in an inert solvent such as 1,4-dioxane.

Suitable inert solvents for the process steps (VI)+(VII)→(I-A) and (XII)+(VII)→(I-B) are, for example, ethers such as diethyl ether, diisopropyl ether, methyl tert-butyl ether, tetrahydrofuran, 1,4-dioxane, 1,2-dimethoxyethane or bis(2-methoxyethyl)ether, hydrocarbons such as benzene, toluene, xylene, ethylbenzene, pentane, hexane, cyclohexane or mineral oil fractions, or dipolar aprotic solvents such as N,N-dimethylformamide (DMF), N,N-dimethylacetamide (DMA), dimethyl sulphoxide (DMSO), N,N′-dimethylpropyleneurea (DMPU) or N-methylpyrrolidinone (NMP). It is also possible to use mixtures of the solvents mentioned. Preference is given to using tetrahydrofuran or 1,4-dioxane.

Suitable bases for the process steps (VI)+(VII)→(I-A) and (XII)+(VII)→(I-B) are in particular alkali metal hydroxides such as sodium hydroxide or potassium hydroxide, alkali metal alkoxides such as sodium tert-butoxide or potassium tert-butoxide, alkali metal hydrides such as sodium hydride or potassium hydride, or alkali metal amides such as lithium diisopropylamide or lithium bis(trimethylsilyl)amide, sodium bis(trimethylsilyl)amide or potassium bis(trimethylsilyl)amide. Preference is given to using potassium tert-butoxide. The addition of an alkylation catalyst, such as, for example, lithium bromide, sodium iodide or potassium iodide, tetra-n-butylammonium bromide or benzyltriethylammonium chloride, is advantageous. The reactions are generally carried out in a temperature range of from −20° C. to +100° C., preferably at from 0° C. to +65° C.

The reactions mentioned can be carried out at atmospheric, elevated or reduced pressure (for example from 0.5 to 5 bar); in general, the reactions are carried out at atmospheric pressure.

Further compounds of the formula (I) according to the invention can, if expedient, also be prepared by conversion of functional groups of individual radicals and substituents, in particular those listed under R² and R³, where other compounds of the formula (I) or precursors thereof obtained by the above processes are used as starting materials. These conversions are carried out by customary methods known to the person skilled in the art and include, for example, reactions such as nucleophilic or electrophilic substitution reactions, transition metal-catalysed coupling reactions (for example Ullmann or Buchwald-Hartwig reaction), additions of organometal compounds (for example Grignard compounds or organolithium compounds) to carbonyl compounds, oxidation and reduction reactions, hydrogenations, alkylations, acylations, sulphonylations, aminations, hydroxylations, the formation of nitriles, carboxylic esters and carboxamides, ester cleavage and hydrolysis and also the introduction and removal of temporary protective groups.

It is also possible, if expedient, to prepare compounds of the formula (I) according to the invention by introducing into the starting materials of the process variants described above, instead of the substituents R² and/or R³, initially other functional groups not included in the scope of the meaning of R² and R³, respectively, which are then converted by subsequent transformations (as listed above in an exemplary manner) known to the person skilled in the art into the respective substituents R² and R³. Examples of such functional groups serving as “precursor” to R² and/or R³ are radicals such as nitro, hydroxyl, methanesulphonate (mesylate), trifluoromethanesulphonate (triflate), formyl, alkylcarbonyl, hydroxycarbonyl and alkoxycarbonyl [cf. also the preparation, described in detail in the Experimental Part below, of the working examples and precursors thereof].

The α-fluorinated benzothiazolylsulphones of the formulae (II), (IV) and (VIII) can be prepared by reacting a compound of the formula (XIII)

M-CH₇—Y (XIII),

in which
M represents a group of the formula

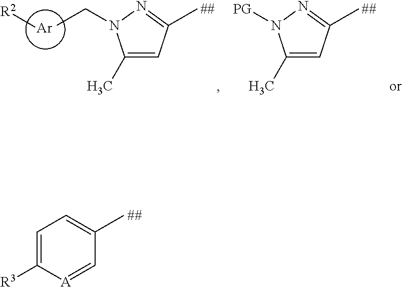

in which ## denotes the point of attachment to the CH₂ group and A, Ar, PG, R² and R³ each have the meanings given above,
   and
Y represents a leaving group such as, for example, chlorine, bromine, iodine, mesylate, triflate or tosylate,
   in an inert solvent with the sodium salt of 2-mercapto-1,3-benzothiazole (XIV)

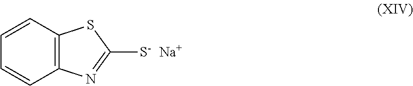

to give a compound of the formula (XV)

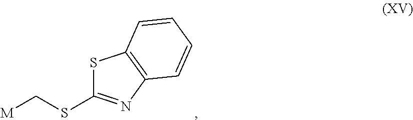

in which M has the meaning given above,
then oxidizing with a peroxide or a peracid to give a sulfone derivative of the formula (XVI)

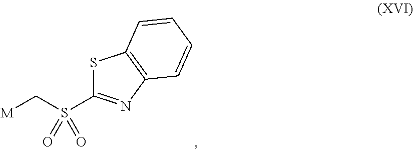

in which M has the meaning given above,
and, after α-deprotonation with a base, converting this with a suitable fluorinating agent such as, for example, N-fluorobenzenesulphonimide, into a compound of the formula (XVII)

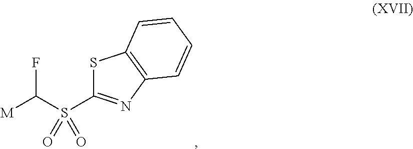

in which M has the meaning given above.

The reaction sequence (XIII)+(XIV)→(XV)→(XVI)→(XVII) is carried out analogously to processes described in the literature for the preparation of fluorine-substituted benzothiazolylsulphones [see, for example, P. R. Blakemore, J. Chem. Soc. Perkin Trans. 1, 2563-2585 (2002); E. Pfund et al., J. Org. Chem. 72, 7871-7877 (2007), and further literature cited therein].

Suitable inert solvents for the reaction (XIII)+(XIV)→(XV) are in particular dipolar aprotic solvents such as N,N-dimethylformamide (DMF), N,N-dimethylacetamide (DMA), dimethyl sulphoxide (DMSO), N,N′-dimethylpropyleneurea (DMPU) or N-methylpyrrolidinone (NMP); preference is given to N,N-dimethylformamide.

Suitable oxidizing agents for the process step (XV)→(XVI) are peracids such as peroxyacetic acid or m-chloroperoxybenzoic acid (mCPBA), peroxides such as hydrogen peroxide, optionally in the presence of a molybdenum(VI) or tungsten(VI) catalyst, or persalts such as Oxone® or potassium permanganate; preference is given to using m-chloroperbenzoic acid.

Suitable bases for the α-deprotonation of the compound (XVI) are non-nucleophilic bases such as sodium tert-butoxide or potassium tert-butoxide, lithium bis(trimethylsilyl)amide, sodium bis(trimethylsilyl)amide or potassium bis(trimethylsilyl)amide or lithium diisopropylamide; preference is given to using lithium diisopropylamide.

The subsequent fluorination to compound (XVII) is preferably carried out with the aid of N-fluorobenzenesulphonimide (NFSI). Alternatively, it is also possible to use other electrophilic fluorination agents such as, for example, Selectfluor™ (F-TEDA), 1-fluoropyridinium tetrafluoroborate or 1-fluoropyridinium trifluoromethanesulphonate.

The compounds of the formulae (III), (VII), (IX), (X), (XIII) and (XIV) are commercially available or described as such in the literature, or they can be prepared in a manner obvious to the person skilled in the art analogously to the methods published in the literature. Numerous detailed procedures and literature references for preparing the starting materials can also be found in the Experimental Part in the section on the preparation of the starting materials and intermediates.

The preparation of the compounds according to the invention can be illustrated in an exemplary manner by the reaction schemes below:

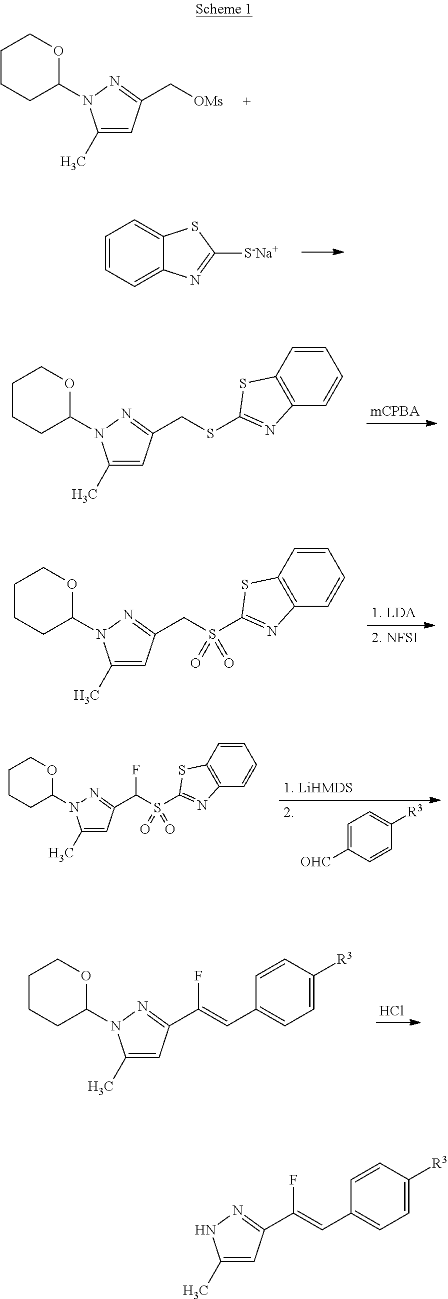

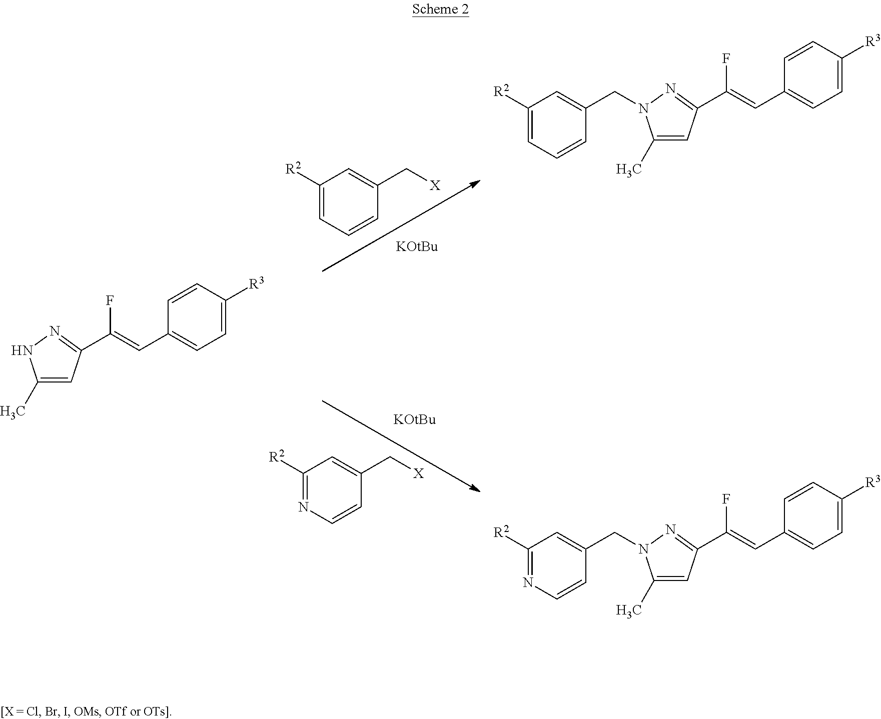

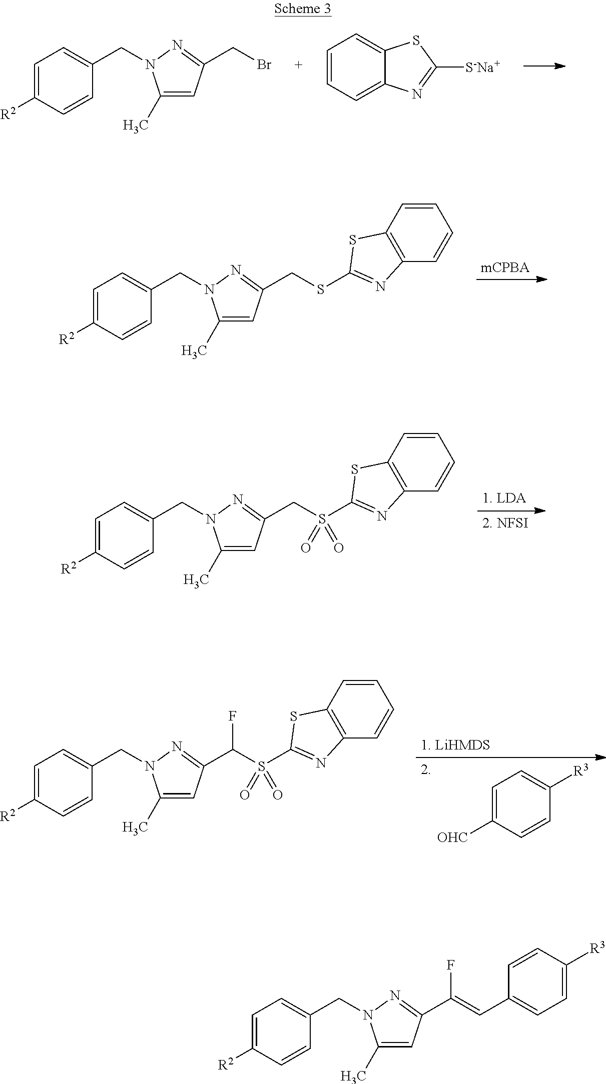

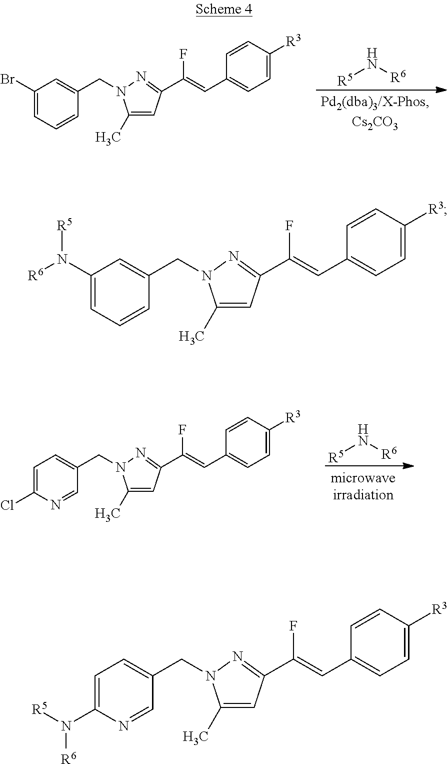

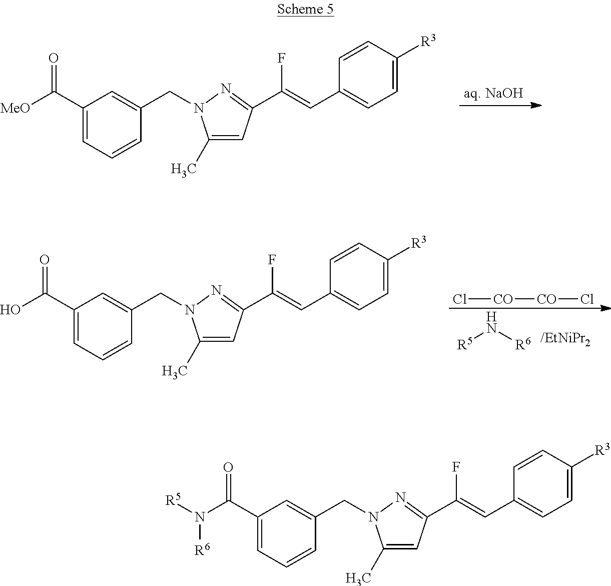

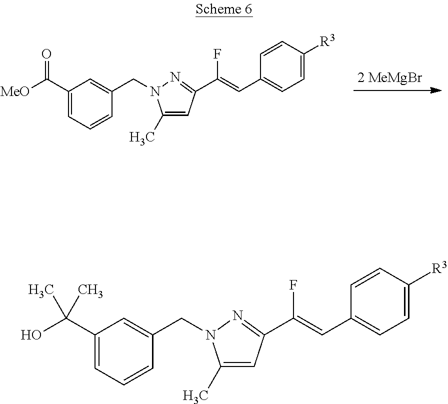

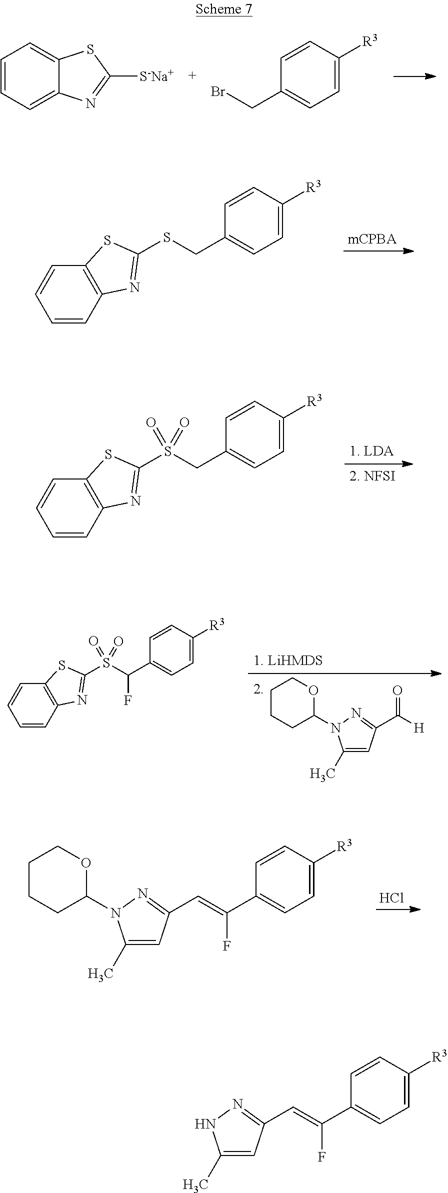

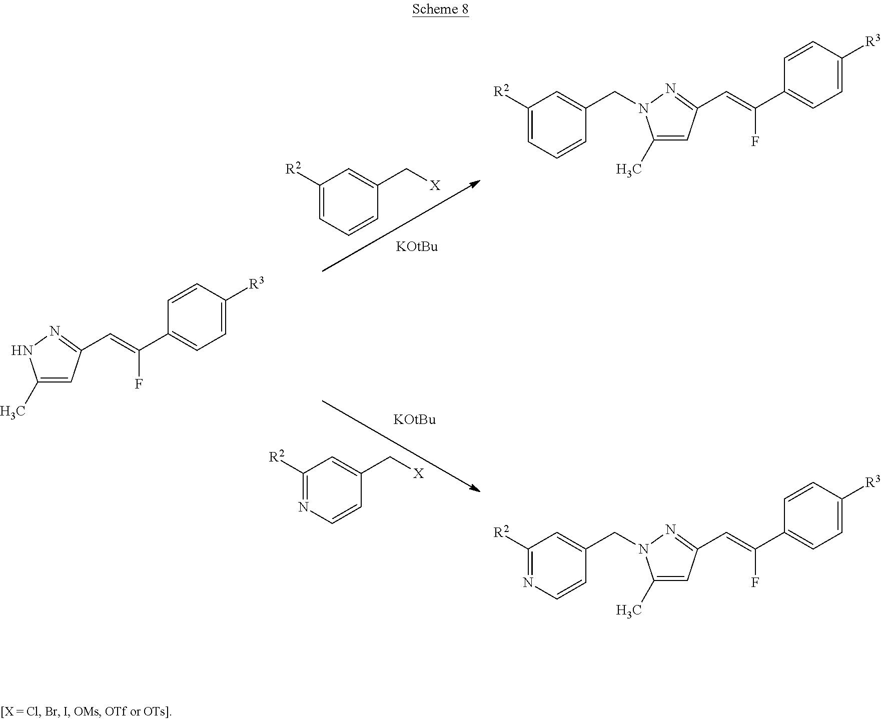

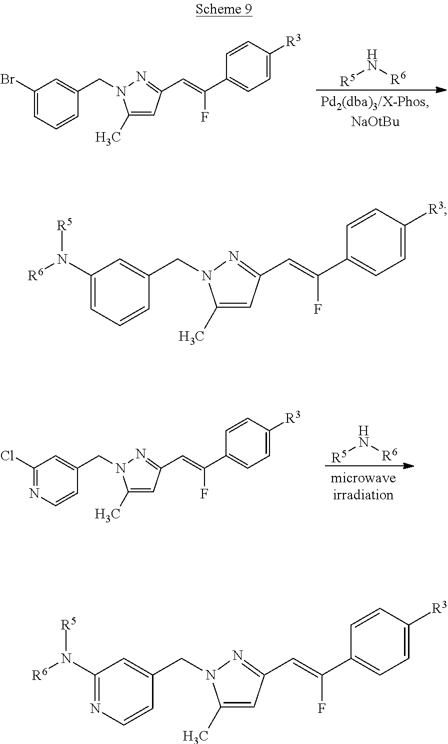

The compounds according to the invention have valuable pharmacological properties and can be used for prevention and treatment of diseases in humans and animals.

The compounds according to the invention are highly potent inhibitors of the HIF regulation pathway. In addition, the compounds according to the invention have an advantageous pharmacokinetic profile suitable for oral administration.

On the basis of their action profile, the compounds according to the invention are suitable in particular for treatment of hyperproliferative diseases in humans and in mammals generally. The compounds can inhibit, block, reduce or lower cell proliferation and cell division and on the other hand increase apoptosis.

The hyperproliferative diseases for the treatment of which the compounds according to the invention can be employed include, inter alia, psoriasis, keloids, formation of scars and other proliferative diseases of the skin, benign diseases, such as benign prostate hyperplasia (BPH), and in particular the group of tumour diseases. In the context of the present invention, these are understood as meaning, in particular, the following diseases, but without being limited to them: mammary carcinomas and mammary tumours (ductal and lobular forms, also in situ), tumours of the respiratory tract (parvicellular and non-parvicellular carcinoma, bronchial carcinoma), cerebral tumours (e.g. of the brain stem and of the hypothalamus, astrocytoma, medulloblastoma, ependymoma and neuro-ectodermal and pineal tumours), tumours of the digestive organs (oesophagus, stomach, gall bladder, small intestine, large intestine, rectum), liver tumours (inter alia hepatocellular carcinoma, cholangiocellular carcinoma and mixed hepatocellular and cholangiocellular carcinoma), tumours of the head and neck region (larynx, hypopharynx, nasopharynx, oropharynx, lips and oral cavity), skin tumours (squamous epithelial carcinoma, Kaposi sarcoma, malignant melanoma, Merkel cell skin cancer and nonmelanomatous skin cancer), tumours of soft tissue (inter alia soft tissue sarcomas, osteosarcomas, malignant fibrous histiocytomas, lymphosarcomas and rhabdomyosarcomas), tumours of the eyes (inter alia intraocular melanoma and retinoblastoma), tumours of the endocrine and exocrine glands (e.g. thyroid and parathyroid glands, pancreas and salivary gland), tumours of the urinary tract (tumours of the bladder, penis, kidney, renal pelvis and ureter) and tumours of the reproductive organs (carcinomas of the endometrium, cervix, ovary, vagina, vulva and uterus in women and carcinomas of the prostate and testicles in men). These also include proliferative blood diseases in solid form and as circulating blood cells, such as lymphomas, leukaemias and myeloproliferative diseases, e.g. acute myeloid, acute lymphoblastic, chronic lymphocytic, chronic myelogenic and hair cell leukaemia, and AIDS-correlated lymphomas, Hodgkin's lymphomas, non-Hodgkin's lymphomas, cutaneous T cell lymphomas, Burkitt's lymphomas and lymphomas in the central nervous system.

These well-described diseases in humans can also occur with a comparable aetiology in other mammals and can be treated there with the compounds of the present invention.

In the context of this invention the term “treatment” or “treat” is used in the conventional sense and means attending to, caring for and nursing a patient with the aim of combating, reducing, attenuating or alleviating a disease or health abnormality and improving the living conditions impaired by this disease, such as, for example, with a cancer disease.

The compounds according to the invention act as modulators of the HIF regulation pathway and are therefore also suitable for treatment of diseases associated with a harmful expression of the HIF transcription factor. This applies in particular to the transcription factors HIF-1α and HIF-2α. The term “harmful expression of HIF” here means a non-normal physiological presence of HIF protein. This can be due to excessive synthesis of the protein (mRNA- or translation-related), reduced degradation or inadequate counter-regulation in the functioning of the transcription factor.

HIF-1α and HIF-2α regulate more than 100 genes. This applies to proteins which play a role in angiogenesis and are therefore directly relevant to tumours, and also those which influence glucose, amino acid and lipid metabolism as well as cell migration, metastasis and DNA repair, or improve the survival of tumour cells by suppressing apoptosis. Others act more indirectly via inhibition of the immune reaction and upwards regulation of angiogenic factors in inflammation cells. HIF also plays an important role in stem cells, and here in particular tumour stem cells, which are reported to have increased HIF levels. By the inhibition of the HIF regulation pathway by the compounds of the present invention, tumour stem cells, which do not have a high proliferation rate and therefore are affected only inadequately by cytotoxic substances, are therefore also influenced therapeutically (cf. Semenza, 2007; Weidemann and Johnson, 2008).

Changes in cell metabolism by HIF are not exclusive to tumours, but also occur with other hypoxic pathophysiological processes, whether chronic or transient. HIF inhibitors—such as the compounds of the present invention—are therapeutically helpful in those connections in which, for example, additional damage arises from adaptation of cells to hypoxic situations, since damaged cells can cause further damage if they do not function as intended. One example of this is the formation of epileptic foci in partly destroyed tissue following strokes. A similar situation is found with cardiovascular diseases if ischaemic processes occur in the heart or in the brain as a consequence of thromboembolic events, inflammations, wounds, intoxications or other causes. These can lead to damage such as a locally retarded action potential, which in turn can bring about arrhythmias or chronic heart failure. In a transient form, e.g. due to apnoea, under certain circumstances an essential hypertension may occur, which can lead to known secondary diseases, such as, for example, stroke and cardiac infarction.

Inhibition of the HIF regulation pathway such as is achieved by the compounds according to the invention can therefore also be helpful for diseases such as cardiac insufficiency, arrhythmia, cardiac infarction, apnoea-induced hypertension, pulmonary hypertension, transplant ischaemia, reperfusion damage, stroke and macular degeneration, as well as for recovery of nerve function after traumatic damage or severance.

Since HIF is one of the factors which control the transition from an epithelial to a mesenchymal cell type, which is of importance specifically for the lung and kidney, the compounds according to the invention can also be employed for preventing or controlling fibroses of the lung and kidney associated with HIF.

Further diseases for the treatment of which the compounds according to the invention can be used are inflammatory joint diseases, such as various forms of arthritis, and inflammatory intestinal diseases, such as, for example, Crohn's disease.

Chugwash polycythaemia is mediated by HIF-2α activity during erythropoiesis inter alia in the spleen. The compounds according to the invention, as inhibitors of the HIF regulation pathway, are therefore also suitable here for suppressing excessive erythrocyte formation and therefore for alleviating the effects of this disease.

The compounds of the present invention can furthermore be used for treatment of diseases associated with excessive or abnormal angiogenesis. These include, inter alia, diabetic retinopathy, ischaemic retinal vein occlusion and retinopathy in premature babies (cf. Aiello et al., 1994; Peer et al., 1995), age-related macular degeneration (AMD; cf. Lopez et al., 1996), neovascular glaucoma, psoriasis, retrolental fibroplasia, angiofibroma, inflammation, rheumatic arthritis (RA), restenosis, in-stent restenosis and restenosis following vessel implantation.

An increased blood supply is furthermore associated with cancerous, neoplastic tissue and leads here to an accelerated tumour growth. The growth of new blood and lymph vessels moreover facilitates the formation of metastases and therefore the spread of the tumour. New lymph and blood vessels are also harmful for allografts in immunoprivileged tissues, such as the eye, which, for example, increases the susceptibility to rejection reactions. Compounds of the present invention can therefore also be employed for therapy of one of the abovementioned diseases, e.g. by an inhibition of the growth or a reduction in the number of blood vessels. This can be achieved via inhibition of endothelial cell proliferation or other mechanisms for preventing or lessening the formation of vessels and via a reduction of neoplastic cells by apoptosis.

In the case of adiposity, there is an accumulation of HIF-1α in fatty tissue and thus an HIF-mediated shift of the energy metabolism towards glycolysis, so that increasingly glucose is consumed as energy source. Simultaneously, this leads to reduced fat metabolism and thus to fats being stored in the tissue. Accordingly, the substances according to the invention are also suitable for treating the HIF-1α-mediated accumulation of fats in tissue, in particular in the case of an adiposity disorder.

The present invention furthermore provides the use of the compounds according to the invention for treatment and/or prevention of diseases, in particular the abovementioned diseases.

The present invention furthermore provides the use of the compounds according to the invention for the preparation of a medicament for treatment and/or prevention of diseases, in particular the abovementioned diseases.

The present invention furthermore provides the use of the compounds according to the invention in a method for treatment and/or prevention of diseases, in particular the abovementioned diseases.

The present invention furthermore provides a method for treatment and/or prevention of diseases, in particular the abovementioned diseases, using an active amount of at least one of the compounds according to the invention.

The compounds according to the invention can be employed by themselves or, if required, in combination with one or more other pharmacologically active substances, as long as this combination does not lead to undesirable and unacceptable side effects. The present invention furthermore therefore provides medicaments containing at least one of the compounds according to the invention and one or more further active compounds, in particular for treatment and/or prevention of the abovementioned diseases.

For example, the compounds of the present invention can be combined with known antihyperproliferative, cytostatic or cytotoxic substances for treatment of cancer diseases. The combination of the compounds according to the invention with other substances customary for cancer therapy or also with radiotherapy is therefore indicated in particular, since hypoxic regions of a tumour respond only weakly to the conventional therapies mentioned, whereas the compounds of the present invention display their activity there in particular.

Suitable active compounds in the combination which may be mentioned by way of example are:

aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulfan, calcitonin, campath, capecitabine, carboplatin, casodex, cefesone, celmoleukin, cerubidin, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, kytril, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofuran, sobuzoxane, solu-medrol, streptozocin, strontium-89 chloride, synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example—without this list being conclusive:

aminoglutethimide, L-asparaginase, azathioprine, 5-azacytidine, bleomycin, busulfan, camptothecin, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenine, ethinylestradiol, etoposide, fludarabin phosphate, 5-fluorodeoxyuridine, 5-fluorodeoxyuridine monophosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphonoacetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

The compounds according to the invention can also be combined in a very promising manner with biological therapeutics, such as antibodies (e.g. avastin, rituxan, erbitux, herceptin) and recombinant proteins, which additively or synergistically intensify the effects of inhibition of the HIF signal pathway transmission.

Inhibitors of the HIF regulation pathway, such as the compounds according to the invention, can also achieve positive effects in combination with other therapies directed against angiogenesis, such as, for example, with avastin, axitinib, recentin, regorafenib, sorafenib or sunitinib. Combinations with inhibitors of the proteasome and of mTOR and antihormones and steroidal metabolic enzyme inhibitors are particularly suitable because of their favourable profile of side effects.

Generally, the following aims can be pursued with the combination of compounds of the present invention with other agents having a cytostatic or cytotoxic action:
an improved activity in slowing down the growth of a tumour, in reducing its size or even in its complete elimination compared with treatment with an individual active compound;
the possibility of employing the chemotherapeutics used in a lower dosage than in monotherapy;
the possibility of a more tolerable therapy with fewer side effects compared with individual administration;
the possibility of treatment of a broader spectrum of tumour diseases;
achievement of a higher rate of response to the therapy;
a longer survival time of the patient compared with present-day standard therapy.

The compounds according to the invention can moreover also be employed in combination with radiotherapy and/or surgical intervention.

The present invention furthermore provides medicaments which comprise at least one compound according to the invention, conventionally together with one or more inert, non-toxic, pharmaceutically suitable auxiliary substances, and the use thereof for the above-mentioned purposes.

The compounds according to the invention can act systemically and/or locally. They can be administered in a suitable manner for this purpose, such as e.g. orally, parenterally, pulmonally, nasally, sublingually, lingually, buccally, rectally, dermally, transdermally, conjunctivally, otically or as an implant or stent.

The compounds according to the invention can be administered in suitable administration forms for these administration routes.

Administration forms which function according to the prior art, release the compounds according to the invention rapidly and/or in a modified manner and contain the compounds according to the invention in crystalline and/or amorphized and/or dissolved form are suitable for oral administration, such as e.g. tablets (non-coated or coated tablets, for example with coatings which are resistant to gastric juice or dissolve in a delayed manner or are insoluble and control the release of the compound according to the invention), tablets or films/oblates, films/lyophilisates or capsules which disintegrate rapidly in the oral cavity (for example hard or soft gelatine capsules), sugar-coated tablets, granules, pellets, powders, emulsions, suspensions, aerosols or solutions, are suitable for oral administration.

Parenteral administration can be effected with bypassing of an absorption step (e.g. intravenously, intraarterially, intracardially, intraspinally or intralumbally) or with inclusion of an absorption (e.g. intramuscularly, subcutaneously, intracutaneously, percutaneously or intraperitoneally). Administration forms which are suitable for parenteral administration are, inter alia, injection and infusion formulations in the form of solutions, suspensions, emulsions, lyophilisates or sterile powders.

For the other administration routes e.g. inhalation medicament forms (inter alia powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents are suitable.

Oral or parenteral administration is preferred, in particular oral and intravenous administration.

The compounds according to the invention can be converted into the administration forms mentioned. This can be effected in a manner known per se by mixing with inert, non-toxic, pharmaceutically suitable auxiliary substances. These auxiliary substances include inter alia carrier substances (for example microcrystalline cellulose, lactose, mannitol), solvents (e.g. liquid polyethylene glycols), emulsifiers and dispersing or wetting agents (for example sodium dodecyl sulphate, polyoxysorbitan oleate), binders (for example polyvinylpyrrolidone), synthetic and natural polymers (for example albumin), stabilizers (e.g. antioxidants, such as, for example, ascorbic acid), dyestuffs (e.g. inorganic pigments, such as, for example, iron oxides) and flavour and/or smell correctants.

In general, it has proven advantageous in the case of parenteral administration to administer amounts of from about 0.001 to 1 mg/kg, preferably about 0.01 to 0.5 mg/kg of body weight to achieve effective results. In the case of oral administration the dosage is about 0.01 to 100 mg/kg, preferably about 0.01 to 20 mg/kg and very particularly preferably 0.1 to 10 mg/kg of body weight.

Nevertheless it may be necessary to deviate from the amounts mentioned, and in particular depending on the body weight, administration route, individual behaviour towards the active compound, nature of the formulation and point in time or interval at which administration takes place. Thus in some cases it may be sufficient to manage with less than the abovementioned minimum amount, while in other cases the upper limit mentioned must be exceeded. In the case where relatively large amounts are administered, it may be advisable to spread these into several individual doses over the day.

The following working examples illustrate the invention. The invention is not limited to the examples.

The percentage data in the following tests and examples are percentages by weight, unless stated otherwise; parts are parts by weight. The solvent ratios, dilution ratios and concentration data of liquid/liquid solutions in each case relate to the volume.

### A. EXAMPLES

### Abbreviations and Acronyms

abs. absolute
Ac acetyl
AIBN 2,2′-azobis(isobutyronitrile)
aq. aqueous, aqueous solution
br. broad (in NMR)
Ex. Example
Bu butyl
CDI 1,1′-carbonyldiimidazole
CI chemical ionization (in MS)
d doublet (in NMR)
d day(s)
DAST diethylaminosulphur trifluoride
dba dibenzylideneacetone
TLC thin layer chromatography
DCI direct chemical ionization (in MS)
dd doublet of doublet (in NMR)
DMAP 4-N,N-dimethylaminopyridine
DME 1,2-dimethoxyethane
DMF N,N-dimethylformamide
DMSO dimethyl sulphoxide
dt doublet of triplet (in NMR)
EDC N′-(3-dimethylaminopropyl)-N-ethylcarbodiimide hydrochloride
ee enantiomeric excess
EI electron impact ionization (in MS)
eq. equivalent(s)
ESI electrospray ionization (in MS)
Et ethyl
GC gas chromatography
GC/MS Gas chromatography-coupled mass spectrometry
h hour(s)
HOBt 1-hydroxy-1H-benzotriazole hydrate
HPLC high pressure, high performance liquid chromatography
ⁱPr isopropyl
LC/MS liquid chromatography-coupled mass spectrometry
LDA lithium diisopropylamide
LiHMDS lithium hexamethyldisilazide
Lit. literature (reference)
m multiplet (in NMR)
mCPBA meta-chloroperoxybenzoic acid
Me methyl
min minute(s)
MPLC medium pressure liquid chromatography (on silica gel; also called “flash chromatography”)
Ms methanesulphonyl (mesyl)
MS mass spectrometry
NBS N-bromosuccinimide
NFSI N-fluorobenzenesulphonimide
NMP N-methyl-2-pyrrolidinone
NMR nuclear magnetic resonance spectrometry
Pd/C palladium on activated carbon
PEG polyethylene glycol
Pr propyl
quart quartet (in NMR)
quint quintet (in NMR)
R_{f} retention index (in TLC)
RT room temperature
Rₜ retention time (in HPLC)
singlet (in NMR)
sept septet (in NMR)
t triplet (in NMR)
TBAF tetra-n-butylammonium fluoride
^{t}Bu tert-butyl
Tf trifluoromethylsulphonyl (triflyl)
TFA trifluoroacetic acid
THF tetrahydrofuran
THP tetrahydro-2H-pyran-2-yl
TIPS triisopropylsilyl
Ts para-tolylsulphonyl (tosyl)
UV ultraviolet spectrometry
v/v volume to volume ratio (of a solution)
X-Phos 2-dicyclohexylphosphino-2′,4′,6′-triisopropylbiphenyl
tog. together

### HPLC, LC/MS and GC/MS Methods:

### Method 1 (Analytical HPLC):

Instrument: HP 1100 with DAD detection; column: Kromasil 100 RP-18, 60 mm×2.1 mm, 3.5 μm; mobile phase A: 5 ml of perchloric acid (70% strength)/1 of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210

### Method 2 (LC/MS):

Instrument: Micromass Quattro Premier with Waters UPLC Acquity; column: Thermo Hypersil GOLD 1.9 μm, 50 mm×1 mm; mobile phase A: 1 l of water+0.5 ml of 50% strength formic acid, mobile phase B: 1 l of acetonitrile+0.5 ml of 50% strength formic acid; gradient 0.0 min 90% A 0.1 min 90% A→1.5 min 10% A→2.2 min 10% A; flow rate: 0.33 ml/min; oven: 50° C.; UV detection: 210 nm.

### Method 3 (LC/MS):

MS instrument type: Micromass ZQ; HPLC instrument type: HP 1100 Series; UV DAD; column: Phenomenex Gemini 3 μm, 30 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min 2.5 min/3.0 min/4.5 min 2 ml/min; oven: 50° C.; UV detection: 210 nm.

### Method 4 (LC/MS):

MS instrument type: Micromass ZQ; HPLC instrument type: Waters Alliance 2795; column: Phenomenex Synergi 2.5 μm MAX-RP 100A Mercury, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

### Method 5 (LC/MS):

Instrument: Waters Acquity SQD UPLC System; column: Waters Acquity UPLC HSS T3 1.8 μm, 50 mm×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; flow rate: 0.40 ml/min; oven: 50° C.; UV detection: 210-400 nm.

### Method 6 (LC/MS):

MS instrument type: Micromass Quattro Micro; HPLC instrument type: Agilent Serie 1100; column: Thermo Hypersil GOLD 3 μm, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A; oven: 50° C.; flow rate: 2 ml/min; UV detection: 210 nm.

### Method 7 (LC/MS):

MS instrument type: Waters ZQ; HPLC instrument type: Agilent Serie 1100; UV DAD; column: Thermo Hypersil GOLD 3 μm, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A; oven: 55° C.; flow rate: 2 ml/min; UV detection: 210 nm.

### Method 8 (LC/MS):

Instrument: Waters Acquity SQD UPLC System; column: Waters Acquity UPLC HSS T3 1.8 μm, 30 mm×2 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; flow rate: 0.60 ml/min; oven: 50° C.; UV detection: 208-400 nm.

### Method 9 (LC/MS):

Instrument: Waters Acquity SQD UPLC System; column: Waters Acquity UPLC HSS T3 1.8 μm, 50 mm×1 mm; mobile phase A: 1 l of water+0.25 ml of 99% strength formic acid, mobile phase B: 1 l of acetonitrile+0.25 ml of 99% strength formic acid; gradient: 0.0 min 95% A→6.0 min 5% A→7.5 min 5% A; flow rate: 0.35 ml/min; oven: 50° C.; UV detection: 210-400 nm.

### Method 10 (GC/MS):

Instrument: Micromass GCT, GC 6890; column: Restek RTX-35, 15 m×200 μm×0.33 μm; constant helium flow: 0.88 ml/min; oven: 70° C.; inlet: 250° C.; gradient: 70° C., 30° C./min 310° C. (maintained for 3 min).

### Method 11 (GC/MS):

Instrument: Thermo DFS, Trace GC Ultra; column: Restek RTX-35, 15 m×200 μm×0.33 μm; constant helium flow: 1.20 ml/min; oven: 60° C.; inlet: 220° C.; gradient: 60° C., 30° C./min 300° C. (maintained for 3.33 min).

### Method 12 (Preparative HPLC):

Column: Reprosil C18, 10 μm, 250 mm×30 mm; mobile phase: acetonitrile/0.1% aq. TFA; gradient: 10:90→90:10.

### Method 13 (Preparative HPLC):

Column: YMC-ODS-AQ, C18, 10 μm, 250 mm×30 mm; mobile phase: methanol/0.1% aq. TFA; gradient: 50:50 (0.00-4.25 min)→70:30 (4.25-4.50 min)→90:10 (4.50-11.50 min)→100:0 (11.50-12.00 min)→100:0 (12.00-14.50 min)→50:50 (14.50-14.75 min)→50:50 (14.75-18.00 min).

### Method 14 (Preparative HPLC):

Column: Reprosil-Pur C18, 10 μm, 250 mm×30 mm; mobile phase: acetonitrile/0.1% aq. formic acid; gradient: 10:90→90:10.

### Method 15 (Preparative HPLC):

Column: Daiso C18 Bio Spring Column, 10 μm, 300 mm×100 mm; mobile phase: methanol/water; gradient: 20:80 (0-5 min)→80:20 (5-65 min)→80:20 (65-129 min)→90:10 (129-139 min); flow rate: 250 ml/min.

### Method 16 (Preparative HPLC):

Column: YMC-ODS-AQ, C18, 10 μm, 250 mm×30 mm; mobile phase: methanol/0.1% aq. TFA; gradient: 60:40 (0.00-4.25 min)→80:20 (4.25-4.50 min)→100:0 (4.50-11.50 min)→100:0 (11.50-14.50 min)→60:40 (14.50-14.75 min)→60:40 (14.75-18.00 min).

### Method 17 (Preparative HPLC):

Column: Daiso C18 Bio DAN, 10 μm, 300 mm×100 mm; mobile phase: methanol/water; gradient: 40:60 (0-5 min)→75:25 (5-65 min)→75:25 (65-152 min)→90:10 (152-180 min); flow rate: 250 ml/min.

### Method 18 (Preparative HPLC):

Column: Waters Sunfire C18, 5 μm, 250 mm×30 mm; mobile phase: acetonitrile/water 35:65; flow rate: 56 ml/min.

### Method 19 (Preparative HPLC):

Column: Waters Sunfire C18, 5 μm, 250 mm×30 mm; mobile phase: acetonitrile/water 75:25; flow rate: 56 ml/min.

### Method 20 (Preparative HPLC):

Column: YMC-ODS-AQ, C18, 10 μm, 250 mm×30 mm; mobile phase: methanol/0.1% aq. TFA; gradient: 40:60 (0.00-4.25 min)→60:40 (4.25-4.50 min)→80:20 (4.50-11.50 min)→100:0 (11.50-12.00 min)→100:0 (12.00-14.50 min)→40:60 (14.50-14.75 min)→40:60 (14.75-18.00 min).

### Method 21 (Preparative HPLC):

Column: XBridge C18, 5 μm, 150 mm×19 mm; mobile phase: acetonitrile/water/1% aq. diethylamine 60:35:5.

### Method 22 (Preparative HPLC):

Column: Daicel Chiralcel OD-H, 5 μm, 250 mm×20 mm; mobile phase: isohexane/isopropanol 50:50; flow rate: 15 ml/min; temperature: 40° C.; UV detection: 220 nm.

### Method 23 (Preparative HPLC):

Column: Daicel Chiralpak IA, 5 μm, 250 mm×20 mm; mobile phase: methanol/acetonitrile 70:30; flow rate: 15 ml/min.

### Method 24 (Preparative HPLC):

Column: Daicel Chiralpak AD-H, 5 μm, 250 mm×20 mm; mobile phase: isohexane/propanol 25:75; flow rate: 15 ml/min.

### Method 25 (Preparative HPLC):

Column: YMC-ODS-AQ, C18, 10 μm, 250 mm×30 mm; mobile phase: methanol/0.1% aq. TFA; gradient: 30:70 (0.00-4.25 min)→50:50 (4.25-4.50 min)→70:30 (4.50-11.50 min)→100:0 (11.50-12.00 min)→100:0 (12.00-14.50 min)→30:70 (14.50-14.75 min)→30:70 (14.75-18.00 min).

### Method 26 (Preparative HPLC):

Column: Waters Sunfire C18 OBD, 5 μm, 150 mm×19 mm; mobile phase: acetonitrile/water 86:14; flow rate: 25 ml/min.

### Method 27 (Preparative HPLC):

Column: Reprosil C18, 10 μm, 250 mm×30 mm; mobile phase: acetonitrile/0.1% aq. TFA; gradient: 10:90 (0.00-5.00 min) (sample injection at 3.00 min)→95:5 (5.00-20.00 min)→95:5 (20.00-30.00 min)→10:90 (30.00-30.50 min)→10:90 (30.50-31.20 min).

### Method 28 (Preparative HPLC):

Column: Daicel Chiralpak AD-H, 5 μm, 250 mm×20 mm; mobile phase: isohexane/ethanol 60:40; flow rate: 20 ml/min; temperature: 25° C.; UV detection: 230 nm.

### Method 29 (Preparative HPLC):

Column: Daicel Chiralpak AD-H, 5 μm, 250 mm×20 mm; mobile phase: isohexane/ethanol 70:30; flow rate: 20 ml/min.

### Method 30 (Preparative HPLC):

Column: Daicel Chiralpak AD-H, 5 μm, 250 mm×20 mm; mobile phase: isohexane/ethanol 40:60; flow rate: 20 ml/min.

### Method 31 (Preparative HPLC):

Column: Waters Sunfire C18, 5 μm, 250 mm×30 mm; mobile phase: acetonitrile/water/1% aq. TFA 45:44:11; flow rate: 25 ml/min.

### Method 32 (Preparative HPLC):

Column: Daicel Chiralpak AD-H, 5 μm, 250 mm×20 mm; mobile phase: isohexane/isopropanol 60:40; flow rate: 20 ml/min.

### Method 33 (Preparative HPLC):

Column: Daicel Chiralcel OD-H, 5 μm, 250 mm×20 mm; mobile phase: isohexane/ethanol 60:40; flow rate: 20 ml/min; temperature: 25° C.; UV detection: 230 nm.

### Method 34 (Preparative HPLC):

Column: GromSil ODS-4HE, 10 μm, 250 mm×30 mm; mobile phase: acetonitrile/0.1% aq. formic acid; gradient: 10:90→90:10.

### Method 35 (Preparative HPLC):

Column: Daicel Chiralpak AD-H, 5 μm, 250 mm×20 mm; mobile phase: isohexane/ethanol 50:50; flow rate: 15 ml/min.

### Method 36 (Preparative HPLC):

Column: Daicel Chiralpak AZ-H, 5 μm, 250 mm×20 mm; mobile phase: isohexane/propanol 50:50; flow rate: 15 ml/min; temperature: 40° C.; UV detection: 210 nm.

### Method 37 (Preparative HPLC):

Column: YMC-ODS-AQ, C18, 10 μm, 250 mm×30 mm; mobile phase: methanol/0.1% aq. TFA; gradient: 20:80 (0.00-4.25 min)→40:60 (4.25-4.50 min)→60:40 (4.50-11.50 min)→100:0 (11.50-12.00 min)→100:0 (12.00-14.50 min)→20:80 (14.50-14.75 min)→20:80 (14.75-18.00 min).

### Method 38 (Preparative HPLC):

Column: Daicel Chiralpak IA, 5 μm, 250 mm×20 mm; mobile phase: methanol/acetonitrile 90:10; flow rate: 15 ml/min.

### Method 39 (Preparative HPLC):

Column: Daicel Chiralpak AD-H, 5 μm, 250 mm×20 mm; mobile phase: isohexane/ethanol 50:50; flow rate: 20 ml/min; temperature: 25° C.; UV detection: 230 nm.

### Method 40 (Preparative HPLC):

Column: Waters Sunfire C18 OBD, 5 μm, 150 mm×19 mm; mobile phase: acetonitrile/water/1% aq. TFA 35:52:13; flow rate: 25 ml/min.

### Method 41 (Preparative HPLC):

Column: Daicel Chiralpak AZ-H, 5 μm; 250 mm×30 mm; mobile phase: isohexane/ethanol 50:50; flow rate: 30 ml/min; temperature: 25° C.; UV detection: 230 nm.

### Method 42 (Preparative HPLC):

Column: Daicel Chiralpak AZ-H, 5 μm; 250 mm×20 mm; mobile phase: isohexane/ethanol 50:50; flow rate: 20 ml/min; temperature: 25° C.; UV detection: 230 nm.

### Method 43 (Preparative HPLC):

Column: Daicel Chiralpak IA, 5 μm; 250 mm×20 mm; mobile phase: methanol/acetonitrile 50:50; flow rate: 20 ml/min; temperature: 25° C.; UV detection: 220 nm.

### Method 44 (Preparative HPLC):

Column: Daicel Chiralpak AZ-H, 5 μm; 250 mm×20 mm; mobile phase: isohexane/propanol 50:50; flow rate: 15 ml/min; temperature: 40° C.; UV detection: 220 nm.

### Method 45 (Preparative HPLC):

Column: Reprosil-Pur C18, 10 μm, 250 mm×30 mm; mobile phase: acetonitrile/water with 0.1% formic acid; gradient: 30:70→90:10.

### Method 46 (Preparative HPLC):

Column: Daicel Chiralpak AZ-H, 5 μm, 250 mm×30 mm; mobile phase: isohexane/ethanol 60:40; flow rate: 40 ml/min; temperature: 25° C.; UV detection: 220 nm.

The following descriptions of the coupling patterns of ¹H NMR signals are based on the optical appearance of the signals in question and do not necessarily correspond to a strict, physically accurate interpretation. In general, the stated chemical shift refers to the centre of the signal in question; in the case of broad multiplets, a range is stated.

Melting points and melting ranges are, if stated, uncorrected.

All reactants or reagents whose preparation is not explicitly described hereinbelow were obtained from generally accessible sources. For all reactants or reagents whose preparation is likewise not described hereinbelow and which were not commercially available or which were obtained from sources not generally accessible, a reference to the published literature describing their preparation is given.

### Starting Materials and Intermediates

### Example 1A

### 2-({Fluoro[5-methyl-1-(4-methylbenzyl)-1H-pyrazol-3-yl]methyl}sulphonyl)-1,3-benzothiazole (racemate)

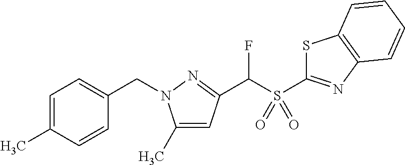 [SMILES: Cc1ccc(Cn2nc(C(F)S(=O)(=O)c3nc4ccccc4s3)cc2C)cc1]

### Step 1: Methyl 5-methyl-1-(4-methylbenzyl)-1H-pyrazole-3-carboxylate

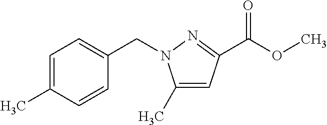

A solution of 22.7 g (155 mmol, purity 98%) of methyl 2,4-dioxopentanoate and 35.6 g (170 mmol) of (4-methylbenzyl)hydrazine in 225 ml of acetic acid was stirred at 90° C. for 4 h. The acetic acid was then removed on a rotary evaporator and the residue was purified by column chromatography (silica gel, mobile phase cyclohexane/ethyl acetate 10:1→2:1). Drying under high vacuum gave 18.2 g (48% of theory) of the title compound.

¹H NMR (400 MHz, CDCl₃, δ/ppm): 7.12 (d, 2H), 7.02 (d, 2H), 6.61 (s, 1H), 5.34 (s, 2H), 3.93 (s, 3H), 2.32 (s, 3H), 2.19 (s, 3H).

HPLC (Method 1): Rₜ=4.31 min.

MS (DCI): m/z=245 [M+H]⁺, 262 [M+NH₄]⁺.

### Step 2: 5-Methyl-1-(4-methylbenzyl)-1H-pyrazole-3-carboxylic acid

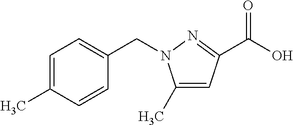 [SMILES: Cc1ccc(Cn2nc(C(=O)O)cc2C)cc1]

183 ml (183 mmol) of 1 M aqueous sodium hydroxide solution were added to a solution of 22.3 g (91.4 mmol) of the compound from Example 1A/Step 1 in 560 ml of ethanol, and the reaction mixture was stirred at an internal temperature of 70° C. overnight. After cooling to RT, the mixture was concentrated on a rotary evaporator to a volume of about 180 ml, and about 100 ml of 3 M hydrochloric acid were added with ice cooling. The resulting precipitate was filtered off and washed in each case twice with water and methyl tert-butyl ether. Drying gave 20.3 g (97% of theory) of the title compound.

¹H NMR (400 MHz, DMSO-d₆, δ/ppm): 7.15 (d, 2H), 7.03 (d, 2H), 6.51 (s, 1H), 5.31 (s, 2H), 2.27 (s, 3H), 2.21 (s, 3H).

LC/MS (Method 3, ESIpos): Rₜ=1.88 min, m/z=231 [M+H]⁺.

### Step 3: [5-Methyl-1-(4-methylbenzyl)-1H-pyrazol-3-yl]methanol

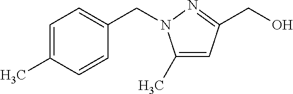 [SMILES: Cc1ccc(Cn2nc(CO)cc2C)cc1]

Under argon and at 0° C., 165 mg (4.34 mmol) of a 1 M solution of lithium aluminium hydride in diethyl ether were added slowly to a suspension of 500 mg (2.17 mmol) of the compound from Example 1A/Step 2 in 10 ml of THF. The mixture was stirred at 0° C. for 1 h and then at RT for a further 2 h. 5 ml of water were then added slowly, and the mixture was taken up in 50 ml of ethyl acetate and 50 ml of 1 M hydrochloric acid. After phase separation, the aqueous phase was extracted twice with in each case 50 ml of ethyl acetate, and the combined organic phases were dried over sodium sulphate, filtered and concentrated. The aqueous phase was then re-extracted three more times with in each case 30 ml of dichloromethane, and these combined extracts were likewise dried over sodium sulphate, filtered and concentrated. The two crude product batches obtained in this manner were combined and purified by column chromatography (silica gel, mobile phase first cyclohexane/ethyl acetate 2:1, then ethyl acetate). Drying under high vacuum gave 331 mg (70% of theory) of the title compound.

¹H NMR (400 MHz, DMSO-d₆, δ/ppm): 7.12 (d, 2H), 6.99 (d, 2H), 6.00 (s, 1H), 5.16 (s, 2H), 4.90 (t, 1H), 4.34-4.31 (m, 2H), 2.26 (s, 3H), 2.16 (s, 3H).

LC/MS (Method 4, ESIpos): Rₜ=1.38 min, m/z=217 [M+H]⁺.

### Step 4: 3-(Bromomethyl)-5-methyl-1-(4-methylbenzyl)-1H-pyrazole

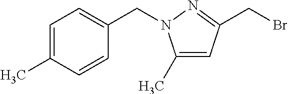 [SMILES: Cc1ccc(Cn2nc(CBr)cc2C)cc1]

At RT, 600 mg (1.81 mmol) of carbon tetrabromide and 593 mg (2.26 mmol) of triphenylphosphine were added to a solution of 326 mg (1.51 mmol) of the compound from Example 1A/Step 3 in 10 ml of dichloromethane, and the mixture was stirred at RT for 8 h. After the addition of a further 300 mg of carbon tetrabromide, the mixture was stirred at RT for a further 24 h. A further 295 mg of triphenylphosphine were then added, and the mixture was stirred at RT for another 2 h. The mixture was then concentrated on a rotary evaporator and the residue was purified by column chromatography (silica gel, mobile phase first cyclohexane/ethyl acetate 9:1, then cyclohexane/ethyl acetate 3:1, finally ethyl acetate). Drying under high vacuum gave 107 mg (25% of theory, purity 94%) of the title compound.

¹H NMR (400 MHz, DMSO-d₆, δ/ppm): 7.13 (d, 2H), 7.00 (d, 2H), 6.14 (s, 1H), 5.20 (s, 2H), 4.55 (s, 2H), 2.26 (s, 3H), 2.17 (s, 3H).

LC/MS (Method 2, ESIpos): Rₜ=1.28 min, m/z=279/281 [M+H]⁺.

### Step 5: 2-({[5-Methyl-1-(4-methylbenzyl)-1H-pyrazol-3-yl]methyl}sulphanyl)-1,3-benzothiazole

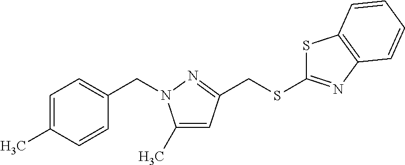 [SMILES: Cc1ccc(Cn2nc(CSc3nc4ccccc4s3)cc2C)cc1]

85 mg (0.449 mmol) of 2-mercapto-1,3-benzothiazole sodium salt were added to a solution of 105 mg (0.374 mmol) of the compound from Example 1A/Step 4 in 1.6 ml of DMF, and the mixture was stirred at RT for 1 h. 40 ml of water and 20 ml of ethyl acetate were then added to the mixture, and the phases were separated. The aqueous phase was extracted twice with in each case 20 ml of ethyl acetate, and the combined organic phases were dried over sodium sulphate, filtered and concentrated. The residue was purified by column chromatography (silica gel, mobile phase cyclohexane/ethyl acetate 7:1). Drying under high vacuum thus gave 132 mg (89% of theory, purity 92%) of the title compound.

¹H NMR (400 MHz, DMSO-d₆, δ/ppm): 8.01 (d, 1H), 7.87 (d, 1H), 7.51-7.44 (m, 1H), 7.42-7.34 (m, 1H), 7.08 (d, 2H), 6.98 (d, 2H), 6.11 (s, 1H), 5.18 (s, 2H), 4.52 (s, 2H), 2.25 (s, 3H), 2.14 (s, 3H).

LC/MS (Method 3, ESIpos): Rₜ=2.80 min, m/z=366 [M+H]⁺.

### Step 6: 2-({[5-Methyl-1-(4-methylbenzyl)-1H-pyrazol-3-yl]methyl}sulphonyl)-1,3-benzothiazole

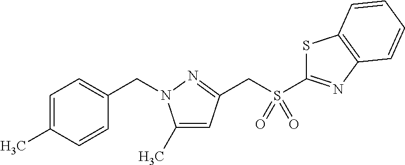 [SMILES: Cc1ccc(Cn2nc(CS(=O)(=O)c3nc4ccccc4s3)cc2C)cc1]

With cooling using an ice/acetone bath, 185 mg (0.752 mmol) of 3-chloroperbenzoic acid (water-moist, content 70%) were added slowly to a solution of 125 mg (0.342 mmol) of the compound from Example 1A/Step 5 in 4 ml of dichloromethane. After 1 d of stirring at RT, 20 ml of saturated aqueous sodium bicarbonate solution were added and the mixture was stirred vigorously for 15 min. After subsequent addition of 15 ml of dichloromethane, the phases were separated and the aqueous phase was extracted twice with in each case 20 ml of dichloromethane. The combined organic phases were dried over sodium sulphate, filtered and concentrated. Drying under reduced pressure gave 124 mg (76% of theory, purity 83%) of the title compound.

¹H NMR (400 MHz, DMSO-d₆, δ/ppm): 8.34-8.25 (m, 2H), 7.77-7.67 (m, 2H), 6.92 (d, 2H), 6.70 (d, 2H), 6.10 (s, 1H), 5.06 (s, 2H), 4.97 (s, 2H), 3.32 (s, 1H), 3.30 (s, 1H).

LC/MS (Method 3, ESIpos): Rₜ=2.48 min, m/z=398 [M+H]⁺.

### Step 7: 2-({Fluoro[5-methyl-1-(4-methylbenzyl)-1H-pyrazol-3-yl]methyl}sulphonyl)-1,3-benzothiazole (racemate)

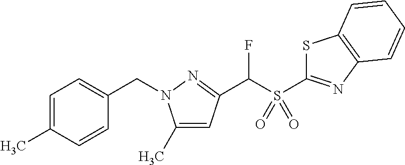 [SMILES: Cc1ccc(Cn2nc(C(F)S(=O)(=O)c3nc4ccccc4s3)cc2C)cc1]

At a bath temperature of −78° C. and under argon, 181 μl (0.362 mmol) of a 2 M solution of lithium diisopropylamide (LDA) in THF/heptane/ethylbenzene were added slowly to a solution of 120 mg (0.302 mmol) of the compound from Example 1A/Step 6 in 5 ml of toluene. The mixture was stirred at this temperature for a few minutes. 190 mg (0.604 mmol) of solid N-fluorobenzenesulphonimide were then added, and the mixture was stirred at −78° C. for a further hour. The mixture was then allowed to warm slowly to RT, and 15 ml of dilute aqueous ammonium chloride solution and 10 ml of ethyl acetate were then added. After phase separation, the aqueous phase was extracted twice with ethyl acetate and the combined organic phases were washed once with 40 ml of saturated sodium bicarbonate solution, dried over sodium sulphate, filtered and concentrated. The residue was purified by preparative HPLC (Method 12). Concentration and drying of the combined product fractions gave 52 mg (41% of theory) of the title compound.

¹H NMR (400 MHz, CDCl₃, δ/ppm): 8.30 (m, 1H), 8.04 (m, 1H), 7.66 (m, 2H), 7.11 (d, 2H), 6.99 (d, 2H), 6.70 (d, 1H), 6.53 (s, 1H), 5.34-5.22 (m, 2H), 2.33 (s, 3H), 2.23 (s, 3H).

LC/MS (Method 2, ESIpos): Rₜ=1.43 min, m/z=416 [M+H]⁺.

### Example 2A

### 2-({Fluoro[5-methyl-1-(tetrahydro-2H-pyran-2-yl)-1H-pyrazol-3-yl]methyl}sulphonyl)-1,3-benzothiazole (diastereomer and enantiomer mixture)

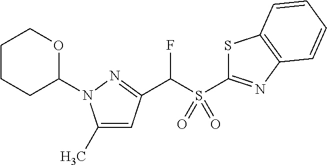 [SMILES: Cc1cc(C(F)S(=O)(=O)c2nc3ccccc3s2)nn1C1CCCCO1]

### Step 1: Ethyl 5-methyl-1-(tetrahydro-2H-pyran-2-yl)-1H-pyrazole-3-carboxylate (racemate)

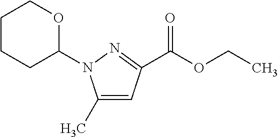 [SMILES: CCOC(=O)c1cc(C)n(C2CCCCO2)n1]

At 0° C., 28 ml (0.311 mol) of 3,4-dihydro-2H-pyran and 4.94 g (0.026 mol) of solid p-toluenesulphonic acid were added to a solution of 40 g (0.259 mol) of ethyl 5-methyl-1H-pyrazole-3-carboxylate in 800 ml of dichloromethane. After removal of the cooling bath, the reaction mixture was stirred at RT for 16 h. The mixture was then extracted successively with in each case about 800 ml of semisaturated aqueous sodium bicarbonate solution and water. The organic phase was dried over anhydrous magnesium sulphate, filtered and freed from the solvent on a rotary evaporator. The residue obtained was purified by filtration with suction on silica gel using the mobile phase cyclohexane/ethyl acetate 2:1. Concentration of the product fractions gave 42 g (68% of theory) of the title compound.

¹H NMR (400 MHz, CDCl₃, δ/ppm): 6.57 (s, 1H), 5.37 (dd, 1H), 4.38 (quart, 2H), 4.06-4.01 (m, 1H), 3.68-3.61 (m, 1H), 2.50-2.40 (m, 1H), 2.39 (s, 3H), 2.14-2.09 (m, 1H), 2.02-1.97 (m, 1H), 1.73-1.63 (m, 2H), 1.62-1.57 (m, 1H), 1.38 (t, 3H).

LC/MS (Method 5, ESIpos): Rₜ=0.91 min, m/z=239 [M+H]⁺.

### Step 2: [5-Methyl-1-(tetrahydro-2H-pyran-2-yl)-1H-pyrazol-3-yl]methanol (racemate)

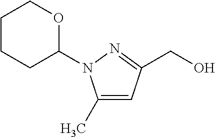

42 g (0.176 mol) of the compound from Example 2A/Step 1 were dissolved in 850 ml of anhydrous THF, and 147 ml (0.352 mol) of a 2.4 M solution of lithium aluminium hydride in THF were added dropwise at 0° C. The rate of addition was adjusted such that during the highly exothermic reaction the temperature of the reaction mixture did not exceed 10° C. After the addition had ended, the mixture was stirred at 0° C. for another 1 h and then at RT for 16 h. The mixture was then once more cooled to 0° C., and 14 ml of water, 14 ml of 15% strength aqueous sodium hydroxide solution and 600 ml of ethyl acetate were added carefully in succession. After brief stirring at RT, the resulting precipitate was filtered off and washed with ethyl acetate, and the combined filtrates were freed from the solvent on a rotary evaporator. The residue obtained was triturated with dichloromethane. Filtration and drying of this filter residue gave 31.89 g of the title compound. Partial concentration of the filtrate and another filtration gave, after drying, a further 1.0 g of the target compound. This gave a total of 32.89 g (95% of theory) of the title compound.

¹H NMR (400 MHz, CDCl₃, δ/ppm): 6.04 (s, 1H), 5.21 (dd, 1H), 4.63 (d, 2H), 4.08-4.03 (m, 1H), 3.68-3.61 (m, 1H), 2.49-2.39 (m, 1H), 2.33 (s, 3H), 2.12-2.06 (m, 1H), 1.95 (t, 1H), 1.97-1.89 (m, 1H), 1.73-1.63 (m, 2H), 1.60-1.54 (m, 1H).

LC/MS (Method 5, ESIpos): Rₜ=0.60 min, m/z=197 [M+H]⁺.

### Step 3: [5-Methyl-1-(tetrahydro-2H-pyran-2-yl)-1H-pyrazol-3-yl]methylmethane-sulphonate (racemate)

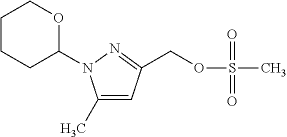 [SMILES: Cc1cc(COS(C)(=O)=O)nn1C1CCCCO1]

51.2 g (0.261 mol) of the compound from Example 2A/Step 2 (from 2 reactions) and 47 ml (0.339 mol) of triethylamine were suspended in 400 ml of THF, and a solution of 24 ml (0.313 mol) of methanesulphonyl chloride in 150 ml of THF were added at 0° C. The rate of addition was adjusted such that during the exothermic reaction the temperature of the reaction mixture did not exceed 10° C. After the addition had ended, the mixture was stirred at 0° C. for another 2 h. About 800 ml of semisaturated aqueous ammonium chloride solution were then added. The mixture was extracted three times with in each case about 500 ml of ethyl acetate. The combined organic extracts were dried over anhydrous magnesium sulphate, filtered and freed from the solvent on a rotary evaporator. Drying under high vacuum gave 72 g (95% of theory, purity about 95%) of the title compound.

¹H NMR (400 MHz, CDCl₃, δ/ppm): 6.19 (s, 1H), 5.24 (dd, 1H), 5.21 (s, 2H), 4.07-4.02 (m, 1H), 3.68-3.62 (m, 1H), 2.97 (s, 3H), 2.46-2.37 (m, 1H), 2.33 (s, 3H), 2.13-2.07 (m, 1H), 1.95-1.89 (m, 1H), 1.74-1.64 (m, 2H), 1.62-1.56 (m, 1H).

MS (DCI): m/z=275 [M+H]⁺.

### Step 4: 2-({[5-Methyl-1-(tetrahydro-2H-pyran-2-yl)-1H-pyrazol-3-yl]methyl}sulphanyl)-1,3-benzothiazole (racemate)

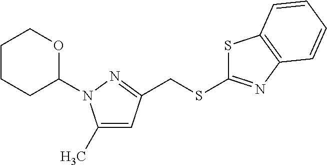 [SMILES: Cc1cc(CSc2nc3ccccc3s2)nn1C1CCCCO1]

72 g (0.262 mol) of the compound from Example 2A/Step 3 were dissolved in 1000 ml of DMF, and 49.7 g (0.262 mol) of solid sodium 1,3-benzothiazole-2-thiolate were added at RT. After 1 h of stirring at RT, most of the solvent was removed on a rotary evaporator. About 300 ml of water were added to the residue, and the mixture was extracted with in each case about 200 ml of ethyl acetate. The combined organic extracts were dried over anhydrous magnesium sulphate, filtered and freed from the solvent on a rotary evaporator. The residue obtained was purified by filtration with suction on silica gel using the mobile phase cyclohexane/ethyl acetate 6:1. Concentration of the product fractions gave 64.5 g (71% of theory) of the title compound.

¹H NMR (400 MHz, CDCl₃, δ/ppm): 7.88 (d, 1H), 7.75 (d, 1H), 7.41 (dd, 1H), 7.29 (dd, 1H), 6.11 (s, 1H), 5.20 (dd, 1H), 4.57 (s, 2H), 4.07-4.01 (m, 1H), 3.67-3.60 (m, 1H), 2.47-2.38 (m, 1H), 2.29 (s, 3H), 2.13-2.07 (m, 1H), 1.96-1.90 (m, 1H), 1.78-1.60 (m, 2H), 1.60-1.53 (m, 1H, partially obscured by the water signal).

LC/MS (Method 5, ESIpos): Rₜ=1.21 min, m/z=346 [M+H]⁺.

### Step 5: 2-({[5-Methyl-1-(tetrahydro-2H-pyran-2-yl)-1H-pyrazol-3-yl]methyl}sulphonyl)-1,3-benzothiazole (racemate)

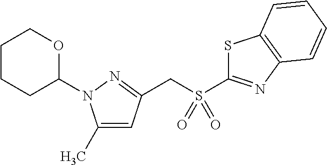 [SMILES: Cc1cc(CS(=O)(=O)c2nc3ccccc3s2)nn1C1CCCCO1]

39.9 g (0.115 mol) of the compound from Example 2A/Step 4 were dissolved in 1.4 liters of dichloromethane, and 85.4 g (0.346 mol) of solid m-chloroperoxybenzoic acid were added a little at a time at 0° C. After the slightly exothermic reaction had ended, the mixture was stirred at RT for another 3 h. About 500 ml of semisaturated aqueous sodium bicarbonate solution were added, and the mixture was stirred vigorously for 15 min. After phase separation, the aqueous phase was extracted two more times with in each case about 300 ml of dichloromethane. The combined organic extracts were washed with water and subsequently dried over anhydrous magnesium sulphate, filtered and freed from the solvent on a rotary evaporator. The residue obtained was by filtration with suction on silica gel using the mobile phase cyclohexane/ethyl acetate 85:15. Concentration of the product fractions gave 32.1 g (74% of theory) of the title compound.

¹H NMR (400 MHz, CDCl₃, δ/ppm): 8.25 (d, 1H), 7.95 (d, 1H), 7.62 (dd, 1H), 7.57 (dd, 1H), 6.17 (s, 1H), 5.10 (dd, 1H), 4.77 (pseudo-quart, 2H), 3.78-3.72 (m, 1H), 3.51-3.45 (m, 1H), 2.27 (s, 3H), 1.94-1.85 (m, 1H), 1.81-1.75 (m, 1H), 1.53-1.36 (m, 4H).

LC/MS (Method 5, ESIpos): Rₜ=1.04 min, m/z=378 [M+H]⁺.

### Step 6: 2-({Fluoro[5-methyl-1-(tetrahydro-2H-pyran-2-yl)-1H-pyrazol-3-yl]methyl}-sulphonyl)-1,3-benzothiazole (diastereomer and enantiomer mixture)

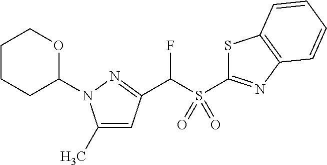 [SMILES: Cc1cc(C(F)S(=O)(=O)c2nc3ccccc3s2)nn1C1CCCCO1]

20 g (53.0 mmol) of the compound from Example 2A/Step 5 were dissolved in 900 ml of toluene, and 35 ml (63.6 mmol) of a 1.8 M solution of lithium diisopropylamide in a THF/hexane/toluene mixture were added dropwise at −78° C. After the addition had ended, the mixture was stirred for a further 30 min, and 33.4 g (0.106 mol) of solid N-fluoro-N-(phenylsulphonyl)benzene-sulphonamide were then added. The mixture was initially stirred further at −78° C. for 1 h. Over a period of 15 h, the mixture was then warmed to RT. About 500 ml of semisaturated aqueous ammonium chloride solution were then added dropwise. After phase separation, the aqueous phase was extracted two more times with in each case about 300 ml of ethyl acetate. The combined organic extracts were washed successively with water and saturated sodium chloride solution and then dried over anhydrous sodium sulphate, filtered and freed from the solvent on a rotary evaporator. The residue obtained was taken up in a little dichloromethane and purified by filtration with suction on silica gel using the mobile phase cyclohexane/ethyl acetate 85:15. Concentration of the product fractions gave 16.2 g (77% of theory) of the title compound.

¹H NMR (400 MHz, CDCl₃, δ/ppm): 8.30 and 8.29 (2 d, tog. 1H), 8.02 (d, 1H), 7.69-7.61 (m, 2H), 6.67 and 6.66 (2 d, tog. 1H), 6.52 (s, 1H), 5.34 and 5.30 (2 dd, tog. 1H), 4.02-3.97 and 3.89-3.84 (2 m, tog. 1H), 3.68-3.57 (m, 1H), 2.39 (s, 3H), 2.40-2.21 (m, 1H), 2.12-2.03 (m, 1H), 1.95-1.86 (m, 1H), 1.70-1.54 (m, 3H, partially obscured by the water signal).

LC/MS (Method 5, ESIpos): Rₜ=1.15 min, m/z=396 [M+H]⁺.

### Example 3A

### 3-{(Z)-1-Fluoro-2-[4-(trifluoromethoxy)phenyl]vinyl}-5-methyl-1H-pyrazole

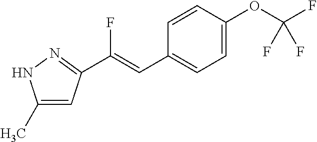 [SMILES: Cc1cc(/C(F)=C/c2ccc(OC(F)(F)F)cc2)n[nH]1]

At a temperature of 0-5° C., 15.2 ml (15.2 mmol) of a 1 M solution of lithium hexamethyldisilazide in THF were added dropwise to a solution of 2.50 g (6.32 mmol) of the compound from Example 2A and 1.20 g (6.32 mmol) of 4-(trifluoromethoxy)benzaldehyde in 120 ml of anhydrous THF. After the addition had ended, the reaction mixture was stirred at 0° C. for another 3 h. 300 ml of semisaturated aqueous ammonium chloride solution were added, and the mixture was extracted three times with in each case about 200 ml of ethyl acetate. The combined organic extracts were dried over anhydrous magnesium sulphate, and after filtration the solvent was removed under reduced pressure. The residue that remained was dissolved in 30 ml of a 4 M solution of hydrogen chloride in dioxane. After 16 h of stirring at RT, the mixture was diluted by addition of 100 ml of methyl tert-butyl ether. 100 ml of semisaturated aqueous sodium bicarbonate solution were then added. After vigorous stirring, the phases were separated and the organic phase was washed once with about 100 ml of semisaturated aqueous sodium bicarbonate solution and then dried over anhydrous magnesium sulphate. The crude product obtained after filtration and evaporation of the solvent was purified by MPLC (silica gel, mobile phase cyclohexane/ethyl acetate 10:1→5:1). What was isolated first was a minor fraction which, after removal of the solvent, gave 940 mg of a mixture which consisted to about 70% of the title compound and to about 30% of the isomeric (E) compound. The main fraction gave, after removal of the solvent and drying under high vacuum, 1.23 g (68% of theory) of the isomerically pure title compound.

¹H NMR (400 MHz, CDCl₃, δ/ppm): 7.62 (d, 2H), 7.19 (d, 2H), 6.34 (d, 1H), 6.29 (s, 1H), 2.36 (s, 3H).

LC/MS (Method 5, ESIpos): Rₜ=1.16 min, m/z=287 [M+H]⁺.

### Example 4A

### 3-{(Z)-1-Fluoro-2-[3-fluoro-4-(trifluoromethoxy)phenyl]vinyl}-5-methyl-1H-pyrazole

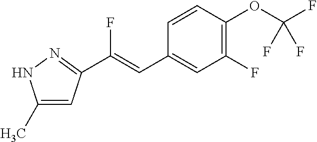 [SMILES: Cc1cc(/C(F)=C/c2ccc(OC(F)(F)F)c(F)c2)n[nH]1]

### Step 1: 3-{(Z)-1-Fluoro-2-[3-fluoro-4-(trifluoromethoxy)phenyl]vinyl}-5-methyl-1-(tetrahydro-2H-pyran-2-yl)-1H-pyrazole (racemate)

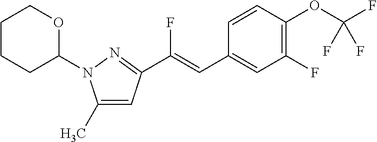 [SMILES: Cc1cc(/C(F)=C/c2ccc(OC(F)(F)F)c(F)c2)nn1C1CCCCO1]

Under argon and at 0° C., 48.0 ml (48.0 mmol) of a 1 M solution of lithium hexamethyldisilazide in THF were added to a solution of 7.91 g (20.0 mmol) of the compound from Example 2A and 4.16 g (20.0 mmol) of 3-fluoro-4-(trifluoromethoxy)benzaldehyde in 350 ml of THF. After 1 h of stirring at 0° C., 600 ml of saturated aqueous ammonium chloride solution were added, and the mixture was extracted twice with ethyl acetate. The combined organic phases were washed once with saturated sodium chloride solution, dried over magnesium sulphate, filtered and concentrated. The residue was purified by column chromatography (silica gel, mobile phase cyclohexane/ethyl acetate 9:1). Drying under high vacuum gave 4.40 g (55% of theory, purity 98%) of the title compound.

¹H NMR (400 MHz, CDCl₃, δ/ppm): 7.50 (d, 1H), 7.35-7.21 (m, 2H), 6.39 (d, 1H), 6.29 (s, 1H), 5.30 (dd, 1H), 4.08 (d, 1H), 3.71-3.63 (m, 1H), 2.55-2.42 (m, 1H), 2.38 (s, 3H), 2.18-2.09 (m, 1H), 2.01-1.93 (m, 1H), 1.80-1.56 (m, 3H).

LC/MS (Method 2, ESIpos): Rₜ=1.71 min, m/z=389 [M+H]⁺.

### Step 2: 3-{(Z)-1-Fluoro-2-[3-fluoro-4-(trifluoromethoxy)phenyl]vinyl}-5-methyl-1H-pyrazole

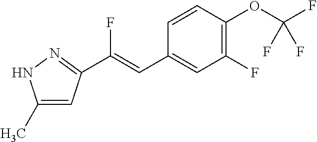 [SMILES: Cc1cc(/C(F)=C/c2ccc(OC(F)(F)F)c(F)c2)n[nH]1]

28.3 ml (113 mmol) of a 4 M solution of hydrogen chloride in dioxane were added to 4.40 g (11.3 mmol) of the compound from Example 4A/Step 1. The mixture was stirred at RT for 1 h. After addition of ethyl acetate, the mixture was washed with saturated aqueous sodium bicarbonate solution until neutral and then dried over magnesium sulphate, filtered and concentrated. The residue was triturated with pentane and the resulting solid was filtered off and dried under high vacuum. This gave 2.70 g (75% of theory, purity 96%) of the title compound.

¹H NMR (400 MHz, CDCl₃, δ/ppm): 10.6 (br. s, 1H), 7.50 (d, 1H), 7.28 (m, 2H), 6.31 (s, 1H), 6.31 (d, 1H), 2.36 (s, 3H).

LC/MS (Method 2, ESIpos): Rₜ=1.51 min, m/z=305 [M+H]⁺.

### Example 5A

### 3-{(Z)-2-[3-Chloro-4-(trifluoromethoxy)phenyl]-1-fluorovinyl}-5-methyl-1H-pyrazole

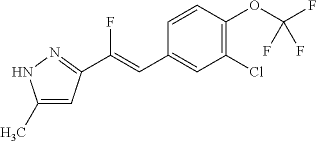 [SMILES: Cc1cc(/C(F)=C/c2ccc(OC(F)(F)F)c(Cl)c2)n[nH]1]

Analogously to the process described under Example 7A/Step 5 (see below), 1.50 g (3.79 mmol) of the compound from Example 2A and 840 μl (3.79 mmol) of 3-chloro-4-(trifluoromethoxy)benzaldehyde gave 282 mg (23% of theory) of the title compound. In this case, the reaction mixture was stirred at RT for 3 h, and the crude product was purified by preparative HPLC according to Method 13.

¹H NMR (400 MHz, CDCl₃, δ/ppm): 7.72 (d, 1H), 7.49 (dd, 1H), 7.29 (dd, 1H), 6.30 (s, 1H), 6.30 (d, 1H), 2.36 (s, 3H).

LC/MS (Method 6, ESIpos): Rₜ=2.59 min, m/z=321/323 [M+H]⁺.

### Example 6A

### 3-[(Z)-1-Fluoro-2-{4-[(trifluoromethyl)sulphanyl]phenyl}vinyl]-5-methyl-1H-pyrazole

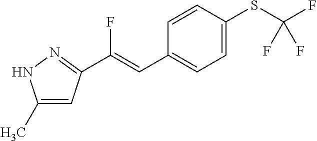 [SMILES: Cc1cc(/C(F)=C/c2ccc(SC(F)(F)F)cc2)n[nH]1]

Analogously to the process described under Example 3A, 2.08 g (5.00 mmol) of the compound from Example 2A and 1.03 g (5.00 mmol) of 4-[(trifluoromethyl)sulphanyl]benzaldehyde gave 550 mg (36% of theory) of the title compound. In this case, the reaction time in the first partial step of the reaction was only 30 min (instead of 3 h). The first purification of the crude product by silica gel MPLC was followed by a further purification step using preparative HPLC (Method 14).

¹H NMR (400 MHz, CDCl₃, δ/ppm): 7.63 (s, 4H), 6.37 (d, 1H), 6.32 (s, 1H), 2.37 (s, 3H).

LC/MS (Method 5, ESIpos): Rₜ=1.19 min, m/z=303 [M+H]⁺.

### Example 7A

### 3-{(Z)-1-Fluoro-2-[4-(1,1,1-trifluoro-2-methylpropan-2-yl)phenyl]vinyl}-5-methyl-1H-pyrazole

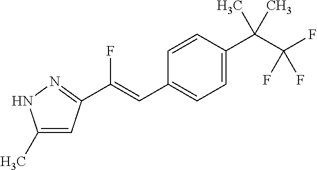 [SMILES: Cc1cc(/C(F)=C/c2ccc(C(C)(C)C(F)(F)F)cc2)n[nH]1]

### Step 1: 2-(4-Bromophenyl)-1,1,1-trifluoropropan-2-ol (racemate)

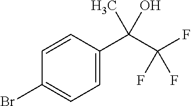

Initially, a suspension of dichloro(dimethyl)titanium in a heptane/dichloromethane mixture was prepared as follows: 100 ml (100 mmol) of a 1 M solution of titanium tetrachloride in dichloromethane were cooled to −30° C., 100 ml (100 mmol) of a 1 M solution of dimethylzinc in heptane were added dropwise and the mixture was stirred at −30° C. for 30 min. This suspension was then cooled to −40° C., and a solution of 10 g (39.5 mmol) of 1-(4-bromophenyl)-2,2,2-trifluoroethanone in 50 ml of dichloromethane was added. The mixture was stirred at −40° C. for another 5 min, the temperature was then allowed to reach RT and stirring was continued at RT for a further 2 h. With ice cooling, 50 ml of water were slowly added dropwise, and the mixture was then diluted with a further 300 ml of water. The mixture was extracted twice with dichloromethane, the combined dichloromethane phases were washed once with water, dried over anhydrous magnesium sulphate and filtered, and the solvent was removed on a rotary evaporator. The residue was purified by column chromatography on silica gel (mobile phase: cyclohexane/ethyl acetate 85:15). This gave 10.5 g (100% of theory) of the title compound which, according to ¹H NMR, contained residual solvent.

¹H NMR (400 MHz, CDCl₃, δ/ppm): 7.52 (d, 2H), 7.47 (d, 2H), 1.76 (s, 3H).

LC/MS (Method 3, ESIpos): Rₜ=2.27 min, m/z=251/253 [M−H₂O+H]⁺.

### Step 2: 2-(4-Bromophenyl)-1,1,1-trifluoropropan-2-yl methanesulphonate (racemate)

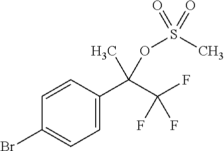 [SMILES: CC(OS(C)(=O)=O)(c1ccc(Br)cc1)C(F)(F)F]

Under argon, 3.12 g (78.1 mmol, 60% strength in mineral oil) of sodium hydride were initially charged in 45 ml of THF, and a solution of 10.5 g (39.0 mmol) of the compound obtained in Example 7A/Step 1 in 20 ml of THF was added dropwise at RT. After 1 h of stirring at RT and 30 min at 40° C., a solution of 8.94 g (78.1 mmol) of methanesulphonyl chloride in 45 ml of THF was added dropwise and the reaction mixture was stirred at 40° C. for a further 60 min. 50 ml of water were then slowly added dropwise, and the mixture was diluted with saturated aqueous sodium bicarbonate solution and extracted twice with ethyl acetate. The combined ethyl acetate phases were dried over anhydrous magnesium sulphate and filtered, and the solvent was removed on a rotary evaporator. The residue was triturated with hexane and the residue obtained was filtered off and dried under reduced pressure. This gave 12.4 g (92% of theory) of the title compound.

¹H NMR (400 MHz, CDCl₃, δ/ppm): 7.58 (d, 2H), 7.43 (d, 2H), 3.16 (s, 3H), 2.28 (s, 3H).

LC/MS (Method 6, ESIpos): Rₜ=2.32 min, m/z=364/366 [M+NH₄]⁺.

### Step 3: 1-Bromo-4-(1,1,1-trifluoro-2-methylpropan-2-yl)benzene

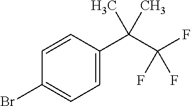

12.4 g (35.72 mmol) of the compound obtained in Example 7A/Step 2 were initially charged in 250 ml of dichloromethane, and the mixture was cooled to 0° C. With stirring, 35.7 ml (71.4 mmol) of a 2 M solution of trimethylaluminium in heptane were slowly added dropwise at 0° C., and the mixture was then allowed to warm to RT and stirred at RT for a further 1.5 h. 120 ml of a saturated aqueous sodium bicarbonate solution were slowly added dropwise to the mixture, followed by 40 ml of a saturated aqueous sodium chloride solution. The mixture was filtered through kieselguhr and the kieselguhr was washed twice with dichloromethane. The combined dichloromethane phases were washed once with saturated aqueous sodium chloride solution and dried over anhydrous magnesium sulphate, and the solvent was removed on a rotary evaporator. This gave 8.69 g (87% of theory) of the title compound in a purity of 95%.

¹H NMR (400 MHz, CDCl₃, δ/ppm): 7.49 (d, 2H), 7.33 (d, 2H), 1.55 (s, 6H).

LC/MS (Method 4, ESIpos): Rₜ=2.54 min, no ionization.

GC/MS (Method 10, EIpos): Rₜ=3.48 min, m/z=266 [M]⁺.

### Step 4: 4-(1,1,1-Trifluoro-2-methylpropan-2-yl)benzaldehyde

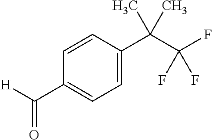

Under argon and at an internal temperature of 0-5° C., 31.2 ml (46.8 mmol) of a 1.5 M solution of butyllithium in hexane were added over a period of 30 min to a solution of 12.5 g (46.8 mmol) of the compound from Example 7A/Step 3 in 75 ml of diethyl ether, and the reaction mixture was stirred at 0° C. for a further 30 min. A solution of 5.76 ml (74.9 mmol) of anhydrous DMF in 25 ml of anhydrous diethyl ether was then added at an internal temperature of 0-10° C., and the reaction mixture was stirred for a further hour. 200 ml of 10% strength hydrochloric acid were then added, and the phases were separated. After extraction of the aqueous phase with 100 ml of diethyl ether, the combined organic phases were washed with in each case 200 ml of saturated sodium bicarbonate solution and saturated sodium chloride solution, dried over sodium sulphate, filtered and concentrated at not too greatly reduced pressure (owing to the volatility of the title compound). Purification of the residue by column chromatography (silica gel, mobile phase petroleum ether/dichloromethane 7:3) gave 6.78 g (67% of theory) of the title compound.

¹H NMR (400 MHz, CDCl₃, δ/ppm): 10.04 (s, 1H), 7.89 (d, 2H), 7.69 (d, 2H), 1.63 (s, 6H).

LC/MS (Method 6, ESIpos): Rₜ=2.33 min, m/z=217 [M+H]⁺.

GC/MS (Method 10, EIpos): Rₜ=3.66 min, m/z=216 [M]⁺.

### Step 5: 3-{(Z)-1-Fluoro-2-[4-(1,1,1-trifluoro-2-methylpropan-2-yl)phenyl]vinyl}-5-methyl-1H-pyrazole

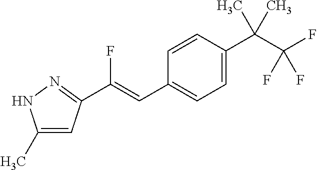 [SMILES: Cc1cc(/C(F)=C/c2ccc(C(C)(C)C(F)(F)F)cc2)n[nH]1]

Under argon, 1.66 g (7.70 mmol) of the compound from Example 7A/Step 4 were added to a solution of 2.77 g (7.0 mmol) of the compound from Example 2A in 90 ml of THF, and the mixture was cooled to 0° C. 16.8 ml of a 1.5 M solution of lithium hexamethyldisilazide in THF were then added dropwise at an internal temperature of 0-5° C., and the reaction mixture was stirred at 0° C. for another 2 h. 200 ml of dilute aqueous ammonium chloride solution and 200 ml of ethyl acetate were then added to the mixture, and the phases were separated. The aqueous phase was extracted once with 200 ml of ethyl acetate, and the combined organic phases were dried over sodium sulphate, filtered and concentrated. 35 ml of a 4 N solution of hydrogen chloride in dioxane were added to the residue, and the mixture was stirred at RT overnight. 100 ml of ethyl acetate were then added, and the mixture was washed twice with in each case 100 ml of dilute aqueous sodium bicarbonate solution. The organic phase was dried over sodium sulphate, filtered and concentrated. The residue was purified by preparative HPLC (Method 15). Drying under high vacuum gave 1.37 g (62% of theory) of the title compound.

¹H NMR (400 MHz, CDCl₃, δ/ppm): 7.59 (d, 2H), 7.48 (d, 2H), 6.33 (d, 1H), 6.30 (s, 1H), 2.35 (s, 3H), 1.58 (s, 6H).

LC/MS (Method 5, ESIpos): Rₜ=1.19 min, m/z=313 [M+H]⁺.

### Example 8A

### 3-{(Z)-1-Fluoro-2-[3-fluoro-4-(1,1,1-trifluoro-2-methylpropan-2-yl)phenyl]vinyl}-5-methyl-1H-pyrazole

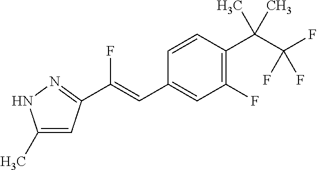 [SMILES: Cc1cc(/C(F)=C/c2ccc(C(C)(C)C(F)(F)F)c(F)c2)n[nH]1]

### Step 1: 1-[4-Bromo-2-fluoro-3-(trimethylsilyl)phenyl]-2,2,2-trifluoroethanone

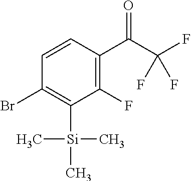 [SMILES: C[Si](C)(C)c1c(Br)ccc(C(=O)C(F)(F)F)c1F]

Under argon and at a bath temperature of −20° C., 78 ml (125 mmol) of a 1.6 M solution of n-butyllithium in hexane were slowly added dropwise to a solution of 17.6 g (124 mmol) of 2,2,6,6-tetramethylpiperidine in 110 ml of THF. After 30 min of stirring at −20° C., the mixture was cooled further to a bath temperature of −70° C., and a solution of 28.0 g (113 mmol) of (2-bromo-6-fluorophenyl)(trimethyl)silane [obtained from 1-bromo-3-fluorobenzene and chloro(trimethyl)silane according to S. Lulinski et al., J. Org. Chem. 2003, 68 (24), 9384-9388] in 30 ml of THF was added. After 1 h of stirring at a bath temperature of −70° C., 17.7 g (125 mmol) of ethyl trifluoroacetate were added dropwise at −70° C. The mixture was then allowed to warm slowly to RT and stirred at RT for another hour. Saturated aqueous ammonium chloride solution was then added, and the mixture was extracted twice with ethyl acetate. The combined ethyl acetate phases were washed with saturated sodium chloride solution, dried over magnesium sulphate, filtered and concentrated. This gave 42.0 g (82% pure, 89% of theory) of the title compound.

GC/MS (Method 10, EIpos): Rₜ=3.92 min, m/z=342/344 [M]⁺.

### Step 2: 1-(4-Bromo-2-fluorophenyl)-2,2,2-trifluoroethanone

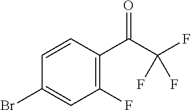 [SMILES: O=C(c1ccc(Br)cc1F)C(F)(F)F]

At RT, 120 ml (120 mmol) of a 1 M solution of tetra-n-butylammonium fluoride in THF were added to a solution of 42.0 g (100 mmol, purity 82%) of the compound from Example 8A/Step 1 in 140 ml of THF. After 30 min of stirring at RT, the mixture was diluted with ethyl acetate and washed once with water. The aqueous phase was re-extracted once with ethyl acetate. The combined organic phases were then washed once with saturated sodium chloride solution, dried over magnesium sulphate, filtered and concentrated. The residue obtained was purified by flash chromatography (silica gel, mobile phase cyclohexane→cyclohexane/ethyl acetate 95:5). Removal of the solvent gave 18.9 g (92% pure, 64% of theory) of the title compound.

¹H NMR (400 MHz, CDCl₃, δ/ppm): 7.78 (t, 1H), 7.49 (dd, 1H), 7.45 (dd, 1H).

GC/MS (Method 10, EIpos): Rₜ=2.63 min, m/z=270/272 [M]⁺.

### Step 3: 2-(4-Bromo-2-fluorophenyl)-1,1,1-trifluoropropan-2-ol (racemate)

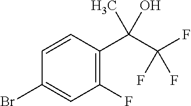

Initially, a suspension of dichloro(dimethyl)titanium in a heptane/dichloromethane mixture was prepared as follows: 160 ml (160 mmol) of a 1 M solution of titanium tetrachloride in dichloromethane were cooled to −30° C., 160 ml (160 mmol) of a 1 M solution of dimethylzinc in heptane were then added and the mixture was stirred at −30° C. for another 30 min. The suspension was then cooled to −40° C., and a solution of 19.4 g (65.9 mmol, purity 92%) of the compound from Example 8A/Step 2 in 80 ml of dichloromethane was added. The mixture was stirred at −40° C. for another 5 min, the bath temperature was then allowed to rise to RT and stirring at RT was continued for another 2 h. With ice cooling, 80 ml of water were slowly added dropwise, and the mixture was then diluted with a further 250 ml of water. The mixture was extracted twice with in each case 250 ml of dichloromethane, the combined dichloromethane phases were washed once with 350 ml of water, dried over anhydrous magnesium sulphate and filtered and the solvent was removed on a rotary evaporator. This gave 23.7 g (>100% of theory) of a residue which comprised the title compound in a purity of 92% according to ¹H NMR and was reacted further in this form.

¹H NMR (400 MHz, CDCl₃, δ/ppm): 7.52 (t, 1H), 7.34 (dd, 1H), 7.29 (dd, 1H), 3.06-2.99 (m, 1H), 1.86 (s, 3H).

LC/MS (Method 5, ESIneg): Rₜ=1.08 min, m/z=331/333 [M−H+HCO₂H]⁻.

GC/MS (Method 11, EIpos): Rₜ=3.61 min, m/z=286/288 [M]⁺.

### Step 4: 2-(4-Bromo-2-fluorophenyl)-1,1,1-trifluoropropan-2-yl methanesulphonate (racemate)

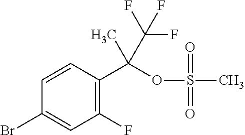 [SMILES: CC(OS(C)(=O)=O)(c1ccc(Br)cc1F)C(F)(F)F]

At RT, a solution of 23.7 g (75.9 mmol, purity 92%) of the compound from Example 8A/Step 3 in 40 ml of THF was added dropwise to a suspension of 6.08 g of sodium hydride (60% pure in mineral oil, 152 mmol) in 90 ml of THF. After 1 h of stirring at RT and a further 30 min at 40° C., a solution of 11.8 ml (152 mmol) of methanesulphonyl chloride in 90 ml of THF was added dropwise, and the mixture was then stirred at 40° C. for 1 h. 100 ml of water were then added slowly. The mixture was diluted with saturated aqueous sodium bicarbonate solution and extracted twice with ethyl acetate. The combined organic phases were dried over magnesium sulphate, filtered and concentrated. The residue obtained in this manner was triturated with pentane. The solid was filtered off, washed once with pentane and air-dried. This gave 25.6 g (92% of theory) of the title compound.

¹H NMR (400 MHz, CDCl₃, δ/ppm): 7.42 (t, 1H), 7.37 (dd, 1H), 7.32 (dd, 1H), 3.19 (s, 3H), 2.33 (s, 3H).

LC/MS (Method 2, ESIpos): Rₜ=1.34 min, m/z=382/384 [M+NH₄]⁺.

### Step 5: 4-Bromo-2-fluoro-1-(1,1,1-trifluoro-2-methylpropan-2-yl)benzene

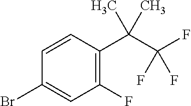

At 0° C., 70 ml (140 mmol) of a 2 M solution of trimethylaluminium in heptane were added slowly with stirring to a solution of 25.6 g (70.1 mmol) of the compound from Example 8A/Step 4 in 480 ml of dichloromethane. The bath temperature was allowed to rise to RT, and the mixture was stirred at RT for another 1 h. 230 ml of a saturated aqueous sodium bicarbonate solution and 75 ml of a saturated aqueous sodium chloride solution were then added slowly. The mixture was filtered slowly through kieselguhr and the filter residue was washed twice with dichloromethane. The filtrate was combined with the wash solution and washed once with saturated aqueous sodium chloride solution, dried over magnesium sulphate, filtered and concentrated. This gave 18.8 g (94% of theory) of the title compound.

¹H NMR (400 MHz, CDCl₃, δ/ppm): 7.32-7.24 (m, 3H), 1.63 (s, 6H).

GC/MS (Method 10, EIpos): Rₜ=2.99 min, m/z=283/285 [M]⁺.

Step 6: 3-Fluoro-4-(1,1,1-trifluoro-2-methylpropan-2-yl)benzaldehyde

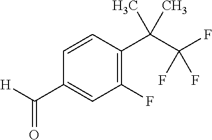 [SMILES: CC(C)(c1ccc(C=O)cc1F)C(F)(F)F]

At an internal temperature of 0-5° C. and under argon, 18.6 ml (29.8 mmol) of a 1.6 M solution of butyllithium in hexane were added over a period of 30 min to a solution of 8.5 g (29.8 mmol) of the compound from Example 8A/Step 5 in 50 ml of diethyl ether, and the reaction mixture was stirred at 0° C. for a further 30 min. A solution of 3.7 ml (47.7 mmol) of anhydrous DMF in 15 ml of anhydrous diethyl ether was then added at an internal temperature of 0-10° C., and the reaction mixture was stirred for another hour. 50 ml of 1 M hydrochloric acid were then added, followed by a little water and some tert-butyl methyl ether. The phases were separated, and, after extraction of the aqueous phase with 100 ml of tert-butyl methyl ether, the combined organic phases were washed with saturated sodium chloride solution, dried over magnesium sulphate, filtered and concentrated. Purification of the residue by column chromatography (silica gel, mobile phase cyclohexane/ethyl acetate 95:5) gave 1.50 g (15% of theory, purity about 70%) of the title compound.

¹H NMR (400 MHz, CDCl₃, δ/ppm): 9.99 (s, 1H), 7.69-7.53 (m, 3H), 1.69 (s, 6H).

GC/MS (Method 10, EIpos): Rₜ=3.22 min, m/z=234 [M]⁺.

Step 7: 3-{(Z)-1-Fluoro-2-[3-fluoro-4-(1,1,1-trifluoro-2-methylpropan-2-yl)phenyl]vinyl}-5-methyl-1-(tetrahydro-2H-pyran-2-yl)-1H-pyrazole (racemate)

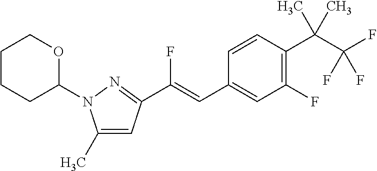 [SMILES: Cc1cc(/C(F)=C/c2ccc(C(C)(C)C(F)(F)F)c(F)c2)nn1C1CCCCO1]

Under argon, a solution of 1.77 g (4.48 mmol) of the compound from Example 2A and 1.50 g (4.48 mmol, purity about 70%) of the compound from Example 8A/Step 6 in 75 ml of THF was cooled to a bath temperature of 0° C., and 10.8 ml (10.8 mmol) of a 1 M solution of lithium hexamethyldisilazide in THF were added slowly with stirring. The reaction mixture was stirred at 0° C. for 30 min, and 70 ml of saturated aqueous ammonium chloride solution were then added at 0° C. After warming to RT, the mixture was diluted with water and extracted twice with ethyl acetate. The combined organic phases were washed once with saturated sodium chloride solution, dried over magnesium sulphate and concentrated. The residue was purified first by column chromatography (silica gel, mobile phase cyclohexane/ethyl acetate 95:5) and then by preparative HPLC (Method 16). The combined product fractions of the preparative HPLC were neutralized with solid sodium bicarbonate and concentrated to a residual volume of aqueous phase. After two extractions with ethyl acetate, the combined organic phases were dried over magnesium sulphate, filtered and concentrated. Drying of the residue under high vacuum was followed by another column chromatography (silica gel, mobile phase cyclohexane/ethyl acetate 9:1). This gave 469 mg (25% of theory) of the title compound.

¹H NMR (400 MHz, CDCl₃, δ/ppm): 7.40-7.28 (m, 3H), 6.38 (d, 1H), 6.29 (s, 1H), 5.29 (dd, 1H), 4.11-4.04 (m, 1H), 3.71-3.63 (m, 1H), 2.57-2.42 (m, 1H), 2.37 (s, 3H), 2.18-2.09 (m, 1H), 2.01-1.93 (m, 1H), 1.82-1.60 (m, 3H), 1.65 (s, 6H).

LC/MS (Method 5, ESIpos): Rₜ=1.45 min, m/z=415 [M+H]⁺.

### Step 8: 3-{(Z)-1-Fluoro-2-[3-fluoro-4-(1,1,1-trifluoro-2-methylpropan-2-yl)phenyl]vinyl}-5-methyl-1H-pyrazole

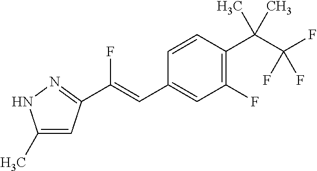 [SMILES: Cc1cc(/C(F)=C/c2ccc(C(C)(C)C(F)(F)F)c(F)c2)n[nH]1]

At RT, 2.7 ml (10.9 mmol) of a 4 M solution of hydrogen chloride in dioxane were added to 450 mg (1.09 mmol) of the compound from Example 8A/Step 7. After 1 h of stirring at RT, the reaction mixture was diluted with ethyl acetate and extracted with saturated aqueous sodium bicarbonate solution. After phase separation, the aqueous phase was extracted once with ethyl acetate, and the combined organic phases were washed once with saturated sodium chloride solution, dried over magnesium sulphate, filtered and concentrated. The residue was triturated with pentane, and the solid obtained was filtered off and dried under high vacuum. This gave 302 mg (84% of theory) of the title compound.

¹H NMR (400 MHz, CDCl₃, δ/ppm): 7.34 (m, 3H), 6.31 (s, 1H), 6.31 (d, 1H), 2.36 (s, 3H), 1.65 (s, 6H).

LC/MS (Method 5, ESIpos): Rₜ=1.21 min, m/z=331 [M+H]⁺.

### Example 9A

### 3-[(Z)-1-Fluoro-2-{4-[1-(trifluoromethyl)cyclopropyl]phenyl}vinyl]-5-methyl-1H-pyrazole

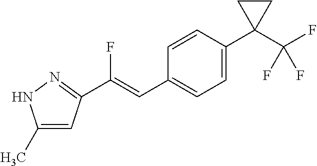 [SMILES: Cc1cc(/C(F)=C/c2ccc(C3(C(F)(F)F)CC3)cc2)n[nH]1]

### Step 1: 1-Bromo-4-[1-(trifluoromethyl)cyclopropyl]benzene

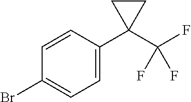 [SMILES: FC(F)(F)C1(c2ccc(Br)cc2)CC1]

Initially, activated zinc bromide on montmorillonite was prepared as follows: 1.40 g (6.22 mmol) of zinc bromide were initially charged in 56 ml of methanol, 5.64 g of montmorillonite K₁₀ were added and the mixture was stirred at RT for 1 h. After removal of the methanol, the powder that remained was heated in a sand bath at a bath temperature of 200° C. for 1 h and then allowed to cool under argon.

The title compound was then prepared as follows: 10.0 g (53.7 mmol) of 1-phenyl-1-(trifluoromethyl)cyclopropane were initially charged in 50 ml of pentane. 6.1 g (5.37 mmol) of the activated zinc bromide on montmorillonite obtained above were added, and 27.7 ml (537 mmol) of bromine were then slowly added dropwise with stirring in the dark. The mixture was stirred at RT in the dark overnight. 150 ml of a saturated aqueous sodium sulphite solution were then slowly added dropwise, and stirring at RT was continued for a further about 30 min until discoloration of the mixture occurred. The solid was filtered off and washed twice with pentane. After separation of the filtrate phases, the aqueous phase was extracted twice with in each case 200 ml of pentane. The combined organic phases were dried over sodium sulphate, filtered and concentrated gently (significant volatility of the target compound). In this manner, 17.1 g (>100% of theory) of the title compound which, according to ¹H NMR, still contained pentane, were obtained.

¹H NMR (400 MHz, CDCl₃, δ/ppm): 7.47 (d, 2H), 7.32 (s, 2H), 1.39-1.30 (m, 2H), 1.04-0.95 (m, 2H).

GC/MS (Method 10, EIpos): Rₜ=3.45 min, m/z=264/266 [M]⁺.

### Step 2: 4-[1-(Trifluoromethyl)cyclopropyl]benzaldehyde

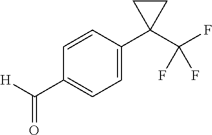 [SMILES: O=Cc1ccc(C2(C(F)(F)F)CC2)cc1]

Under argon and at 0° C., 37.7 ml (56.6 mmol) of a 1.5 M butyllithium solution in hexane were slowly added dropwise to a solution of 15.0 g (56.6 mmol) of the compound from Example 9A/Step 1 in 135 ml of diethyl ether, and the reaction mixture was stirred at 0° C. for 30 min. At 0° C., a solution of 7.0 ml (90.6 mmol) of anhydrous DMF in 35 ml of anhydrous diethyl ether was then added, and the reaction mixture was stirred at 0° C. for a further 30 min. The reaction mixture was then warmed to RT, 300 ml of 10% strength hydrochloric acid were added and the phases were separated. The aqueous phase was extracted with 150 ml of diethyl ether, and the combined organic phases were washed successively with in each case 200 ml of saturated sodium bicarbonate solution and saturated sodium chloride solution, dried over sodium sulphate, filtered and concentrated under not too strongly reduced pressure. This gave 16.30 g (>100% of theory, purity 96%) of the title compound, which still contained solvent residues.

¹H NMR (400 MHz, CDCl₃, δ/ppm): 10.04 (s, 1H), 7.88 (d, 2H), 7.64 (d, 2H), 1.47-1.41 (m, 2H), 1.12-1.06 (m, 2H).

LC/MS (Method 5, ESIpos): Rₜ=1.01 min, no ionization.

GC/MS (Method 10, EIpos): Rₜ=3.67 min, m/z=214 [M]⁺.

### Step 3: 3-[(Z)-1-Fluoro-2-{4-[1-(trifluoromethyl)cyclopropyl]phenyl}vinyl]-5-methyl-1H-pyrazole

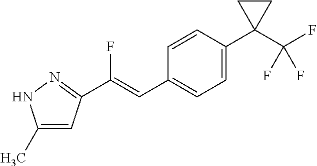 [SMILES: Cc1cc(/C(F)=C/c2ccc(C3(C(F)(F)F)CC3)cc2)n[nH]1]

### Method 1:

Under argon, 7.15 g (33.4 mmol) of the compound from Example 9A/Step 2, dissolved in 12 ml of THF, were added to a solution of 12.0 g (30.3 mmol) of the compound from Example 2A in 30 ml of THF, and the mixture was cooled to 0° C. 72.8 ml (72.8 mmol) of a 1 M lithium hexamethyldisilazide solution in THF were then added dropwise at an internal temperature of 0-5° C. The mixture was stirred at 0° C. for a further 3 h. After warming to RT, 600 ml of dilute aqueous ammonium chloride solution and 200 ml of tert-butyl methyl ether were added. After phase separation, the aqueous phase was extracted once with 300 ml of ethyl acetate. The combined organic phases were dried over sodium sulphate, filtered and concentrated. The residue was purified initially by column chromatography (silica gel, mobile phase cyclohexane/ethyl acetate 9:1) and then by preparative HPLC (Method 17). This gave two main fractions which corresponded to the two E/Z double bond isomers. 15.7 ml of a 4 N solution of hydrogen chloride in dioxane were added to the larger of these two fractions, which corresponded to the desired Z double bond isomer, and the mixture was stirred at RT for 1 h. The solid formed was filtered off and washed twice with in each case 4 ml of dioxane. The filtrate was kept. The solid was then taken up in 50 ml of ethyl acetate, and 50 ml of saturated aqueous sodium bicarbonate solution were added. After phase separation, the organic phase was dried over sodium sulphate, filtered and concentrated. Drying of the residue under reduced pressure gave 1.46 g (16% of theory) of the title compound. The filtrate which had been kept gave, after concentration, addition of another 21 ml of 4 N hydrogen chloride solution in dioxane, one hour of stirring at RT, removal by filtration of the solid formed, analogous aqueous work-up and drying under high vacuum of the substance obtained, a further 2.0 g (21% of theory) of the title compound. In this manner, a total of 3.46 g (37% of theory) of the title compound were obtained.

### Method 2:

According to Method 1 described above, initially 957 mg (2.42 mmol) of the compound from Example 2A and 570 mg (2.66 mmol) of the compound from Example 9A/Step 2 were reacted with one another. After analogous aqueous work-up, 10 ml of 4 N hydrogen chloride solution in dioxane were added to the residue obtained, and the mixture was stirred at RT overnight. 100 ml of tert-butyl methyl ether were then added, and the mixture was washed twice with in each case 150 ml of dilute aqueous sodium bicarbonate solution. The organic phase was dried over sodium sulphate, filtered and concentrated. The residue was purified by preparative HPLC (Method 18). Drying under reduced pressure gave 570 mg (57% of theory) of the title compound.

¹H NMR (400 MHz, CDCl₃, δ/ppm): 7.57 (d, 2H), 7.44 (d, 2H), 6.32 (d, 1H), 6.30 (s, 1H), 2.35 (s, 3H), 1.37-1.33 (m, 2H), 1.06-1.00 (m, 2H).

GC/MS (Method 5, ESIpos): Rₜ=1.17 min, m/z=311 [M+H]⁺.

### Example 10A

### 3-{(Z)-1-Fluoro-2-[4-(trifluoromethyl)phenyl]vinyl}-5-methyl-1H-pyrazole

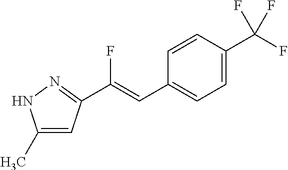 [SMILES: Cc1cc(/C(F)=C/c2ccc(C(F)(F)F)cc2)n[nH]1]

Analogously to the process described under Example 3A, 2.50 g (6.02 mmol) of the compound from Example 2A and 1.05 g (6.02 mmol) of 4-(trifluoromethyl)benzaldehyde gave 701 mg (43% of theory) of the title compound. In this case, the reaction time in the first partial step of the reaction was only 30 min (instead of 3 h). Moreover, in the present case the silica gel-MPLC was followed by two more purification steps: The product obtained from the MPLC was initially triturated with pentane. The solid was filtered off with suction and gave, after drying under high vacuum, a first partial amount of 566 mg of the title compound. The pentane filtrate was concentrated further to dryness and the residue was purified once more by preparative HPLC (Method 14). In this manner, a second partial amount of 135 mg (95% pure) of the title compound was obtained.

¹H NMR (400 MHz, CDCl₃, δ/ppm): 10.25 (very broad, 1H), 7.70 (d, 2H), 7.59 (d, 2H), 6.40 (d, 1H), 6.33 (s, 1H), 2.37 (s, 3H).

LC/MS (Method 2, ESIpos): Rₜ=1.30 min, m/z=271 [M+H]⁺.

### Example 11A

### 3-{(Z)-1-Fluoro-2-[4-(trimethylsilyl)phenyl]vinyl}-5-methyl-1H-pyrazole

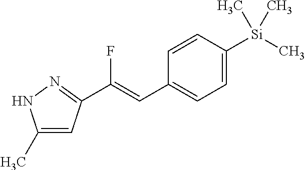

Under argon, 1.19 g (6.67 mmol) of 4-(trimethylsilyl)benzaldehyde [for the preparation, see, for example, US 2007/0185058-A1, Example S6-A], dissolved in 45 ml of THF, were added to a solution of 2.40 g (6.07 mmol) of the compound from Example 2A in 70 ml of THF. The mixture was cooled to 0° C., 14.6 ml (14.6 mmol) of a 1 M lithium hexamethyldisilazide solution in THF were then added dropwise at an internal temperature of 0-5° C. and the reaction mixture was stirred at 0° C. for 3 h. 300 ml of dilute aqueous ammonium chloride solution and 200 ml of ethyl acetate were then added to the mixture, and the phases were separated. The aqueous phase was extracted once with 200 ml of ethyl acetate, and the combined organic phases were dried over sodium sulphate, filtered and concentrated. 30 ml of a 4 N solution of hydrogen chloride in dioxane were added to the residue, and the mixture was stirred at RT overnight. 150 ml of tert-butyl methyl ether were then added, and the mixture was washed twice with in each case 200 ml of dilute aqueous sodium bicarbonate solution. The organic phase was dried over sodium sulphate, filtered and concentrated. The residue was purified by preparative HPLC (Method 19). Drying under high vacuum gave 820 mg (49% of theory) of the title compound.

¹H NMR (400 MHz, CDCl₃, δ/ppm): 7.58 (d, 2H), 7.52 (d, 2H), 6.32 (d, 2H), 6.30 (s, 1H), 2.35 (s, 3H), 0.27 (s, 9H).

LC/MS (Method 2, ESIpos): Rₜ=1.47 min, m/z=275 [M+H]⁺.

### Example 12A

### 3-[(Z)-2-(4-tert-Butylphenyl)-1-fluorovinyl]-5-methyl-1H-pyrazole

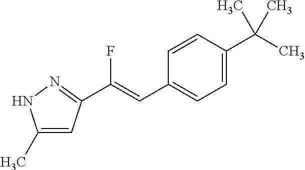 [SMILES: Cc1cc(/C(F)=C/c2ccc(C(C)(C)C)cc2)n[nH]1]

At a temperature of 0-5° C., 9.1 ml (9.1 mmol) of a 1 M solution of lithium hexamethyldisilazide in THF were added dropwise to a solution of 1.50 g (3.79 mmol) of the compound from Example 2A and 615 mg (3.79 mmol) of 4-tert-butylbenzaldehyde in 75 ml of anhydrous THF. After the addition had ended, the reaction mixture was stirred at 0° C. for 30 min. 75 ml of saturated aqueous ammonium chloride solution were then added, and the mixture was extracted three times with in each case about 50 ml of ethyl acetate. The combined organic extracts were washed with saturated sodium chloride solution and then dried over anhydrous magnesium sulphate. After filtration, the solvent was removed on a rotary evaporator. From the residue that remained, the THP-protected intermediate of the reaction was isolated by MPLC (silica gel, cyclohexane/ethyl acetate 10:1→5:1). This intermediate was then dissolved in 5 ml of a 4 M solution of hydrogen chloride in dioxane. After 60 min of stirring at RT, the solution was diluted by addition of about 100 ml of ethyl acetate. About 50 ml of saturated aqueous sodium bicarbonate solution were then added. After vigorous stirring, the phases were separated and the organic phase was washed once with saturated sodium chloride solution. The organic phase was dried over anhydrous magnesium sulphate. The crude product obtained after filtration and evaporation of the solvent was purified by preparative HPLC (Method 14). This gave three fractions: 398 mg (41% of theory) of the isomerically pure title compound, 208 mg of a mixed fraction of the title compound and the isomeric (E) compound and 116 mg of the isomerically pure (E) compound.

¹H NMR (400 MHz, CDCl₃, δ/ppm): 10.15 (very broad, 1H), 7.55 (d, 2H), 7.39 (d, 2H), 6.31 (d, 1H), 6.28 (s, 1H), 2.35 (s, 3H), 1.33 (s, 9H).

LC/MS (Method 5, ESIpos): Rₜ=1.19 min, m/z=259 [M+H]⁺.

### Example 13A

### 3-[(Z)-2-(4-Cyclohexylphenyl)-1-fluorovinyl]-5-methyl-1H-pyrazole

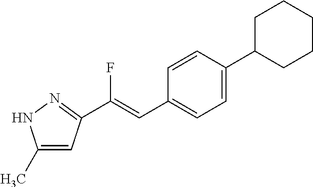 [SMILES: Cc1cc(/C(F)=C/c2ccc(C3CCCCC3)cc2)n[nH]1]

### Step 1: 3-[(Z)-2-(4-Cyclohexylphenyl)-1-fluorovinyl]-5-methyl-1-(tetrahydro-2H-pyran-2-yl)-1H-pyrazole (racemate)

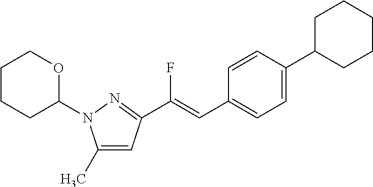 [SMILES: Cc1cc(/C(F)=C/c2ccc(C3CCCCC3)cc2)nn1C1CCCCO1]

Analogously to the process described under Example 4A/Step 1, 791 mg (2.00 mmol) of the compound from Example 2A and 418 mg (2.00 mmol, purity 90%) of 4-cyclohexylbenzaldehyde gave 367 mg (48% of theory, purity 97%) of the title compound. In this case, the reaction time was 30 min (instead of 1 h), and the crude product was purified by column chromatography on silica gel using the mobile phase mixture cyclohexane/ethyl acetate 95:5.

¹H NMR (400 MHz, CDCl₃, δ/ppm): 7.53 (d, 2H), 7.19 (d, 2H), 6.38 (d, 1H), 6.26 (s, 1H), 5.29 (dd, 1H), 4.10-4.02 (m, 1H), 3.70-3.61 (m, 1H), 2.55-2.44 (m, 2H), 2.36 (s, 3H), 2.17-2.10 (m, 1H), 2.01-1.93 (m, 1H), 1.91-1.80 (m, 4H), 1.78-1.68 (m, 3H), 1.68-1.59 (m, 1H), 1.45-1.35 (m, 4H), 1.31-1.20 (m, 1H).

LC/MS (Method 5, ESIpos): Rₜ=1.56 min, m/z=369 [M+H]⁺.

### Step 2: 3-[(Z)-2-(4-Cyclohexylphenyl)-1-fluorovinyl]-5-methyl-1H-pyrazole

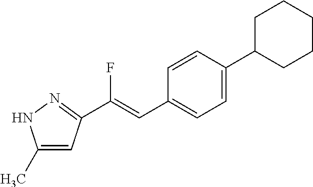 [SMILES: Cc1cc(/C(F)=C/c2ccc(C3CCCCC3)cc2)n[nH]1]

Analogously to the process described under Example 4A/Step 2, 360 mg (0.948 mmol, purity 97%) of the compound from Example 13A/Step 1 and 2.4 ml (9.48 mmol) of a 4 M solution of hydrogen chloride in dioxane gave 224 mg (83% of theory) of the title compound.

¹H NMR (400 MHz, CDCl₃, δ/ppm): 7.53 (d, 2H), 7.20 (d, 2H), 6.30 (d, 1H), 6.28 (s, 1H), 2.55-2.45 (m, 1H), 2.35 (s, 3H), 1.91-1.81 (m, 4H), 1.78-1.71 (m, 1H), 1.48-1.33 (m, 4H), 1.32-1.20 (m, 1H).

LC/MS (Method 2, ESIpos): Rₜ=1.56 min, m/z=285 [M+H]⁺.

### Example 14A

### 3-[(Z)-1-Fluoro-2-(4-isopropylphenyl)vinyl]-5-methyl-1H-pyrazole

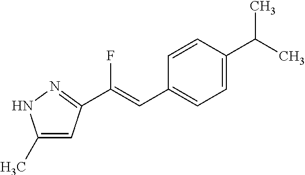 [SMILES: Cc1cc(/C(F)=C/c2ccc(C(C)C)cc2)n[nH]1]

### Step 1: 3-[(Z)-1-Fluoro-2-(4-isopropylphenyl)vinyl]-5-methyl-1-(tetrahydro-2H-pyran-2-yl)-1H-pyrazole (racemate)

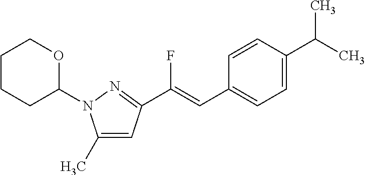 [SMILES: Cc1cc(/C(F)=C/c2ccc(C(C)C)cc2)nn1C1CCCCO1]

Analogously to the process described under Example 8A/Step 7, 1.0 g (2.53 mmol) of the compound from Example 2A and 386 mg (2.53 mmol, purity 97%) of 4-isopropylbenzaldehyde gave 539 mg (65% of theory) of the title compound. In this case, the reaction mixture was stirred at 0° C. for 3 h (instead of 30 min). The crude product obtained was triturated with warm cyclohexane/ethyl acetate 9:1, and the solid that remained was filtered off, washed twice with cyclohexane/ethyl acetate 9:1 and then discarded. The filtrate and the wash solutions were combined and concentrated, and the residue was purified by column chromatography (silica gel, cyclohexane/ethyl acetate 9:1).

¹H NMR (400 MHz, CDCl₃, δ/ppm): 7.54 (d, 2H), 7.21 (d, 2H), 6.39 (d, 1H), 6.26 (s, 1H), 5.29 (dd, 1H), 4.10-4.04 (m, 1H), 3.71-3.62 (m, 1H), 2.90 (sept, 1H), 2.56-2.44 (m, 1H), 2.37 (s, 3H), 2.17-2.10 (m, 1H), 2.01-1.93 (m, 1H), 1.78-1.65 (m, 2H), 1.64-1.57 (m, 1H), 1.25 (d, 6H).

LC/MS (Method 2, ESIpos): Rₜ=1.71 min, m/z=329 [M+H]⁺.

### Step 2: 3-[(Z)-1-Fluoro-2-(4-isopropylphenyl)vinyl]-5-methyl-1H-pyrazole

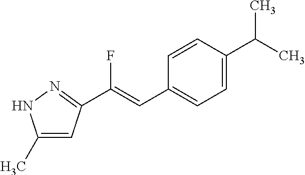 [SMILES: Cc1cc(/C(F)=C/c2ccc(C(C)C)cc2)n[nH]1]

4 ml (15.9 mmol) of a 4 M solution of hydrogen chloride in dioxane were added to 522 mg (1.59 mmol) of the compound from Example 14A/Step 1, and the mixture was stirred at RT for 1 h. 100 ml of saturated aqueous sodium bicarbonate solution were then added. The solid formed was filtered off and washed with water. Drying under reduced pressure gave 351 mg (84% of theory, purity 92%) of the title compound.

¹H NMR (400 MHz, CDCl₃, δ/ppm): 10.2 (very broad, 1H), 7.54 (d, 2H), 7.23 (d, 2H), 6.31 (d, 1H), 6.28 (s, 1H), 2.91 (sept, 1H), 2.35 (s, 3H), 1.26 (d, 6H).

LC/MS (Method 5, ESIpos): Rₜ=1.14 min, m/z=245 [M+H]⁺.

### Example 15A

### 3-[(Z)-1-Fluoro-2-(4-isobutylphenyl)vinyl]-5-methyl-1H-pyrazole

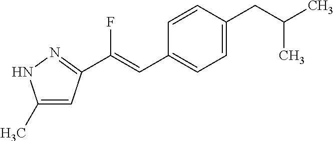 [SMILES: Cc1cc(/C(F)=C/c2ccc(CC(C)C)cc2)n[nH]1]

### Step 1: 3-[(Z)-1-Fluoro-2-(4-isobutylphenyl)vinyl]-5-methyl-1-(tetrahydro-2H-pyran-2-yl)-1H-pyrazole (racemate)

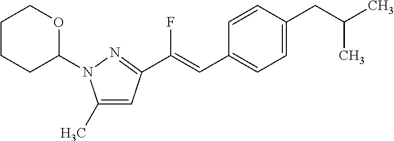 [SMILES: Cc1cc(/C(F)=C/c2ccc(CC(C)C)cc2)nn1C1CCCCO1]

Analogously to the process described under Example 14A/Step 1, 1.0 g (2.53 mmol) of the compound from Example 2A and 423 mg (2.53 mmol, purity 97%) of 4-isobutylbenzaldehyde gave 610 mg (69% of theory, purity 98%) of the title compound.

¹H NMR (400 MHz, CDCl₃, δ/ppm): 7.52 (d, 2H), 7.12 (d, 2H), 6.38 (d, 1H), 6.26 (s, 1H), 5.29 (dd, 1H), 4.10-4.03 (m, 1H), 3.70-3.62 (m, 1H), 2.46 (d, 2H), 2.56-2.43 (m, 1H), 2.37 (s, 3H), 2.17-2.10 (m, 1H), 2.02-1.94 (m, 1H), 1.93-1.81 (m, 1H), 1.78-1.65 (m, 2H), 1.65-1.59 (m, 1H), 0.91 (d, 6H).

LC/MS (Method 2, ESIpos): Rₜ=1.79 min, m/z=343 [M+H]⁺.

### Step 2: 3-[(Z)-1-Fluoro-2-(4-isobutylphenyl)vinyl]-5-methyl-1H-pyrazole

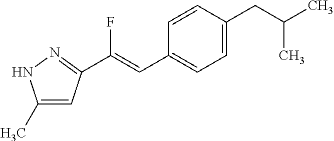 [SMILES: Cc1cc(/C(F)=C/c2ccc(CC(C)C)cc2)n[nH]1]

Analogously to the process described under Example 14A/Step 2, 593 mg (1.73 mmol) of the compound from Example 2A and 4.3 ml (17.3 mmol) of a 4 M solution of hydrogen chloride in dioxane gave 393 mg (85% of theory, purity 97%) of the title compound.

¹H NMR (400 MHz, CDCl₃, δ/ppm): 10.0 (very broad, 1H), 7.52 (d, 2H), 7.14 (d, 2H), 6.31 (d, 1H), 6.28 (s, 1H), 2.47 (d, 1H), 2.35 (s, 1H), 1.87 (m, 1H), 0.91 (d, 6H).

LC/MS (Method 5, ESIpos): Rₜ=1.23 min, m/z=259 [M+H]⁺.

### Example 16A

### 1,1,1,3,3,3-Hexafluoro-2-{4-[(Z)-2-fluoro-2-(5-methyl-1H-pyrazol-3-yl)vinyl]phenyl}propan-2-ol

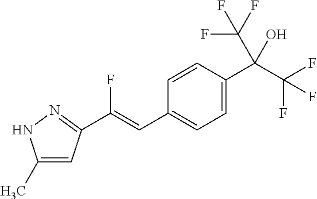 [SMILES: Cc1cc(/C(F)=C/c2ccc(C(O)(C(F)(F)F)C(F)(F)F)cc2)n[nH]1]

### Step 1: 1,1,1,3,3,3-Hexafluoro-2-[4-(hydroxymethyl)phenyl]propan-2-ol

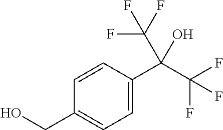 [SMILES: OCc1ccc(C(O)(C(F)(F)F)C(F)(F)F)cc1]

Under argon and at 0° C., 2.39 ml (5.73 mmol) of a 2.4 M lithium aluminium hydride solution in THF were added to a solution of 1.10 g (3.82 mmol) of 4-(2-hydroxyhexafluoroisopropyl)benzoic acid in 33 ml of THF. The mixture was stirred initially at 0° C. for 30 min and then at RT for 1.5 h. A further 0.7 ml (1.68 mmol) of the 2.4 M lithium aluminium hydride solution in THF was then added, and the mixture was stirred at RT for a further hour. The mixture was then heated at 75° C. for another 4.5 h. After cooling to RT, 10 ml of water were added slowly. Ethyl acetate was then added, and the mixture was washed with 5% strength aqueous citric acid. The aqueous phase was re-extracted once with ethyl acetate. The combined organic phases were washed with saturated sodium chloride solution, dried over magnesium sulphate, filtered and concentrated. The residue was dried under high vacuum. This gave 1.31 g (>100% of theory, purity about 93%) of the title compound.

¹H NMR (400 MHz, CDCl₃, δ/ppm): 7.72 (d, 2H), 7.46 (d, 2H), 4.76 (s, 2H), 4.12 (br. s, 1H).

LC/MS (Method 5, ESIneg): Rₜ=0.87 min, m/z=273 [M−H]⁻.

### Step 2: 4-(1,1,1,3,3,3-Hexafluoro-2-hydroxypropan-2-yl)benzaldehyde

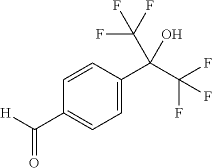 [SMILES: O=Cc1ccc(C(O)(C(F)(F)F)C(F)(F)F)cc1]

3.83 g (44.1 mmol) of manganese dioxide were added to a solution of 1.30 g (4.41 mmol, purity 93%) of the compound from Example 16A/Step 1 in 20 ml of a 1:1 mixture of dichloromethane and acetone. The mixture was stirred initially at RT for 3 h and then under reflux for 1 h. A further 3.83 g (44.1 mmol) of manganese dioxide were then added, and stirring of the mixture was continued overnight. After cooling to RT, the mixture was filtered through kieselguhr, and the solids that had been filtered off were washed with dichloromethane. Filtrate and wash solution were combined and concentrated, and the residue was dried under high vacuum. This gave 734 mg (61% of theory) of the title compound.

¹H NMR (400 MHz, CDCl₃, δ/ppm): 10.08 (s, 1H), 7.96 (m, 4H), 4.55 (br. s, 1H).

LC/MS (Method 5, ESIneg): Rₜ=0.96 min, m/z=271 [M−H]⁻.

### Step 3: 1,1,1,3,3,3-Hexafluoro-2-{4-[(Z)-2-fluoro-2-(5-methyl-1H-pyrazol-3-yl)vinyl]phenyl}propan-2-ol

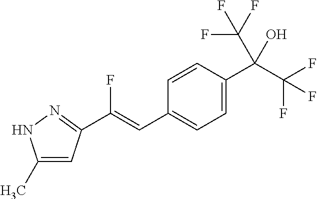 [SMILES: Cc1cc(/C(F)=C/c2ccc(C(O)(C(F)(F)F)C(F)(F)F)cc2)n[nH]1]

Analogously to the process described in Example 11A, 1.06 g (2.69 mmol) of the compound from Example 2A and 733 mg (2.69 mmol) of the compound from Example 16A/Step 2 gave 112 mg (11% of theory) of the title compound. In this case, the reaction time was 4 h (instead of 3 h). The crude product was purified by two column chromatographies (silica gel, mobile phase cyclohexane/ethyl acetate).

¹H NMR (400 MHz, CDCl₃, δ/ppm): 7.69 (m, 4H), 6.36 (d, 1H), 6.30 (s, 1H), 2.36 (s, 3H).

LC/MS (Method 2, ESIpos): Rₜ=1.22 min, m/z=369 [M+H]⁺.

### Example 17A

### 3-{(Z)-1-Fluoro-2-[4-(4-fluorotetrahydro-2H-pyran-4-yl)phenyl]vinyl}-5-methyl-1H-pyrazole

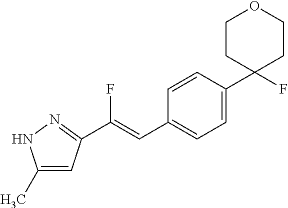 [SMILES: Cc1cc(/C(F)=C/c2ccc(C3(F)CCOCC3)cc2)n[nH]1]

### Step 1: 4-(4-Hydroxytetrahydro-2H-pyran-4-yl)benzonitrile

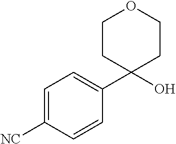 [SMILES: N#Cc1ccc(C2(O)CCOCC2)cc1]

At a temperature of −40° C., 109 ml (218 mmol) of a 2 M solution of isopropylmagnesium chloride in diethyl ether were added dropwise to a solution of 50.0 g (218 mmol) of 4-iodobenzonitrile in 1000 ml of anhydrous THF. After 1.5 h of stirring at the same temperature, a solution of 32.8 g (327 mmol) of tetrahydro-4H-pyran-4-one in 250 ml of anhydrous THF was quickly added dropwise at −40° C. After the addition had ended, the mixture was stirred at −40° C. for a further 10 min The temperature was then raised to 0° C. After a further 30 min, the cooling bath was finally removed and stirring was continued at RT. After 1 h, the reaction mixture was once more cooled to about −20° C., and about 500 ml of saturated aqueous ammonium chloride solution were added. Most of the THF was then removed on a rotary evaporator. The aqueous residue that remained was diluted with 1000 ml of water, and the mixture was extracted three times with in each case about 500 ml of dichloromethane. The combined organic extracts were dried over anhydrous magnesium sulphate, filtered, and freed from the solvent on a rotary evaporator. The crude product was then triturated with a mixture of diethyl ether, cyclohexane and ethyl acetate. Filtration and drying of the solid under high vacuum gave 19.3 g (44% of theory) of the title compound.

¹H NMR (400 MHz, CDCl₃, δ/ppm): 7.68 (d, 2H), 7.62 (d, 2H), 3.95-3.89 (m, 4H), 2.22-2.12 (m, 2H), 1.69 (s, 1H), 1.67-1.62 (m, 2H).

LC/MS (Method 2, ESIpos): Rₜ=0.71 min, m/z=204 [M+H]⁺.

### Step 2: 4-(4-Fluorotetrahydro-2H-pyran-4-yl)benzonitrile

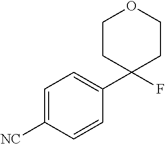 [SMILES: N#Cc1ccc(C2(F)CCOCC2)cc1]

Under inert conditions and at −78° C., a solution of 15.1 g (93.9 mmol) of diethylaminosulphur trifluoride (DAST) in 250 ml of dichloromethane was added dropwise to a suspension of 15.9 g (78.2 mmol) of the compound from Example 17A/Step 1 in 1000 ml of dichloromethane. After 30 min at −78° C., the reaction mixture was very quickly warmed to −20° to −10° C. using an ice/water bath and then stirred in this temperature range for 30 min. The cooling bath was then removed, and the mixture stirred at RT for 30 min and then once more cooled to about −20° C., and 400 ml of saturated aqueous sodium bicarbonate solution were added. After warming to RT, the mixture was diluted with about 500 ml of water and extracted twice with in each case about 200 ml of dichloromethane. The combined organic extracts were washed with water and dried over anhydrous magnesium sulphate. After filtration, the solvent was removed on a rotary evaporator. The crude product was triturated with 50 ml of ice-cold acetonitrile. Filtration and drying of the solid under high vacuum gave a first fraction (11.41 g) of the title compound. The mother liquor was evaporated to a residual volume of about 5-10 ml. This resulted in the precipitation of a second fraction of the title compound which was filtered off and dried under high vacuum (1.08 g). In total, this gave 12.5 g (78% of theory) of the title compound.

¹H NMR (400 MHz, CDCl₃, δ/ppm): 7.69 (d, 2H), 7.51 (d, 2H), 4.00-3.94 (m, 2H), 3.91-3.84 (m, 2H), 2.24-2.05 (m, 2H), 1.92-1.84 (m, 2H).

LC/MS (Method 2, ESIpos): Rₜ=1.01 min, m/z=206 [M+H]⁺.

### Step 3: 4-(4-Fluorotetrahydro-2H-pyran-4-yl)benzaldehyde

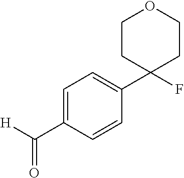 [SMILES: O=Cc1ccc(C2(F)CCOCC2)cc1]

At a temperature of −78° C., 15.3 ml (15.3 mmol) of a 1 M solution of diisobutylaluminium hydride in heptane were added dropwise to a solution of 3.0 g (14.6 mmol) of the compound from Example 17A/Step 2 in 17 ml of anhydrous THF. After 1 h at −78° C., the reaction was terminated by dropwise addition of 60 ml of 1 M hydrochloric acid. After warming to RT, the mixture was extracted three times with in each case about 50 ml of ethyl acetate. The combined organic extracts were washed successively with water and saturated sodium chloride solution. After drying over anhydrous magnesium sulphate and filtration, the solvent was removed on a rotary evaporator. The residue obtained was purified by MPLC (silica gel, mobile phase cyclohexane/ethyl acetate 5:1). After evaporation of the product fractions, the residue was triturated in a pentane/diethyl ether mixture. Filtration and drying of the solid under high vacuum gave 1.69 g (56% of theory) of the title compound.

¹H NMR (400 MHz, CDCl₃, δ/ppm): 10.03 (s, 1H), 7.61 (d, 2H), 7.57 (d, 2H), 4.00-3.94 (m, 2H), 3.93-3.85 (m, 2H), 2.28-2.09 (m, 2H), 1.95-1.87 (m, 2H).

LC/MS (Method 2, ESIpos): Rₜ=0.97 min, m/z=209 [M+H]⁺.

### Step 4: 3-{(Z)-1-Fluoro-2-[4-(4-fluorotetrahydro-2H-pyran-4-yl)phenyl]vinyl}-5-methyl-1H-pyrazole

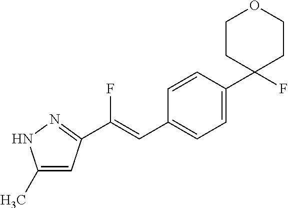 [SMILES: Cc1cc(/C(F)=C/c2ccc(C3(F)CCOCC3)cc2)n[nH]1]

Analogously to the process described under Example 3A, 2.0 g (5.05 mmol) of the compound from Example 2A and 1.05 g (5.05 mmol) of the compound from Example 17A/Step 3 gave 382 mg (25% of theory) of the title compound. Here, the reaction time in the first partial step of the reaction was 30 min (instead of 3 h). For the final MPLC, a mobile phase gradient of cyclohexane/ethyl acetate 10:1→1:1 was used.

¹H NMR (400 MHz, DMSO-d₆, δ/ppm): 7.55 (d, 2H), 7.49 (d, 2H), 6.44 (d, 1H), 6.32 (s, 1H), 3.82-3.67 (m, 4H), 2.25 (s, 3H), 2.01-1.91 (m, 2H), 1.56-1.50 (m, 2H).

LC/MS (Method 5, ESIpos): Rₜ=0.76 min, m/z=304 [M]⁺.

### Example 18A

### 3-{(Z)-1-Fluoro-2-[4-(pentafluoro-λ⁶-sulphanyl)phenyl]vinyl}-5-methyl-1H-pyrazole

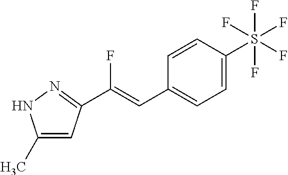 [SMILES: Cc1cc(/C(F)=C/c2ccc(S(F)(F)(F)(F)F)cc2)n[nH]1]

Analogously to the process described under Example 7A/Step 5, 1.50 g (3.79 mmol) of the compound from Example 2A and 880 mg (3.79 mmol) of 4-(pentafluoro-λ⁶-sulphanyl)-benzaldehyde gave 1.24 g (47% of theory, purity 97%) of the title compound. In this case, the reaction mixture was stirred at RT for 3 h (instead of 2 h). Here, Method 13 was used to purify the crude product.

¹H NMR (400 MHz, CDCl₃, δ/ppm): 9.5 (very broad, 1H), 7.73 (d, 2H), 7.66 (d, 2H), 6.39 (d, 1H), 6.33 (s, 1H), 2.37 (s, 3H).

LC/MS (Method 5, ESIpos): Rₜ=1.19 min, m/z=329 [M+H]⁺.

### Example 19A

### Methyl 2-({4-[(Z)-2-fluoro-2-(5-methyl-1H-pyrazol-3-yl)vinyl]phenyl}sulphanyl)-2-methyl-propanoate

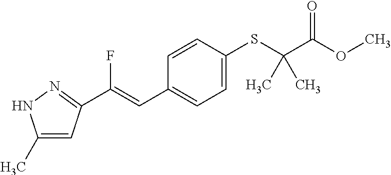

### Step 1: tert-Butyl 2-[(4-{(Z)-2-fluoro-2-[5-methyl-1-(tetrahydro-2H-pyran-2-yl)-1H-pyrazol-3-yl]vinyl}phenyl)sulphanyl]-2-methylpropanoate (racemate)

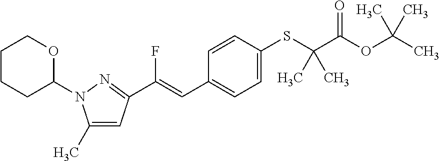 [SMILES: Cc1cc(/C(F)=C/c2ccc(SC(C)(C)C(=O)OC(C)(C)C)cc2)nn1C1CCCCO1]

At 0° C., 7.2 ml (7.19 mmol) of a 1 M lithium hexamethyldisilazide solution in THF were added to a solution of 1.18 g (3.0 mmol) of the compound from Example 2A and 1.40 g (3.0 mmol, purity 60%) of tert-butyl 2-[(4-formylphenyl)sulphanyl]-2-methylpropanoate [for the preparation, see WO 02/28821-A2, Example II-2] in 55 ml of THF. The mixture was stirred at 0° C. for 30 min. 100 ml of saturated aqueous ammonium chloride solution were then added, and after warming to RT the mixture was extracted twice with ethyl acetate. The combined organic phases were washed once with saturated sodium chloride solution, dried over magnesium sulphate, filtered and concentrated. The residue was purified by column chromatography (silica gel, mobile phase cyclohexane/ethyl acetate 9:1). Drying under high vacuum gave 937 mg (65% of theory, purity 95%) of the title compound.

¹H NMR (400 MHz, CDCl₃, δ/ppm): 7.54 (d, 2H), 7.47 (d, 2H), 6.41 (d, 1H), 6.28 (s, 1H), 5.30 (dd, 1H), 4.10-4.03 (m, 1H), 3.71-3.62 (m, 1H), 2.56-2.43 (m, 1H), 2.37 (s, 3H), 2.18-2.10 (m, 1H), 2.02-1.92 (m, 1H), 1.78-1.66 (m, 2H), 1.65-1.56 (m, 1H), 1.45 (s, 6H), 1.41 (s, 9H).

LC/MS (Method 6, ESIpos): Rₜ=3.14 min, m/z=461 [M+H]⁺.

### Step 2: 2-({4-[(Z)-2-Fluoro-2-(5-methyl-1H-pyrazol-3-yl)vinyl]phenyl}sulphanyl)-2-methylpropanoic acid

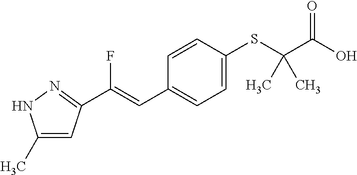 [SMILES: Cc1cc(/C(F)=C/c2ccc(SC(C)(C)C(=O)O)cc2)n[nH]1]

4.64 ml (18.56 mmol) of a 4 M solution of hydrogen chloride in dioxane were added to 900 mg (1.85 mmol, purity 95%) of the compound from Example 19A/Step 1, and the mixture was stirred at RT overnight. The solvent was then removed on a rotary evaporator and the residue was triturated with water. The solid that remained was filtered off, washed with water and dried under high vacuum. This gave 524 mg (76% of theory, purity 86%) of the title compound.

¹H NMR (400 MHz, CDCl₃, δ/ppm): 7.60-7.52 (m, 4H), 6.34 (s, 1H), 6.23 (d, 1H), 2.37 (s, 3H), 1.53 (s, 6H).

LC/MS (Method 5, ESIpos): Rₜ=0.99 min, m/z=321 [M+H]⁺.

### Step 3: Methyl 2-({4-[(Z)-2-fluoro-2-(5-methyl-1H-pyrazol-3-yl)vinyl]phenyl}sulphanyl)-2-methylpropanoate

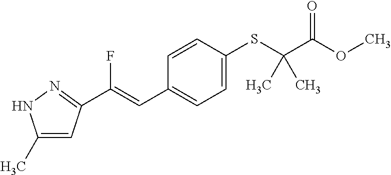

At 0° C., 180 μl (2.46 mmol) of thionyl chloride were added to a solution of 415 mg (1.23 mmol) of the compound from Example 19A/Step 2 in 5 ml of methanol, and the mixture was stirred at RT overnight. The solvent was then removed on a rotary evaporator and the residue was triturated with pentane. The solid that remained was filtered off, triturated twice with pentane and dried under high vacuum. This gave 399 mg (97% of theory) of the title compound.

¹H NMR (400 MHz, CDCl₃, δ/ppm): 7.65 (d, 2H), 7.47 (d, 2H), 7.09 (d, 1H), 6.51 (s, 1H), 3.69 (s, 3H), 2.60 (s, 3H), 1.51 (s, 6H).

LC/MS (Method 2, ESIpos): Rₜ=1.28 min, m/z=335 [M+H]⁺.

### Example 20A

### N-{4-[(Z)-2-Fluoro-2-(5-methyl-1H-pyrazol-3-yl)vinyl]benzyl}-N-isopropylpropane-2-amine

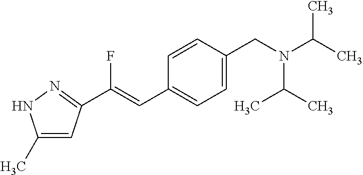 [SMILES: Cc1cc(/C(F)=C/c2ccc(CN(C(C)C)C(C)C)cc2)n[nH]1]

### Step 1: 3-{(Z)-2-[4-(Bromomethyl)phenyl]-1-fluorovinyl}-5-methyl-1-(tetrahydro-2H-pyran-2-yl)-1H-pyrazole (racemate)

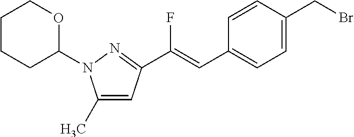 [SMILES: Cc1cc(/C(F)=C/c2ccc(CBr)cc2)nn1C1CCCCO1]

Under argon and at 0° C., 3.03 ml (3.03 mmol) of a 1 M lithium hexamethyldisilazide solution in THF were added to a solution of 500 mg (1.26 mmol) of the compound from Example 2A and 252 mg (1.26 mmol) of 4-(bromomethyl)benzaldehyde in 23 ml of THF. The mixture was stirred at 0° C. for 3 h. 100 ml of saturated aqueous ammonium chloride solution and 100 ml of ethyl acetate were then added. After phase separation, the organic phase was washed once with 100 ml of saturated sodium chloride solution, dried over sodium sulphate, filtered and concentrated. The residue was purified by column chromatography (silica gel, mobile phase cyclohexane/ethyl acetate 9:1). Drying under high vacuum gave 132 mg (28% of theory) of the title compound and 116 mg of a mixed fraction of the (E/Z) double bond isomers.

¹H NMR (400 MHz, CDCl₃, δ/ppm): 7.57 (d, 2H), 7.36 (d, 2H), 6.41 (d, 1H), 6.28 (s, 1H), 5.30 (dd, 1H), 4.50 (s, 2H), 4.10-4.03 (m, 1H), 3.70-3.62 (m, 1H), 2.55-2.43 (m, 1H), 2.18-2.10 (m, 1H), 2.01-1.94 (m, 1H), 1.80-1.66 (m, 2H), 1.65-1.59 (m, 1H).

LC/MS (Method 5, ESIpos): Rₜ=1.31 min, m/z=379/381 [M+H]⁺.

### Step 2: N-(4-{(Z)-2-Fluoro-2-[5-methyl-1-(tetrahydro-2H-pyran-2-yl)-1H-pyrazol-3-yl]vinyl}benzyl)-N-isopropylpropane-2-amine (racemate)

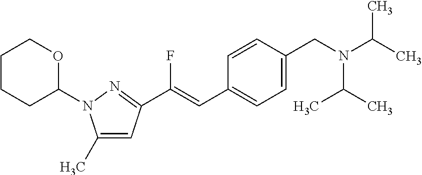 [SMILES: Cc1cc(/C(F)=C/c2ccc(CN(C(C)C)C(C)C)cc2)nn1C1CCCCO1]

A solution of 720 mg (1.90 mmol) of the compound from Example 20A/Step 1 and 798 μl (5.695 mmol) of diisopropylamine in 7.2 ml of toluene was heated in a microwave oven (Biotage Initiator with dynamic irradiation power control) at 150° C. for 1 h. After cooling to RT, the solid components were filtered off and washed once with ethyl acetate. Filtrate and wash solution were then combined and concentrated, and the residue was purified by preparative HPLC (Method 20). The combined product fractions were concentrated to a small residual volume on a rotary evaporator, saturated aqueous sodium bicarbonate solution was added and the mixture was extracted twice with ethyl acetate. The combined ethyl acetate phases were dried over magnesium sulphate, filtered and concentrated. The residue was dried under high vacuum. This gave 626 mg (83% of theory) of the title compound.

¹H NMR (400 MHz, CDCl₃, δ/ppm): 7.53 (d, 2H), 7.35 (d, 2H), 6.39 (d, 1H), 6.26 (s, 1H), 5.29 (dd, 1H), 4.10-4.03 (m, 1H), 3.70-3.60 (m, 3H), 3.02 (sept, 2H), 2.56-2.44 (m, 1H), 2.36 (s, 3H), 2.17-2.10 (m, 1H), 2.01-1.94 (m, 1H), 1.81-1.56 (m, 3H), 1.02 (d, 12H).

LC/MS (Method 5, ESIpos): Rₜ=0.88 min, m/z=400 [M+H]⁺.

### Step 3: N-{4-[(Z)-2-Fluoro-2-(5-methyl-1H-pyrazol-3-yl)vinyl]benzyl}-N-isopropylpropane-2-amine

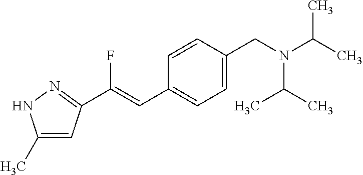 [SMILES: Cc1cc(/C(F)=C/c2ccc(CN(C(C)C)C(C)C)cc2)n[nH]1]

A solution of 730 mg (1.83 mmol) of the compound from Example 20A/Step 2 in 4.6 ml (18.3 mmol) of a 4 M solution of hydrogen chloride in dioxane was stirred at RT overnight. The mixture was then diluted with ethyl acetate and extracted twice with water. The combined aqueous phases were made slightly basic using sodium bicarbonate and extracted twice with ethyl acetate. The combined ethyl acetate phases were dried over magnesium sulphate, filtered and concentrated. The residue was triturated with pentane, and the solid that remained was filtered off and dried under high vacuum. This gave 446 mg (77% of theory) of the title compound.

¹H NMR (400 MHz, CDCl₃, δ/ppm): 7.53 (d, 2H), 7.37 (d, 2H), 6.30 (d, 1H), 6.28 (s, 1H), 3.64 (s, 2H), 3.07-2.97 (m, 2H), 2.35 (s, 3H), 1.02 (d, 12H).

LC/MS (Method 7, ESIpos): Rₜ=1.44 min, m/z=316 [M+H]⁺.

### Example 21A

### 4-{5-[(Z)-2-Fluoro-2-(5-methyl-1H-pyrazol-3-yl)vinyl]pyridin-2-yl}-2,6-dimethylmorpholine

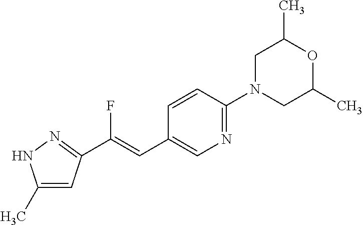

### Step 1: 4-(5-{(Z)-2-Fluoro-2-[5-methyl-1-(tetrahydro-2H-pyran-2-yl)-1H-pyrazol-3-yl]vinyl}pyridin-2-yl)-2,6-dimethylmorpholine (racemate)

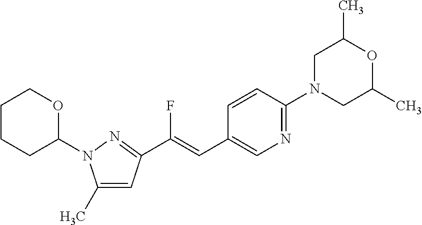 [SMILES: Cc1cc(/C(F)=C/c2ccc(N3CC(C)OC(C)C3)nc2)nn1C1CCCCO1]

Under argon, 1.0 g (4.54 mmol) of 6-(2,6-dimethylmorpholino)nicotinaldehyde was added to a solution of 1.80 g (4.54 mmol) of the compound from Example 2A in 75 ml of THF. With stirring, the mixture was cooled to 0° C. 10.9 ml (10.9 mmol) of a 1 M lithium hexamethyldisilazide solution in THF/ethylbenzene were then added slowly. With ice-cooling, stirring was continued for a further 30 min. 70 ml of saturated aqueous ammonium chloride solution and water were then added, and the mixture was extracted twice with ethyl acetate. The combined organic phases were washed once with saturated sodium chloride solution, dried over magnesium sulphate, filtered and concentrated. The residue was taken up in cyclohexane/ethyl acetate 8:2, which resulted in the precipitation of a solid which was filtered off and discarded. The filtrate was then purified by column chromatography (silica gel, mobile phase cyclohexane/ethyl acetate 8:2). The combined product fractions were concentrated and the residue was purified once more by preparative HPLC (Method 21). Drying under high vacuum gave 500 mg (26% of theory, purity 96%) of the title compound.

¹H NMR (400 MHz, CDCl₃, δ/ppm): 8.31 (d, 1H), 7.90 (dd, 1H), 6.64 (d, 1H), 6.30 (d, 1H), 6.25 (s, 1H), 5.29 (dd, 1H), 4.11-4.04 (m, 3H), 3.78-3.62 (m, 3H), 2.60-2.45 (m, 3H), 2.37 (s, 3H), 2.17-2.10 (m, 1H), 2.01-1.94 (m, 1H), 1.79-1.65 (m, 2H), 1.64-1.56 (m, 2H), 1.29 (s, 3H), 1.27 (s, 3H).

LC/MS (Method 5, ESIpos): Rₜ=1.08 min, m/z=401 [M+H]⁺.

### Step 2: 4-{5-[(Z)-2-Fluoro-2-(5-methyl-1H-pyrazol-3-yl)vinyl]pyridin-2-yl}-2,6-dimethylmorpholine

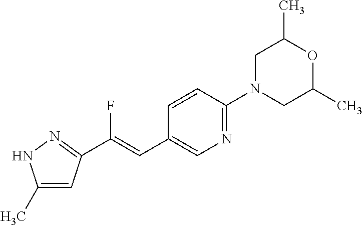

3.0 ml (12.0 mmol) of a 4 M solution of hydrogen chloride in dioxane were added to 481 mg (1.20 mmol) of the compound from Example 21A/Step 1, and the mixture was stirred at RT for 1 h. Ethyl acetate was then added, and the mixture was extracted once with saturated aqueous sodium bicarbonate solution. After phase separation, the aqueous phase was re-extracted once with ethyl acetate, and the combined organic phases were washed once with saturated sodium chloride solution, dried over magnesium sulphate, filtered and concentrated. The residue was dried under reduced pressure, and pentane was then added, resulting in the formation of a crystalline solid. The solid was filtered off, washed once with pentane and dried under high vacuum. This gave 330 mg (84% of theory, purity 97%) of the title compound.

¹H NMR (400 MHz, CDCl₃, δ/ppm): 8.31 (s, 1H), 7.88 (d, 1H), 6.64 (d, 1H), 6.26 (s, 1H), 6.16 (d, 1H), 4.09 (d, 2H), 3.72 (m, 2H), 2.56 (t, 2H), 2.35 (s, 3H), 1.29 (s, 3H), 1.27 (s, 3H).

LC/MS (Method 8, ESIpos): Rₜ=0.81 min, m/z=317 [M+H]⁺.

### Example 22A

### 4-{[tert-Butyl(diphenyl)silyl]oxy}piperidine

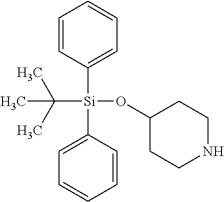 [SMILES: CC(C)(C)[Si](OC1CCNCC1)(c1ccccc1)c1ccccc1]

### Step 1: tert-Butyl 4-{[tert-butyl(diphenyl)silyl]oxy}piperidine-1-carboxylate

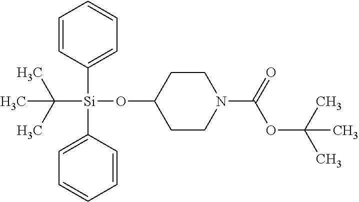 [SMILES: CC(C)(C)OC(=O)N1CCC(O[Si](c2ccccc2)(c2ccccc2)C(C)(C)C)CC1]

10.0 g (49.7 mmol) of tert-butyl 4-hydroxypiperidine-1-carboxylate and 4.06 g (59.7 mmol) of imidazole were initially charged in 100 ml of anhydrous DMF, and 15.02 g (54.7 mmol) of tert-butyl(diphenyl)silyl chloride were added at 0° C. The reaction mixture was stirred at RT for 48 h, then poured into 1.6 litres of water and subsequently extracted three times with in each case about 500 ml of diethyl ether. The combined organic extracts were washed successively with saturated sodium bicarbonate solution, water and saturated sodium chloride solution. The mixture was dried over anhydrous magnesium sulphate and then filtered, and the solvent was removed on a rotary evaporator. The residue that remained was subjected to coarse purification by filtration with suction (about 300 g of silica gel, mobile phase: cyclohexane cyclohexane/ethyl acetate 2:1). This gave 22.21 g (91% of theory at a purity of about 90%) of the title compound.

¹H NMR (400 MHz, CDCl₃, δ/ppm): 7.67 (d, 4H), 7.43-7.37 (m, 6H), 3.93-3.87 (m, 1H), 3.68-3.60 (m, 2H), 3.22-3.14 (m, 2H), 1.63-1.48 (m, 4H), 1.43 (s, 9H), 1.07 (s, 9H).

LC/MS (Method 5, ESIpos): Rₜ=1.68 min, m/z=440 [M+H]⁺.

### Step 2: 4-{[tert-Butyl(diphenyl)silyl]oxy}piperidine

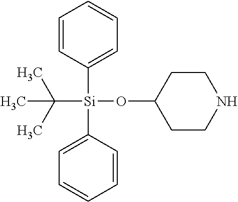 [SMILES: CC(C)(C)[Si](OC1CCNCC1)(c1ccccc1)c1ccccc1]

At RT, 10 ml of trifluoroacetic acid were added to a solution of 2.5 g (5.12 mmol, 90% pure) of the compound from Example 22A/Step 1 in 10 ml of dichloromethane. The reaction mixture was stirred at RT for 30 min, and 1 M aqueous sodium hydroxide solution was then added until the mixture gave an alkaline reaction. The mixture was extracted three times with in each case about 100 ml of ethyl acetate. The combined organic extracts were dried over anhydrous magnesium sulphate, filtered and concentrated to dryness on a rotary evaporator. The product was isolated by MPLC (about 50 g of silica gel, ethyl acetate ethyl acetate/triethylamine 9:1). Evaporation of the product fractions and drying under high vacuum gave 1.45 g (83% of theory) of the title compound.

¹H NMR (400 MHz, CDCl₃, δ/ppm): 7.68 (d, 4H), 7.45-7.35 (m, 6H), 3.83-3.77 (m, 1H), 3.07-3.01 (m, 2H), 2.52-2.47 (m, 2H), 1.72-1.66 (m, 2H), 1.53-1.45 (m, 2H), 1.07 (s, 9H).

LC/MS (Method 8, ESIpos): Rₜ=0.87 min, m/z=340 [M+H]⁺.

### Example 23A

### 3-{[tert-Butyl(diphenyl)silyl]oxy}azetidine

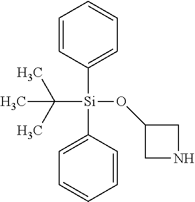 [SMILES: CC(C)(C)[Si](OC1CNC1)(c1ccccc1)c1ccccc1]

### Step 1: tert-Butyl 3-{[tert-butyl(diphenyl)silyl]oxy}azetidine-1-carboxylate

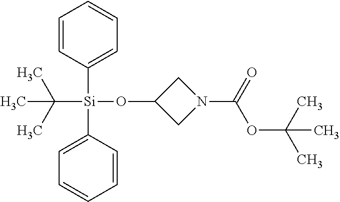 [SMILES: CC(C)(C)OC(=O)N1CC(O[Si](c2ccccc2)(c2ccccc2)C(C)(C)C)C1]

20.0 g (115 mmol) of tert-butyl 3-hydroxyazetidine-1-carboxylate and 9.43 g (139 mmol) of imidazole were initially charged in 200 ml of anhydrous DMF, and 34.91 g (127 mmol) of tert-butyl(diphenyl)silyl chloride were added at RT. After the reaction mixture had been stirred at RT for 18 h, it was poured into 3.2 litres of water and then extracted three times with in each case about 1 litre of diethyl ether. The combined organic extracts were washed successively with saturated aqueous sodium bicarbonate solution, water and saturated aqueous sodium chloride solution. After drying over anhydrous magnesium sulphate the mixture was filtered and the solvent was removed on a rotary evaporator. The residue that remained was triturated with 100 ml of pentane for a few minutes. The mixture was then filtered off with suction, the filtrate was discarded and the residue was dried under high vacuum. This gave 29.18 g (61% of theory) of the title compound.

¹H NMR (400 MHz, CDCl₃, δ/ppm): 7.60 (d, 4H), 7.46-7.37 (m, 6H), 4.53-4.49 (m, 1H), 3.93 (dd, 2H), 3.87 (dd, 2H), 1.41 (s, 9H), 1.04 (s, 9H).

LC/MS (Method 5, ESIpos): Rₜ=1.65 min, m/z=412 [M+H]⁺, 823 [2M+H]⁺.

### Step 2: 3-{[tert-Butyl(diphenyl)silyl]oxy}azetidine

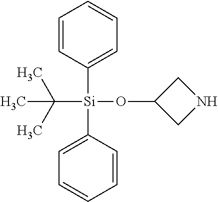 [SMILES: CC(C)(C)[Si](OC1CNC1)(c1ccccc1)c1ccccc1]

At RT, 70 ml of trifluoroacetic acid were added dropwise to a solution of 20.0 g (48.6 mmol) of the compound from Example 23A/Step 1 in 70 ml of dichloromethane. After the reaction mixture had been stirred at RT for 30 min, all volatile components were removed on a rotary evaporator. 1 litre of 1 M aqueous sodium hydroxide solution was added to the residue that remained, and the mixture was extracted three times with in each case about 200 ml of dichloromethane. The combined organic extracts were dried over anhydrous magnesium sulphate, filtered and concentrated to dryness on a rotary evaporator. Drying of the residue under high vacuum gave 14.85 g (98% of theory) of the title compound.

¹H NMR (400 MHz, CDCl₃, δ/ppm): 7.61 (d, 4H), 7.45-7.36 (m, 6H), 4.64-4.58 (m, 1H), 3.68 (dd, 2H), 3.53 (dd, 2H), 2.19 (broad, 1H), 1.03 (s, 9H).

LC/MS (Method 5, ESIpos): Rₜ=0.90 min, m/z=312 [M+H]⁺.

### Example 24A

### 1-(3-{[(Methylsulphonyl)oxy]methyl}phenyl)cyclopropyl acetate

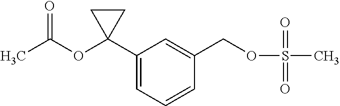 [SMILES: CC(=O)OC1(c2cccc(COS(C)(=O)=O)c2)CC1]

### Step 1: 1-[3-({[tert-Butyl(dimethyl)silyl]oxy}methyl)phenyl]cyclopropanol

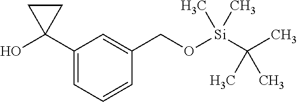 [SMILES: CC(C)(C)[Si](C)(C)OCc1cccc(C2(O)CC2)c1]

### Preparation of Solution A:

60 ml of methanol and a drop of concentrated hydrochloric acid were added to 12.32 g (70.7 mmol) of [(1-ethoxycyclopropyl)oxy](trimethyl)silane, and the mixture was stirred at RT overnight. The solvent was then removed on a rotary evaporator at RT and a pressure of not less than 30 mbar. This gave 6.26 g (61.27 mmol) of 1-ethoxycyclopropanol, which were dissolved in 80 ml of THF. Under argon, this solution was then cooled to −70° C., and 30.6 ml (61.27 mmol) of a 2 M solution of ethylmagnesium chloride in THF were added. The cooling bath was then removed, and the solution was stirred without cooling until an internal temperature of 0° C. had been reached.

### Preparation of Solution B:

Under argon and at −40° C., 47.1 ml (61.27 mmol) of a 1.3 M solution of isopropylmagnesium chloride/lithium chloride complex in THF were added to a solution of 19.40 g (55.70 mmol) of tert-butyl[(3-iodbenzyl)oxy]dimethylsilane in 280 ml of THF, and the mixture was stirred at −40° C. for 1 h.

After the two solutions had been prepared, solution A was added at 0° C. to solution B. The reaction mixture was then heated under reflux for 1 h. After cooling to RT, saturated aqueous ammonium chloride solution was added and the mixture was extracted twice with tert-butyl methyl ether. The combined organic phases were washed once with saturated aqueous sodium chloride solution, dried over magnesium sulphate, filtered and concentrated. The residue was purified by flash chromatography (silica gel, cyclohexane/ethyl acetate 100:0→85:15). Removal of the solvent gave 9.55 g (60% of theory, purity 97%) of the title compound.

¹H NMR (400 MHz, CDCl₃, δ/ppm): 7.32-7.24 (m, 2H), 7.22-7.16 (m, 2H), 4.74 (s, 2H), 2.36 (s, 1H), 1.26 (dd, 2H), 1.06 (dd, 2H), 0.94 (s, 9H), 0.10 (s, 6H).

MS (DCI, NH₃): m/z=296 [M+NH₄]⁺.

### Step 2: 1-[3-({[tert-Butyl(dimethyl)silyl]oxy}methyl)phenyl]cyclopropyl acetate

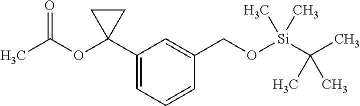 [SMILES: CC(=O)OC1(c2cccc(CO[Si](C)(C)C(C)(C)C)c2)CC1]

At RT, 3.81 g (42.87 mmol) of a 2 M solution of ethylmagnesium chloride in THF, directly followed by 3.0 ml (42.87 mmol) of acetyl chloride, were added to a solution of 9.55 g (34.3 mmol) of the compound from Example 24A/Step 1 in 100 ml of THF. After 5 min of stirring at RT, saturated aqueous ammonium chloride solution was added, and the mixture was then extracted twice with ethyl acetate. The combined organic phases were dried over magnesium sulphate, filtered and concentrated. This gave 11.25 g (96% of theory, purity 94%) of the title compound.

¹H NMR (400 MHz, CDCl₃, δ/ppm): 7.30-7.24 (m, 2H), 7.20-7.13 (m, 2H), 4.72 (s, 2H), 2.04 (s, 3H), 1.31-1.25 (m, 2H), 1.24-1.18 (m, 2H), 0.94 (s, 9H), 0.09 (s, 6H).

MS (DCI, NH₃): m/z=338 [M+NH₄]⁺.

### Step 3: 1-[3-(Hydroxymethyl)phenyl]cyclopropyl acetate

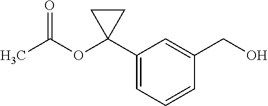

At RT, 65.6 ml (65.6 mmol) of a 1 M solution of tetra-n-butylammonium fluoride in THF were added to a solution of 11.25 g (32.82 mmol, purity 94%) of the compound from Example 24A/Step 2. The mixture was stirred at RT for 30 min and then diluted with ethyl acetate and washed once with water. The aqueous phase was re-extracted once with ethyl acetate. The combined organic phases were washed once with saturated aqueous sodium chloride solution, dried over magnesium sulphate and concentrated. This gave 8.0 g (95% of theory, purity 80%) of the title compound.

¹H NMR (400 MHz, CDCl₃, δ/ppm): 7.24-7.12 (m, 5H), 4.58 (s, 2H), 1.95 (s, 3H), 1.22-1.17 (m, 2H), 1.16-1.10 (m, 2H).

### Step 4: 1-(3-{[(Methylsulphonyl)oxy]methyl}phenyl)cyclopropyl acetate

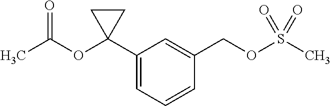 [SMILES: CC(=O)OC1(c2cccc(COS(C)(=O)=O)c2)CC1]

At 0° C., 2.8 ml (37.2 mmol) methanesulphonyl chloride were added dropwise to a solution of 8.0 g (31.0 mmol, purity 80%) of the compound from Example 24A/Step 3 and 5.6 ml (40.3 mmol) of triethylamine in 90 ml of THF. The mixture was then slowly warmed to RT, stirred at RT for a further 10 min and then diluted with ethyl acetate. The mixture was washed once with water and the aqueous phase was re-extracted once with ethyl acetate. The combined ethyl acetate phases were washed once with saturated aqueous sodium chloride solution, dried over magnesium sulphate and concentrated. The residue obtained was purified by flash chromatography (silica gel, cyclohexane/ethyl acetate 95:5→70:30). Removal of the solvent and drying under reduced pressure gave 8.45 g (91% of theory, purity 95%) of the title compound.

¹H NMR (400 MHz, CDCl₃, δ/ppm): 7.39-7.27 (m, 4H), 5.22 (s, 2H), 2.90 (s, 3H), 2.06 (s, 3H), 1.35-1.28 (m, 2H), 1.27-1.20 (m, 2H).

LC/MS (Method 2, ESIpos): Rₜ=1.02 min, m/z=285 [M+H]⁺.

### Example 25A

### 3-(2-Hydroxypropan-2-yl)benzylmethanesulphonate

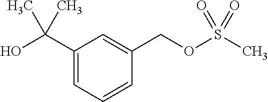 [SMILES: CC(C)(O)c1cccc(COS(C)(=O)=O)c1]

### Step 1: 2-[3-(Hydroxymethyl)phenyl]propan-2-ol

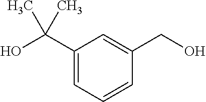

1.5 ml (3.47 mmol) of a 2.4 M solution of lithium aluminium hydride in THF were added slowly to a suspension of 500 mg (2.78 mmol) of 3-(2-hydroxypropan-2-yl)benzoic acid in 10 ml of THF. The mixture was then heated at a bath temperature of 80° C. for 2 h. After cooling to RT, 50 ml of 1 N hydrochloric acid were added and the mixture was extracted three times with in each case 30 ml of tert-butyl methyl ether. The combined organic phases were dried over sodium sulphate, filtered and concentrated. Drying of the residue under reduced pressure gave 455 mg (97% pure, 99% of theory) of the title compound.

¹H NMR (400 MHz, CDCl₃, δ/ppm): 7.51 (s, 1H), 7.42 (d, 1H), 7.34 (t, 1H), 7.25 (d, 1H), 4.71 (s, 2H), 1.59 (s, 6H).

LC/MS (Method 5, ESIpos): Rₜ=0.57 min, m/z=149 [M+H-H₂O]⁺.

### Step 2: 3-(2-Hydroxypropan-2-yl)benzylmethanesulphonate

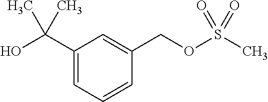 [SMILES: CC(C)(O)c1cccc(COS(C)(=O)=O)c1]

Under argon, 1.1 ml (7.88 mmol) of triethylamine were added at RT to a solution of 873 mg (5.25 mmol) of the compound from Example 25A/Step 1 in 50 ml of dichloromethane, followed by 1.01 g (5.78 mmol) methanesulphonic anhydride at 0° C. After 1 h of stirring at RT, the mixture was washed successively with 100 ml of aqueous ammonium chloride solution and 100 ml of sodium chloride solution. The organic phase was dried over magnesium sulphate, filtered and concentrated. Drying of the residue under reduced pressure gave 1.13 g (88% of theory) of the title compound.

¹H NMR (400 MHz, CDCl₃, δ/ppm): 7.56 (s, 1H), 7.51 (d, 1H), 7.39 (t, 1H), 7.32 (d, 1H), 5.25 (s, 2H), 2.94 (s, 3H), 1.59 (s, 6H).

LC/MS (Method 5, ESIpos): Rₜ=0.74 min, m/z=227 [M+H-H₂O]⁺.

### Example 26A

### 2-[3-(Bromomethyl)phenyl]propan-2-ol

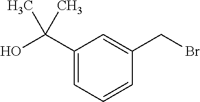

At at most 5° C., 456 μl (4.80 mmol) of phosphorus tribromide were added slowly to a solution of 665 mg (4.00 mmol) of the compound from Example 25A/Step 1 in 13 ml of toluene. After three hours of stirring at RT, the reaction mixture was poured into 30 ml of ice-water and extracted three times with in each case 20 ml of ethyl acetate. The combined organic phases were dried over magnesium sulphate, filtered and concentrated. Drying under reduced pressure gave 803 mg (about 53% of theory, purity about 60% according to ¹H NMR) of the title compound, which was used in this form in the subsequent reactions.

¹H NMR (400 MHz, CDCl₃, δ/ppm): 7.64-7.61 (m, 1H), 7.59-7.53 (m, 1H), 7.35-7.30 (m, 2H), 4.52 (s, 2H), 2.20 (s, 6H).

GC/MS (Method 10): Rₜ=4.42 min, m/z=210/212 [M−H₂O]⁺.

### Example 27A

### 3-(1-{[(Triisopropylsilyl)oxy]methyl}cyclopropyl)benzylmethanesulphonate

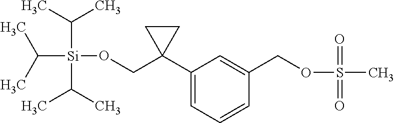 [SMILES: CC(C)[Si](OCC1(c2cccc(COS(C)(=O)=O)c2)CC1)(C(C)C)C(C)C]

### Step 1: Methyl 1-(3-bromophenyl)cyclopropanecarboxylate

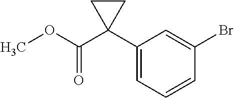

At 0° C., 48 ml (48.0 mmol) of a 1 M solution of lithium hexamethyldisilazide (LiHMDS) in THF were added to a solution of 10.0 g (43.6 mmol) of methyl (3-bromophenyl)acetate in 250 ml of anhydrous THF. After 15 min at 0° C., 4.9 ml (56.7 mmol) of 1,2-dibromoethane were added. The ice/water bath was removed, and the mixture was stirred at RT for another 1 h. The mixture was then once more cooled to 0° C., and a further 48 ml (48.0 mmol) of the LiHMDS solution were added. After the addition had ended, the mixture was stirred at RT for 63 h. About 250 ml of saturated aqueous ammonium chloride solution were then added, and the reaction mixture was extracted three times with in each case about 200 ml of ethyl acetate. The combined organic extracts were washed successively with water and saturated aqueous sodium chloride solution, dried over anhydrous magnesium sulphate, filtered and finally freed from the solvent under reduced pressure. The residue obtained was purified by filtration with suction on silica gel using the mobile phase cyclohexane/ethyl acetate 20:1. This gave 6.24 g (56% of theory) of the title compound.

¹H NMR (400 MHz, CDCl₃, δ/ppm): 7.50 (m, 1H), 7.39 (m, 1H), 7.27 (m, 1H, partially obscured by the CHCl₃ signal), 7.19 (m, 1H), 3.63 (s, 3H), 1.62-1.60 (m, 2H), 1.20-1.17 (m, 2H).

GC/MS (Method 10, EIpos): Rₜ=5.27 min, m/z=254/256 [M]⁺.

### Step 2: [1-(3-Bromophenyl)cyclopropyl]methanol

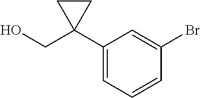 [SMILES: OCC1(c2cccc(Br)c2)CC1]

At −78° C., 13.7 ml (13.7 mmol) of a 1 M solution of lithium aluminium hydride in THF were added to a solution of 3.50 g (13.7 mmol) of the compound from Example 27A/Step 1 in 70 ml of anhydrous THF. After 1 h, about 3 ml of saturated aqueous ammonium chloride solution were added and the reaction mixture was allowed to warm to RT. The mixture was then diluted with about 80 ml of ethyl acetate, and subsequently anhydrous magnesium sulphate was added in the amount required for complete absorption of the aqueous phase. The mixture was filtered and then concentrated, and the residue was purified by MPLC (silica gel, cyclohexane→cyclohexane/ethyl acetate 5:1). This gave 1.37 g (44% of theory) of the title compound.

¹H NMR (400 MHz, CDCl₃, δ/ppm): 7.52 (s, 1H), 7.36 (d, 1H), 7.29 (d, 1H), 7.18 (t, 1H), 3.66 (d, 2H), 1.44 (t, 1H), 0.91-0.84 (m, 4H).

GC/MS (Method 10, EIpos): Rₜ=5.26 min, m/z=226/228 [M]⁺.

### Step 3: {[1-(3-Bromophenyl)cyclopropyl]methoxy}(triisopropyl)silane

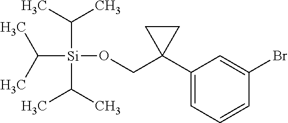 [SMILES: CC(C)[Si](OCC1(c2cccc(Br)c2)CC1)(C(C)C)C(C)C]

At about −50° C., 1.55 ml (6.19 mmol) of triisopropylsilyl triflate were added to a solution of 1.34 g (5.90 mmol) of the compound from Example 27A/Step 2 and 948 mg (8.85 mmol) of 2,6-dimethylpyridine in 25 ml of anhydrous dichloromethane. After 30 min, the cooling bath was removed and stirring was continued at RT for 1 h. About 50 ml of water were then added, and the mixture was extracted three times with in each case about 50 ml of ethyl acetate. The combined organic extracts were washed with saturated aqueous sodium chloride solution, dried over anhydrous magnesium sulphate, filtered and finally freed from the solvent under reduced pressure. The residue obtained was purified by MPLC (silica gel, cyclohexane/ethyl acetate 5:1). This gave 1.93 g (85% of theory) of the title compound.

¹H NMR (400 MHz, CDCl₃, δ/ppm): 7.52 (s, 1H), 7.31 (d, 1H), 7.27 (d, 1H, partially obscured by the CHCl₃ signal), 7.13 (t, 1H), 3.74 (s, 2H), 1.02 (m, 3H), 0.99 (d, 18H), 0.91-0.89 (m, 2H), 0.78-0.75 (m, 2H).

GC/MS (Method 10, EIpos): Rₜ=6.87 min, m/z=339/341 [M-ⁱPr]⁺.

### Step 4: 3-(1-{[(Triisopropylsilyl)oxy]methyl}cyclopropyl)benzaldehyde

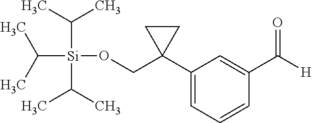 [SMILES: CC(C)[Si](OCC1(c2cccc(C=O)c2)CC1)(C(C)C)C(C)C]

At −78° C., 6.3 ml (10.0 mmol) of an n-butyllithium solution (1.6 M in hexane) were added dropwise to a solution of 1.92 g (5.01 mmol) of the compound from Example 27A/Step 3 in 50 ml of anhydrous THF. After the addition had ended, the mixture was stirred at the same temperature for another 50 min and then, likewise at −78° C., 1.2 ml (15.0 mmol) of anhydrous DMF were added. The cooling bath was then removed, and stirring was continued at RT for 1 h. About 100 ml of saturated aqueous ammonium chloride solution were then added, and the mixture was extracted three times with in each case about 100 ml of ethyl acetate. The combined organic extracts were washed successively with water and saturated sodium chloride solution, dried over anhydrous magnesium sulphate, filtered and finally freed from the solvent under reduced pressure. The crude product obtained was purified by MPLC (silica gel, cyclohexane/ethyl acetate 10:1). This gave 1.48 g (89% of theory) of the title compound.

¹H NMR (400 MHz, CDCl₃, δ/ppm): 10.00 (s, 1H), 7.89 (s, 1H), 7.72 (d, 1H), 7.65 (d, 1H), 7.43 (t, 1H), 3.79 (s, 2H), 1.01 (sept, 3H), 0.98 (d, 18H), 0.96-0.94 (m, 2H), 0.83-0.81 (m, 2H).

GC/MS (Method 10, EIpos): Rₜ=7.00 min, m/z=289 [M-ⁱPr]⁺.

### Step 5: [3-(1-{[(Triisopropylsilyl)oxy]methyl}cyclopropyl)phenyl]methanol

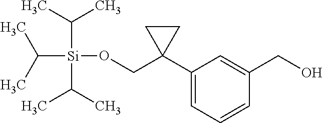 [SMILES: CC(C)[Si](OCC1(c2cccc(CO)c2)CC1)(C(C)C)C(C)C]

At −78° C., 4.2 ml (4.21 mmol) of a 1 M solution of lithium aluminium hydride in THF were added to a solution of 1.40 g (4.21 mmol) of the compound from Example 27A/Step 4 in 25 ml of anhydrous THF. After the addition had ended, the cooling bath was removed and the reaction mixture was stirred at RT for 1 h. About 5 ml of saturated aqueous ammonium chloride solution were then added carefully. The mixture was then diluted with about 25 ml of ethyl acetate, and subsequently anhydrous magnesium sulphate was added in the amount required for complete absorption of the aqueous phase. The mixture was filtered and then concentrated, and the residue was purified by MPLC (silica gel, cyclohexane/ethyl acetate 10:1). This gave 1.10 g (78% of theory) of the title compound.

¹H NMR (400 MHz, CDCl₃, δ/ppm): 7.38 (s, 1H), 7.31-7.25 (m, 2H, partially obscured by the CHCl₃ signal), 7.20 (d, 1H), 4.67 (d, 2H), 3.79 (s, 2H), 1.60 (t, 1H), 1.02 (sept, 3H), 1.00 (d, 18H), 0.93-0.90 (m, 2H), 0.77-0.75 (m, 2H).

GC/MS (Method 10, EIpos): Rₜ=7.18 min, m/z=291 [M-ⁱPr]⁺.

### Step 6: 3-(1-{[(Triisopropylsilyl)oxy]methyl}cyclopropyl)benzylmethanesulphonate

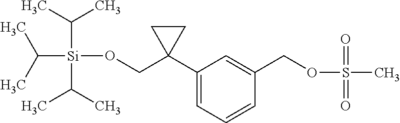 [SMILES: CC(C)[Si](OCC1(c2cccc(COS(C)(=O)=O)c2)CC1)(C(C)C)C(C)C]

At 0° C., 470 mg (2.70 mmol) of methanesulphonic anhydride were added to a solution of 820 mg (2.45 mmol) of the compound from Example 27A/Step 5 and 512 μl (3.68 mmol) of triethylamine in 25 ml of anhydrous dichloromethane. The cooling bath was removed, and the mixture was stirred at RT for another 1 h. The reaction mixture was then transferred into a separating funnel and, in succession, quickly washed with semisaturated aqueous ammonium chloride solution and saturated aqueous sodium chloride solution. After drying over anhydrous magnesium sulphate, the mixture was filtered and the filtrate was freed from the solvent on a rotary evaporator. This gave 1.01 g (100% of theory) of the title compound.

¹H NMR (400 MHz, CDCl₃, δ/ppm): 7.42 (s, 1H), 7.40 (d, 1H), 7.32 (t, 1H), 7.25 (d, 1H, partially obscured by the CHCl₃ signal), 5.21 (s, 2H), 3.77 (s, 2H), 2.91 (s, 3H), 1.02 (sept, 3H), 0.98 (d, 18H), 0.93-0.91 (m, 2H), 0.79-0.76 (m, 2H).

LC/MS (Method 5, ESIpos): Rₜ=1.59 min, m/z=413 [M+H]⁺.

MS (DCI, NH₃): m/z=430 [M+NH₄]⁺.

### Example 28A

### 2-[3-(Bromomethyl)phenyl]-2,2-difluoroethanol

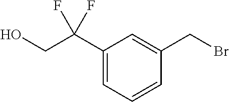 [SMILES: OCC(F)(F)c1cccc(CBr)c1]

### Step 1: Ethyl difluoro(3-methylphenyl)acetate

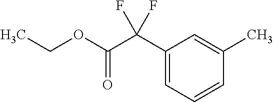

At RT and under argon, 25.0 g (123 mmol) of bromodifluoroethyl acetate and 41.0 g (225 mmol) of copper bronze (Cu/Sn alloy) were added to a solution of 23.35 g (107 mmol) of 3-iodotoluene in 110 ml of DMSO. The reaction mixture was then stirred at 50° C. for 16 h. After cooling to RT, the mixture was introduced into 200 ml of 1 M hydrochloric acid and diluted with 100 ml of ethyl acetate. Any solids present were filtered off and washed twice with in each case 50 ml of 1 M hydrochloric acid and ethyl acetate. The ethyl acetate phases were combined, washed in each case once with 200 ml of water and 200 ml of saturated sodium chloride solution, dried over sodium sulphate, filtered and concentrated. The residue was purified by column chromatography (silica gel, isohexane/ethyl acetate 98:2→90:10). Removal of the solvent gave 21.41 g (54% of theory) of the title compound.

¹H NMR (400 MHz, CDCl₃, δ/ppm): 7.43-7.38 (m, 2H), 7.37-7.21 (m, 2H), 4.30 (quart, 2H), 2.40 (s, 3H), 1.31 (t, 3H).

GC/MS (Method 10, EIpos): Rₜ=3.72 min, m/z=214 [M]⁺.

### Step 2: 2,2-Difluoro-2-(3-methylphenyl)ethanol

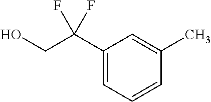

At RT and under argon, 1.51 g (40 mmol) of sodium borohydride were added in small portions to a solution of 8.57 g (40.0 mmol) of the compound from Example 28A/Step 1 in 70 ml of ethanol. After 30 min of stirring at RT, 300 ml of tert-butyl methyl ether and 300 ml of 1 M hydrochloric acid were added slowly to the reaction mixture, and the aqueous phase was then extracted once with 200 ml of tert-butyl methyl ether. The combined organic phases were dried over sodium sulphate, filtered and concentrated on a rotary evaporator at RT and a reduced pressure which was just sufficient. This gave 7.17 g (>100% of theory) of a residue which contained the title compound and residual solvent.

¹H NMR (400 MHz, CDCl₃, δ/ppm): 7.35-7.24 (m, 3H), 3.96 (t, 2H), 2.40 (s, 3H).

GC/MS (Method 10, EIpos): Rₜ=3.32 min, m/z=172 [M]⁺.

### Step 3: 2-[3-(Bromomethyl)phenyl]-2,2-difluoroethanol

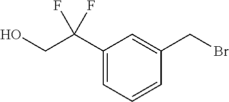 [SMILES: OCC(F)(F)c1cccc(CBr)c1]

At RT, 7.47 g (42.0 mmol) of N-bromosuccinimide and 328 mg (2.00 mmol) of 2,2′-azobis-2-methylpropanenitrile (AIBN) were added to a solution of 6.88 g (about 40 mmol, still comprising solvent) of the compound from Example 28A/Step 2 in 150 ml of acetonitrile. The mixture was heated at a bath temperature of 80° C. for 6 h. After cooling to RT, the solvent was removed and the residue was triturated with a mixture of 100 ml of pentane and 50 ml of ethyl acetate. The solid that remained was filtered off and washed twice with 15 ml of the 2:1 mixture of pentane and ethyl acetate. The filtrate and the wash solution were combined, washed in each case once with 200 ml of saturated aqueous sodium sulphite solution and 200 ml of saturated aqueous sodium chloride solution, dried over sodium sulphate, filtered and finally concentrated. This gave 9.72 g (68% of theory, purity 70%) of the title compound.

¹H NMR (400 MHz, CDCl₃, δ/ppm): 7.56-7.42 (m, 4H), 4.51 (s, 2H), 3.98 (m, 2H).

GC/MS (Method 10, EIpos): Rₜ=5.06 min, m/z=250 [M]⁺.

### Example 29A

### 3-(2-Hydroxy-2-methylpropyl)benzylmethanesulphonate

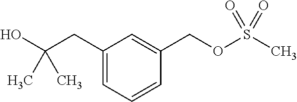 [SMILES: CC(C)(O)Cc1cccc(COS(C)(=O)=O)c1]

### Step 1: 1-(3-Bromophenyl)-2-methylpropan-2-ol

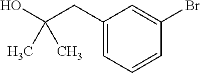

At 0° C., 55 ml (164 mmol) of a 3 M solution of methylmagnesium chloride in THF were added dropwise to a solution of 15.0 g (65.5 mmol) of methyl (3-bromophenyl)acetate in 600 ml of anhydrous THF. After the addition had ended, the mixture was stirred at the same temperature for another 1 h. The ice/water bath was then removed, and stirring was continued overnight at RT. About 1.2 litres saturated aqueous ammonium chloride solution were then added, and the mixture was extracted three times with in each case about 200 ml of ethyl acetate. The combined organic extracts were washed with saturated sodium chloride solution, dried over anhydrous magnesium sulphate, filtered and finally freed from the solvent under reduced pressure. The residue obtained was purified by filtration with suction on silica gel using the mobile phase cyclohexane/ethyl acetate 10:1→1:1. This gave 8.04 g (53% of theory, 98% pure) of the title compound.

¹H NMR (400 MHz, CDCl₃, δ/ppm): 7.41-7.37 (m, 2H), 7.20-7.13 (m, 2H), 2.73 (s, 2H), 1.32 (s, 1H), 1.23 (s, 6H).

GC/MS (Method 10, EIpos): Rₜ=4.56 min, m/z=210/212 [M−H₂O]⁺.

### Step 2: 3-(2-Hydroxy-2-methylpropyl)benzaldehyde

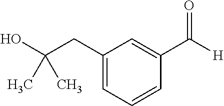

At −78° C., 13.7 ml (21.8 mmol) of n-butyllithium solution (1.6 M in hexane) were added dropwise to a solution of 2.50 g (10.9 mmol) of the compound from Example 29A/Step 1 in 100 ml of anhydrous THF. After the addition had ended, the mixture was stirred at the same temperature for another 30 min after which, likewise at −78° C., 2.6 ml (32.8 mmol) of anhydrous DMF were added. The cooling bath was then removed, and stirring was continued overnight at RT. About 100 ml of saturated aqueous ammonium chloride solution were then added, and the mixture was extracted three times with in each case about 100 ml of ethyl acetate. The combined organic extracts were washed successively with water and saturated sodium chloride solution, dried over anhydrous magnesium sulphate, filtered and finally freed from the solvent under reduced pressure. The crude product obtained in this manner was purified by MPLC (silica gel, cyclohexane/ethyl acetate 2:1). This gave 1.15 g (59% of theory) of the title compound.

¹H NMR (400 MHz, CDCl₃, δ/ppm): 10.01 (s, 1H), 7.79-7.74 (m, 2H), 7.53-7.47 (m, 2H), 2.86 (s, 2H), 1.25 (s, 6H).

GC/MS (Method 10, EIpos): Rₜ=4.76 min, m/z=160 [M−H₂O]⁺.

### Step 3: 1-[3-(Hydroxymethyl)phenyl]-2-methylpropan-2-ol

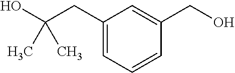

At 0° C., 6.0 ml (6.0 mmol) lithium aluminium hydride solution (1.0 M in THF) were added dropwise to a solution of 1.07 g (6.00 mmol) of the compound from Example 29A/Step 2 in 30 ml of anhydrous THF. After the addition had ended, the mixture was stirred at RT for another 1 h. 1-2 ml of saturated aqueous ammonium chloride solution were then added carefully, followed by about 30 ml of ethyl acetate. Anhydrous magnesium sulphate was added in the amount required for complete absorption of the aqueous phase. After filtration, the filtrate was freed from the solvent on a rotary evaporator and the residue dried under high vacuum. This gave 1.09 g (100% of theory) of the title compound.

¹H NMR (400 MHz, CDCl₃, δ/ppm): 7.31 (t, 1H), 7.25 (dd, 1H, partially obscured by the CHCl₃ signal), 7.22 (dd, 1H), 7.14 (dd, 1H), 4.69 (s, broad, 2H), 2.78 (s, 2H), 1.79 (broad, 1H), 1.41 (s, broad, 1H), 1.23 (s, 6H).

GC/MS (Method 10, EIpos): Rₜ=5.00 min, m/z=162 [M−H₂O]⁺.

### Step 4: 3-(2-Hydroxy-2-methylpropyl)benzyl methanesulphonate

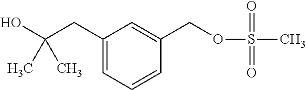 [SMILES: CC(C)(O)Cc1cccc(COS(C)(=O)=O)c1]

At 0° C., 1.12 g (6.41 mmol) of methanesulphonic anhydride were added to a solution of 1.05 g (5.83 mmol) of the compound from Example 29A/Step 3 and 1.2 ml (8.74 mmol) of triethylamine in 60 ml of anhydrous dichloromethane. The mixture was stirred at RT for another 1 h. The reaction mixture was then transferred into a separating funnel and, in succession, quickly washed with semisaturated aqueous ammonium chloride solution and saturated aqueous sodium chloride solution. After drying over anhydrous magnesium sulphate, the mixture was filtered and the filtrate was freed from the solvent on a rotary evaporator. This gave 1.5 g (99% of theory) of the title compound.

MS (DCI, NH₃): m/z=276 [M+NH₄]⁺.

### Example 30A

### (6-Fluoropyridin-3-yl)methyl methanesulphonate

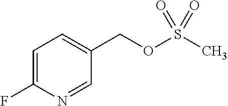

At 0° C., 3.4 ml (43.4 mmol) of methanesulphonyl chloride were added slowly to a solution of 4.60 g (36.2 mmol) of (6-fluoropyridin-3-yl)methanol and 6.6 ml (47.0 mmol) of triethylamine in 100 ml of THF. The cooling bath was removed and the mixture was stirred at RT for 5 min. Water, saturated aqueous sodium bicarbonate solution and ethyl acetate were then added to the mixture. After phase separation, the aqueous phase was extracted once with ethyl acetate. The combined organic phases were washed once with saturated sodium chloride solution, dried over magnesium sulphate, filtered and concentrated. This gave 7.44 g (93% of theory, purity 93%) of the title compound.

¹H NMR (400 MHz, CDCl₃, δ/ppm): 8.29 (d, 1H), 7.91 (td, 1H), 7.01 (dd, 1H), 5.25 (s, 2H), 3.04 (s, 3H).

LC/MS (Method 5, ESIpos): Rₜ=0.53 min, m/z=206 [M+H]⁺.

### Example 31A

### 5-(Chloromethyl)-N-(3,4-dimethoxybenzyl)-N-methylpyridine-2-amine dihydrochloride

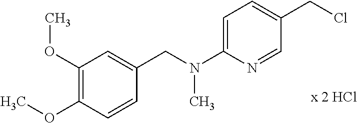 [SMILES: COc1ccc(CN(C)c2ccc(CCl)cn2)cc1OC]

### Step 1: 6-[(3,4-Dimethoxybenzyl)(methyl)amino]nicotinic acid

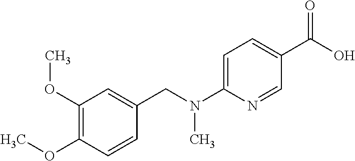 [SMILES: COc1ccc(CN(C)c2ccc(C(=O)O)cn2)cc1OC]

With stirring, a mixture of 5.0 g (31.7 mmol) of 6-chloronicotinic acid and 15.1 ml (79.4 mmol) of 3,4-dimethoxy-N-methylbenzylamine was heated at 150° C. overnight. After cooling to RT, 300 ml of ethyl acetate and 600 ml of water were added. The solid formed was removed during phase separation and dried under reduced pressure. This gave 7.38 g (77% of theory) of the title compound.

¹H NMR (400 MHz, CDCl₃, δ/ppm): 8.91 (d, 1H), 8.07-8.02 (dd, 1H), 6.81 (d, 1H), 6.78-6.73 (m, 2H), 6.52 (d, 1H), 4.82 (d, 2H), 3.86 (s, 3H), 3.82 (s, 3H), 3.12 (s, 3H).

LC/MS (Method 5, ESIpos): Rₜ=0.74 min, m/z=303 [M+H]⁺.

### Step 2: {6-[(3,4-Dimethoxybenzyl)(methyl)amino]pyridin-3-yl}methanol

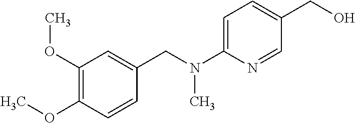 [SMILES: COc1ccc(CN(C)c2ccc(CO)cn2)cc1OC]

At 0° C. and under argon, 7.38 g (24.4 mmol) of the compound from Example 31A/Step 1 were initially charged in 225 ml of THF, 20.3 ml (48.8 mmol) of a 2.4 M solution of lithium aluminium hydride in THF were slowly added dropwise and the mixture was then stirred at RT for 2 h. With ice-cooling, 2 ml of water and 2 ml of 15% strength aqueous sodium hydroxide solution were then added slowly. The mixture was diluted with 200 ml of tert-butyl methyl ether, and the solid present was filtered off and washed three times with in each case 100 ml of tert-butyl methyl ether. Filtrate and wash solutions were combined and concentrated, and the residue obtained was dried under reduced pressure. This gave 6.20 g (87% of theory) of the title compound.

¹H NMR (400 MHz, CDCl₃, δ/ppm): 8.15 (d, 1H), 7.51-7.48 (dd, 1H), 6.81-6.72 (m, 3H), 6.52 (d, 1H), 4.72 (s, 2H), 4.54 (d, 2H), 3.85 (s, 3H), 3.82 (s, 3H), 3.05 (s, 3H), 1.65-1.60 (m, 1H).

LC/MS (Method 5, ESIpos): Rₜ=0.48 min, m/z=289 [M+H]⁺.

### Step 3: 5-(Chloromethyl)-N-(3,4-dimethoxybenzyl)-N-methylpyridine-2-amine dihydrochloride

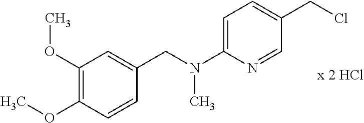 [SMILES: COc1ccc(CN(C)c2ccc(CCl)cn2)cc1OC]

At RT, 1.8 ml (24.5 mmol) of thionyl chloride were added to a solution of 3.54 g (12.3 mmol) of the compound from Example 31A/Step 2 in 22 ml of dichloromethane, and the mixture was stirred at this temperature for 2 h. The reaction was then concentrated and the residue was dried under reduced pressure. This gave 4.64 g (99% of theory) of the title compound.

¹H NMR (400 MHz, CDCl₃, δ/ppm): 15.7 (s, broad, 1H), 8.31 (s, 1H), 7.85 (d, 1H), 6.90 (d, 1H), 6.84 (d, 1H), 6.80-6.72 (m, 2H), 4.84 (s, 2H), 4.49 (s, 2H), 3.88 (s, 6H), 3.55 (s, 3H).

LC/MS (Method 6, ESIpos): Rₜ=1.05 min, m/z=289/291 [M+H]⁺.

### Example 32A

### 1-[4-(Chloromethyl)pyridin-2-yl]-4-cyclopropylpiperazine

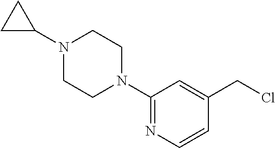 [SMILES: ClCc1ccnc(N2CCN(C3CC3)CC2)c1]

### Step 1: [2-(piperazin-1-yl)pyridin-4-yl]methanol

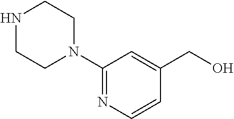 [SMILES: OCc1ccnc(N2CCNCC2)c1]

Under argon, 120 g (1.39 mol) of piperazine were added to 10.0 g (69.6 mmol) of (2-chloropyridin-4-yl)methanol. With stirring, the mixture was heated at 150° C. overnight. After cooling to RT, the excess piperazine which had formed a deposit in the upper part of the reaction vessel was removed, and the resinous content of the flask was taken up in 700 ml of dichloromethane and stirred at RT for 30 min. The solid formed was filtered off, washed with dichloromethane and discarded, and the filtrate was concentrated. The residue was dried under reduced pressure. This gave 13.3 g (about 99% of theory) of the title compound which, according to ¹H NMR, still contained piperazine.

¹H NMR (400 MHz, CDCl₃, δ/ppm): 8.14 (d, 1H), 6.67 (s, 1H), 6.58 (d, 1H), 4.64 (s, 2H), 3.55-3.45 (m, 4H), 3.01-2.94 (m, 4H).

LC/MS (Method 6, ESIpos): Rₜ=0.19 min, m/z=194 [M+H]⁺.

### Step 2: [2-(4-Cyclopropylpiperazin-1-yl)pyridin-4-yl]methanol

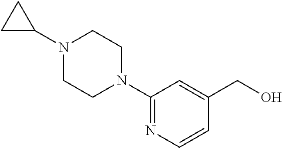 [SMILES: OCc1ccnc(N2CCN(C3CC3)CC2)c1]

13.1 g (67.9 mmol) of the compound from Example 32A/Step 1 were dissolved in a mixture of 535 ml of methanol and 39 ml (679 mmol) of acetic acid. 9.2 g of molecular sieve (3 Å) and 82 ml (407 mmol) of [(1-ethoxycyclopropyl)oxy](trimethyl)silane were added. After 10 min of stirring at RT, 12.8 g (203 mmol) sodium cyanoborohydride were added and, with stiffing, the mixture was heated at reflux for 2 h. After cooling to RT, the solid formed was filtered off and washed twice with in each case 20 ml of methanol. The filtrate was concentrated and the residue was taken up in 550 ml of dichloromethane. The mixture was washed twice with in each case 500 ml of saturated aqueous sodium bicarbonate solution and once with 500 ml of saturated aqueous sodium chloride solution, dried over magnesium sulphate, filtered and concentrated. The residue was purified by column chromatography (silica gel, mobile phase dichloromethane/methanol 95:5). Drying under reduced pressure gave 9.59 g (61% of theory) of the title compound.

¹H NMR (400 MHz, CDCl₃, δ/ppm): 8.13 (d, 1H), 6.67 (s, 1H), 6.57 (d, 1H), 4.63 (s, 2H), 3.58-3.46 (m, 4H), 2.77-2.66 (m, 4H), 1.70-1.60 (m, 1H), 0.55-0.41 (m, 4H).

LC/MS (Method 5, ESIpos): Rₜ=0.17 min, m/z=234 [M+H]⁺.

### Step 3: 1-[4-(Chloromethyl)pyridin-2-yl]-4-cyclopropylpiperazine

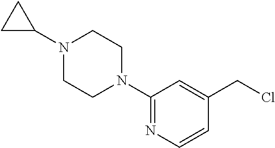 [SMILES: ClCc1ccnc(N2CCN(C3CC3)CC2)c1]

9.59 g (41.1 mmol) of the compound from Example 32A/Step 2 were initially charged in 60 ml of dichloromethane. 15 ml (205 mmol) of thionyl chloride were slowly added at RT, and the mixture was stirred initially at RT for 10 min and then under reflux for 4.5 h. After cooling to RT, 40 ml of water were added, and the mixture was made basic using 460 ml of saturated aqueous sodium bicarbonate solution and extracted three times with in each case 500 ml of dichloromethane. The combined dichloromethane phases were dried over magnesium sulphate, filtered and concentrated. The residue was purified by column chromatography (silica gel, mobile phase cyclohexane/ethyl acetate 7:3). Drying under reduced pressure gave 5.47 g (53% of theory) of the title compound.

¹H NMR (400 MHz, CDCl₃, δ/ppm): 8.16 (d, 1H), 6.68-6.56 (m, 2H), 4.45 (s, 2H), 3.61-3.45 (m, 4H), 2.79-2.67 (m, 4H), 1.69-1.62 (m, 1H), 0.58-0.35 (m, 4H).

LC/MS (Method 5, ESIpos): Rₜ=0.43 min, m/z=252/254 [M+H]⁺.

### Example 33A

### 3-[(3-{(Z)-1-Fluoro-2-[4-(trifluoromethoxy)phenyl]vinyl}-5-methyl-1H-pyrazol-1-yl)methyl]benzoic acid

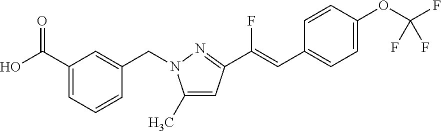 [SMILES: Cc1cc(/C(F)=C/c2ccc(OC(F)(F)F)cc2)nn1Cc1cccc(C(=O)O)c1]

700 mg (1.61 mmol) of the compound from Example 18 were suspended in 15 ml of methanol, and 4.8 ml (4.83 mmol) 1 M aqueous sodium hydroxide solution were added. The mixture was heated under reflux for 1 h and most of the methanol was then removed on a rotary evaporator. 6.4 ml (6.45 mmol) of 1 M hydrochloric acid were added to the aqueous residue and the mixture was stirred at RT for a few minutes, whereupon the product precipitated out. The solid was filtered off with suction, washed with cold water and dried under high vacuum. This gave 603 mg (89% of theory) of the title compound.

¹H NMR (400 MHz, DMSO-d₆, δ/ppm): 13.03 (very broad, 1H), 7.86 (d, 1H), 7.72 (d, 2H), 7.71 (s, 1H), 7.50 (t, 1H), 7.38 (2 d, tog. 3H), 6.56 (d, 1H), 6.49 (s, 1H), 5.45 (s, 2H), 2.26 (s, 3H).

LC/MS (Method 5, ESIpos): Rₜ=1.25 min, m/z=421 [M+H]⁺, 841 [2M+H]⁺.

### Example 34A

### 3-({3-[(Z)-1-Fluoro-2-{4-[(trifluoromethyl)sulphanyl]phenyl}vinyl]-5-methyl-1H-pyrazol-1-yl}methyl)benzoic acid

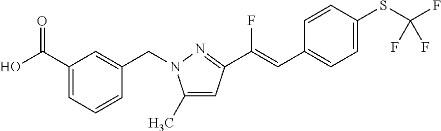 [SMILES: Cc1cc(/C(F)=C/c2ccc(SC(F)(F)F)cc2)nn1Cc1cccc(C(=O)O)c1]

Analogously to the process described under Example 33A, 530 mg (1.18 mmol) of the compound from Example 19 gave 379 mg (74% of theory) of the title compound. In this case, the product obtained after filtration with suction was purified by preparative HPLC (Method 14). This gave a first partial amount of 184 mg of the pure title compound and 236 mg of a mixed fraction which was re-purified by another preparative HPLC (Method 22).

¹H NMR (400 MHz, DMSO-d₆, δ/ppm): 13.05 (broad, 1H), 7.87 (d, 1H), 7.76-7.71 (m, 5H), 7.50 (t, 1H), 7.39 (d, 1H), 6.60 (d, 1H), 6.53 (s, 1H), 5.47 (s, 2H), 2.26 (s, 3H).

LC/MS (Method 5, ESIpos): Rₜ=1.28 min, m/z=437 [M+H]⁺, 873 [2M+H]⁺.

### Example 35A

### 3-[(3-{(Z)-1-Fluoro-2-[4-(1,1,1-trifluoro-2-methylpropan-2-yl)phenyl]vinyl}-5-methyl-1H-pyrazol-1-yl)methyl]benzoic acid

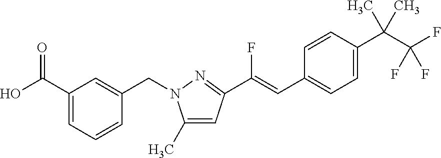 [SMILES: Cc1cc(/C(F)=C/c2ccc(C(C)(C)C(F)(F)F)cc2)nn1Cc1cccc(C(=O)O)c1]

Analogously to the process described under Example 33A, 192 mg (0.417 mmol) of the compound from Example 20 gave 172 mg (92% of theory) of the title compound. In this case, the product obtained after filtration with suction was purified by preparative HPLC (Method 14).

¹H NMR (400 MHz, DMSO-d₆, δ/ppm): 13.05 (broad, 1H), 7.87 (d, 1H), 7.72 (s, 1H), 7.62 (d, 2H), 7.55 (d, 2H), 7.50 (t, 1H), 7.39 (d, 1H), 6.51 (d, 1H), 6.49 (s, 1H), 5.45 (s, 2H), 2.25 (s, 3H), 1.56 (s, 6H).

LC/MS (Method 5, ESIpos): Rₜ=1.26 min, m/z=447 [M+H]⁺, 893 [2M+H]⁺.

### Example 36A

### 3-({3-[(Z)-1-Fluoro-2-{4-[1-(trifluoromethyl)cyclopropyl]phenyl}vinyl]-5-methyl-1H-pyrazol-1-yl}methyl)benzoic acid

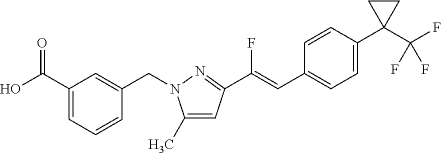 [SMILES: Cc1cc(/C(F)=C/c2ccc(C3(C(F)(F)F)CC3)cc2)nn1Cc1cccc(C(=O)O)c1]

Analogously to the process described under Example 33A, 243 mg (0.512 mmol) of the compound from Example 21 gave 225 mg (98% of theory, 90% pure) of the title compound.

¹H NMR (400 MHz, DMSO-d₆, δ/ppm): 13.05 (broad, 1H), 7.87 (d, 1H), 7.71 (s, 1H), 7.61 (d, 2H), 7.48 (t, 1H), 7.46 (d, 2H), 7.39 (d, 1H), 6.51 (d, 1H), 6.49 (s, 1H), 5.45 (s, 2H), 2.25 (s, 3H), 1.36-1.32 (m, 2H), 1.15-1.11 (m, 2H).

LC/MS (Method 5, ESIpos): Rₜ=1.25 min, m/z=445 [M+H]⁺, 889 [2M+H]⁺.

### Example 37A

### 3-[(3-{(Z)-1-Fluoro-2-[4-(trifluoromethyl)phenyl]vinyl}-5-methyl-1H-pyrazol-1-yl)methyl]benzoic acid

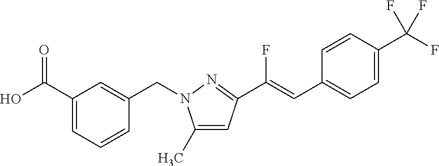 [SMILES: Cc1cc(/C(F)=C/c2ccc(C(F)(F)F)cc2)nn1Cc1cccc(C(=O)O)c1]

Analogously to the process described under Example 33A, 405 mg (0.968 mmol) of the compound from Example 22 gave 378 mg (96% of theory) of the title compound.

¹H NMR (400 MHz, DMSO-d₆, δ/ppm): 7.83 (d, 1H), 7.81 (d, 2H), 7.73 (d, 2H), 7.70 (s, 1H), 7.41 (t, 1H), 7.27 (d, 1H), 6.63 (d, 1H), 6.52 (s, 1H), 5.43 (s, 2H), 2.26 (s, 3H).

LC/MS (Method 5, ESIpos): Rₜ=1.20 min, m/z=405 [M+H]⁺, 809 [2M+H]⁺.

### Example 38A

### 2-({4-[(Z)-2-{1-[(6-Chloropyridin-3-yl)methyl]-5-methyl-1H-pyrazol-3-yl}-2-fluorovinyl]phenyl}sulphanyl)-2-methylpropanoic acid

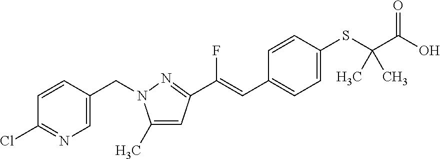 [SMILES: Cc1cc(/C(F)=C/c2ccc(SC(C)(C)C(=O)O)cc2)nn1Cc1ccc(Cl)nc1]

At 0° C., 244 mg (2.17 mmol) potassium tert-butoxide were added to a solution of 484 mg (1.447 mmol) of the compound from Example 19A and 314 mg (1.88 mmol, purity 97%) of 2-chloro-5-(chloromethyl)pyridine in 15 ml of THF. The mixture was stirred initially at RT for 1 h and then under reflux overnight. After addition of a further 100 mg (0.890 mmol) of potassium tert-butoxide, the mixture was stirred under reflux for a further 7 h. After cooling to RT, ethyl acetate was added and the mixture was extracted once with water. The aqueous phase was re-extracted once with ethyl acetate; this ethyl acetate phase was discarded. The aqueous phase was then adjusted to pH 5 using 1 N hydrochloric acid and extracted twice with ethyl acetate. The ethyl acetate extracts were combined with the ethyl acetate-containing mixture obtained above, dried over magnesium sulphate, filtered and concentrated. The residue was purified by column chromatography (silica gel, mobile phase dichloromethane/methanol 95:5). The solid obtained after removal of the solvent was triturated with pentane, filtered off and dried under high vacuum. This gave 313 mg (48% of theory, purity 99%) of the title compound.

¹H NMR (400 MHz, DMSO-d₆, δ/ppm): 12.65 (br. s, 1H), 8.32 (d, 1H), 7.62 (dd, 1H), 7.58 (d, 2H), 7.52 (d, 1H), 7.45 (d, 2H), 6.51 (d, 1H), 6.48 (s, 1H), 5.42 (s, 2H), 2.30 (s, 3H), 1.39 (s, 6H).

LC/MS (Method 5, ESIpos): Rₜ=1.15 min, m/z=446/448 [M+H]⁺.

### Example 39A

### 5-[(3-{(Z)-2-[3-Chloro-4-(trifluoromethoxy)phenyl]-1-fluorovinyl}-5-methyl-1H-pyrazol-1-yl)methyl]-N-(3,4-dimethoxybenzyl)-N-methylpyridine-2-amine

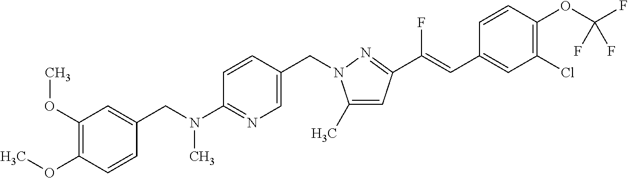 [SMILES: COc1ccc(CN(C)c2ccc(Cn3nc(/C(F)=C/c4ccc(OC(F)(F)F)c(Cl)c4)cc3C)cn2)cc1OC]

At 0° C., 214 mg (1.91 mmol) of potassium tert-butoxide were added to a solution of 186 mg (0.597 mmol) of the compound from Example 5A and 231 mg (0.753 mmol) of the compound from Example 31A in 5.7 ml of THF. The mixture was initially stirred at RT for 18 h. A further 58 mg (0.188 mmol) of the compound from Example 5A and 54 mg (0.482 mmol) potassium tert-butoxide were then added, and the mixture was stirred at RT for two days. 30 ml of water and 30 ml of ethyl acetate were then added to the mixture. After phase separation, the aqueous phase was extracted twice with in each case 30 ml of ethyl acetate. The combined organic phases were dried over sodium sulphate, filtered and concentrated. The residue was purified by preparative HPLC (Method 13). The combined product fractions were neutralized with saturated aqueous sodium bicarbonate solution and concentrated to a small residual volume of aqueous phase. After two extractions with in each case 30 ml of ethyl acetate, the combined organic phases were dried over sodium sulphate, filtered and concentrated. The residue was subjected to another preparative HPLC separation (Method 23). This gave 27 mg (7% of theory) of the title compound.

¹H NMR (400 MHz, CDCl₃, δ/ppm): 8.07 (d, 1H), 7.72 (d, 1H), 7.49 (dd, 1H), 7.33 (dd, 1H), 7.30-7.26 (m, 1H), 6.81-6.72 (m, 2H), 6.49 (d, 1H), 6.31 (d, 1H), 6.27 (s, 1H), 5.17 (s, 2H), 4.71 (s, 2H), 3.85 (s, 3H), 3.81 (s, 3H), 3.04 (s, 3H), 2.27 (s, 3H).

LC/MS (Method 5, ESIpos): Rₜ=1.31 min, m/z=591/593 [M+H]⁺.

### Example 40A

### 5-({3-[(Z)-2-(4-Cyclohexylphenyl)-1-fluorovinyl]-5-methyl-1H-pyrazol-1-yl}methyl)-N-(3,4-dimethoxybenzyl)-N-methylpyridine-2-amine

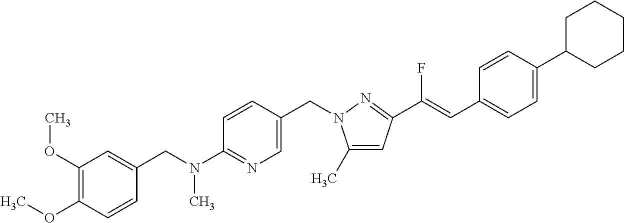 [SMILES: COc1ccc(CN(C)c2ccc(Cn3nc(/C(F)=C/c4ccc(C5CCCCC5)cc4)cc3C)cn2)cc1OC]

Analogously to the process described under Example 39A, 200 mg (0.703 mmol) of the compound from Example 13A and 326 mg (0.774 mmol, purity 90%) of the compound from Example 31A gave 75 mg (17% of theory, purity 98%) of the title compound. In this case, the reaction had ended after 18 h of stirring at RT (no further addition of reagents required). The crude product was purified initially by column chromatography (silica gel, mobile phase cyclohexane/ethyl acetate 6:4), followed by thick-layer chromatography (silica gel, dichloromethane/methanol 50:1). The product zone was extracted with dichloromethane/methanol 95:5. Concentration of the extract and drying of the residue under high vacuum gave the title compound.

¹H NMR (400 MHz, CDCl₃, δ/ppm): 8.07 (d, 1H), 7.53 (d, 2H), 7.33 (dd, 1H), 7.19 (d, 2H), 6.81-6.70 (m, 3H), 6.48 (d, 1H), 6.32 (d, 1H), 6.24 (s, 1H), 5.17 (s, 2H), 4.70 (s, 2H), 3.84 (s, 3H), 3.81 (s, 3H), 3.03 (s, 3H), 2.54-2.45 (m, 1H), 2.25 (s, 3H), 1.92-1.80 (m, 4H), 1.78-1.70 (m, 1H), 1.48-1.32 (m, 4H), 1.31-1.20 (m, 1H).

LC/MS (Method 5, ESIpos): Rₜ=1.42 min, m/z=555 [M+H]⁺.

### Example 41A

### N-(3,4-Dimethoxybenzyl)-5-[(3-{(Z)-1-fluoro-2-[4-(pentafluoro-λ⁶-sulphanyl)phenyl]vinyl}-5-methyl-1H-pyrazol-1-yl)methyl]-N-methylpyridine-2-amine

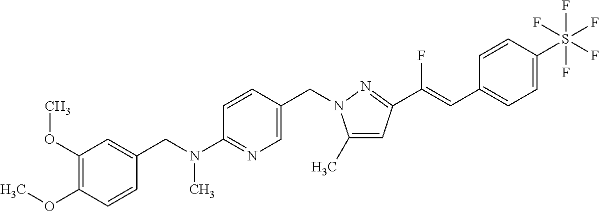 [SMILES: COc1ccc(CN(C)c2ccc(Cn3nc(/C(F)=C/c4ccc(S(F)(F)(F)(F)F)cc4)cc3C)cn2)cc1OC]

Analogously to the process described under Example 39A, 300 mg (0.914 mmol) of the compound from Example 18A and 364 mg (1.19 mmol) of the compound from Example 31A gave 105 mg (19% of theory, purity 97%) of the title compound. In this case, the reaction had ended after 18 h of stirring at RT (no further addition of reagents required). The crude product was purified initially by preparative HPLC (Method 16), followed by column chromatography (silica gel, mobile phase cyclohexane/ethyl acetate 7:3) and finally another preparative HPLC (Method 24).

¹H NMR (400 MHz, CDCl₃, δ/ppm): 8.30 (br. s, 1H), 7.79-7.70 (m, 3H), 7.68-7.63 (m, 2H), 6.88 (d, 1H), 6.82 (d, 1H), 6.72-6.66 (m, 2H), 6.37 (d, 1H), 6.32 (s, 1H), 5.21 (s, 2H), 4.72 (s, 2H), 3.85 (d, 6H), 3.38 (s, 3H), 2.31 (s, 3H).

LC/MS (Method 6, ESIpos): Rₜ=2.42 min, m/z=599 [M+H]⁺.

### Example 42A

### 5-({3-[(Z)-2-{4-[(Diisopropylamino)methyl]phenyl}-1-fluorovinyl]-5-methyl-1H-pyrazol-1-yl}methyl)-N-(3,4-dimethoxybenzyl)-N-methylpyridine-2-amine

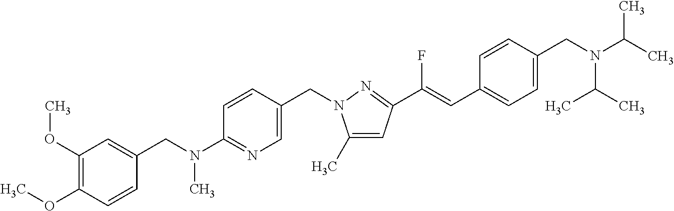 [SMILES: COc1ccc(CN(C)c2ccc(Cn3nc(/C(F)=C/c4ccc(CN(C(C)C)C(C)C)cc4)cc3C)cn2)cc1OC]

Analogously to the process described under Example 39A, 240 mg (0.761 mmol) of the compound from Example 20A and 376 mg (0.989 mmol) of the compound from Example 31A gave 96 mg (22% of theory) of the title compound. In this case, the reaction time was 2.5 h at RT (no further addition of reagents required). The crude product was purified initially by preparative HPLC (Method 25), followed by column chromatography (silica gel, mobile phase dichloro-methane/methanol 100:4).

¹H NMR (400 MHz, CDCl₃, δ/ppm): 8.07 (d, 1H), 7.54 (d, 2H), 7.40-7.31 (m, 3H), 6.81-6.71 (m, 3H), 6.48 (d, 1H), 6.34 (d, 1H), 6.25 (s, 1H), 5.17 (s, 2H), 4.70 (s, 2H), 3.85 (s, 3H), 3.81 (s, 3H), 3.63 (br. s, 2H), 3.03 (s, 3H), 3.03-2.98 (m, 2H), 2.25 (s, 3H), 1.02 (d, 12H).

LC/MS (Method 5, ESIpos): Rₜ=0.81 min, m/z=586 [M+H]⁺.

### Example 43A

### 2-Fluoro-1-[5-methyl-1-(4-methylbenzyl)-1H-pyrazol-3-yl]-2-[4-(trifluoromethoxy)phenyl]ethanol (diastereomer and enantiomer mixture)

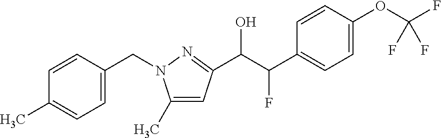 [SMILES: Cc1ccc(Cn2nc(C(O)C(F)c3ccc(OC(F)(F)F)cc3)cc2C)cc1]

### Step 1: 5-Methyl-1-(4-methylbenzyl)-1H-pyrazole-3-carbaldehyde

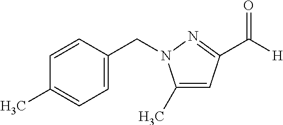 [SMILES: Cc1ccc(Cn2nc(C=O)cc2C)cc1]

Under argon and at −78° C., 3.57 ml (50.3 mmol) of DMSO, dissolved in 5 ml of dichloromethane, were added slowly to a solution of 1.75 ml (20.1 mmol) of oxalyl chloride in 10 ml of dichloromethane. 4.35 g (20.1 mmol) of the compound from Example 1A/Step 3, dissolved in 50 ml of dichloromethane, were then added slowly. After 1.5 h of stirring at −78° C., 14 ml (100 mmol) of triethylamine, dissolved in 10 ml of dichloromethane, were added, and the mixture was allowed to warm to 0° C. After 20 min of stirring at 0° C., the mixture was diluted with 300 ml of dichloromethane, washed in each case once with water and saturated sodium chloride solution, dried over magnesium sulphate and filtered. The solution was then filtered through about 50 g of silica gel which was washed with a mixture of cyclohexane and ethyl acetate (1:1). Filtrate and wash solution were combined and concentrated. Drying of the residue under reduced pressure gave 4.41 g (97% of theory, purity 95%) of the title compound.

¹H NMR (400 MHz, DMSO-d₆, δ/ppm): 9.83 (s, 1H), 7.16 (d, 2H), 7.08 (d, 2H), 6.60 (s, 1H), 5.39 (s, 2H), 2.27 (s, 3H), 2.26 (s, 3H).

LC/MS (Method 3, ESIpos): Rₜ=2.29 min, m/z=215 [M+H]⁺.

### Step 2: 5-Methyl-1-(4-methylbenzyl)-3-{(E/Z)-2-[4-(trifluoromethoxy)phenyl]vinyl}-1H-pyrazole

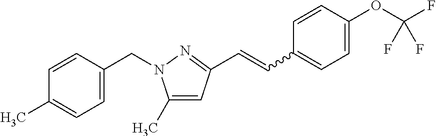 [SMILES: Cc1ccc(Cn2nc(C=Cc3ccc(OC(F)(F)F)cc3)cc2C)cc1]

### Method 1:

5.6 ml (14.9 mmol) of a 21% strength sodium ethylate solution in ethanol, diluted with a further 15 ml of ethanol, were added slowly to a boiling solution of 3.20 g (14.9 mmol) of the compound from Example 43A/Step 1 and 8.13 g (14.9 mmol, purity 95%) of triphenyl[4-(trifluoromethoxy)-benzyl]phosphonium bromide [for the preparation, see, for example, WO 2008/076046-A1, Example 43] in 35 ml of ethanol. After 4 h of stirring and subsequent cooling to RT, the mixture was concentrated on a rotary evaporator. The residue was purified by column chromatography (silica gel, mobile phase cyclohexane/ethyl acetate 8:2). Drying under high vacuum gave 1.39 g (25% of theory) of the title compound as an (E/Z) isomer mixture.

### Method 2:

By the process described under Method 1, 214 mg (1.0 mmol) of the compound from Example 43A/Step 1 were reacted with 517 mg (1.0 mmol) of triphenyl[4-(trifluoromethoxy)-benzyl]phosphonium bromide. In this case, the reaction time was 2 h (instead of 4 h) at 100° C. Work-up and purification were carried out as follows: after cooling of the reaction mixture to RT, the precipitate present was filtered off. The filtrate was concentrated, the residue was taken up in 100 ml of water and the pH was adjusted to 1 using 1 N hydrochloric acid. After three extractions with in each case 70 ml of ethyl acetate, the combined organic phases were dried over sodium sulphate, filtered and concentrated. The residue was purified initially by column chromatography (silica gel, mobile phase cyclohexane/ethyl acetate 4:1) and then by preparative HPLC (Method 26), the (E/Z) double bond isomers of the title compound being separated in the process. Drying of the respective fractions under high vacuum gave 23 mg (6% of theory) of the pure (E) isomer and 27 mg (7% of theory) of the pure (Z) isomer.

¹H NMR (400 MHz, CDCl₃, δ/ppm): (E) isomer: 7.48 (d, 2H), 7.17 (d, 2H), 7.14-7.05 (m, 3H), 7.04-6.97 (m, 3H), 6.28 (s, 1H), 5.24 (s, 2H), 2.32 (s, 3H), 2.20 (s, 3H).

LC/MS (Method 5, ESIpos): Rₜ=1.43 min, m/z=373 [M+H]⁺.

### Step 3: 1-[5-Methyl-1-(4-methylbenzyl)-1H-pyrazol-3-yl]-2-[4-(trifluoromethoxy)-phenyl]ethane-1,2-dione

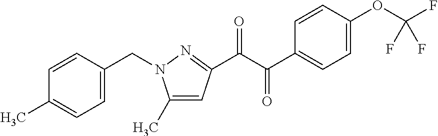 [SMILES: Cc1ccc(Cn2nc(C(=O)C(=O)c3ccc(OC(F)(F)F)cc3)cc2C)cc1]

623 mg (5.32 mmol) of N-methylmorpholine N-oxide and 1.5 ml (0.121 mmol) of a 2.5% strength solution of osmium tetroxide in tert-butanol were added to a solution of 900 mg (2.42 mmol) of the compound from Example 43A/Step 2 [(E/Z) isomer mixture] in 13.5 ml of acetone. The mixture was stirred at RT overnight, ethyl acetate and water were then added and, after phase separation, the aqueous phase was extracted once with ethyl acetate. The combined organic phases were dried over magnesium sulphate, filtered and concentrated. The residue was purified by preparative HPLC (Method 16). Drying under high vacuum gave 467 mg (58% of theory) of the title compound.

¹H NMR (400 MHz, CDCl₃, δ/ppm): 8.04 (d, 2H), 7.32 (d, 2H), 7.09 (d, 2H), 6.96 (d, 2H), 6.75 (s, 1H), 5.27 (s, 2H), 2.31 (s, 3H), 2.22 (s, 3H).

LC/MS (Method 5, ESIpos): Rₜ=1.33 min, m/z=403 [M+H]⁺.

### Step 4: 2-Hydroxy-1-[5-methyl-1-(4-methylbenzyl)-1H-pyrazol-3-yl]-2-[4-(trifluoromethoxy)phenyl]ethanone (racemate)

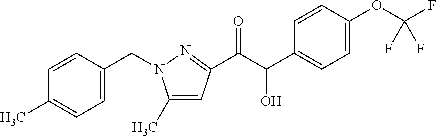 [SMILES: Cc1ccc(Cn2nc(C(=O)C(O)c3ccc(OC(F)(F)F)cc3)cc2C)cc1]

At 100° C., a solution of 606 mg (3.48 mmol) sodium dithionite in 2.8 ml of water was added slowly to a solution of 350 mg (0.870 mmol) of the compound from Example 43A/Step 3 in a mixture of 5.6 ml of DMF and 1.4 ml of water. The mixture was stirred at 100 C for 1.5 h. After cooling to RT, the mixture was concentrated on a rotary evaporator and ethyl acetate and water were added to the residue. After phase separation, the aqueous phase was extracted once with ethyl acetate. The combined organic phases were washed once with saturated sodium chloride solution, dried over magnesium sulphate, filtered and concentrated. The residue was purified by column chromatography (silica gel, mobile phase cyclohexane/ethyl acetate 7:3). This gave, in separated form, 246 mg (70% of theory) of the title compound and 103 mg (28% of theory) of the positional isomer 2-hydroxy-2-[5-methyl-1-(4-methylbenzyl)-1H-pyrazol-3-yl]-1-[4-(trifluoromethoxy)-phenyl]ethanone (as a racemate).

¹H NMR (400 MHz, CDCl₃, δ/ppm): 7.50 (d, 2H), 7.13-7.06 (m, 4H), 6.91 (d, 2H), 6.56 (s, 1H), 6.09 (d, 1H), 5.31-5.18 (m, 2H), 4.54 (d, 1H), 2.34 (s, 3H), 2.19 (s, 3H).

LC/MS (Method 5, ESIpos): Rₜ=1.26 min, m/z=405 [M+H]⁺.

### Step 5: 2-Fluoro-1-[5-methyl-1-(4-methylbenzyl)-1H-pyrazol-3-yl]-2-[4-(trifluoromethoxy)phenyl]ethanone (racemate)

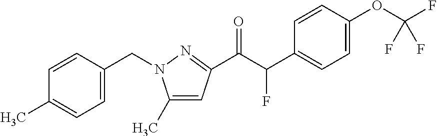 [SMILES: Cc1ccc(Cn2nc(C(=O)C(F)c3ccc(OC(F)(F)F)cc3)cc2C)cc1]

At 0° C., 59 μl (0.445 mmol) of diethylaminosulphur trifluoride (DAST) were added to a solution of 150 mg (0.371 mmol) of the compound from Example 43A/Step 4 in 1 ml of dichloromethane. The mixture was stirred at 0° C. for 15 min. After dilution with dichloromethane, the mixture was washed with saturated aqueous sodium bicarbonate solution and the aqueous phase was re-extracted once with dichloromethane. The combined organic phases were dried over magnesium sulphate, filtered and concentrated. The residue was purified initially by column chromatography (silica gel, mobile phase cyclohexane/ethyl acetate 9:1) and then by preparative HPLC (Method 16). The product fractions of the preparative HPLC were concentrated to a small residual volume of aqueous phase on a rotary evaporator, and saturated aqueous sodium bicarbonate solution was added. The solid formed was filtered off, washed three times with water and dried under high vacuum. This gave 74 mg (49% of theory) of the title compound.

¹H NMR (400 MHz, CDCl₃, δ/ppm): 7.58 (d, 2H), 7.13 (m, 4H), 6.93 (d, 2H), 6.87 (d, 1H), 6.59 (s, 1H), 5.26 (s, 2H), 2.34 (s, 3H), 2.20 (s, 3H).

LC/MS (Method 5, ESIpos): Rₜ=1.34 min, m/z=407 [M+H]⁺.

### Step 6: 2-Fluoro-1-[5-methyl-1-(4-methylbenzyl)-1H-pyrazol-3-yl]-2-[4-(trifluoromethoxy)phenyl]ethanol (diastereomer and enantiomer mixture)

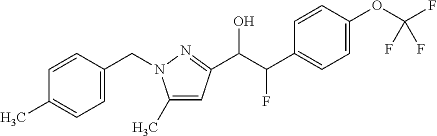 [SMILES: Cc1ccc(Cn2nc(C(O)C(F)c3ccc(OC(F)(F)F)cc3)cc2C)cc1]

At 0° C., 3 mg (0.080 mmol) sodium borohydride were added to a mixture of 33 mg (0.080 mmol) of the compound from Example 43A/Step 5 and 1 ml of ethanol. After 5 min of stirring at 0° C., the mixture was allowed to warm to RT and stirred at this temperature for a further 30 min. Saturated aqueous ammonium chloride solution was then added, and the mixture was extracted twice with ethyl acetate. The combined organic phases were dried over magnesium sulphate, filtered and concentrated. The residue was dried under high vacuum. This gave 32 mg (97% of theory) of the title compound as a diastereomer mixture.

LC/MS (Method 5, ESIpos): Rₜ=1.20 and 1.23 min, in each case m/z=409 [M+H]⁺.

### Example 44A

### 2-({Fluoro[4-(trifluoromethoxy)phenyl]methyl}sulphonyl)-1,3-benzothiazole (racemate)

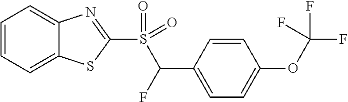 [SMILES: O=S(=O)(c1nc2ccccc2s1)C(F)c1ccc(OC(F)(F)F)cc1]

### Step 1: 2-{[4-(Trifluoromethoxy)benzyl]sulphanyl}-1,3-benzothiazole

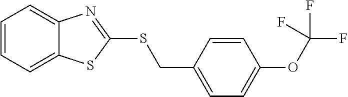 [SMILES: FC(F)(F)Oc1ccc(CSc2nc3ccccc3s2)cc1]

Analogously to the process described under Example 2A/Step 4, 15.0 g (58.8 mmol) 4-(trifluoromethoxy)benzyl bromide and 11.1 g (58.8 mmol) sodium 1,3-benzothiazole-2-thiolate gave 18.8 g (94% of theory) of the title compound.

¹H NMR (400 MHz, CDCl₃, δ/ppm): 7.90 (d, 1H), 7.76 (d, 1H), 7.49 (d, 2H), 7.44 (dt, 1H), 7.31 (dt, 1H), 7.17 (d, 2H), 4.60 (s, 2H).

LC/MS (Method 8, ESIpos): Rₜ=1.44 min, m/z=342 [M+H]⁺.

### Step 2: 2-{[4-(Trifluoromethoxy)benzyl]sulphonyl}-1,3-benzothiazole

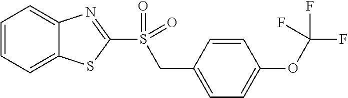 [SMILES: O=S(=O)(Cc1ccc(OC(F)(F)F)cc1)c1nc2ccccc2s1]

Analogously to the process described under Example 2A/Step 5, 18.5 g (54.2 mmol) of the compound from Example 44A/Step 1 and 40.1 g (163 mmol, content 70%) of meta-chloroperoxybenzoic acid gave 13.1 g (65% of theory) of the title compound.

¹H NMR (400 MHz, CDCl₃, δ/ppm): 8.26 (d, 1H), 7.97 (d, 1H), 7.67 (dt, 1H), 7.60 (dt, 1H), 7.33 (d, 2H), 7.14 (d, 2H), 4.76 (s, 2H).

LC/MS (Method 8, ESIpos): Rₜ=1.19 min, m/z=374 [M+H]⁺.

### Step 3: 2-({Fluoro[4-(trifluoromethoxy)phenyl]methyl}sulphonyl)-1,3-benzothiazole (racemate)

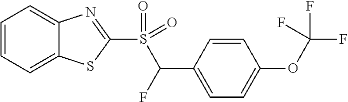 [SMILES: O=S(=O)(c1nc2ccccc2s1)C(F)c1ccc(OC(F)(F)F)cc1]

Analogously to the process described under Example 2A/Step 6, 6.50 g (17.4 mmol) of the compound from Example 44A/Step 2 and 11 g (34.8 mmol) of N-fluorobenzenesulphonimide (NFSI) gave 4.1 g (61% of theory) of the title compound. The crude product was purified by silica gel chromatography using the mobile phase cyclohexane/ethyl acetate 10:1.

¹H NMR (400 MHz, CDCl₃, δ/ppm): 8.30 (d, 1H), 8.06 (d, 1H), 7.70 (dt, 1H), 7.69 (d, 2H), 7.66 (dt, 1H), 7.34 (d, 2H), 6.65 (d, 1H).

LC/MS (Method 5, ESIpos): Rₜ=1.24 min, m/z=392 [M+H]⁺.

### Example 45A

### 3-{(Z)-2-Fluoro-2-[4-(trifluoromethoxy)phenyl]vinyl}-5-methyl-1H-pyrazole

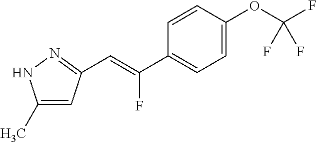 [SMILES: Cc1cc(/C=C(\F)c2ccc(OC(F)(F)F)cc2)n[nH]1]

### Step 1: 5-Methyl-1-(tetrahydro-2H-pyran-2-yl)-1H-pyrazole-3-carbaldehyde

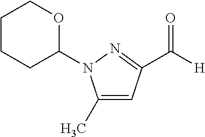

At a bath temperature of about −78° C., a solution of 5.4 ml (75.7 mmol) of DMSO in 16 ml of anhydrous dichloromethane was added dropwise to a solution of 2.9 ml (33.3 mmol) oxalyl chloride in 16 ml of anhydrous dichloromethane. A solution of 5.94 g (30.3 mmol) of the compound from Example 2A/Step 2 in 80 ml of anhydrous dichloromethane was then added dropwise over a period of 30 min. The reaction mixture was stirred at a bath temperature of −78° C. for 1.5 h, a solution of 21 ml (151 mmol) of triethylamine in 13 ml of anhydrous dichloromethane was then added dropwise and the acetone/dry ice bath was then replaced with an ice/water bath. After 20 min at 0° C., the mixture was diluted with about 500 ml of dichloromethane and extracted in succession in each case once with water and saturated aqueous sodium chloride solution. After drying of the organic phase over anhydrous magnesium sulphate, filtration and evaporation, the residue was purified by MPLC (silica gel, mobile phase cyclohexane/ethyl acetate 9:1→8:2). Concentration of the product fractions and drying of the residue under high vacuum gave 5.35 g (91% of theory) of the title compound.

¹H NMR (400 MHz, CDCl₃, δ/ppm): 9.94 (s, 1H), 6.57 (s, 1H), 5.37 (dd, 1H), 4.08-4.02 (m, 1H), 3.72-3.65 (m, 1H), 2.52-2.42 (m, 1H), 2.39 (s, 1H), 2.19-2.13 (m, 1H), 2.03-1.97 (m, 1H), 1.78-1.68 (m, 2H), 1.67-1.62 (m, 1H).

### Step 2: 3-{(Z)-2-Fluoro-2-[4-(trifluoromethoxy)phenyl]vinyl}-5-methyl-1-(tetrahydro-2H-pyran-2-yl)-1H-pyrazole

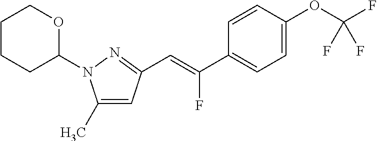 [SMILES: Cc1cc(/C=C(\F)c2ccc(OC(F)(F)F)cc2)nn1C1CCCCO1]

At a temperature of 0-5° C., 18.4 ml (18.4 mmol) of a 1 M solution of lithium hexamethyldisilazide (LiHMDS) in THF were added dropwise to a solution of 3.0 g (7.67 mmol) of the compound from Example 44A and 1.49 g (7.67 mmol) of the compound from Example 45A/Step 1 in 145 ml of anhydrous THF. After the addition had ended, the reaction mixture was stirred at 0° C. for 3 h. 400 ml of semisaturated aqueous ammonium chloride solution were then added, and the mixture was extracted twice with in each case about 200 ml of ethyl acetate. The combined organic extracts were dried over anhydrous sodium sulphate. After filtration, the solvent was removed on a rotary evaporator. The residue that remained was purified by MPLC (100 g silica gel, mobile phase cyclohexane/ethyl acetate 10:1→5:1). This gave one fraction which comprised 1.44 g (51% of theory) of the title compound in pure form, and a second fraction of 0.87 g which consisted of a mixture of the title compound and the corresponding (E) isomer.

LC/MS (Method 8, ESIpos): Rₜ=1.37 min, m/z=371 [M+H]⁺.

### Step 3: 3-{(Z)-2-Fluoro-2-[4-(trifluoromethoxy)phenyl]vinyl}-5-methyl-1H-pyrazole

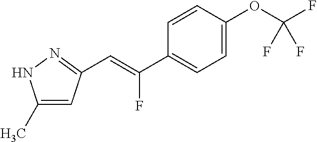 [SMILES: Cc1cc(/C=C(\F)c2ccc(OC(F)(F)F)cc2)n[nH]1]

1.44 g (3.89 mmol) of the compound from Example 45A/Step 2 were dissolved in 30 ml of a 4 M solution of hydrogen chloride in dioxane. After 16 h of stirring at RT, the reaction mixture was diluted with 400 ml of ethyl acetate and then washed successively with in each case about 100 ml of water, semisaturated aqueous sodium bicarbonate solution and saturated aqueous sodium chloride solution. After drying over anhydrous magnesium sulphate, the mixture was filtered and the solvent was removed on a rotary evaporator. The crude product obtained was purified by MPLC (silica gel, mobile phase cyclohexane/ethyl acetate 2:1→1:1). Evaporation of the product fractions and drying of the residue under high vacuum gave 1.05 g (94% of theory) of the title compound.

¹H NMR (400 MHz, CDCl₃, δ/ppm): 9.89 (broad, 1H), 7.62 (d, 2H), 7.22 (d, 2H), 6.39 (d, 1H), 5.76 (s, 1H), 2.24 (s, 3H).

LC/MS (Method 5, ESIpos): Rₜ=1.04 min, m/z=287 [M+H]⁺.

### Example 46A

### 3-(Pyrrolidin-1-ylcarbonyl)benzyl methanesulphonate

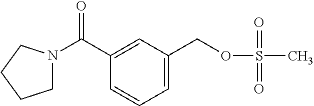 [SMILES: CS(=O)(=O)OCc1cccc(C(=O)N2CCCC2)c1]

### Step 1: Methyl 3-(pyrrolidin-1-ylcarbonyl)benzoate

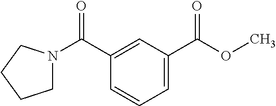 [SMILES: COC(=O)c1cccc(C(=O)N2CCCC2)c1]

5.0 g (25.2 mmol) of methyl 3-(chlorocarbonyl)benzoate were dissolved in 25 ml of anhydrous dichloromethane, and a solution of 4.2 ml (50.4 mmol) pyrrolidine in 25 ml of anhydrous dichloromethane was quickly added dropwise at RT. After a reaction time of 4 h, about 100 ml of water were added. The phases were separated, and the aqueous phase was extracted twice with in each case about 20 ml of dichloromethane. The combined organic extracts were dried over anhydrous magnesium sulphate, filtered and concentrated on a rotary evaporator. The residue obtained was purified by MPLC (silica gel, mobile phase cyclohexane/ethyl acetate 1:1). Evaporation of the product fractions and drying of the residue under high vacuum gave 5.57 g (95% of theory) of the title compound.

¹H NMR (400 MHz, CDCl₃, δ/ppm): 8.19 (s, 1H), 8.09 (d, 1H), 7.73 (d, 1H), 7.50 (t, 1H), 3.93 (s, 3H), 3.66 (t, 2H), 3.43 (t, 2H), 2.02-1.95 (m, 2H), 1.93-1.86 (m, 2H).

LC/MS (Method 8, ESIpos): Rₜ=0.76 min, m/z=234 [M+H]⁺, 467 [2M+H]⁺.

### Step 2: [3-(Hydroxymethyl)phenyl](pyrrolidin-1-yl)methanone

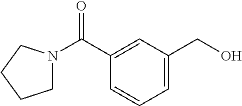 [SMILES: O=C(c1cccc(CO)c1)N1CCCC1]

At 0° C., 14.2 ml (14.2 mmol) of a 1 M solution of lithium aluminium hydride in THF were added dropwise to a solution of 5.53 g (23.7 mmol) of the compound from Example 46A/Step 1 in 140 ml of anhydrous THF. After the reaction mixture had been stirred at 0° C. for 1 h, the reaction was terminated by careful addition of a few ml of saturated aqueous ammonium chloride solution. The mixture was diluted with ethyl acetate, and subsequently anhydrous magnesium sulphate was added in the amount required for complete absorption of the aqueous phase. After filtration the filtrate was concentrated on a rotary evaporator and the residue obtained was purified by MPLC (silica gel, mobile phase ethyl acetate). Evaporation of the product fractions and drying of the residue under high vacuum gave 4.28 g (88% of theory) of the title compound.

¹H NMR (400 MHz, CDCl₃, δ/ppm): 7.47 (s, 1H), 7.40-7.33 (m, 3H), 4.66 (s, broad, 2H), 3.63 (t, 2H), 3.40 (t, 2H), 2.94 (broad, 1H), 1.99-1.92 (m, 2H), 1.89-1.83 (m, 2H).

LC/MS (Method 8, ESIpos): Rₜ=0.55 min, m/z=206 [M+H]⁺, 411 [2M+H]⁺.

### Step 3: 3-(Pyrrolidin-1-ylcarbonyl)benzyl methanesulphonate

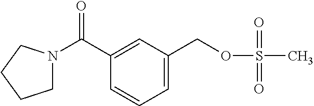 [SMILES: CS(=O)(=O)OCc1cccc(C(=O)N2CCCC2)c1]

First 510 μl (3.65 mmol) of anhydrous triethylamine and then, at 0° C. and dropwise, 467 mg (2.68 mmol) of methanesulphonic anhydride were added to a solution of 500 mg (2.44 mmol) of the compound from Example 46A/Step 2 in 25 ml of anhydrous dichloromethane. The ice/water bath was then removed. The reaction mixture was stirred at RT for 1 h and then transferred into a separating funnel and washed successively with semisaturated aqueous ammonium chloride solution and saturated aqueous sodium chloride solution. The organic phase was dried over anhydrous magnesium sulphate and filtered, and the filtrate was then freed from the solvent on a rotary evaporator. Drying of the residue under high vacuum gave 685 mg (99% of theory) of the title compound.

¹H NMR (400 MHz, CDCl₃, δ/ppm): 7.59 (s, 1H), 7.55 (td, 1H), 7.48-7.43 (m, 2H), 5.26 (s, 2H), 3.65 (t, 2H), 3.42 (t, 2H), 2.98 (s, 3H), 2.01-1.95 (m, 2H), 1.93-1.86 (m, 2H).

LC/MS (Method 5, ESIpos): Rₜ=0.67 min, m/z=284 [M+H]⁺, 567 [2M+H]⁺.

### Working Examples

### Example 1

### 1-Benzyl-3-{(Z)-1-fluoro-2-[4-(1,1,1-trifluoro-2-methylpropan-2-yl)phenyl]vinyl}-5-methyl-1H-pyrazole

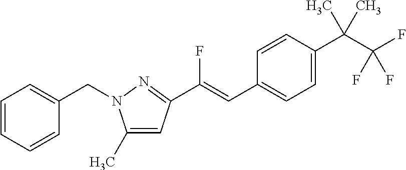 [SMILES: Cc1cc(/C(F)=C/c2ccc(C(C)(C)C(F)(F)F)cc2)nn1Cc1ccccc1]

At RT and under argon, 198 mg (1.95 mmol) of 4-hydroxypiperidine, 60 mg (0.065 mmol) of tris(dibenzylideneacetone)dipalladium, 93 mg (0.195 mmol) of 2-dicyclohexylphosphino-2′,4′,6′-triisopropylbiphenyl (X-Phos) and 795 mg (2.441 mmol) of caesium carbonate were added to a solution of 470 mg (0.976 mmol) of the compound from Example 7 in 9 ml of DMF. The reaction mixture was stirred at a bath temperature of 80° C. for 16 h and then allowed to cool to RT and filtered through Celite, and the filter cake was washed with DMF. The filtrate was concentrated, and the residue was purified by preparative HPLC (Method 13). This gave two main fractions which, according to analytical LC/MS consisted firstly of the title compound and secondly of the compound described under Example 13 (see there). The fraction of the title compound was freed from the methanol of the HPLC separation on a rotary evaporator, adjusted to a pH of 7-8 using saturated aqueous sodium bicarbonate solution and extracted twice with ethyl acetate. The combined organic phases were dried over sodium sulphate and concentrated. Drying of the residue under high vacuum gave 168 mg (43% of theory) of the title compound.

¹H NMR (400 MHz, CDCl₃, δ/ppm): 7.61 (d, 2H), 7.47 (d, 2H), 7.36-7.27 (m, 3H), 7.11 (d, 2H), 6.38 (d, 1H), 6.31 (s, 1H), 5.33 (s, 2H), 2.21 (s, 3H), 1.58 (s, 6H).

LC/MS (Method 5, ESIpos): Rₜ=1.44 min, m/z=403 [M+H]⁺.

### Example 2

### 3-{(Z)-1-Fluoro-2-[4-(trifluoromethoxy)phenyl]vinyl}-5-methyl-1-(4-methylbenzyl)-1H-pyrazole

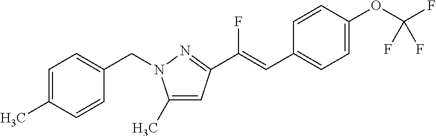 [SMILES: Cc1ccc(Cn2nc(/C(F)=C/c3ccc(OC(F)(F)F)cc3)cc2C)cc1]

At 0° C., 85 mg (0.760 mmol) of potassium tert-butoxide were added to a solution of 150 mg (0.524 mmol) of the compound from Example 3A and 126 mg (0.681 mmol) of 4-methylbenzyl bromide in 5 ml of THF. The mixture was stirred at RT for 3 days. After removal of the solvent, 50 ml of water were added and the mixture was extracted three times with in each case 50 ml of ethyl acetate. The combined organic phases were dried over sodium sulphate, filtered and concentrated. The residue was purified by preparative HPLC (Method 27). Drying under high vacuum gave 157 mg (69% of theory, purity 90%) of the title compound.

¹H NMR (400 MHz, CDCl₃, δ/ppm): 7.63 (d, 2H), 7.19 (d, 2H), 7.13 (d, 2H), 7.02 (d, 2H), 6.38 (d, 1H), 6.30 (s, 1H), 5.28 (s, 2H), 2.32 (s, 3H), 2.21 (s, 3H).

LC/MS (Method 5, ESIpos): Rₜ=1.45 min, m/z=391 [M+H]⁺.

### Example 3

### 3-[(Z)-1-Fluoro-2-{4-[(trifluoromethyl)sulphanyl]phenyl}vinyl]-5-methyl-1-(4-methylbenzyl)-1H-pyrazole

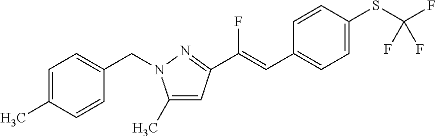 [SMILES: Cc1ccc(Cn2nc(/C(F)=C/c3ccc(SC(F)(F)F)cc3)cc2C)cc1]

Under argon, 24 mg (0.118 mmol) of 4-[(trifluoromethyl)sulphanyl]benzaldehyde were added to a solution of 49 mg (0.118 mmol) of the compound from Example 1A in 2.2 ml of THF. With stirring, the mixture was cooled to 0° C. 283 μl (0.283 mmol) of a 1 M solution of lithium hexamethyldisilazide in THF/ethylbenzene were then added, and the mixture was stirred with ice bath cooling for a further 3 h. Dilute aqueous ammonium chloride solution and ethyl acetate were then added to the mixture, and the phases were separated. The aqueous phase was extracted three times with ethyl acetate, and the combined organic phases were dried over sodium sulphate, filtered and concentrated. The residue was dissolved in 3 ml of acetonitrile, and 2 ml of water were added. The resulting precipitate was filtered off and dried under high vacuum. This gave 26 mg (53% of theory) of the title compound.

¹H NMR (400 MHz, DMSO-d₆, δ/ppm): 7.68-7.57 (m, 4H), 7.13 (d, 2H), 7.02 (d, 2H), 6.41 (d, 1H), 6.31 (s, 1H), 5.29 (s, 2H), 2.32 (s, 3H), 2.21 (s, 3H).

LC/MS (Method 2, ESIpos): Rₜ=1.70 min, m/z=407 [M+H]⁺.

### Example 4

### 4-(5-{(Z)-2-Fluoro-2-[5-methyl-1-(4-methylbenzyl)-1H-pyrazol-3-yl]vinyl}pyridin-2-yl)-2,6-dimethylmorpholine

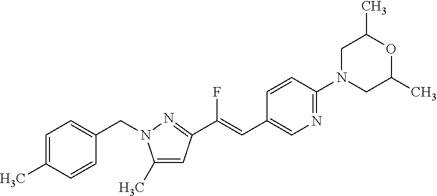

58 mg (0.303 mmol, purity 97%) of 4-methylbenzyl bromide were added to a solution of 80 mg (0.253 mmol) of the compound from Example 21A in 2.5 ml of THF. The mixture was cooled to 0° C. 37 mg (0.329 mmol) of potassium tert-butoxide were then added, and the mixture was stirred initially at 0° C. for a few minutes and then at RT for 4 h. The mixture was then diluted with ethyl acetate and extracted once with water. The aqueous phase was re-extracted once with ethyl acetate. The combined organic phases were washed once with saturated sodium chloride solution, dried over magnesium sulphate, filtered and concentrated. The residue was purified by thick-layer chromatography (silica gel, mobile phase cyclohexane/ethyl acetate 7:3). The product-containing zone was extracted with dichloromethane/methanol 95:5. The solvent was removed, and pentane was then added to the residue. The solid formed was filtered off and dried under high vacuum. This gave 74 mg (69% of theory) of the title compound.

¹H NMR (400 MHz, CDCl₃, δ/ppm): 8.32 (s, 1H), 7.89 (d, 1H), 7.12 (d, 2H), 7.01 (d, 2H), 6.64 (d, 1H), 6.26 (s, 1H), 6.26 (d, 1H), 5.27 (s, 2H), 4.08 (d, 2H), 3.78-3.67 (m, 2H), 2.55 (t, 2H), 2.32 (s, 3H), 2.19 (s, 3H), 1.27 (d, 6H).

LC/MS (Method 8, ESIpos): Rₜ=1.29 min, m/z=421 [M+H]⁺.

### Example 5

### 3-{(Z)-1-Fluoro-2-[4-(1,1,1-trifluoro-2-methylpropan-2-yl)phenyl]vinyl}-5-methyl-1-[3-(prop-1-en-2-yl)benzyl]-1H-pyrazole

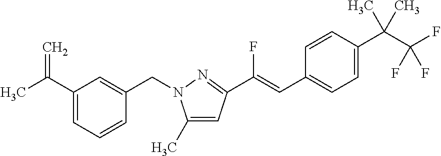 [SMILES: C=C(C)c1cccc(Cn2nc(/C(F)=C/c3ccc(C(C)(C)C(F)(F)F)cc3)cc2C)c1]

At RT, 72 mg (0.640 mmol) potassium tert-butoxide were added to a solution of 125 mg (0.40 mmol) of the compound from Example 7A and 150 mg (0.520 mmol, purity about 80%) of the compound from Example 26A in 3.5 ml of THF. The reaction mixture was stirred at a bath temperature of 80° C. for 3 h. After cooling to RT, 30 ml of water were added and the mixture was extracted three times with in each case 30 ml of ethyl acetate. The combined organic phases were dried over sodium sulphate, filtered and concentrated. The residue was purified by preparative HPLC (Method 16). The product fractions were concentrated on a rotary evaporator to a small residual volume of water and then adjusted to a pH of 7 with saturated aqueous sodium bicarbonate solution. The mixture was then extracted twice with in each case 30 ml of ethyl acetate, and the combined organic phases were dried over sodium sulphate, filtered and concentrated. Drying under high vacuum gave 85 mg (45% of theory, purity 93%) of the title compound.

¹H NMR (400 MHz, CDCl₃, δ/ppm): 7.61 (d, 2H), 7.49-7.45 (m, 2H), 7.38 (d, 1H), 7.29 (d, 1H), 7.23 (s, 1H), 6.99 (d, 1H), 6.38 (d, 1H), 6.31 (s, 1H), 5.33 (s, 3H), 5.08 (t, 1H), 2.22 (s, 3H), 2.12 (s, 3H), 1.58 (s, 6H).

LC/MS (Method 5, ESIpos): Rₜ=1.54 min, m/z=443 [M+H]⁺.

### Example 6

### 1-(3-Bromobenzyl)-3-{(Z)-1-fluoro-2-[4-(trifluoromethoxy)phenyl]vinyl}-5-methyl-1H-pyrazole

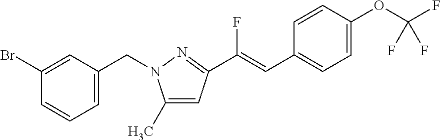 [SMILES: Cc1cc(/C(F)=C/c2ccc(OC(F)(F)F)cc2)nn1Cc1cccc(Br)c1]

With ice cooling and under argon, 682 mg (6.08 mmol) of potassium tert-butoxide were added to a solution of 1.20 g (4.19 mmol) of the compound from Example 3A in 40 ml of THF. After 30 min, 1.26 g (5.03 mmol) of 1-bromo-3-(bromomethyl)benzene were added, and the mixture was stirred at RT for a further 3 h. 70 ml each of water and ethyl acetate were then added, and after phase separation the aqueous phase was extracted once with ethyl acetate. The combined organic phases were dried over sodium sulphate, filtered and concentrated. The crude product was purified initially by column chromatography (silica gel, mobile phase cyclohexane/ethyl acetate 4:1), and the product fractions obtained in this manner were then re-purified by preparative HPLC (Method 28). Drying under reduced pressure gave 1.38 g (72% of theory) of the title compound.

¹H NMR (400 MHz, CDCl₃, δ/ppm): 7.63 (d, 2H), 7.42 (d, 1H), 7.28-7.26 (m, 1H), 7.24-7.16 (m, 3H), 7.02 (d, 1H), 6.38 (d, 1H), 6.33 (s, 1H), 5.29 (s, 2H), 2.22 (s, 3H).

LC/MS (Method 5, ESIpos): Rₜ=1.49 min, m/z=455/457 [M+H]⁺.

### Example 7

### 1-(3-Bromobenzyl)-3-{(Z)-1-fluoro-2-[4-(1,1,1-trifluoro-2-methylpropan-2-yl)phenyl]vinyl}-5-methyl-1H-pyrazole

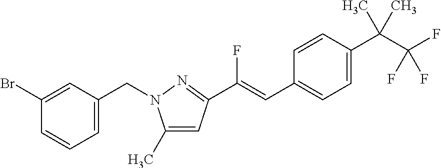 [SMILES: Cc1cc(/C(F)=C/c2ccc(C(C)(C)C(F)(F)F)cc2)nn1Cc1cccc(Br)c1]

Analogously to the process described under Example 6, 1.0 g (3.20 mmol) of the compound from Example 7A and 960 mg (3.84 mmol) of 1-bromo-3-(bromomethyl)benzene gave 1.47 g (86% of theory, purity 90%) of the title compound. In this case, the reaction mixture was stirred at RT for 16 h (instead of 3 h). The crude product was purified by column chromatography (silica gel, mobile phase cyclohexane/ethyl acetate 4:1).

¹H NMR (400 MHz, CDCl₃, δ/ppm): 7.61 (d, 2H), 7.48 (d, 2H), 7.42 (d, 1H), 7.28-7.26 (m, 1H), 7.20 (t, 1H), 7.02 (d, 1H), 6.38 (d, 1H), 6.32 (s, 1H), 5.30 (s, 2H), 2.21 (s, 3H), 1.58 (s, 6H).

LC/MS (Method 5, ESIpos): Rₜ=1.50 min, m/z=481/483 [M+H]⁺.

### Example 8

### 1-(3-Bromobenzyl)-3-[(Z)-1-fluoro-2-{4-[1-(trifluoromethyl)cyclopropyl]phenyl}vinyl]-5-methyl-1H-pyrazole

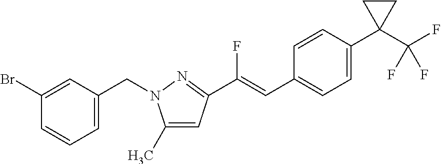 [SMILES: Cc1cc(/C(F)=C/c2ccc(C3(C(F)(F)F)CC3)cc2)nn1Cc1cccc(Br)c1]

Analogously to the process described under Example 6, 695 mg (2.24 mmol) of the compound from Example 9A and 672 mg (2.69 mmol) of 1-bromo-3-(bromomethyl)benzene were reacted with one another. In this case, the reaction mixture was stirred at RT for 16 h (instead of 3 h). The crude product was purified by column chromatography (silica gel, mobile phase cyclohexane/ethyl acetate 4:1). This gave 923 mg (about 86% of theory) of the title compound (batch 1). The reaction described was repeated with a second batch, giving in this case 987 mg (about 92% of theory) of the title compound (batch 2). Both product batches were combined and re-purified by preparative HPLC (Method 29). Drying under high vacuum gave, from the two batches together, 1.57 g (73% of theory) of the title compound.

¹H NMR (400 MHz, CDCl₃, δ/ppm): 7.58 (d, 2H), 7.46-7.39 (m, 3H), 7.28-7.25 (m, 1H), 7.20 (t, 1H), 7.02 (d, 1H), 6.37 (d, 1H), 6.32 (s, 1H), 5.29 (s, 2H), 2.21 (s, 3H), 1.37-1.33 (m, 2H), 1.07-1.00 (m, 2H).

LC/MS (Method 5, ESIpos): Rₜ=1.48 min, m/z=479/480 [M+H]⁺.

### Example 9

### 1-{3-[(3-{(Z)-1-Fluoro-2-[4-(trifluoromethoxy)phenyl]vinyl}-5-methyl-1H-pyrazol-1-yl)-methyl]phenyl}piperidine-4-carbonitrile

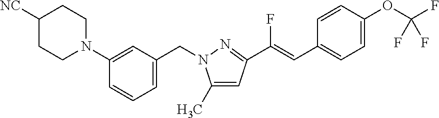 [SMILES: Cc1cc(/C(F)=C/c2ccc(OC(F)(F)F)cc2)nn1Cc1cccc(N2CCC(C#N)CC2)c1]

Under argon, a mixture of 238 mg (0.522 mmol) of the compound from Example 6, 115 mg (1.04 mmol) of 4-cyanopiperidine, 32 mg (0.035 mmol) of tris(dibenzylideneacetone)dipalladium, 50 mg (0.104 mmol) of 2-dicyclohexylphosphino-2′,4′,6′-triisopropylbiphenyl (X-Phos) and 425 mg (1.31 mmol) of caesium carbonate in 4.8 ml of DMF was heated in a microwave oven (Biotage Initiator with dynamic irradiation power control) at 120° C. for 2 h. After cooling to RT, 50 ml of water and 50 ml of ethyl acetate were added, and after phase separation the aqueous phase was extracted three times with in each case 50 ml of ethyl acetate. The combined organic phases were dried over sodium sulphate, filtered and concentrated. The residue was purified initially by column chromatography (silica gel, mobile phase cyclohexane/ethyl acetate 7:3) and then by preparative HPLC (Method 30). Drying under high vacuum thus gave 155 mg (61% of theory) of the title compound.

¹H NMR (400 MHz, CDCl₃, δ/ppm): 7.63 (d, 2H), 7.24-7.16 (m, 3H), 6.83 (dd, 1H), 6.67 (s, 1H), 6.61 (d, 1H), 6.37 (d, 1H), 6.31 (s, 1H), 5.28 (s, 2H), 3.42-3.32 (m, 2H), 3.11-3.02 (m, 2H), 2.82-2.74 (m, 1H), 2.22 (s, 3H), 2.09-1.92 (m, 4H).

LC/MS (Method 2, ESIpos): Rₜ=1.60 min, m/z=485 [M+H]⁺.

### Example 10

### 1-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}piperidine-4-carbonitrite

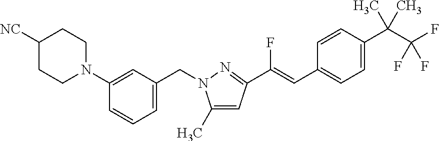 [SMILES: Cc1cc(/C(F)=C/c2ccc(C(C)(C)C(F)(F)F)cc2)nn1Cc1cccc(N2CCC(C#N)CC2)c1]

Under argon, a mixture of 470 mg (0.976 mmol) of the compound from Example 7, 215 mg (1.95 mmol) of 4-cyanopiperidine, 60 mg (0.065 mmol) of tris(dibenzylideneacetone)dipalladium, 93 mg (0.195 mmol) of 2-dicyclohexylphosphino-2′,4′,6′-triisopropylbiphenyl (X-Phos) and 785 mg (2.44 mmol) of caesium carbonate in 9 ml of DMF was stirred at 80° C. for 17 h. After cooling to RT, 50 ml of water and 50 ml of ethyl acetate were added, and after phase separation the organic phase was washed once with 50 ml of water, dried over sodium sulphate, filtered and concentrated. The residue was purified by column chromatography (silica gel, mobile phase cyclohexane/ethyl acetate 7:3). Drying under high vacuum gave 383 mg (74% of theory, purity 96%) of the title compound.

¹H NMR (400 MHz, CDCl₃, δ/ppm): 7.61 (d, 2H), 7.48 (d, 2H), 7.21 (t, 1H), 6.83 (dd, 1H), 6.67 (s, 1H), 6.61 (d, 1H), 6.37 (d, 1H), 6.31 (s, 1H), 5.28 (s, 2H), 3.42-3.34 (m, 2H), 3.10-3.03 (m, 2H), 2.81-2.74 (m, 1H), 2.21 (s, 3H), 2.09-1.90 (m, 4H), 1.58 (s, 6H).

LC/MS (Method 5, ESIpos): Rₜ=1.41 min, m/z=511 [M+H]⁺.

### Example 11

### 1-[3-({3-[(Z)-1-Fluoro-2-{4-[1-(trifluoromethyl)cyclopropyl]phenyl}vinyl]-5-methyl-1H-pyrazol-1-yl}methyl)phenyl]piperidine-4-carbonitrile

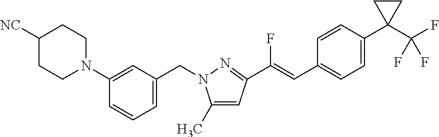 [SMILES: Cc1cc(/C(F)=C/c2ccc(C3(C(F)(F)F)CC3)cc2)nn1Cc1cccc(N2CCC(C#N)CC2)c1]

Analogously to the process described under Example 9, 250 mg (0.522 mmol) of the compound from Example 8 and 115 mg (1.04 mmol) of 4-cyanopiperidine gave 186 mg (70% of theory) of the title compound. Here, however, the mixture was heated in the microwave for only 1 h instead of 2 h.

¹H NMR (400 MHz, CDCl₃, δ/ppm): 7.58 (d, 2H), 7.44 (d, 2H), 7.21 (t, 1H), 6.83 (dd, 1H), 6.67 (s, 1H), 6.61 (d, 1H), 6.37 (d, 1H), 6.30 (s, 1H), 5.27 (s, 2H), 3.41-3.33 (m, 2H), 3.11-3.02 (m, 2H), 2.82-2.74 (m, 1H), 2.21 (s, 3H), 2.09-1.91 (m, 4H), 1.37-1.32 (m, 2H), 1.06-1.00 (m, 2H).

LC/MS (Method 2, ESIpos): Rₜ=1.62 min, m/z=509 [M+H]⁺.

### Example 12

### 1-{3-[(3-{(Z)-1-Fluoro-2-[4-(trifluoromethoxy)phenyl]vinyl}-5-methyl-1H-pyrazol-1-yl)methyl]phenyl}piperidin-4-ol

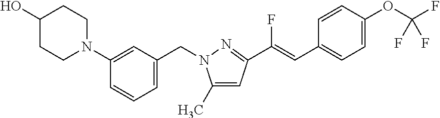 [SMILES: Cc1cc(/C(F)=C/c2ccc(OC(F)(F)F)cc2)nn1Cc1cccc(N2CCC(O)CC2)c1]

Under argon, a mixture of 238 mg (0.522 mmol) of the compound from Example 6, 301 mg (0.887 mmol) of the compound from Example 22A, 32 mg (0.035 mmol) of tris(dibenzylideneacetone)dipalladium, 50 mg (0.104 mmol) of 2-dicyclohexylphosphino-2′,4′,6′-triisopropylbiphenyl (X-Phos) and 425 mg (1.31 mmol) of caesium carbonate in 4.8 ml of DMF was heated in a microwave oven (Biotage Initiator with dynamic irradiation power control) at 120° C. for 1 h. After cooling to RT, 100 ml of water and 100 ml of ethyl acetate were added, and after phase separation the aqueous phase was extracted once with 50 ml of ethyl acetate. The combined organic phases were dried over sodium sulphate, filtered and concentrated. The residue was dissolved in 10 ml of THF, 1.3 ml of a 1 M tetra-n-butylammonium fluoride solution in THF were added and the mixture was stirred at RT for 2 h. 50 ml of water and 50 ml of ethyl acetate were then added, and after phase separation the aqueous phase was extracted once with 50 ml of ethyl acetate. The combined organic phases were dried over sodium sulphate, filtered and concentrated. The residue was purified by column chromatography (silica gel, mobile phase cyclohexane/ethyl acetate 1:1). Drying under high vacuum gave 173 mg (70% of theory) of the title compound.

¹H NMR (400 MHz, CDCl₃, δ/ppm): 7.63 (d, 2H), 7.22-7.16 (m, 3H), 6.85 (dd, 1H), 6.69 (s, 1H), 6.55 (d, 1H), 6.37 (d, 1H), 6.30 (s, 1H), 5.27 (s, 2H), 3.89-3.79 (m, 1H), 3.56-3.47 (m, 2H), 2.90 (ddd, 2H), 2.21 (s, 3H), 2.03-1.93 (m, 2H), 1.71-1.60 (m, 2H), 1.46 (br. s, 1H).

LC/MS (Method 5, ESIpos): Rₜ=1.20 min, m/z=476 [M+H]⁺.

### Example 13

### 1-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}piperidin-4-ol

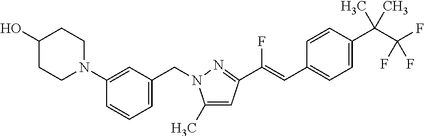 [SMILES: Cc1cc(/C(F)=C/c2ccc(C(C)(C)C(F)(F)F)cc2)nn1Cc1cccc(N2CCC(O)CC2)c1]

At RT and under argon, 198 mg (1.95 mmol) of 4-hydroxypiperidine, 60 mg (0.065 mmol) of tris(dibenzylideneacetone)dipalladium, 93 mg (0.195 mmol) of 2-dicyclohexylphosphino-2′,4′,6′-triisopropylbiphenyl (X-Phos) and 795 mg (2.44 mmol) of caesium carbonate were added to a solution of 470 mg (0.976 mmol) of the compound from Example 7 in 9 ml of DMF. The reaction mixture was stirred at a bath temperature of 80° C. for 16 h and then allowed to cool to RT and filtered through Celite, and the filter cake was washed with DMF. The filtrate was concentrated, and the residue was purified by preparative HPLC (Method 13). This gave two main fractions which, according to analytical LC/MS, consisted firstly of the title compound and secondly of the compound described under Example 1 (see there). The fraction of the title compound was freed on a rotary evaporator from the methanol of the HPLC separation, adjusted to a pH of 7-8 with saturated aqueous sodium bicarbonate solution and extracted twice with ethyl acetate. The combined organic phases were dried over sodium sulphate, filtered and concentrated. The residue (66 mg) was re-purified once more by preparative HPLC (Method 31). The substance obtained in this manner, which was identified as the trifluoroacetic acid ester of the title compound, was dissolved in 4 ml of methanol, 2-3 mg (a spatula tip) of potassium hydroxide powder were added and the mixture was stirred at RT for 1 h. 20 ml of water were then added, the mixture was then extracted three times with in each case 20 ml of tert-butyl methyl ether, and the combined organic phases were dried over sodium sulphate, filtered and concentrated. Drying under high vacuum gave 47 mg (9% of theory) of the title compound.

¹H NMR (400 MHz, CDCl₃, δ/ppm): 7.61 (d, 2H), 7.47 (d, 2H), 7.19 (t, 1H), 6.84 (dd, 1H), 6.70 (s, 1H), 6.55 (d, 1H), 6.38 (d, 1H), 6.30 (s, 1H), 5.27 (s, 2H), 3.88-3.79 (m, 1H), 3.55-3.47 (m, 2H), 2.94-2.85 (m, 2H), 2.21 (s, 3H), 2.03-1.94 (m, 2H), 1.66 (d, 2H), 1.58 (s, 6H), 1.50 (br. s, 1H).

LC/MS (Method 5, ESIpos): Rₜ=1.23 min, m/z=502 [M+H]⁺.

### Example 14

### 1-[3-({3-[(Z)-1-Fluoro-2-{4-[1-(trifluoromethyl)cyclopropyl]phenyl}vinyl]-5-methyl-1H-pyrazol-1-yl}methyl)phenyl]piperidin-4-ol

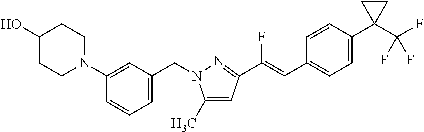 [SMILES: Cc1cc(/C(F)=C/c2ccc(C3(C(F)(F)F)CC3)cc2)nn1Cc1cccc(N2CCC(O)CC2)c1]

Analogously to the process described under Example 12, 250 mg (0.522 mmol) of the compound from Example 8 and 301 mg (0.887 mmol) of the compound from Example 22A gave 159 mg (61% of theory) of the title compound.

¹H NMR (400 MHz, CDCl₃, δ/ppm): 7.58 (d, 2H), 7.43 (d, 2H), 7.19 (t, 1H), 6.84 (dd, 1H), 6.69 (s, 1H), 6.55 (d, 1H), 6.37 (d, 1H), 6.29 (s, 1H), 5.27 (s, 2H), 3.88-3.79 (m, 1H), 3.55-3.46 (m, 2H), 2.89 (ddd, 2H), 2.21 (s, 3H), 2.03-1.93 (m, 2H), 1.70-1.60 (m, 2H), 1.48 (br. s, 1H), 1.37-1.32 (m, 2H), 1.06-1.00 (m, 2H).

LC/MS (Method 5, ESIpos): Rₜ=1.23 min, m/z=500 [M+H]⁺.

### Example 15

### 1-{3-[(3-{(Z)-1-Fluoro-2-[4-(trifluoromethoxy)phenyl]vinyl}-5-methyl-1H-pyrazol-1-yl)methyl]phenyl}azetidin-3-ol

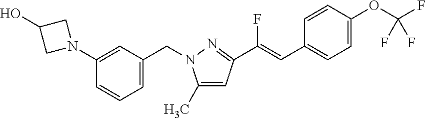 [SMILES: Cc1cc(/C(F)=C/c2ccc(OC(F)(F)F)cc2)nn1Cc1cccc(N2CC(O)C2)c1]

Analogously to the process described under Example 12, 250 mg (0.549 mmol) of the compound from Example 6 and 291 mg (0.934 mmol) of the compound from Example 23A gave 181 mg (74% of theory) of the title compound. Here, the mobile phase used for column chromatography on silica gel was cyclohexane/ethyl acetate 3:2.

¹H NMR (400 MHz, CDCl₃, δ/ppm): 7.63 (d, 2H), 7.19 (d, 2H), 7.15 (t, 1H), 6.48 (d, 1H), 6.38 (dd, 1H), 6.37 (d, 1H), 6.29 (s, 1H), 6.20 (s, 1H), 5.25 (s, 2H), 4.73 (m, 1H), 4.13 (t, 2H), 3.63 (dd, 2H), 2.33 (br. s, 1H), 2.21 (s, 3H).

LC/MS (Method 5, ESIpos): Rₜ=1.26 min, m/z=448 [M+H]⁺.

### Example 16

### 1-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}azetidin-3-ol

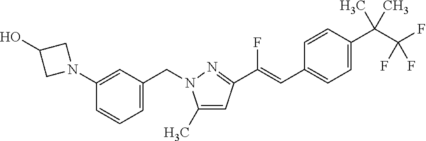 [SMILES: Cc1cc(/C(F)=C/c2ccc(C(C)(C)C(F)(F)F)cc2)nn1Cc1cccc(N2CC(O)C2)c1]

Analogously to the process described under Example 10, 470 mg (0.976 mmol) of the compound from Example 7 and 214 mg (1.95 mmol) of 3-hydroxyazetidine hydrochloride gave 75 mg (16% of theory) of the title compound. In this case, 3.5 equivalents of caesium carbonate, corresponding to 1.11 g (3.42 mmol), were employed, and the reaction mixture was heated for 30 h (instead of 17 h) at a bath temperature of 80° C.

¹H NMR (400 MHz, CDCl₃, δ/ppm): 7.61 (d, 2H), 7.47 (d, 2H), 7.15 (t, 1H), 6.48 (d, 1H), 6.37 (dd, 1H), 6.37 (d, 1H), 6.29 (s, 1H), 6.21 (s, 1H), 5.26 (s, 2H), 4.77-4.68 (m, 1H), 4.13 (t, 2H), 3.63 (dd, 2H), 2.20 (s, 3H), 2.17 (br. s, 1H), 1.58 (s, 6H).

LC/MS (Method 5, ESIpos): Rₜ=1.30 min, m/z=474 [M+H]⁺.

### Example 17

### 1-[3-({3-[(Z)-1-Fluoro-2-{4-[1-(trifluoromethyl)cyclopropyl]phenyl}vinyl]-5-methyl-1H-pyrazol-1-yl}methyl)phenyl]azetidin-3-ol

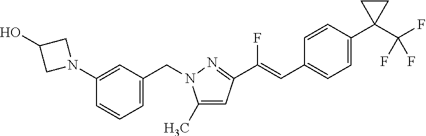 [SMILES: Cc1cc(/C(F)=C/c2ccc(C3(C(F)(F)F)CC3)cc2)nn1Cc1cccc(N2CC(O)C2)c1]

Analogously to the process described under Example 12, 100 mg (0.209 mmol) of the compound from Example 8 and 110 mg (0.355 mmol) of the compound from Example 23A gave 56 mg (57% of theory) of the title compound. Here, the intermediate resulting from the first aqueous work-up was dissolved in 5 ml of THF and stirred at RT with 0.5 ml of a 1 M tetra-n-butylammonium fluoride solution in THF for 2 h. The mobile phase used for column chromatography on silica gel was cyclohexane/ethyl acetate 3:2.

¹H NMR (400 MHz, CDCl₃, δ/ppm): 7.58 (d, 2H), 7.43 (d, 2H), 7.15 (t, 1H), 6.48 (d, 1H), 6.37 (dd, 1H), 6.37 (d, 1H), 6.29 (s, 1H), 6.21 (s, 1H), 5.25 (s, 2H), 4.73 (quint, 1H), 4.13 (t, 2H), 3.63 (dd, 2H), 2.20 (s, 3H), 2.15 (br. s, 1H), 1.37-1.32 (m, 2H), 1.06-1.00 (m, 2H).

LC/MS (Method 5, ESIpos): Rₜ=1.29 min, m/z=472 [M+H]⁺.

### Example 18

### Methyl 3-[(3-{(Z)-1-fluoro-2-[4-(trifluoromethoxy)phenyl]vinyl}-5-methyl-1H-pyrazol-1-yl)methyl]benzoate

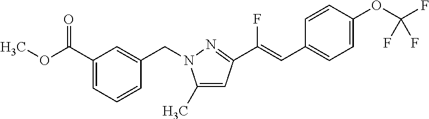 [SMILES: COC(=O)c1cccc(Cn2nc(/C(F)=C/c3ccc(OC(F)(F)F)cc3)cc2C)c1]

At a temperature of 0° C., 622 mg (5.54 mmol) of solid potassium tert-butoxide were added to a solution of 1.22 g (4.26 mmol) of the compound from Example 3A and 1.27 g (5.54 mmol) of methyl 3-(bromomethyl)benzoate in 40 ml of anhydrous THF. After removal of the ice/water bath, the reaction mixture was stirred at RT for 16 h. 200 ml of water were then added, and the mixture was extracted three times with in each case about 200 ml of ethyl acetate. The combined organic extracts were washed with saturated sodium chloride solution, dried over anhydrous magnesium sulphate, filtered and freed from the solvent on a rotary evaporator. The crude product obtained in this manner was purified by MPLC (silica gel, mobile phase cyclohexane/ethyl acetate 10:1). This gave 840 mg (45% of theory) of the title compound.

¹H NMR (400 MHz, CDCl₃, δ/ppm): 7.96 (d, 1H), 7.86 (s, 1H), 7.63 (d, 2H), 7.41 (t, 1H), 7.27 (d, 1H, partially obscured by the CHCl₃ signal), 7.19 (d, 2H), 6.38 (d, 1H), 6.32 (s, 1H), 5.37 (s, 2H), 3.91 (s, 3H), 2.22 (s, 3H).

LC/MS (Method 5, ESIpos): Rₜ=1.39 min, m/z=435 [M+H]⁺.

### Example 19

### Methyl 3-({3-[(Z)-1-fluoro-2-{4-[(trifluoromethyl)sulphanyl]phenyl}vinyl]-5-methyl-1H-pyrazol-1-yl}methyl)benzoate

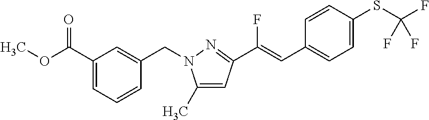 [SMILES: COC(=O)c1cccc(Cn2nc(/C(F)=C/c3ccc(SC(F)(F)F)cc3)cc2C)c1]

Analogously to the process described under Example 18, 540 mg (1.79 mmol) of the compound from Example 6A and 532 mg (2.32 mmol) methyl 3-(bromomethyl)benzoate gave 535 mg (60% of theory, 90% pure) of the title compound.

¹H NMR (400 MHz, CDCl₃, δ/ppm): 7.97 (d, 1H), 7.86 (s, 1H), 7.66-7.60 (m, 4H), 7.41 (t, 1H), 7.27 (d, 1H, partially obscured by the CHCl₃ signal), 6.41 (d, 1H), 6.34 (s, 1H), 5.37 (s, 2H), 3.91 (s, 3H), 2.22 (s, 3H).

LC/MS (Method 2, ESIpos): Rₜ=1.63 min, m/z=451 [M+H]⁺.

### Example 20

### Methyl 3-[(3-{(Z)-1-fluoro-2-[4-(1,1,1-trifluoro-2-methylpropan-2-yl)phenyl]vinyl}-5-methyl-1H-pyrazol-1-yl)methyl]benzoate

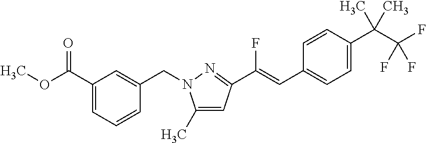 [SMILES: COC(=O)c1cccc(Cn2nc(/C(F)=C/c3ccc(C(C)(C)C(F)(F)F)cc3)cc2C)c1]

Analogously to the process described under Example 18, 450 mg (1.44 mmol) of the compound from Example 7A and 429 mg (1.87 mmol) of methyl 3-(bromomethyl)benzoate gave 430 mg (65% of theory) of the title compound. In this case, the title compound was isolated by preparative HPLC (Method 14).

¹H NMR (400 MHz, CDCl₃, δ/ppm): 7.98 (d, 1H), 7.88 (s, 1H), 7.63 (d, 2H), 7.49 (d, 2H), 7.43 (t, 1H), 7.29 (d, 1H, partially obscured by the CHCl₃ signal), 6.40 (d, 1H), 6.34 (s, 1H), 5.39 (s, 2H), 3.93 (s, 3H), 2.23 (s, 3H), 1.60 (s, 6H).

LC/MS (Method 2, ESIpos): Rₜ=1.61 min, m/z=461 [M+H]⁺.

### Example 21

### Methyl 3-({3-[(Z)-1-fluoro-2-{4-[1-(trifluoromethyl)cyclopropyl]phenyl}vinyl]-5-methyl-1H-pyrazol-1-yl}methyl)benzoate

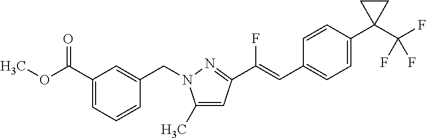 [SMILES: COC(=O)c1cccc(Cn2nc(/C(F)=C/c3ccc(C4(C(F)(F)F)CC4)cc3)cc2C)c1]

Analogously to the process described under Example 18, 250 mg (0.806 mmol) of the compound from Example 9A and 240 mg (1.05 mmol) of methyl 3-(bromomethyl)benzoate gave 250 mg (68% of theory) of the title compound. In this case, the title compound was isolated by preparative HPLC (Method 14).

¹H NMR (400 MHz, CDCl₃, δ/ppm): 7.96 (d, 1H), 7.86 (s, 1H), 7.58 (d, 2H), 7.43 (d, 2H), 7.41 (t, 1H), 7.27 (d, 1H, partially obscured by the CHCl₃ signal), 6.38 (d, 1H), 6.32 (s, 1H), 5.37 (s, 2H), 3.91 (s, 3H), 2.21 (s, 3H), 1.37-1.33 (m, 2H), 1.05-1.01 (m, 2H).

LC/MS (Method 2, ESIpos): Rₜ=1.61 min, m/z=459 [M+H]⁺.

### Example 22

### Methyl 3-[(3-{(Z)-1-fluoro-2-[4-(trifluoromethyl)phenyl]vinyl}-5-methyl-1H-pyrazol-1-yl)methyl]benzoate

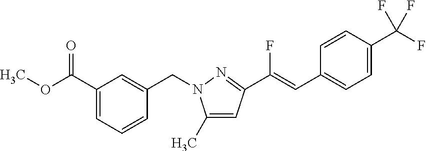 [SMILES: COC(=O)c1cccc(Cn2nc(/C(F)=C/c3ccc(C(F)(F)F)cc3)cc2C)c1]

Analogously to the process described under Example 18, 690 mg (2.55 mmol) of the compound from Example 10A and 760 mg (3.32 mmol) of methyl 3-(bromomethyl)benzoate gave 410 mg (38% of theory) of the title compound. In this case, the title compound was isolated by preparative HPLC (Method 14).

¹H NMR (400 MHz, CDCl₃, δ/ppm): 7.97 (d, 1H), 7.86 (s, 1H), 7.71 (d, 2H), 7.59 (d, 2H), 7.42 (t, 1H), 7.28 (d, 1H, partially obscured by the CHCl₃ signal), 6.44 (d, 1H), 6.35 (s, 1H), 5.37 (s, 2H), 3.91 (s, 3H), 2.23 (s, 3H).

LC/MS (Method 5, ESIpos): Rₜ=1.40 min, m/z=419 [M+H]⁺.

### Example 23

### Methyl 3-[(3-{(Z)-1-fluoro-2-[4-(trimethylsilyl)phenyl]vinyl}-5-methyl-1H-pyrazol-1-yl)methyl]benzoate

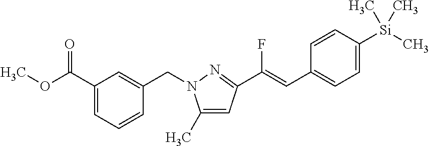

Analogously to the process described under Example 18, 300 mg (1.09 mmol) of the compound from Example 11A were reacted with 326 mg (1.42 mmol) of methyl 3-(bromomethyl)benzoate. 1 ml of water and 4 ml of methanol were added to the reaction mixture obtained after 18 h of stirring at RT, and the mixture was pre-purified directly by preparative HPLC (Method 27). The combined product fractions were freed from the acetonitrile on a rotary evaporator and adjusted to pH 8 by addition of saturated aqueous sodium bicarbonate solution. The mixture was then extracted three times with ethyl acetate, and the combined organic phases were dried over sodium sulphate, filtered and concentrated. The residue was re-purified by another preparative HPLC (Method 32). Drying under high vacuum gave 239 mg (52% of theory) of the title compound.

¹H NMR (400 MHz, CDCl₃, δ/ppm): 7.96 (d, 1H), 7.86 (s, 1H), 7.60 (d, 2H), 7.51 (d, 2H), 7.41 (t, 1H), 7.28 (s, 1H), 6.38 (d, 1H), 6.32 (s, 1H), 5.37 (s, 2H), 3.91 (s, 3H), 2.21 (s, 3H), 0.27 (s, 9H).

LC/MS (Method 2, ESIpos): Rₜ=1.68 min, m/z=423 [M+H]⁺.

### Example 24

### 3-[(3-{(Z)-1-Fluoro-2-[4-(trifluoromethoxy)phenyl]vinyl}-5-methyl-1H-pyrazol-1-yl)methyl]benzamide

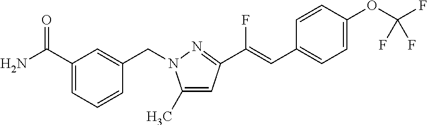 [SMILES: Cc1cc(/C(F)=C/c2ccc(OC(F)(F)F)cc2)nn1Cc1cccc(C(N)=O)c1]

At RT, 83 μl (0.952 mmol) of oxalyl chloride and a drop of DMF were added to a solution of 80 mg (0.190 mmol) of the compound from Example 33A in 3 ml of anhydrous dichloromethane. After the reaction mixture had been stirred at RT for 1 h, all volatile components were removed on a rotary evaporator and the intermediate (acid chloride) obtained was freed from the last remaining solvent and reagent residues by about 30 min under high vacuum. The intermediate was then dissolved in 2 ml of THF and, at RT, added dropwise to 1.2 ml of ammonia solution (25% in water). The reaction mixture was stirred at RT for 16 h. This resulted in the precipitation of a white solid was filtered off and washed with cold water. Drying under high vacuum gave 69 mg (83% of theory) of the title compound.

¹H NMR (400 MHz, CDCl₃, δ/ppm): 7.71 (d, 1H), 7.64 (s, 1H), 7.63 (d, 2H), 7.42 (t, 1H), 7.26 (d, 1H, partially obscured by the CHCl₃ signal), 7.19 (d, 2H), 6.37 (d, 1H), 6.32 (s, 1H), 6.02 (very broad, 1H), 5.60 (very broad, 1H), 5.37 (s, 2H), 2.22 (s, 3H).

LC/MS (Method 6, ESIpos): Rₜ=2.43 min, m/z=420 [M+H]⁺, 839 [2M+H]⁺.

### Example 25

### 3-[(3-{(Z)-1-Fluoro-2-[4-(trifluoromethoxy)phenyl]vinyl}-5-methyl-1H-pyrazol-1-yl)methyl]-N,N-dimethylbenzamide

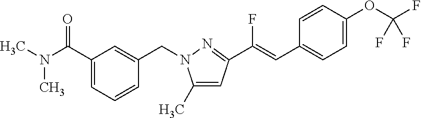 [SMILES: Cc1cc(/C(F)=C/c2ccc(OC(F)(F)F)cc2)nn1Cc1cccc(C(=O)N(C)C)c1]

At RT, 83 μl (0.952 mmol) of oxalyl chloride and a drop of DMF were added to a solution of 80 mg (0.190 mmol) of the compound from Example 33A in 3 ml of anhydrous dichloromethane. After the reaction mixture had been stirred at RT for 1 h, all volatile components were removed on a rotary evaporator and the intermediate (acid chloride) obtained was freed from the last remaining solvent and reagent residues by about 30 min under high vacuum. A solution of 66 μl (0.381 mmol) of N,N-diisopropylethylamine and 285 μl (0.571 mmol) of a 2 M solution of dimethylamine in THF was then initially charged in a further 2 ml of anhydrous THF, and a solution of the intermediate in 1 ml of anhydrous THF was added dropwise at RT. The reaction mixture was stirred at RT for 16 h. The mixture was then diluted with in each case about 1.5 ml of methanol and DMF and directly separated into its components by preparative HPLC (Method 14). Evaporation of the product fractions and drying of the residue under high vacuum gave 80 mg (85% of theory) of the title compound.

¹H NMR (400 MHz, CDCl₃, δ/ppm): 7.62 (d, 2H), 7.37 (t, 1H), 7.33 (d, 1H), 7.19 (d, 2H), 7.16 (s, 1H), 7.13 (d, 1H), 6.36 (d, 1H), 6.32 (s, 1H), 5.34 (s, 2H), 3.09 (s, broad, 3H), 2.93 (s, broad, 3H), 2.22 (s, 3H).

LC/MS (Method 6, ESIpos): Rₜ=2.56 min, m/z=448 [M+H]⁺, 895 [2M+H]⁺.

### Example 26

### {3-[(3-{(Z)-1-Fluoro-2-[4-(trifluoromethoxy)phenyl]vinyl}-5-methyl-1H-pyrazol-1-yl)-methyl]phenyl}(pyrrolidin-1-yl)methanone

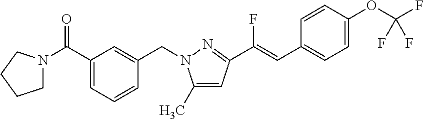 [SMILES: Cc1cc(/C(F)=C/c2ccc(OC(F)(F)F)cc2)nn1Cc1cccc(C(=O)N2CCCC2)c1]

At RT, 83 μl (0.952 mmol) of oxalyl chloride and a drop of DMF were added to a solution of 80 mg (0.190 mmol) of the compound from Example 33A in 3 ml of anhydrous dichloromethane. After the reaction mixture had been stirred at RT for 1 h, all volatile components were removed on a rotary evaporator and the intermediate (acid chloride) obtained was freed from the last remaining solvent and reagent residues by about 30 min under high vacuum. A solution of 24 μl (0.285 mmol) of pyrrolidine and 66 μl (0.381 mmol) of N,N-diisopropylethylamine in 2 ml of anhydrous THF was then initially charged, and a solution of the intermediate in 1 ml of anhydrous THF was added dropwise at RT. The reaction mixture was stirred at RT for 16 h. The mixture was then diluted with in each case about 1.5 ml of methanol and DMF and directly separated into its components by preparative HPLC (Method 14). Evaporation of the product fractions and drying of the residue under high vacuum gave 74 mg (82% of theory) of the title compound.

¹H NMR (400 MHz, CDCl₃, δ/ppm): 7.62 (d, 2H), 7.44 (d, 1H), 7.36 (t, 1H), 7.26 (s, 1H, partially obscured by the CHCl₃ signal), 7.19 (d, 2H), 7.14 (d, 1H), 6.36 (d, 1H), 6.32 (s, 1H), 5.34 (s, 2H), 3.62 (t, 2H), 3.35 (t, 2H), 2.22 (s, 3H), 1.94 (quint, 2H), 1.85 (quint, 2H).

LC/MS (Method 6, ESIpos): Rₜ=2.64 min, m/z=474 [M+H]⁺, 947 [2M+H]⁺.

### Example 27

### [3-({3-[(Z)-1-Fluoro-2-{4-[(trifluoromethyl)sulphanyl]phenyl}vinyl]-5-methyl-1H-pyrazol-1-yl}methyl)phenyl](pyrrolidin-1-yl)methanone

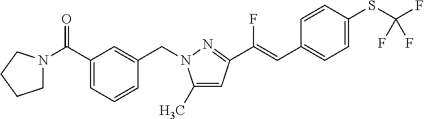 [SMILES: Cc1cc(/C(F)=C/c2ccc(SC(F)(F)F)cc2)nn1Cc1cccc(C(=O)N2CCCC2)c1]

Analogously to the process described under Example 26, 95 mg (0.218 mmol) of the compound from Example 34A and 27 μl (0.327 mmol) of pyrrolidine gave 91 mg (86% of theory) of the title compound.

¹H NMR (400 MHz, CDCl₃, δ/ppm): 7.64 (d, 2H), 7.62 (d, 2H), 7.44 (d, 1H), 7.37 (t, 1H), 7.26 (s, 1H, obscured by the CHCl₃ signal), 7.14 (d, 1H), 6.39 (d, 1H), 6.34 (s, 1H), 5.35 (s, 2H), 3.62 (t, 2H), 3.35 (t, 2H), 2.23 (s, 3H), 1.94 (quint, 2H), 1.85 (quint, 2H).

LC/MS (Method 5, ESIpos): Rₜ=1.32 min, m/z=490 [M+H]⁺, 979 [2M+H]⁺.

### Example 28

### {3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}(pyrrolidin-1-yl)methanone

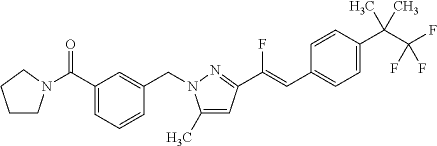 [SMILES: Cc1cc(/C(F)=C/c2ccc(C(C)(C)C(F)(F)F)cc2)nn1Cc1cccc(C(=O)N2CCCC2)c1]

Analogously to the process described under Example 26, 100 mg (0.224 mmol) of the compound from Example 35A and 28 μl (0.336 mmol) of pyrrolidine gave 77 mg (68% of theory) of the title compound. Here, the isolation of the product by preparative HPLC was followed by another preparative HPLC (Method 33) for further purification.

¹H NMR (400 MHz, CDCl₃, δ/ppm): 7.60 (d, 2H), 7.48 (d, 2H), 7.44 (d, 1H), 7.36 (t, 1H), 7.25 (s, 1H, partially obscured by the CHCl₃ signal), 7.14 (d, 1H), 6.36 (d, 1H), 6.32 (s, 1H), 5.35 (s, 2H), 3.62 (t, 2H), 3.35 (t, 2H), 2.22 (s, 3H), 1.94 (quint, 2H), 1.85 (quint, 2H), 1.58 (s, 6H, partially obscured by the water signal).

LC/MS (Method 5, ESIpos): Rₜ=1.32 min, m/z=500 [M+H]⁺, 999 [2M+H]⁺.

### Example 29

### [3-({3-[(Z)-1-Fluoro-2-{4-[1-(trifluoromethyl)cyclopropyl]phenyl}vinyl]-5-methyl-1H-pyrazol-1-yl}methyl)phenyl](pyrrolidin-1-yl)methanone

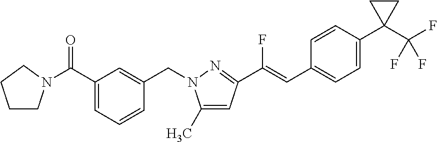 [SMILES: Cc1cc(/C(F)=C/c2ccc(C3(C(F)(F)F)CC3)cc2)nn1Cc1cccc(C(=O)N2CCCC2)c1]

Analogously to the process described under Example 26, 70 mg (0.158 mmol) of the compound from Example 36A and 20 μl (0.236 mmol) of pyrrolidine gave 34 mg (43% of theory) of the title compound. Here, the isolation of the product by preparative HPLC was followed by another preparative HPLC (Method 33) for further purification.

¹H NMR (400 MHz, CDCl₃, δ/ppm): 7.58 (d, 2H), 7.44 (2 d, tog. 3H), 7.36 (t, 1H), 7.25 (s, 1H, partially obscured by the CHCl₃ signal), 7.14 (d, 1H), 6.36 (d, 1H), 6.32 (s, 1H), 5.34 (s, 2H), 3.62 (t, 2H), 3.35 (t, 2H), 2.22 (s, 3H), 1.94 (quint, 2H), 1.84 (quint, 2H), 1.36-1.33 (m, 2H), 1.05-1.02 (m, 2H).

LC/MS (Method 5, ESIpos): Rₜ=1.30 min, m/z=498 [M+H]⁺, 995 [2M+H]⁺.

### Example 30

### {3-[(3-{(Z)-1-Fluoro-2-[4-(trifluoromethyl)phenyl]vinyl}-5-methyl-1H-pyrazol-1-yl)methyl]-phenyl}(pyrrolidin-1-yl)methanone

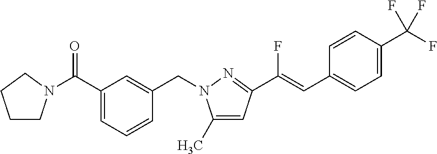 [SMILES: Cc1cc(/C(F)=C/c2ccc(C(F)(F)F)cc2)nn1Cc1cccc(C(=O)N2CCCC2)c1]

Analogously to the process described under Example 26, 80 mg (0.198 mmol) of the compound from Example 37A and 25 μl (0.297 mmol) of pyrrolidine gave 68 mg (75% of theory) of the title compound.

¹H NMR (400 MHz, CDCl₃, δ/ppm): 7.70 (d, 2H), 7.59 (d, 2H), 7.44 (d, 1H), 7.37 (t, 1H), 7.26 (s, 1H, obscured by the CHCl₃ signal), 7.14 (d, 1H), 6.42 (d, 1H), 6.34 (s, 1H), 5.35 (s, 2H), 3.62 (t, 2H), 3.35 (t, 2H), 2.23 (s, 3H), 1.94 (quint, 2H), 1.85 (quint, 2H).

LC/MS (Method 5, ESIpos): Rₜ=1.26 min, m/z=458 [M+H]⁺, 915 [2M+H]⁺.

### Example 31

### {3-[(3-{(Z)-1-Fluoro-2-[4-(trifluoromethoxy)phenyl]vinyl}-5-methyl-1H-pyrazol-1-yl)methyl]-phenyl}(morpholin-4-yl)methanone

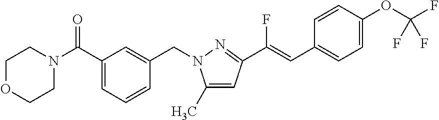 [SMILES: Cc1cc(/C(F)=C/c2ccc(OC(F)(F)F)cc2)nn1Cc1cccc(C(=O)N2CCOCC2)c1]

Analogously to the process described under Example 26, 80 mg (0.190 mmol) of the compound from Example 33A and 25 μl (0.285 mmol) of morpholine gave 84 mg (91% of theory) of the title compound.

¹H NMR (400 MHz, CDCl₃, δ/ppm): 7.62 (d, 2H), 7.39 (t, 1H), 7.33 (d, 1H), 7.19 (d, 2H), 7.16 (d, 1H), 7.12 (s, 1H), 6.35 (d, 1H), 6.32 (s, 1H), 5.35 (s, 2H), 3.81-3.32 (broad, 8H), 2.23 (s, 3H).

LC/MS (Method 5, ESIpos): Rₜ=1.22 min, m/z=490 [M+H]⁺, 979 [2M+H]⁺.

### Example 32

### {3-[(3-{(Z)-1-Fluoro-2-[4-(trifluoromethoxy)phenyl]vinyl}-5-methyl-1H-pyrazol-1-yl)-methyl]phenyl}(4-hydroxypiperidin-1-yl)methanone

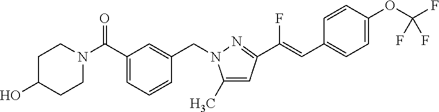 [SMILES: Cc1cc(/C(F)=C/c2ccc(OC(F)(F)F)cc2)nn1Cc1cccc(C(=O)N2CCC(O)CC2)c1]

Analogously to the process described under Example 26, 80 mg (0.190 mmol) of the compound from Example 33A and 29 mg (0.285 mmol) of 4-hydroxypiperidine gave 51 mg (54% of theory) of the title compound.

¹H NMR (400 MHz, CDCl₃, δ/ppm): 7.63 (d, 2H), 7.38 (t, 1H), 7.32 (d, 1H), 7.19 (d, 2H), 7.14 (d, 1H), 7.11 (s, 1H), 6.36 (d, 1H), 6.32 (s, 1H), 5.34 (s, 2H), 4.16 (broad, 1H), 3.99-3.92 (m, 1H), 3.59 (broad, 1H), 3.36 (broad, 1H), 3.14 (broad, 1H), 2.23 (s, 3H), 1.95 (broad, 1H), 1.78 (broad, 1H), 1.60 (broad, 1H), 1.50-1.47 (m, 1H), 1.46 (broad, 1H).

LC/MS (Method 5, ESIpos): Rₜ=1.15 min, m/z=504 [M+H]⁺, 1007 [2M+H]⁺.

### Example 33

### [3-({3-[(Z)-1-Fluoro-2-{4-[(trifluoromethyl)sulphanyl]phenyl}vinyl]-5-methyl-1H-pyrazol-1-yl}-methyl)phenyl](4-hydroxypiperidin-1-yl)methanone

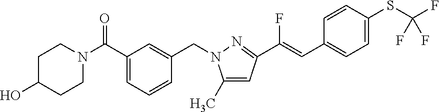 [SMILES: Cc1cc(/C(F)=C/c2ccc(SC(F)(F)F)cc2)nn1Cc1cccc(C(=O)N2CCC(O)CC2)c1]

Analogously to the process described under Example 26, 95 mg (0.218 mmol) of the compound from Example 34A and 33 mg (0.327 mmol) of 4-hydroxypiperidine gave 93 mg (83% of theory) of the title compound.

¹H NMR (400 MHz, CDCl₃, δ/ppm): 7.64 (d, 2H), 7.62 (d, 2H), 7.38 (t, 1H), 7.32 (d, 1H), 7.14 (d, 1H), 7.12 (s, 1H), 6.39 (d, 1H), 6.34 (s, 1H), 5.35 (s, 2H), 4.16 (broad, 1H), 3.99-3.92 (m, 1H), 3.60 (broad, 1H), 3.37 (broad, 1H), 3.14 (broad, 1H), 2.23 (s, 3H), 1.96 (broad, 1H), 1.79 (broad, 1H), 1.64-1.42 (m, 3H).

LC/MS (Method 5, ESIpos): Rₜ=1.16 min, m/z=520 [M+H]⁺, 1039 [2M+H]⁺.

### Example 34

### {3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}(4-hydroxypiperidin-1-yl)methanone

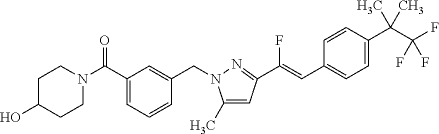 [SMILES: Cc1cc(/C(F)=C/c2ccc(C(C)(C)C(F)(F)F)cc2)nn1Cc1cccc(C(=O)N2CCC(O)CC2)c1]

Analogously to the process described under Example 26, 75 mg (0.168 mmol) of the compound from Example 35A and 26 mg (0.252 mmol) of 4-hydroxypiperidine gave 59 mg (66% of theory) of the title compound. Here, the isolation of the product by preparative HPLC was followed by another preparative HPLC (Method 33) for further purification.

¹H NMR (400 MHz, CDCl₃, δ/ppm): 7.60 (d, 2H), 7.48 (d, 2H), 7.38 (t, 1H), 7.32 (d, 1H), 7.15 (d, 1H), 7.10 (s, 1H), 6.36 (d, 1H), 6.32 (s, 1H), 5.35 (s, 2H), 4.16 (broad, 1H), 3.98-3.92 (m, 1H), 3.59 (broad, 1H), 3.36 (broad, 1H), 3.13 (broad, 1H), 2.23 (s, 3H), 1.96 (broad, 1H), 1.79 (broad, 1H), 1.58 (s, 6H, partially obscured by the water signal), 1.47 (broad, 1H), 1.30 (broad, 1H), 0.95-0.86 (m, 1H).

LC/MS (Method 5, ESIpos): Rₜ=1.15 min, m/z=530 [M+H]⁺, 1059 [2M+H]⁺.

### Example 35

### [3-({3-[(Z)-1-Fluoro-2-{4-[1-(trifluoromethyl)cyclopropyl]phenyl}vinyl]-5-methyl-1H-pyrazol-1-yl}methyl)phenyl](4-hydroxypiperidin-1-yl)methanone

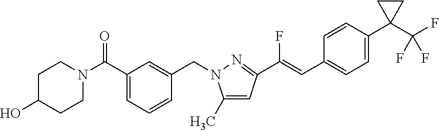 [SMILES: Cc1cc(/C(F)=C/c2ccc(C3(C(F)(F)F)CC3)cc2)nn1Cc1cccc(C(=O)N2CCC(O)CC2)c1]

Analogously to the process described under Example 26, 70 mg (0.158 mmol) of the compound from Example 36A and 24 mg (0.236 mmol) of 4-hydroxypiperidine gave 55 mg (67% of theory) of the title compound. Here, the isolation of the product by preparative HPLC was followed by another preparative HPLC (Method 33) for further purification.

¹H NMR (400 MHz, CDCl₃, δ/ppm): 7.58 (d, 2H), 7.44 (d, 2H), 7.38 (t, 1H), 7.32 (d, 1H), 7.14 (d, 1H), 7.10 (s, 1H), 6.36 (d, 1H), 6.32 (s, 1H), 5.34 (s, 2H), 4.16 (broad, 1H), 3.98-3.92 (m, 1H), 3.59 (broad, 1H), 3.36 (broad, 1H), 3.13 (broad, 1H), 2.22 (s, 3H), 1.95 (broad, 1H), 1.79 (broad, 1H), 1.55 (broad, 1H), 1.45 (broad, 1H), 1.36-1.33 (m, 2H), 1.05-1.01 (m, 2H).

LC/MS (Method 5, ESIpos): Rₜ=1.14 min, m/z=528 [M+H]⁺, 1055 [2M+H]⁺.

### Example 36

### {3-[(3-{(Z)-1-Fluoro-2-[4-(trifluoromethyl)phenyl]vinyl}-5-methyl-1H-pyrazol-1-yl)methyl]-phenyl}(4-hydroxypiperidin-1-yl)methanone

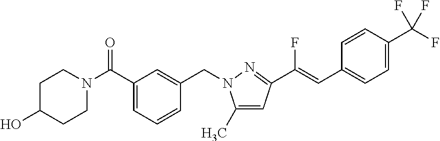 [SMILES: Cc1cc(/C(F)=C/c2ccc(C(F)(F)F)cc2)nn1Cc1cccc(C(=O)N2CCC(O)CC2)c1]

Analogously to the process described under Example 26, 80 mg (0.198 mmol) of the compound from Example 37A and 30 mg (0.297 mmol) of 4-hydroxypiperidine gave 87 mg (88% of theory) of the title compound.

¹H NMR (400 MHz, CDCl₃, δ/ppm): 7.70 (d, 2H), 7.60 (d, 2H), 7.38 (t, 1H), 7.32 (d, 1H), 7.14 (d, 1H), 7.12 (s, 1H), 6.42 (d, 1H), 6.35 (s, 1H), 5.35 (s, 2H), 4.16 (broad, 1H), 3.99-3.92 (m, 1H), 3.60 (broad, 1H), 3.37 (broad, 1H), 3.14 (broad, 1H), 2.24 (s, 3H), 1.95 (broad, 1H), 1.79 (broad, 1H), 1.64-1.52 (m, 3H, partially obscured by the water signal), 1.45 (broad, 1H).

LC/MS (Method 5, ESIpos): Rₜ=1.08 min, m/z=488 [M+H]⁺, 975 [2M+H]⁺.

### Example 37

### (4-Cyclopropylpiperazin-1-yl) {3-[(3-{(Z)-1-fluoro-2-[4-(trifluoromethoxy)phenyl]vinyl}-5-methyl-1H-pyrazol-1-yl)methyl]phenyl}methanone

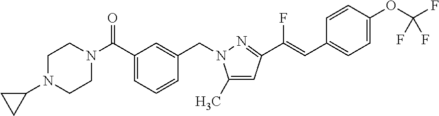 [SMILES: Cc1cc(/C(F)=C/c2ccc(OC(F)(F)F)cc2)nn1Cc1cccc(C(=O)N2CCN(C3CC3)CC2)c1]

At RT, 73 μl (0.833 mmol) of oxalyl chloride and a drop of DMF were added to a solution of 70 mg (0.167 mmol) of the compound from Example 33A in 3 ml of anhydrous dichloromethane. After the reaction mixture had been stirred at RT for 1 h, all volatile components were removed on a rotary evaporator and the intermediate (acid chloride) obtained was freed from the last remaining solvent and reagent residues by about 30 min under high vacuum. A solution of 66 mg (0.333 mmol) of 1-cyclopropylpiperazine dihydrochloride and 145 μl (0.833 mmol) of N,N-diisopropyl-ethylamine in 2 ml of anhydrous THF was then initially charged, and a solution of the intermediate in 1 ml of anhydrous THF was added dropwise at RT. The reaction mixture was stirred at RT for 16 h. About 2 ml of water were then added, and the mixture was separated directly into its components by preparative HPLC (Method 34). After evaporation of the product fractions, the product obtained was dissolved in about 5 ml of methanol and passed through an ion exchanger column (Polymerlabs, Stratospheres SPE, PL-HCO₃ MP SPE, capacity 0.9 mmol) to convert the formic acid salt (from the HPLC) into the free acid. Evaporation and drying under high vacuum gave 62 mg (70% of theory) of the title compound.

¹H NMR (400 MHz, CDCl₃, δ/ppm): 7.62 (d, 2H), 7.39 (t, 1H), 7.34 (d, 1H), 7.19 (d, 2H), 7.15 (d, 1H), 7.07 (s, 1H), 6.36 (d, 1H), 6.33 (s, 1H), 5.35 (s, 2H), 3.70 (broad, 2H), 3.29 (broad, 2H), 2.65 (broad, 2H), 2.49 (broad, 2H), 2.23 (s, 3H), 1.59-1.54 (m, 1H), 0.40-0.34 (m, 4H).

LC/MS (Method 5, ESIpos): Rₜ=1.04 min, m/z=529 [M+H]⁺, 1057 [2M+H]⁺.

### Example 38

### (4-Cyclopropylpiperazin-1-yl)[3-({3-[(Z)-1-fluoro-2-{4-[(trifluoromethyl)sulphanyl]phenyl}vinyl]-5-methyl-1H-pyrazol-1-yl}methyl)phenyl]methanone

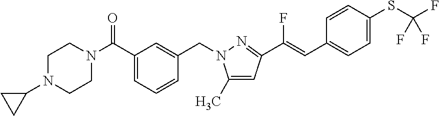 [SMILES: Cc1cc(/C(F)=C/c2ccc(SC(F)(F)F)cc2)nn1Cc1cccc(C(=O)N2CCN(C3CC3)CC2)c1]

Analogously to the process described under Example 37, 80 mg (0.183 mmol) of the compound from Example 34A and 73 mg (0.367 mmol) of 1-cyclopropylpiperazine dihydrochloride gave 74 mg (75% of theory) of the title compound.

¹H NMR (400 MHz, CDCl₃, δ/ppm): 7.64 (d, 2H), 7.61 (d, 2H), 7.39 (t, 1H), 7.34 (d, 1H), 7.16 (d, 1H), 7.08 (s, 1H), 6.40 (d, 1H), 6.35 (s, 1H), 5.35 (s, 2H), 3.70 (broad, 2H), 3.29 (broad, 2H), 2.65 (broad, 2H), 2.49 (broad, 2H), 2.24 (s, 3H), 1.58-1.54 (m, 1H), 0.39-0.34 (m, 4H).

LC/MS (Method 2, ESIpos): Rₜ=1.27 min, m/z=545 [M+H]⁺, 1089 [2M+H]⁺.

### Example 39

### (4-Cyclopropylpiperazin-1-yl) {3-[(3-{(Z)-1-fluoro-2-[4-(1,1,1-trifluoro-2-methylpropan-2-yl)-phenyl]vinyl}-5-methyl-1H-pyrazol-1-yl)methyl]phenyl}methanone

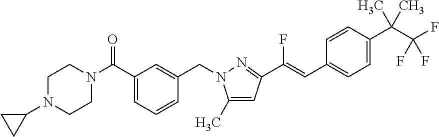 [SMILES: Cc1cc(/C(F)=C/c2ccc(C(C)(C)C(F)(F)F)cc2)nn1Cc1cccc(C(=O)N2CCN(C3CC3)CC2)c1]

Analogously to the process described under Example 37, 58 mg (0.130 mmol) of the compound from Example 35A and 52 mg (0.260 mmol) of 1-cyclopropylpiperazine dihydrochloride gave 59 mg (82% of theory) of the title compound.

¹H NMR (400 MHz, CDCl₃, δ/ppm): 7.60 (d, 2H), 7.47 (d, 2H), 7.38 (t, 1H), 7.32 (d, 1H), 7.16 (d, 1H), 7.06 (s, 1H), 6.37 (d, 1H), 6.33 (s, 1H), 5.35 (s, 2H), 3.70 (broad, 2H), 3.29 (broad, 2H), 2.65 (broad, 2H), 2.48 (broad, 2H), 2.23 (s, 3H), 1.58 (s, 6H, partially superimposed by the water signal), 1.58-1.53 (m, 1H), 0.37-0.33 (m, 4H).

LC/MS (Method 5, ESIpos): Rₜ=1.07 min, m/z=555 [M+H]⁺, 1109 [2M+H]⁺.

### Example 40

### (4-Cyclopropylpiperazin-1-yl)[3-({3-[(Z)-1-fluoro-2-{4-[1-(trifluoromethyl)cyclopropyl]phenyl}-vinyl]-5-methyl-1H-pyrazol-1-yl}methyl)phenyl]methanone

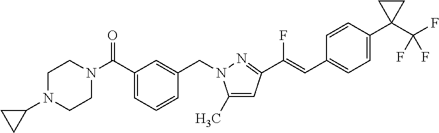 [SMILES: Cc1cc(/C(F)=C/c2ccc(C3(C(F)(F)F)CC3)cc2)nn1Cc1cccc(C(=O)N2CCN(C3CC3)CC2)c1]

Analogously to the process described under Example 37, 80 mg (0.180 mmol) of the compound from Example 36A and 72 mg (0.360 mmol) of 1-cyclopropylpiperazine dihydrochloride gave 77 mg (74% of theory) of the title compound.

¹H NMR (400 MHz, CDCl₃, δ/ppm): 7.57 (d, 2H), 7.43 (d, 2H), 7.38 (t, 1H), 7.34 (d, 1H), 7.15 (d, 1H), 7.06 (s, 1H), 6.36 (d, 1H), 6.32 (s, 1H), 5.35 (s, 2H), 3.70 (broad, 2H), 3.29 (broad, 2H), 2.64 (broad, 2H), 2.48 (broad, 2H), 2.23 (s, 3H), 1.58-1.53 (m, 1H, partially superimposed by the water signal), 1.37-1.32 (m, 2H), 1.05-1.01 (m, 2H), 0.38-0.33 (m, 4H).

LC/MS (Method 5, ESIpos): Rₜ=1.06 min, m/z=553 [M+H]⁺, 1105 [2M+H]⁺.

### Example 41

### (4-Cyclopropylpiperazin-1-yl) {3-[(3-{(Z)-1-fluoro-2-[4-(trifluoromethyl)phenyl]vinyl}-5-methyl-1H-pyrazol-1-yl)methyl]phenyl}methanone

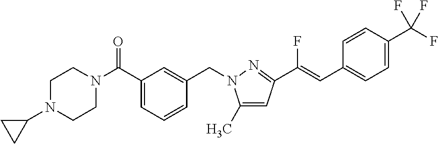 [SMILES: Cc1cc(/C(F)=C/c2ccc(C(F)(F)F)cc2)nn1Cc1cccc(C(=O)N2CCN(C3CC3)CC2)c1]

Analogously to the process described under Example 37, 80 mg (0.198 mmol) of the compound from Example 36A and 79 mg (0.396 mmol) of 1-cyclopropylpiperazine dihydrochloride gave 80 mg (80% of theory) of the title compound.

¹H NMR (400 MHz, CDCl₃, δ/ppm): 7.70 (d, 2H), 7.59 (d, 2H), 7.39 (t, 1H), 7.34 (d, 1H), 7.15 (d, 1H), 7.09 (s, 1H), 6.42 (d, 1H), 6.35 (s, 1H), 5.36 (s, 2H), 3.71 (broad, 2H), 3.30 (broad, 2H), 2.66 (broad, 2H), 2.50 (broad, 2H), 2.24 (s, 3H), 1.60-1.56 (m, 1H), 0.41-0.37 (m, 4H).

LC/MS (Method 2, ESIpos): Rₜ=1.18 min, m/z=513 [M+H]⁺, 1025 [2M+H]⁺.

### Example 42

### {3-[(3-{(Z)-1-Fluoro-2-[4-(trifluoromethoxy)phenyl]vinyl}-5-methyl-1H-pyrazol-1-yl)methyl]-phenyl}(4-methylpiperazin-1-yl)methanone

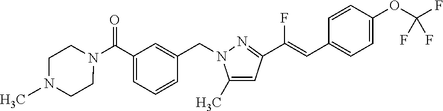 [SMILES: Cc1cc(/C(F)=C/c2ccc(OC(F)(F)F)cc2)nn1Cc1cccc(C(=O)N2CCN(C)CC2)c1]

Analogously to the process described under Example 37, 80 mg (0.190 mmol) of the compound from Example 33A and 29 mg (0.285 mmol) of 1-methylpiperazine gave 72 mg (75% of theory) of the title compound.

¹H NMR (400 MHz, CDCl₃, δ/ppm): 7.62 (d, 2H), 7.38 (t, 1H), 7.33 (d, 1H), 7.19 (d, 2H), 7.15 (d, 1H), 7.09 (s, 1H), 6.36 (d, 1H), 6.32 (s, 1H), 5.35 (s, 2H), 3.76 (broad, 2H), 3.36 (broad, 2H), 2.44 (broad, 2H), 2.28 (broad, 2H), 2.26 (s, 3H), 2.23 (s, 3H).

LC/MS (Method 2, ESIpos): Rₜ=1.08 min, m/z=503 [M+H]⁺, 1005 [2M+H]⁺.

### Example 43

### 1-{3-[(3-{(Z)-1-Fluoro-2-[4-(trifluoromethoxy)phenyl]vinyl}-5-methyl-1H-pyrazol-1-yl)methyl]phenyl}cyclopropyl acetate

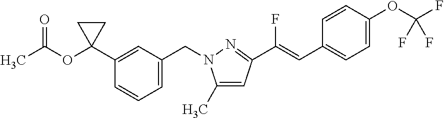 [SMILES: CC(=O)OC1(c2cccc(Cn3nc(/C(F)=C/c4ccc(OC(F)(F)F)cc4)cc3C)c2)CC1]

With stirring at 0° C., 102 mg (0.910 mmol) of potassium tert-butoxide were added to a solution of 200 mg (0.700 mmol) of the compound from Example 3A and 229 mg (0.770 mmol, purity 96%) of the compound from Example 24A in 5 ml of THF. The reaction mixture was then stirred at RT for 4 h. After addition of 100 ml of ethyl acetate, the mixture was washed once with 50 ml of water and the aqueous phase was re-extracted once with ethyl acetate. The combined organic phases were washed once with saturated sodium chloride solution, dried over magnesium sulphate, filtered and concentrated. The residue was purified by thick-layer chromatography (silica gel, mobile phase dichloromethane/methanol 100:1). The product-containing zone was extracted with dichloromethane/methanol 95:5. After removal of the solvent, pentane was added to the residue. The solid formed was filtered off and dried under high vacuum. This gave 148 mg (45% of theory) of the title compound.

¹H NMR (400 MHz, CDCl₃, δ/ppm): 7.63 (d, 2H), 7.29-7.24 (m, 1H), 7.21-7.13 (m, 3H), 7.02 (s, 1H), 6.94 (d, 1H), 6.38 (d, 1H), 6.31 (s, 1H), 5.31 (s, 2H), 2.20 (s, 3H), 2.02 (s, 3H), 1.31-1.23 (m, 2H), 1.23-1.16 (m, 2H).

LC/MS (Method 5, ESIpos): Rₜ=1.40 min, m/z=475 [M+H]⁺.

### Example 44

### 1-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}cyclopropyl acetate

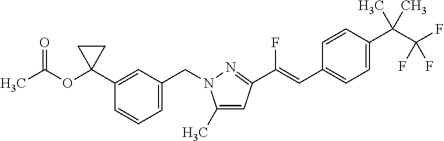 [SMILES: CC(=O)OC1(c2cccc(Cn3nc(/C(F)=C/c4ccc(C(C)(C)C(F)(F)F)cc4)cc3C)c2)CC1]

Analogously to the process described under Example 43, 219 mg (0.700 mmol) of the compound from Example 7A and 229 mg (0.770 mmol, purity 96%) of the compound from Example 24A gave 235 mg (66% of theory, purity 99%) of the title compound.

¹H NMR (400 MHz, CDCl₃ δ/ppm): 7.60 (d, 2H), 7.47 (d, 2H), 7.30-7.23 (m, 1H), 7.15 (d, 1H), 7.02 (s, 1H), 6.95 (d, 1H), 6.38 (d, 1H), 6.31 (s, 1H), 5.31 (s, 2H), 2.20 (s, 3H), 2.02 (s, 3H), 1.58 (s, 6H), 1.31-1.23 (m, 2H), 1.23-1.15 (m, 2H).

LC/MS (Method 2, ESIpos): Rₜ=1.64 min, m/z=501 [M+H]⁺.

### Example 45

### 1-{3-[(3-{(Z)-1-Fluoro-2-[3-fluoro-4-(1,1,1-trifluoro-2-methylpropan-2-yl)phenyl]vinyl}-5-methyl-1H-pyrazol-1-yl)methyl]phenyl}cyclopropyl acetate

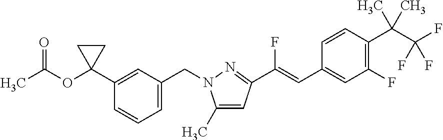 [SMILES: CC(=O)OC1(c2cccc(Cn3nc(/C(F)=C/c4ccc(C(C)(C)C(F)(F)F)c(F)c4)cc3C)c2)CC1]

Analogously to the process described under Example 43, 150 mg (0.454 mmol) of the compound from Example 8A and 149 mg (0.500 mmol, purity 96%) of the compound from Example 24A gave 236 mg (78% of theory, purity 78%) of the title compound. In this case, the reaction mixture was stirred at RT overnight (instead of 4 h).

LC/MS (Method 5, ESIpos): Rₜ=1.48 min, m/z=519 [M+H]⁺.

### Example 46

### 1-[3-({3-[(Z)-1-Fluoro-2-{4-[1-(trifluoromethyl)cyclopropyl]phenyl}vinyl]-5-methyl-1H-pyrazol-1-yl}methyl)phenyl]cyclopropyl acetate

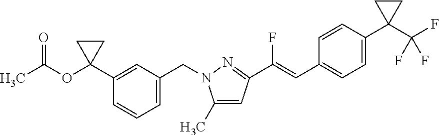 [SMILES: CC(=O)OC1(c2cccc(Cn3nc(/C(F)=C/c4ccc(C5(C(F)(F)F)CC5)cc4)cc3C)c2)CC1]

Analogously to the process described under Example 43, 150 mg (0.483 mmol) of the compound from Example 9A and 158 mg (0.532 mmol, purity 96%) of the compound from Example 24A gave 166 mg (67% of theory) of the title compound.

¹H NMR (400 MHz, CDCl₃, δ/ppm): 7.58 (d, 2H), 7.43 (d, 2H), 7.26 (t, 1H), 7.15 (d, 1H), 7.02 (s, 1H), 6.95 (d, 1H), 6.38 (d, 1H), 6.31 (s, 1H), 5.31 (s, 2H), 2.20 (s, 3H), 2.02 (s, 3H), 1.35 (dd, 2H), 1.31-1.23 (m, 2H), 1.23-1.16 (m, 2H), 1.06-1.00 (m, 2H).

LC/MS (Method 2, ESIpos): Rₜ=1.63 min, m/z=499 [M+H]⁺.

### Example 47

### 1-[3-({3-[(Z)-2-(4-tert-Butylphenyl)-1-fluorovinyl]-5-methyl-1H-pyrazol-1-yl}methyl)phenyl]-cyclopropyl acetate

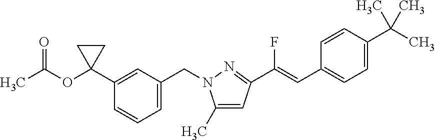 [SMILES: CC(=O)OC1(c2cccc(Cn3nc(/C(F)=C/c4ccc(C(C)(C)C)cc4)cc3C)c2)CC1]

At 0° C., 109 mg (0.972 mmol) potassium tert-butoxide were added to a solution of 193 mg (0.749 mmol) of the compound from Example 12A in 3 ml of THF. After 10 min of stirring at 0° C., 245 mg (0.824 mmol, purity 96%) of the compound from Example 24A were added. After 16 h of stirring at RT, 50 ml of ethyl acetate and 50 ml of water were added to the reaction mixture, and after phase separation the aqueous phase was extracted with 50 ml of ethyl acetate. The combined organic phases were washed once with 100 ml of saturated sodium chloride solution, dried over sodium sulphate, filtered and concentrated. The residue was purified by column chromatography (silica gel, mobile phase cyclohexane/ethyl acetate 4:1). Removal of the solvent and drying under high vacuum gave 293 mg (82% of theory, purity 93%) of the title compound.

¹H NMR (400 MHz, CDCl₃, δ/ppm): 7.56 (d, 2H), 7.38 (d, 2H), 7.29-7.24 (m, 1H, obscured by CHCl₃ signal), 7.14 (d, 1H), 7.02 (s, 1H), 6.95 (d, 1H), 6.34 (d, 2H), 6.31 (s, 1H), 5.31 (s, 2H), 2.19 (s, 3H), 2.02 (s, 3H), 1.33 (s, 9H), 1.31-1.14 (m, 4H).

LC/MS (Method 5, ESIpos): Rₜ=1.50 min, m/z=447 [M+H]⁺.

### Example 48

### 1-{3-[(3-{(Z)-1-Fluoro-2-[4-(trifluoromethoxy)phenyl]vinyl}-5-methyl-1H-pyrazol-1-yl)methyl]-phenyl}cyclopropanol

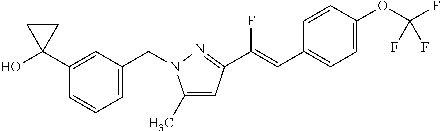 [SMILES: Cc1cc(/C(F)=C/c2ccc(OC(F)(F)F)cc2)nn1Cc1cccc(C2(O)CC2)c1]

At a bath temperature of 0° C., 1.05 ml (2.11 mmol) of a 2 M solution of ethylmagnesium bromide in THF were added slowly to a solution of 100 mg (0.211 mmol) of the compound from Example 43 in 3.5 ml of THF. The mixture was stirred initially at 0° C. for 5 min and then at RT for 25 min. The mixture was once more cooled to 0° C., and first 2.5 ml of water and then 2.5 ml of 1 M hydrochloric acid were then added slowly. The mixture was diluted further with water and extracted twice with ethyl acetate. The combined organic phases were washed once with saturated sodium chloride solution, dried over magnesium sulphate, filtered and concentrated. The residue was purified by thick-layer chromatography (silica gel, mobile phase cyclohexane/ethyl acetate 6:4). The product-containing zone was extracted with dichloromethane/methanol 95:5. After removal of the solvent, pentane was added to the residue. The solid formed was filtered off and dried under high vacuum. This gave 59 mg (63% of theory, purity 98%) of the title compound.

¹H NMR (400 MHz, CDCl₃, δ/ppm): 7.61 (d, 2H), 7.31-7.27 (m, 1H), 7.22-7.12 (m, 4H), 6.92 (d, 1H), 6.37 (d, 1H), 6.30 (s, 1H), 5.31 (s, 2H), 2.47 (s, 1H), 2.21 (s, 3H), 1.29-1.24 (m, 2H), 1.04-0.99 (m, 2H).

LC/MS (Method 5, ESIpos): Rₜ=1.29 min, m/z=433 [M+H]⁺.

### Example 49

### 1-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}cyclopropanol

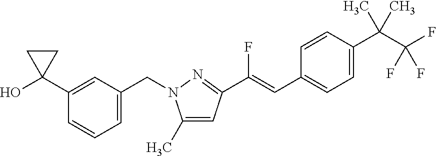 [SMILES: Cc1cc(/C(F)=C/c2ccc(C(C)(C)C(F)(F)F)cc2)nn1Cc1cccc(C2(O)CC2)c1]

Analogously to the process described under Example 48, 210 mg (0.420 mmol) of the compound from Example 44 and 2.1 ml (4.20 mmol) of a 2 Methylmagnesium bromide solution in THF gave 140 mg (68% of theory, purity 94%) of the title compound.

¹H NMR (400 MHz, CDCl₃, δ/ppm): 7.60 (d, 2H), 7.47 (d, 2H), 7.31-7.26 (m, 1H), 7.18-7.12 (m, 2H), 6.93 (d, 1H), 6.37 (d, 1H), 6.30 (s, 1H), 5.31 (s, 2H), 2.47 (br. s, 1H), 2.21 (s, 3H), 1.58 (s, 6H), 1.29-1.24 (m, 2H), 1.04-1.98 (m, 2H).

LC/MS (Method 2, ESIpos): Rₜ=1.54 min, m/z=459 [M+H]⁺.

### Example 50

### 1-{3-[(3-{(Z)-1-Fluoro-2-[3-fluoro-4-(1,1,1-trifluoro-2-methylpropan-2-yl)phenyl]vinyl}-5-methyl-1H-pyrazol-1-yl)methyl]phenyl}cyclopropanol

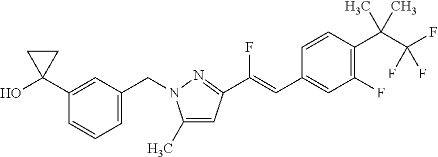 [SMILES: Cc1cc(/C(F)=C/c2ccc(C(C)(C)C(F)(F)F)c(F)c2)nn1Cc1cccc(C2(O)CC2)c1]

Analogously to the process described under Example 48, 235 mg (0.353 mmol, purity 78%) of the compound from Example 45 and 1.8 ml (3.53 mmol) of a 2 Methylmagnesium bromide solution in THF gave 66 mg (38% of theory, purity 96%) of the title compound. In this case, the reaction mixture was stirred at RT for 1 h (instead of 25 min). Here, an additional purification step by preparative HPLC (Method 16) was inserted between aqueous work-up and purification by thick-layer chromatography; the combined product fractions for the work-up were, after neutralization with saturated aqueous sodium bicarbonate solution, concentrated to a small residual volume of aqueous phase and then extracted twice with ethyl acetate, whereupon the combined organic phases were dried over magnesium sulphate, filtered and concentrated.

¹H NMR (400 MHz, CDCl₃, δ/ppm): 7.41-7.27 (m, 4H), 7.17-7.11 (m, 2H), 6.92 (d, 1H), 6.33 (d, 1H), 6.31 (s, 1H), 5.31 (s, 2H), 2.53 (br. s, 1H), 2.21 (s, 3H), 1.65 (s, 6H), 1.29-1.24 (m, 2H), 1.04-0.99 (m, 2H).

LC/MS (Method 2, ESIpos): Rₜ=1.65 min, m/z=477 [M+H]⁺.

### Example 51

### 1-[3-({3-[(Z)-1-Fluoro-2-{4-[1-(trifluoromethyl)cyclopropyl]phenyl}vinyl]-5-methyl-1H-pyrazol-1-yl}methyl)phenyl]cyclopropanol

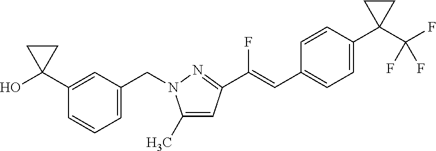 [SMILES: Cc1cc(/C(F)=C/c2ccc(C3(C(F)(F)F)CC3)cc2)nn1Cc1cccc(C2(O)CC2)c1]

Analogously to the process described under Example 48, 120 mg (0.241 mmol) of the compound from Example 46 and 1.20 ml (2.41 mmol) of a 2 M ethylmagnesium bromide solution in THF gave 68 mg (59% of theory, purity 96%) of the title compound. In this case, the crude product was purified not by thick-layer chromatography but by preparative HPLC (Method 13). The combined product fractions were neutralized with sodium bicarbonate and concentrated to a small residual volume of aqueous phase. The solid that precipitated during the concentration was filtered off, washed twice with water and dried under high vacuum, giving the title compound.

¹H NMR (400 MHz, CDCl₃, δ/ppm): 7.57 (d, 2H), 7.43 (d, 2H), 7.30-7.26 (m, 1H), 7.17-7.12 (m, 2H), 6.92 (d, 1H), 6.36 (d, 1H), 6.30 (s, 1H), 5.31 (s, 2H), 2.46 (s, 1H), 2.21 (s, 3H), 1.37-1.32 (m, 2H), 1.29-1.24 (m, 2H), 1.05-0.98 (m, 4H).

LC/MS (Method 5, ESIpos): Rₜ=1.31 min, m/z=457 [M+H]⁺.

### Example 52

### 1-[3-({3-[(Z)-2-(4-tert-Butylphenyl)-1-fluorovinyl]-5-methyl-1H-pyrazol-1-yl}methyl)phenyl]-cyclopropanol

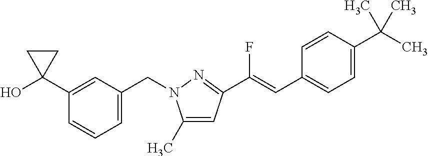 [SMILES: Cc1cc(/C(F)=C/c2ccc(C(C)(C)C)cc2)nn1Cc1cccc(C2(O)CC2)c1]

Analogously to the process described under Example 48, 295 mg (0.614 mmol, purity 93%) of the compound from Example 47 and 3.1 ml (6.14 mmol) of a 2 Methylmagnesium bromide solution in THF gave 103 mg (39% of theory, purity 95%) of the title compound. In this case, the reaction mixture was stirred at RT for 1 h (instead of 25 min). Here, the crude product was purified not by thick-layer chromatography but by preparative HPLC (Method 16). The combined product fractions were neutralized with sodium bicarbonate and concentrated to a small residual volume of aqueous phase. After two extractions with in each case 50 ml of ethyl acetate, the combined organic phases were dried over sodium sulphate, filtered and concentrated and the residue was dried under high vacuum, giving the title compound.

¹H NMR (400 MHz, CDCl₃, δ/ppm): 7.56 (d, 2H), 7.38 (d, 2H), 7.30-7.26 (m, 1H), 7.17-7.13 (m, 2H), 6.93 (d, 1H), 6.35 (d, 1H), 6.29 (s, 1H), 5.32 (s, 2H), 2.41 (br. s, 1H), 2.21 (s, 3H), 1.33 (s, 9H), 1.29-1.24 (m, 2H), 1.04-0.99 (m, 2H).

LC/MS (Method 9, ESIpos): Rₜ=5.89 min, m/z=405 [M+H]⁺.

### Example 53

### 2-{3-[(3-{(Z)-1-Fluoro-2-[4-(trifluoromethoxy)phenyl]vinyl}-5-methyl-1H-pyrazol-1-yl)methyl]-phenyl}propan-2-ol

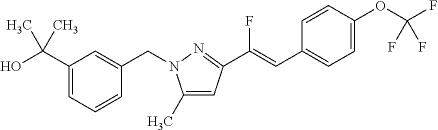 [SMILES: Cc1cc(/C(F)=C/c2ccc(OC(F)(F)F)cc2)nn1Cc1cccc(C(C)(C)O)c1]

At a temperature of 0° C., 506 μl (0.506 mmol) of a 1 M solution of methylmagnesium bromide in dibutyl ether were added dropwise to a solution of 100 mg (0.230 mmol) of the compound from Example 18 in 3 ml of anhydrous THF. The reaction mixture was then warmed to RT and stirred at this temperature for 3 h. 0.5 ml of saturated aqueous ammonium chloride solution was then added, and the mixture was diluted with about 5 ml of ethyl acetate. Anhydrous magnesium sulphate was added, and the mixture was stirred for a few minutes. The mixture was then filtered and the filtrate was concentrated to dryness on a rotary evaporator. The residue obtained was dissolved in 1-2 ml of DMSO and the product was isolated by preparative HPLC (Method 34). Evaporation of the product fractions and drying under high vacuum gave 80 mg (81% of theory) of the title compound.

¹H NMR (400 MHz, CDCl₃, δ/ppm): 7.62 (d, 2H), 7.39 (d, 1H), 7.32 (s, 1H), 7.29 (t, 1H), 7.19 (d, 2H), 6.94 (d, 1H), 6.38 (d, 1H), 6.31 (s, 1H), 5.33 (s, 2H), 2.22 (s, 3H), 1.72 (s, broad, 1H), 1.56 (s, 6H).

LC/MS (Method 5, ESIpos): Rₜ=1.33 min, m/z=435 [M+H]⁺.

### Example 54

### 2-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}propan-2-ol

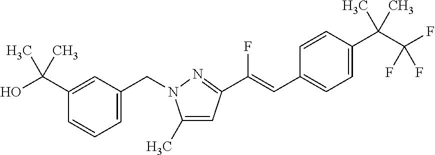 [SMILES: Cc1cc(/C(F)=C/c2ccc(C(C)(C)C(F)(F)F)cc2)nn1Cc1cccc(C(C)(C)O)c1]

72 mg (0.640 mmol) of potassium tert-butoxide and 147 mg (0.60 mmol) of the compound from Example 25A, dissolved in 1.5 ml of THF, were added to a solution of 125 mg (0.40 mmol) of the compound from Example 7A in 2 ml of THF. The reaction mixture was stirred at RT for 1 h. 30 ml of water were then added, and the mixture was extracted three times with in each case 30 ml of ethyl acetate. The combined organic phases were dried over sodium sulphate, filtered and concentrated. The residue was purified by preparative HPLC (Method 16). The combined product fractions were concentrated to a small residual volume of aqueous phase and neutralized with sodium bicarbonate. After two extractions with in each case 30 ml of ethyl acetate, the combined organic phases were dried over sodium sulphate, filtered and concentrated. Drying under high vacuum gave 99 mg (50% of theory, purity 94%) of the title compound.

¹H NMR (400 MHz, CDCl₃, δ/ppm): 7.61 (d, 2H), 7.50-7.45 (m, 3H), 7.38 (d, 1H), 7.33-7.30 (m, 1H), 6.94 (d, 1H), 6.38 (d, 1H), 6.31 (s, 1H), 5.34 (s, 2H), 2.22 (s, 3H), 1.56 (s, 6H).

LC/MS (Method 6, ESIpos): Rₜ=2.82 min, m/z=461 [M+H]⁺.

### Example 55

### (1-{3-[(3-{(Z)-1-Fluoro-2-[4-(trifluoromethoxy)phenyl]vinyl}-5-methyl-1H-pyrazol-1-yl)methyl]-phenyl}cyclopropyl)methanol

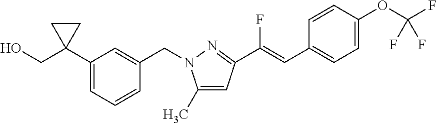 [SMILES: Cc1cc(/C(F)=C/c2ccc(OC(F)(F)F)cc2)nn1Cc1cccc(C2(CO)CC2)c1]

150 mg (0.524 mmol) of the compound from Example 3A and 238 mg (0.576 mmol) of the compound from Example 27A were initially charged in 3.8 ml of dioxane, and 71 mg (0.629 mmol) of solid potassium tert-butoxide were added at 0° C. The reaction mixture was then stored at RT for 4 h. About 50 ml of water were added, and the mixture was extracted three times with in each case about 50 ml of ethyl acetate. The combined organic extracts were washed successively with water and saturated aqueous sodium chloride solution. After drying over anhydrous magnesium sulphate, the mixture was filtered and the filtrate was freed from the solvent on a rotary evaporator. The residue obtained was dissolved in 5 ml of THF, and 786 μl (0.786 mmol) of a 1 M solution of tetra-n-butylammonium fluoride in THF were added. After 1 h at RT, the reaction mixture was diluted with about 2 ml of methanol and directly separated into its components by preparative HPLC (Method 14). After evaporation of the product fractions it was found that they were a mixture of the title compound and the regioisomeric alkylation product (benzylation at the other pyrazole nitrogen atom). This regioisomer mixture was then separated by a second preparative HPLC (Method 28). This gave 118 mg (51% of theory) of the title compound and 42 mg of the regioisomeric benzylation product.

¹H NMR (400 MHz, CDCl₃, δ/ppm): 7.63 (d, 2H), 7.29-7.24 (m, 2H, partially obscured by the CHCl₃ signal), 7.21-7.15 (m, 3H), 6.93 (d, 1H), 6.38 (d, 1H), 6.31 (s, 1H), 5.31 (s, 2H), 3.66 (s, 2H), 2.23 (s, 3H), 0.85 (s, 4H).

LC/MS (Method 2, ESIpos): Rₜ=1.52 min, m/z=447 [M+H]⁺.

### Example 56

### {1-[3-({3-[(Z)-1-Fluoro-2-{4-[(trifluoromethyl)sulphanyl]phenyl}vinyl]-5-methyl-1H-pyrazol-1-yl}methyl)phenyl]cyclopropyl}methanol

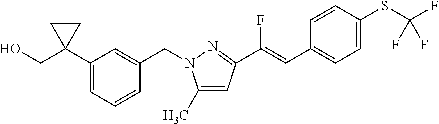 [SMILES: Cc1cc(/C(F)=C/c2ccc(SC(F)(F)F)cc2)nn1Cc1cccc(C2(CO)CC2)c1]

Analogously to the process described under Example 55, 150 mg (0.496 mmol) of the compound from Example 6A and 225 mg (0.546 mmol) of the compound from Example 27A gave 112 mg (49% of theory) of the title compound.

¹H NMR (400 MHz, CDCl₃, δ/ppm): 7.64 (d, 2H), 7.62 (d, 2H), 7.30-7.24 (m, 2H, partially obscured by the CHCl₃ signal), 7.17 (s, 1H), 6.93 (d, 1H), 6.41 (d, 1H), 6.32 (s, 1H), 5.31 (s, 2H), 3.66 (s, 2H), 2.23 (s, 3H), 0.85 (s, 4H).

LC/MS (Method 2, ESIpos): Rₜ=1.58 min, m/z=463 [M+H]⁺.

### Example 57

### (1-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}cyclopropyl)methanol

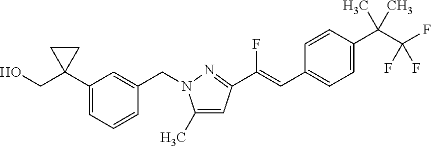 [SMILES: Cc1cc(/C(F)=C/c2ccc(C(C)(C)C(F)(F)F)cc2)nn1Cc1cccc(C2(CO)CC2)c1]

Analogously to the process described under Example 55, 150 mg (0.480 mmol) of the compound from Example 7A and 218 mg (0.528 mmol) of the compound from Example 27A gave 114 mg (50% of theory) of the title compound.

¹H NMR (400 MHz, CDCl₃, δ/ppm): 7.61 (d, 2H), 7.47 (d, 2H), 7.29-7.24 (m, 2H, partially obscured by the CHCl₃ signal), 7.17 (s, 1H), 6.93 (d, 1H), 6.38 (d, 1H), 6.31 (s, 1H), 5.31 (s, 2H), 3.66 (s, 2H), 2.22 (s, 3H), 1.57 (broad, 1H, partially obscured by the water signal), 0.85 (s, 4H).

LC/MS (Method 5, ESIpos): Rₜ=1.35 min, m/z=473 [M+H]⁺.

### Example 58

### (1-{3-[(3-{(Z)-1-Fluoro-2-[3-fluoro-4-(1,1,1-trifluoro-2-methylpropan-2-yl)phenyl]vinyl}-5-methyl-1H-pyrazol-1-yl)methyl]phenyl}cyclopropyl)methanol

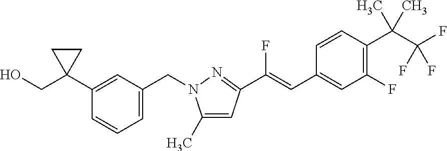 [SMILES: Cc1cc(/C(F)=C/c2ccc(C(C)(C)C(F)(F)F)c(F)c2)nn1Cc1cccc(C2(CO)CC2)c1]

At 0° C., 57 mg (0.512 mmol) of potassium tert-butoxide were added to a solution of 130 mg (0.394 mmol) of the compound from Example 8A and 210 mg (0.433 mmol, purity 85%) of the compound from Example 27A in 3 ml of THF. The reaction mixture was stirred at RT overnight. 0.6 ml (0.60 mmol) of a 1 M solution of tetra-n-butylammonium fluoride in THF was then added, and the reaction mixture was stirred at RT for a further 30 min. After dilution with ethyl acetate, the mixture was washed once with water and the aqueous phase was re-extracted once with ethyl acetate. The combined organic phases were washed with saturated sodium chloride solution, dried over magnesium sulphate, filtered and concentrated. The residue was purified by preparative HPLC (Method 16). The combined product fractions were neutralized with sodium bicarbonate and concentrated to a small residual volume of aqueous phase. After two extractions with ethyl acetate, the combined organic phases were dried over magnesium sulphate, filtered and concentrated. The residue obtained was triturated with pentane and the solid was filtered off and dried under high vacuum. This gave 75 mg (37% of theory, purity 95%) of the title compound.

¹H NMR (400 MHz, CDCl₃, δ/ppm): 7.41-7.23 (m, 5H), 7.17 (s, 1H), 6.95-6.88 (m, 1H), 6.33 (d, 1H), 6.32 (s, 1H), 5.30 (s, 2H), 3.66 (s, 2H), 2.23 (s, 3H), 1.65 (s, 6H), 1.57 (br. s, 1H), 0.85 (s, 4H).

LC/MS (Method 2, ESIpos): Rₜ=1.70 min, m/z=491 [M+H]⁺.

### Example 59

### {1-[3-({3-[(Z)-1-Fluoro-2-{4-[1-(trifluoromethyl)cyclopropyl]phenyl}vinyl]-5-methyl-1H-pyrazol-1-yl}methyl)phenyl]cyclopropyl}methanol

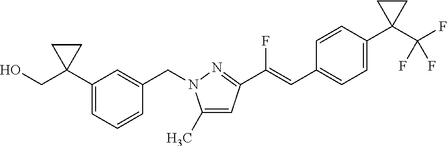 [SMILES: Cc1cc(/C(F)=C/c2ccc(C3(C(F)(F)F)CC3)cc2)nn1Cc1cccc(C2(CO)CC2)c1]

Analogously to the process described under Example 55, 150 mg (0.483 mmol) of the compound from Example 9A and 219 mg (0.532 mmol) of the compound from Example 27A gave 114 mg (49% of theory) of the title compound.

¹H NMR (400 MHz, CDCl₃, δ/ppm): 7.58 (d, 2H), 7.43 (d, 2H), 7.29-7.24 (m, 2H, partially obscured by the CHCl₃ signal), 7.16 (s, 1H), 6.93 (d, 1H), 6.37 (d, 1H), 6.31 (s, 1H), 5.31 (s, 2H), 3.66 (s, 2H), 2.22 (s, 3H), 1.36-1.33 (m, 2H), 1.05-1.01 (m, 2H), 0.85 (s, 4H).

LC/MS (Method 5, ESIpos): Rₜ=1.33 min, m/z=471 [M+H]⁺.

### Example 60

### (1-{3-[(3-{(Z)-1-Fluoro-2-[4-(4-fluorotetrahydro-2H-pyran-4-yl)phenyl]vinyl}-5-methyl-1H-pyrazol-1-yl)methyl]phenyl}cyclopropyl)methanol

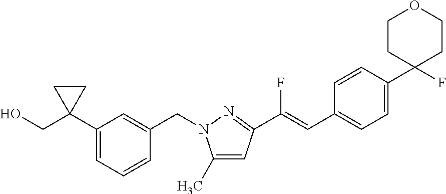 [SMILES: Cc1cc(/C(F)=C/c2ccc(C3(F)CCOCC3)cc2)nn1Cc1cccc(C2(CO)CC2)c1]

Analogously to the process described under Example 55, 150 mg (0.493 mmol) of the compound from Example 17A and 224 mg (0.542 mmol) of the compound from Example 27A gave 123 mg (52% of theory) of the title compound.

¹H NMR (400 MHz, CDCl₃, δ/ppm): 7.63 (d, 2H), 7.38 (d, 2H), 7.29-7.24 (m, 2H, partially obscured by the CHCl₃ signal), 7.17 (s, 1H), 6.93 (d, 1H), 6.39 (d, 1H), 6.31 (s, 1H), 5.31 (s, 2H), 3.97-3.85 (m, 4H), 3.66 (s, 2H), 2.26-2.08 (m, 2H), 2.23 (s, 3H), 1.97-1.90 (m, 2H), 0.85 (s, 4H).

LC/MS (Method 2, ESIpos): Rₜ=1.38 min, m/z=465 [M+H]⁺.

### Example 61

### 2,2-Difluoro-2-{3-[(3-{(Z)-1-fluoro-2-[4-(trifluoromethoxy)phenyl]vinyl}-5-methyl-1H-pyrazol-1-yl)methyl]phenyl}ethanol

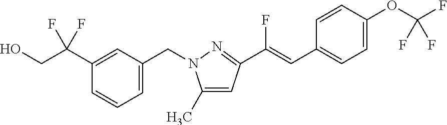 [SMILES: Cc1cc(/C(F)=C/c2ccc(OC(F)(F)F)cc2)nn1Cc1cccc(C(F)(F)CO)c1]

Under argon and with ice cooling, 81 mg (0.725 mmol) potassium tert-butoxide and 377 mg (1.50 mmol) of the compound from Example 28A, dissolved in 2.5 ml of THF, were added to a solution of 143 mg (0.50 mmol) of the compound from Example 3A in 2.5 ml of THF. The reaction mixture was stirred at RT for 16 h. 30 ml of water and 30 ml of ethyl acetate were then added, and after phase separation the aqueous phase was extracted once with 30 ml of ethyl acetate. The combined organic phases were dried over sodium sulphate, filtered and concentrated. The residue was purified initially by column chromatography (silica gel, mobile phase cyclohexane/ethyl acetate 4:1) and then by preparative HPLC (Method 35). Drying under high vacuum gave 8 mg (4% of theory) of the title compound.

¹H NMR (400 MHz, CDCl₃, δ/ppm): 7.63 (d, 2H), 7.47-7.38 (m, 2H), 7.31 (s, 1H), 7.22-7.15 (m, 3H), 6.37 (d, 1H), 6.32 (s, 1H), 5.36 (s, 2H), 3.95 (t, 2H), 2.23 (s, 3H), 2.18 (br. s, 1H).

LC/MS (Method 5, ESIpos): Rₜ=1.27 min, m/z=457 [M+H]⁺.

### Example 62

### 2,2-Difluoro-2-{3-[(3-{(Z)-1-fluoro-2-[4-(1,1,1-trifluoro-2-methylpropan-2-yl)phenyl]vinyl}-5-methyl-1H-pyrazol-1-yl)methyl]phenyl}ethanol

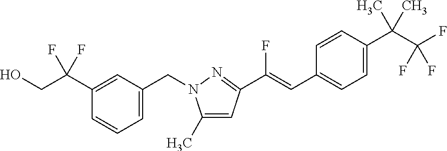 [SMILES: Cc1cc(/C(F)=C/c2ccc(C(C)(C)C(F)(F)F)cc2)nn1Cc1cccc(C(F)(F)CO)c1]

Analogously to the process described under Example 61, 141 mg (0.450 mmol) of the compound from Example 7A and 339 mg (1.35 mmol) of the compound from Example 28A gave 30 mg (13% of theory, purity 96%) of the title compound. In this case, the mobile phase mixture cyclohexane/ethyl acetate 3:1 was used for column chromatography and the mobile phase mixture isohexane/ethanol 70:30 was used for the subsequent preparative HPLC.

¹H NMR (400 MHz, CDCl₃, δ/ppm): 7.61 (d, 2H), 7.50-7.38 (m, 4H), 7.31 (s, 1H), 7.17 (d, 1H), 6.37 (d, 1H), 6.33 (s, 1H), 5.36 (s, 2H), 3.95 (dt, 2H), 2.22 (s, 3H), 1.96 (t, 1H), 1.58 (s, 6H).

LC/MS (Method 5, ESIpos): Rₜ=1.30 min, m/z=483 [M+H]⁺.

### Example 63

### 2,2-Difluoro-2-[3-({3-[(Z)-1-fluoro-2-{4-[1-(trifluoromethyl)cyclopropyl]phenyl}vinyl]-5-methyl-1H-pyrazol-1-yl}methyl)phenyl]ethanol

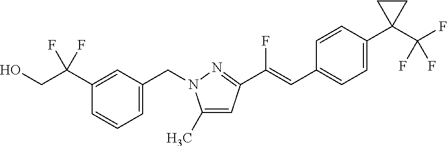 [SMILES: Cc1cc(/C(F)=C/c2ccc(C3(C(F)(F)F)CC3)cc2)nn1Cc1cccc(C(F)(F)CO)c1]

Analogously to the process described under Example 61, 155 mg (0.50 mmol) of the compound from Example 9A and 377 mg (1.50 mmol) of the compound from Example 28A gave 46 mg (19% of theory) of the title compound. In this case, the reaction mixture was initially stirred at RT for 16 h, a further 81 mg (0.725 mmol) of potassium tert-butoxide were added and the mixture was once more stirred at RT overnight. Here, the HPLC purification step was carried out using the mobile phase mixture isohexane/ethanol 80:20 at a flow rate of 20 ml/min.

¹H NMR (400 MHz, CDCl₃, δ/ppm): 7.58 (d, 2H), 7.47-7.38 (m, 4H), 7.31 (s, 1H), 7.17 (d, 1H), 6.37 (d, 1H), 6.33 (s, 1H), 5.36 (s, 2H), 3.94 (dt, 2H), 2.22 (s, 3H), 2.14 (t, 1H), 1.29-1.24 (m, 2H), 1.06-1.00 (m, 2H).

LC/MS (Method 5, ESIpos): Rₜ=1.30 min, m/z=481 [M+H]⁺.

### Example 64

### 1-{3-[(3-{(Z)-1-Fluoro-2-[4-(1,1,1-trifluoro-2-methylpropan-2-yl)phenyl]vinyl}-5-methyl-1H-pyrazol-1-yl)methyl]phenyl}-2-methylpropan-2-ol

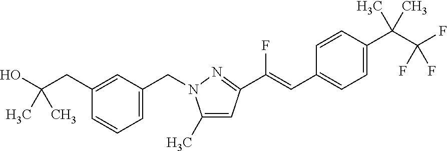 [SMILES: Cc1cc(/C(F)=C/c2ccc(C(C)(C)C(F)(F)F)cc2)nn1Cc1cccc(CC(C)(C)O)c1]

At 0° C., 43 mg (0.380 mmol) of solid potassium tert-butoxide were added to a solution of 100 mg (0.320 mmol) of the compound from Example 7A and 99.2 mg (0.380 mmol) of the compound from Example 29A in 4 ml of dioxane. The cooling bath was then removed, and the reaction mixture was stirred at RT for 30 min. About 50 ml of water were then added, and the mixture was extracted three times with in each case about 50 ml of ethyl acetate. The combined organic extracts were dried over anhydrous magnesium sulphate, filtered and freed from the solvent on a rotary evaporator. The residue obtained was subjected to a first preparative HPLC (Method 14). The product fraction obtained in this manner consisted of a mixture of the title compound with the regioisomeric alkylation product (benzylation at the other pyrazole nitrogen atom). This regioisomer mixture was then separated by a second preparative HPLC (Method 36). This gave 85 mg (56% of theory) of the title compound and 14 mg of the regioisomeric benzylation product.

¹H NMR (400 MHz, CDCl₃, δ/ppm): 7.60 (d, 2H), 7.47 (d, 2H), 7.27 (t, 1H, partially superimposed by the CHCl₃ signal), 7.13 (d, 1H), 6.99 (d, 1H), 6.98 (s, 1H), 6.37 (d, 1H), 6.30 (s, 1H), 5.31 (s, 2H), 2.73 (s, 2H), 2.21 (s, 3H), 1.57 (s, 6H), 1.31 (s, 1H), 1.19 (s, 6H).

LC/MS (Method 5, ESIpos): Rₜ=1.37 min, m/z=475 [M+H]⁺.

### Example 65

### 4-{5-[(Z)-2-Fluoro-2-{1-[(6-fluoropyridin-3-yl)methyl]-5-methyl-1H-pyrazol-3-yl}vinyl]pyridin-2-yl}-2,6-dimethylmorpholine

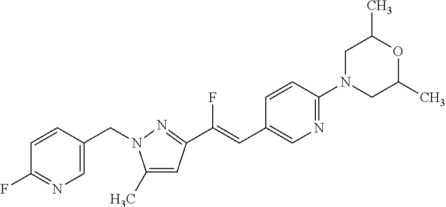 [SMILES: Cc1cc(/C(F)=C/c2ccc(N3CC(C)OC(C)C3)nc2)nn1Cc1ccc(F)nc1]

169 mg (0.765 mmol, purity 93%) of the compound from Example 30A were added to a solution of 220 mg (0.695 mmol) of the compound from Example 21A in 7 ml of THF. The mixture was cooled to 0° C., and 101 mg (0.904 mmol) potassium tert-butoxide were then added. The reaction mixture was stirred initially at 0° C. for a few minutes and then at RT for 4 h. After addition of ethyl acetate, the mixture was extracted once with water, and after phase separation the aqueous phase was re-extracted once with ethyl acetate. The combined organic phases were washed once with saturated sodium chloride solution, dried over magnesium sulphate, filtered and concentrated. The residue was purified by thick-layer chromatography (silica gel, mobile phase cyclohexane/ethyl acetate 1:1). The product-containing zone was extracted with dichloromethane/methanol 95:5. After removal of the solvent, the residue was triturated with pentane and the solid was filtered off and dried under high vacuum. This gave 196 mg (62% of theory, purity 94%) of the title compound.

¹H NMR (400 MHz, CDCl₃, δ/ppm): 8.32 (d, 1H), 8.09 (d, 1H), 7.89 (dd, 1H), 7.60 (td, 1H), 6.91 (dd, 1H), 6.64 (d, 1H), 6.28 (s, 1H), 6.24 (d, 1H), 5.30 (s, 2H), 4.08 (dd, 2H), 3.77-3.68 (m, 2H), 2.56 (dd, 2H), 2.25 (s, 3H), 1.28 (d, 6H).

LC/MS (Method 5, ESIpos): Rₜ=0.98 min, m/z=426 [M+H]⁺.

### Example 66

### 2-Chloro-5-[(3-{(Z)-1-fluoro-2-[4-(trifluoromethoxy)phenyl]vinyl}-5-methyl-1H-pyrazol-1-yl)methyl]pyridine

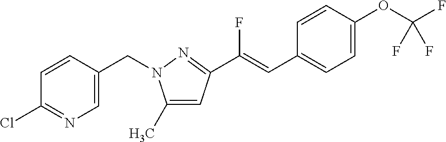 [SMILES: Cc1cc(/C(F)=C/c2ccc(OC(F)(F)F)cc2)nn1Cc1ccc(Cl)nc1]

Analogously to the process described under Example 69, 250 mg (0.873 mmol) of the compound from Example 3A and 192 mg (1.135 mmol, purity 96%) of 2-chloro-5-(chloromethyl)pyridine gave 193 mg (50% of theory, purity 94%) of the title compound. Here, the crude product was purified by column chromatography (silica gel, cyclohexane/ethyl acetate 9:1).

¹H NMR (400 MHz, CDCl₃, δ/ppm): 8.27 (d, 1H), 7.62 (d, 2H), 7.44 (dd, 1H), 7.30 (d, 1H), 7.20 (d, 2H), 6.36 (d, 1H), 6.32 (s, 1H), 5.30 (s, 2H), 2.25 (s, 3H).

LC/MS (Method 6, ESIpos): Rₜ=2.78 min, m/z=412/414 [M+H]⁺.

### Example 67

### 2-Chloro-5-[(3-{(Z)-1-fluoro-2-[3-fluoro-4-(trifluoromethoxy)phenyl]vinyl}-5-methyl-1H-pyrazol-1-yl)methyl]pyridine

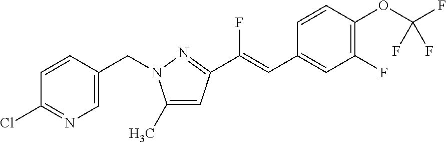 [SMILES: Cc1cc(/C(F)=C/c2ccc(OC(F)(F)F)c(F)c2)nn1Cc1ccc(Cl)nc1]

At 0° C., 719 mg (6.41 mmol) potassium tert-butoxide were added to a solution of 1.50 g (4.93 mmol) of the compound from Example 4A and 1.20 g (5.42 mmol) of (6-chloropyridin-3-yl)methyl methanesulphonate [preparation: see, for example, J. Org. Chem. 64 (23), 8576-8581 (1999)] in 30 ml of THF. The reaction mixture was stirred at RT overnight. After addition of ethyl acetate, the mixture was extracted once with water, and after phase separation the aqueous phase was re-extracted once with ethyl acetate. The combined organic phases were washed once with saturated sodium chloride solution, dried over magnesium sulphate, filtered and concentrated. Methanol was added to the residue, and the solid formed was washed once with pentane and dried under high vacuum. This gave 800 mg (38% of theory) of the title compound.

¹H NMR (400 MHz, CDCl₃, δ/ppm): 8.27 (d, 1H), 7.50 (d, 1H), 7.44 (dd, 1H), 7.35-7.22 (m, 3H), 6.33 (s, 1H), 6.33 (d, 1H), 5.30 (s, 2H), 2.26 (s, 3H).

LC/MS (Method 5, ESIpos): Rₜ=1.35 min, m/z=430/432 [M+H]⁺.

### Example 68

### 2-Chloro-5-({3-[(Z)-1-fluoro-2-{4-[(trifluoromethyl)sulphanyl]phenyl}vinyl]-5-methyl-1H-pyrazol-1-yl}methyl)pyridine

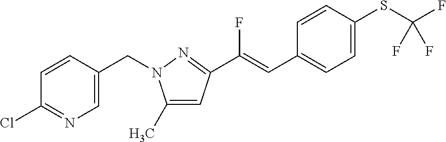 [SMILES: Cc1cc(/C(F)=C/c2ccc(SC(F)(F)F)cc2)nn1Cc1ccc(Cl)nc1]

Analogously to the process described under Example 67, 260 mg (0.860 mmol) of the compound from Example 6A and 210 mg (0.946 mmol) of (6-chloropyridin-3-yl)methyl methanesulphonate [preparation: see, for example, J. Org. Chem. 64 (23), 8576-8581 (1999)] gave 120 mg (33% of theory) of the title compound. Here, the crude product was purified by column chromatography (silica gel, cyclohexane/ethyl acetate 7:3).

¹H NMR (400 MHz, CDCl₃, δ/ppm): 8.27 (s, 1H), 7.67-7.58 (m, 4H), 7.44 (dd, 1H), 7.31 (d, 1H), 6.39 (d, 1H), 6.34 (s, 1H), 5.31 (s, 2H), 2.26 (s, 3H).

LC/MS (Method 2, ESIpos): Rₜ=1.59 min, m/z=428/430 [M+H]⁺.

### Example 69

### 2-Chloro-5-[(3-{(Z)-1-fluoro-2-[4-(1,1,1-trifluoro-2-methylpropan-2-yl)phenyl]vinyl}-5-methyl-1H-pyrazol-1-yl)methyl]pyridine

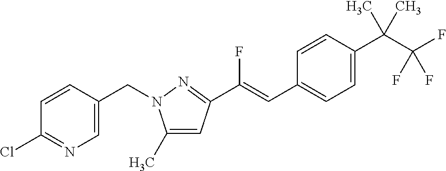 [SMILES: Cc1cc(/C(F)=C/c2ccc(C(C)(C)C(F)(F)F)cc2)nn1Cc1ccc(Cl)nc1]

374 mg (3.33 mmol) of potassium tert-butoxide were added to a solution of 800 mg (2.56 mmol) of the compound from Example 7A and 562 mg (3.33 mmol, purity 96%) of 2-chloro-5-(chlormethyl)pyridin in 23 ml of THF. The mixture was initially stirred at a bath temperature of 70° C. for 3 h. A further 72 mg (0.640 mmol) of potassium tert-butoxide were then added, and the mixture was once more stirred at a bath temperature of 70° C. for 1.5 h. After cooling to RT, 100 ml of water and 100 ml of ethyl acetate were added to the mixture, and after phase separation the aqueous phase was extracted twice with in each case 60 ml of ethyl acetate. The combined organic phases were washed once with 100 ml of saturated sodium chloride solution, dried over sodium sulphate, filtered and concentrated. The residue was triturated with 5 ml of warm methanol, and the precipitate formed was filtered off and washed twice with in each case 1 ml of methanol. Drying under high vacuum gave 231 mg (20% of theory) of the title compound. The filtrate which remained after trituration with methanol was concentrated, and the residue was purified by preparative HPLC (Method 13), followed by two column chromatographies (silica gel, mobile phase cyclohexane/ethyl acetate 7:3 and 85:15, respectively). Drying under high vacuum gave a further 268 mg (24% of theory) of the title compound. This gave a total of 499 mg (44% of theory) of the title compound.

¹H NMR (400 MHz, CDCl₃, δ/ppm): 8.27 (d, 1H), 7.60 (d, 2H), 7.48 (d, 2H), 7.44 (dd, 1H), 7.31 (d, 1H), 6.36 (d, 1H), 6.32 (s, 1H), 5.30 (s, 2H), 2.25 (s, 3H), 1.58 (s, 6H).

LC/MS (Method 5, ESIpos): Rₜ=1.39 min, m/z=438/440 [M+H]⁺.

### Example 70

### 2-Chloro-5-[(3-{(Z)-1-fluoro-2-[4-(1,1,1-trifluoro-2-methylpropan-2-yl)phenyl]vinyl}-5-methyl-1H-pyrazol-1-yl)methyl]pyridine

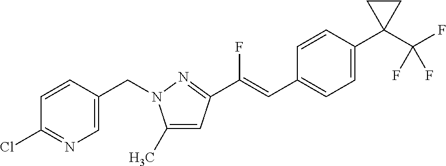 [SMILES: Cc1cc(/C(F)=C/c2ccc(C3(C(F)(F)F)CC3)cc2)nn1Cc1ccc(Cl)nc1]

At 0° C., 75 mg (0.666 mmol) potassium tert-butoxide were added to a solution of 188 mg (0.606 mmol) of the compound from Example 9A and 133 mg (0.788 mmol, purity 96%) of 2-chloro-5-(chloromethyl)pyridine in 5.5 ml of THF. The mixture was stirred initially at RT for 18 h and then at a bath temperature of 80° C. for 2 h. A further 17 mg (0.151 mmol) of potassium tert-butoxide were then added, and the mixture was once more stirred at a bath temperature of 80° C. for 1.5 h. After cooling to RT, 30 ml of water and 30 ml of ethyl acetate were added to the mixture, and after phase separation the aqueous phase was extracted twice with in each case 30 ml of ethyl acetate. The combined organic phases were washed once with 100 ml of saturated sodium chloride solution, dried over sodium sulphate, filtered and concentrated. The residue was purified by preparative HPLC (Method 27). The combined product fractions were concentrated to a small residual volume of aqueous phase and adjusted to pH 8 with saturated aqueous sodium bicarbonate solution. After three extractions with ethyl acetate, the combined organic phases were dried over sodium sulphate, filtered and concentrated. Drying under high vacuum gave 37 mg (12% of theory, purity 89%) of the title compound.

¹H NMR (400 MHz, CDCl₃, δ/ppm): 8.30-8.23 (m, 1H), 7.58 (d, 2H), 7.48-7.41 (m, 3H), 7.30 (d, 1H), 6.35 (d, 1H), 6.32 (s, 1H), 5.30 (s, 2H), 2.25 (s, 3H), 1.37-1.32 (m, 2H), 1.06-1.00 (m, 2H).

LC/MS (Method 5, ESIpos): Rₜ=1.35 min, m/z=436/438 [M+H]⁺.

### Example 71

### 2-Chloro-5-[(3-{(Z)-1-fluoro-2-[4-(trifluoromethyl)phenyl]vinyl}-5-methyl-1H-pyrazol-1-yl)methyl]pyridine

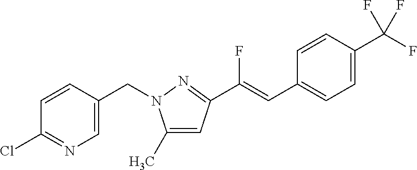 [SMILES: Cc1cc(/C(F)=C/c2ccc(C(F)(F)F)cc2)nn1Cc1ccc(Cl)nc1]

Analogously to the process described under Example 67, 210 mg (0.777 mmol) of the compound from Example 10A were reacted with 189 mg (0.855 mmol) of (6-chloropyridin-3-yl)methyl methanesulphonate [preparation: see, for example, J. Org. Chem. 64 (23), 8576-8581 (1999)]. In this case, the crude product was initially pre-purified by column chromatography (silica gel, cyclohexane/ethyl acetate 7:3), then triturated with pentane, filtered off and then re-purified again by preparative HPLC (Method 16). The HPLC product fractions were neutralized with saturated aqueous sodium bicarbonate solution and concentrated on a rotary evaporator. The solid formed during this operation was filtered off, washed twice with water and dried under high vacuum. This gave 69 mg (22% of theory) of the title compound.

¹H NMR (400 MHz, CDCl₃, δ/ppm): 8.27 (d, 1H), 7.70 (d, 2H), 7.60 (d, 2H), 7.44 (dd, 1H), 7.31 (d, 1H), 6.41 (d, 1H), 6.35 (s, 1H), 5.31 (s, 2H), 2.26 (s, 3H).

LC/MS (Method 5, ESIpos): Rₜ=1.31 min, m/z=396/398 [M+H]⁺.

### Example 72

### 2-Chloro-5-[(3-{(Z)-1-fluoro-2-[4-(trimethylsilyl)phenyl]vinyl}-5-methyl-1H-pyrazol-1-yl)-methyl]pyridine

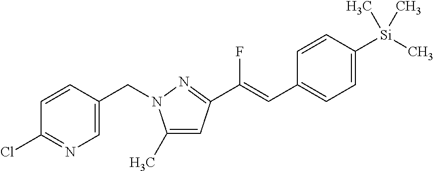 [SMILES: Cc1cc(/C(F)=C/c2ccc([Si](C)(C)C)cc2)nn1Cc1ccc(Cl)nc1]

Analogously to the process described under Example 70, 200 mg (0.729 mmol) of the compound from Example 11A and 160 mg (0.947 mmol) of 2-chloro-5-(chloromethyl)pyridine gave 118 mg (40% of theory) of the title compound.

¹H NMR (400 MHz, CDCl₃, δ/ppm): 8.27 (d, 1H), 7.59 (d, 2H), 7.52 (d, 2H), 7.44 (dd, 1H), 7.30 (d, 1H), 6.36 (d, 1H), 6.32 (s, 1H), 5.30 (s, 2H), 2.24 (s, 3H), 0.27 (s, 9H).

LC/MS (Method 5, ESIpos): Rₜ=1.45 min, m/z=400/402 [M+H]⁺.

### Example 73

### 5-({3-[(Z)-2-(4-tert-Butylphenyl)-1-fluorovinyl]-5-methyl-1H-pyrazol-1-yl}methyl)-2-chloro-pyridine

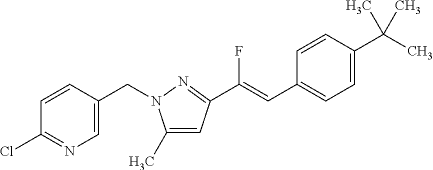 [SMILES: Cc1cc(/C(F)=C/c2ccc(C(C)(C)C)cc2)nn1Cc1ccc(Cl)nc1]

Analogously to the process described under Example 67, 210 mg (0.813 mmol) of the compound from Example 12A and 198 mg (0.894 mmol) of (6-chloropyridin-3-yl)methyl methanesulphonate [preparation: see, for example, J. Org. Chem. 64 (23), 8576-8581 (1999)] gave 179 mg (57% of theory) of the title compound. In this case, the reaction mixture was stirred at RT for 6 h (instead of overnight). The crude product was purified by column chromatography (silica gel, cyclohexane/ethyl acetate 85:15).

¹H NMR (400 MHz, CDCl₃, δ/ppm): 8.27 (d, 1H), 7.56 (d, 2H), 7.44 (dd, 1H), 7.39 (d, 2H), 7.30 (d, 1H), 6.33 (d, 1H), 6.30 (s, 1H), 5.30 (s, 2H), 2.24 (s, 3H), 1.33 (s, 9H).

LC/MS (Method 5, ESIpos): Rₜ=1.41 min, m/z=384/386 [M+H]⁺.

### Example 74

### 2-Chloro-5-({3-[(Z)-1-fluoro-2-(4-isopropylphenyl)vinyl]-5-methyl-1H-pyrazol-1-yl}methyl)-pyridine

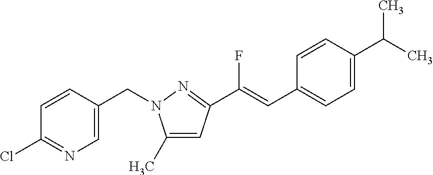 [SMILES: Cc1cc(/C(F)=C/c2ccc(C(C)C)cc2)nn1Cc1ccc(Cl)nc1]

Analogously to the process described under Example 67, 330 mg (1.35 mmol) of the compound from Example 14A and 329 mg (1.49 mmol) of (6-chloropyridin-3-yl)methyl methanesulphonate [preparation: see, for example, J. Org. Chem. 64 (23), 8576-8581 (1999)] gave 313 mg (62% of theory) of the title compound. Here, the crude product was purified by column chromatography (silica gel, cyclohexane/ethyl acetate 4:1).

¹H NMR (400 MHz, CDCl₃, δ/ppm): 7.55 (d, 2H), 7.44 (dd, 1H), 7.30 (d, 1H), 7.22 (d, 2H), 6.32 (d, 1H), 6.31 (s, 1H), 5.30 (s, 2H), 2.91 (sept, 1H), 2.24 (s, 3H), 1.26 (d, 6H).

LC/MS (Method 5, ESIpos): Rₜ=1.37 min, m/z=370/372 [M+H]⁺.

### Example 75

### 2-Chloro-5-({3-[(Z)-1-fluoro-2-(4-isobutylphenyl)vinyl]-5-methyl-1H-pyrazol-1-yl}methyl)-pyridine

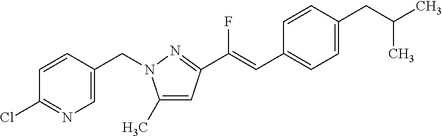 [SMILES: Cc1cc(/C(F)=C/c2ccc(CC(C)C)cc2)nn1Cc1ccc(Cl)nc1]

Analogously to the process described under Example 67, 362 mg (1.40 mmol) of the compound from Example 15A and 341 mg (1.54 mmol) of (6-chloropyridin-3-yl)methyl methanesulphonate [preparation: see, for example, J. Org. Chem. 64 (23), 8576-8581 (1999)] gave 243 mg (43% of theory) of the title compound in a purity of 96% and additionally 168 mg (28% of theory) of the title compound in a purity of 91%. Here, the crude product was purified by column chromatography (silica gel, cyclohexane/ethyl acetate 4:1).

¹H NMR (400 MHz, CDCl₃, δ/ppm): 8.27 (d, 1H), 7.52 (d, 2H), 7.43 (dd, 1H), 7.30 (d, 1H), 7.14 (d, 2H), 6.32 (d, 1H), 6.30 (s, 1H), 5.30 (s, 2H), 2.47 (d, 2H), 2.24 (s, 3H), 1.87 (m, 1H), 0.91 (d, 6H).

LC/MS (Method 5, ESIpos): Rₜ=1.44 min, m/z=384/386 [M+H]⁺.

### Example 76

### 2-({4-[(Z)-2-{1-[(6-Chloropyridin-3-yl)methyl]-5-methyl-1H-pyrazol-3-yl}-2-fluorovinyl]phenyl}-sulphanyl)-N-ethyl-2-methylpropanamide

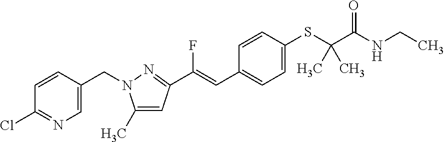 [SMILES: CCNC(=O)C(C)(C)Sc1ccc(/C=C(\F)c2cc(C)n(Cc3ccc(Cl)nc3)n2)cc1]

110 mg (0.212 mmol) of benzotriazol-1-yloxytris(pyrrolidino)phosphonium hexafluorophosphate (PyBOP) and 104 μl (0.598 mmol) of N,N-diisopropylethylamine were added successively to a solution of 86 mg (0.193 mmol) of the compound from Example 38A and a drop of DMF in 2 ml of THF. The mixture was stirred at RT for 1 h. 106 μl (0.212 mmol) of a 2 M solution of ethylamine in THF were then added, and the mixture was stirred at RT for a further 30 min. After addition of ethyl acetate, the mixture was extracted once with water and the aqueous phase was re-extracted once with ethyl acetate. The combined organic phases were washed once with saturated sodium chloride solution, dried over magnesium sulphate, filtered and concentrated. The residue was purified by preparative HPLC (Method 13). The combined product fractions were concentrated on a rotary evaporator to a small residual volume of aqueous phase, and saturated aqueous sodium bicarbonate solution was added. The solid formed during this operation was filtered off, washed twice with water and dried under high vacuum. This gave 64 mg (70% of theory) of the title compound.

¹H NMR (400 MHz, CDCl₃, δ/ppm): 8.27 (s, 1H), 7.53 (d, 2H), 7.44 (d, 1H), 7.36 (d, 2H), 7.30 (d, 1H), 6.92-6.84 (m, 1H), 6.33 (d, 1H), 6.32 (s, 1H), 5.30 (s, 2H), 3.32 (quint, 2H), 2.25 (s, 3H), 1.52 (s, 6H), 1.16 (t, 3H).

LC/MS (Method 5, ESIpos): Rₜ=1.17 min, m/z=473/475 [M+H]⁺.

### Example 77

### 2-({4-[(Z)-2-{1-[(6-Chloropyridin-3-yl)methyl]-5-methyl-1H-pyrazol-3-yl}-2-fluorovinyl]phenyl}-sulphanyl)-2-methyl-1-(pyrrolidin-1-yl)propan-1-one

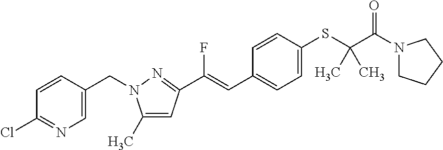 [SMILES: Cc1cc(/C(F)=C/c2ccc(SC(C)(C)C(=O)N3CCCC3)cc2)nn1Cc1ccc(Cl)nc1]

Analogously to the process described under Example 76, 100 mg (0.224 mmol) of the compound from Example 38A and 21 μl (0.247 mmol) of pyrrolidine gave a total of 104 mg (93% of theory) of the title compound in two batches. The first batch was obtained after a little acetonitrile had been added to the crude product prior to the preparative HPLC purification. The addition resulted in the precipitation of a solid which was filtered off and dried under high vacuum, giving 97 mg (87% of theory) of the title compound as the first batch. The second batch was obtained by concentrating the filtrate of said filtration and purifying this residue by preparative HPLC (Method 13). The combined product fractions of the HPLC separation were concentrated on a rotary evaporator to a small residual volume of aqueous phase, and saturated aqueous sodium bicarbonate solution was added. The mixture was extracted twice with dichloromethane, after which the combined organic phases were dried over magnesium sulphate, filtered and concentrated. Drying of the residue under high vacuum thus gave rise to a further 7 mg (6% of theory) of the title compound as second batch.

¹H NMR (400 MHz, CDCl₃, δ/ppm): 8.27 (d, 1H), 7.52 (d, 2H), 7.43 (dd, 1H), 7.31 (m, 3H), 6.31 (s, 1H), 6.31 (d, 1H), 5.30 (s, 2H), 4.03 (br. s, 2H), 3.52 (br. s, 2H), 2.24 (s, 3H), 1.97 (br. s, 2H), 1.84 (br. s, 2H), 1.56 (s, 6H).

LC/MS (Method 2, ESIpos): Rₜ=1.46 min, m/z=499/501 [M+H]⁺.

### Example 78

### 2-{4-[(Z)-2-{1-[(6-Chloropyridin-3-yl)methyl]-5-methyl-1H-pyrazol-3-yl}-2-fluorovinyl]phenyl}-1,1,1,3,3,3-hexafluoropropan-2-ol

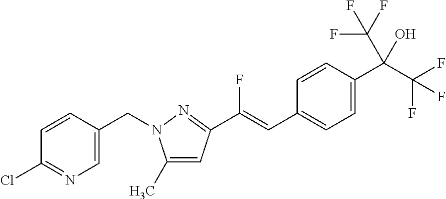 [SMILES: Cc1cc(/C(F)=C/c2ccc(C(O)(C(F)(F)F)C(F)(F)F)cc2)nn1Cc1ccc(Cl)nc1]

Analogously to the process described in Example 69, 240 mg (0.652 mmol) of the compound from Example 16A and 137 mg (0.847 mmol) 2-chloro-5-(chloromethyl)pyridine gave 167 mg of the title compound (52% of theory, purity about 67%, contamination: regioisomeric pyrazole alkylation product). In this case, the reaction mixture was heated under reflux for one day. The crude product was purified by preparative HPLC (Method 16). The combined product fractions from the HPLC were concentrated to a small residue volume of aqueous phase and neutralized with saturated aqueous sodium bicarbonate solution. After three extractions with in each case 40 ml of ethyl acetate, the combined organic phases were dried over sodium sulphate, filtered and concentrated.

LC/MS (Method 7, ESIpos): Rₜ=2.63 min, m/z=494/496 [M+H]⁺.

### Example 79

### 5-[(3-{(Z)-1-Fluoro-2-[4-(trifluoromethoxy)phenyl]vinyl}-5-methyl-1H-pyrazol-1-yl)methyl]-N-methylpyridine-2-amine

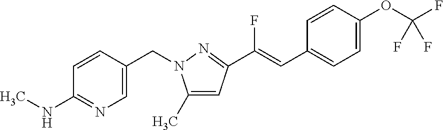 [SMILES: CNc1ccc(Cn2nc(/C(F)=C/c3ccc(OC(F)(F)F)cc3)cc2C)cn1]

A mixture of 182 mg (0.414 mmol) of the compound from Example 66 and 5.5 ml (44.1 mmol) of a 33% strength solution of methylamine in ethanol was heated in a microwave oven (Biotage Initiator with dynamic irradiation power control) at 135° C. for 3 h. After cooling to RT, the volatile components were removed on a rotary evaporator and the residue was purified by preparative HPLC (Method 13). The combined product fractions were concentrated on a rotary evaporator to a small residual volume of aqueous phase, the pH was adjusted to 8 with saturated aqueous sodium bicarbonate solution and the mixture was extracted three times with ethyl acetate. The combined organic phases were dried over sodium sulphate, filtered and concentrated. Drying under high vacuum gave 97 mg (57% of theory) of the title compound.

¹H NMR (400 MHz, CDCl₃, δ/ppm): 7.99 (d, 1H), 7.62 (d, 2H), 7.32 (dd, 1H), 7.19 (d, 2H), 6.36 (d, 1H), 6.35 (d, 1H), 6.26 (s, 1H), 5.16 (s, 2H), 4.63 (br. s, 1H), 2.91 (d, 3H), 2.25 (s, 3H).

LC/MS (Method 2, ESIpos): Rₜ=1.09 min, m/z=407 [M+H]⁺.

### Example 80

### 5-[(3-{(Z)-1-Fluoro-2-[3-fluoro-4-(trifluoromethoxy)phenyl]vinyl}-5-methyl-1H-pyrazol-1-yl)-methyl]-N-methylpyridine-2-amine

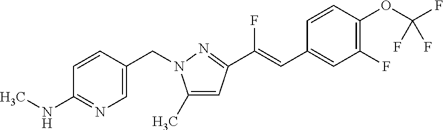 [SMILES: CNc1ccc(Cn2nc(/C(F)=C/c3ccc(OC(F)(F)F)c(F)c3)cc2C)cn1]

Analogously to the process described under Example 79, 300 mg (0.642 mmol, purity 92%) of the compound from Example 67 and 8.0 ml (64.2 mmol) of a 33% strength methylamine solution in ethanol gave 78 mg (29% of theory) of the title compound. In this case, the reaction time in the microwave oven was 5 h at 150° C. For the preparative HPLC purification of the crude product, Method 20 was used.

¹H NMR (400 MHz, CDCl₃, δ/ppm): 7.99 (s, 1H), 7.51 (d, 1H), 7.32 (d, 2H), 7.26 (m, 1H), 6.34 (m, 3H), 5.16 (s, 2H), 4.57 (m, 1H), 2.91 (d, 3H), 2.26 (s, 3H).

LC/MS (Method 5, ESIpos): Rₜ=0.95 min, m/z=425 [M+H]⁺.

### Example 81

### 5-[(3-{(Z)-2-[3-Chloro-4-(trifluoromethoxy)phenyl]-1-fluorovinyl}-5-methyl-1H-pyrazol-1-yl)-methyl]-N-methylpyridine-2-amine

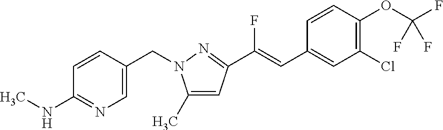 [SMILES: CNc1ccc(Cn2nc(/C(F)=C/c3ccc(OC(F)(F)F)c(Cl)c3)cc2C)cn1]

At RT, 0.5 ml (6.49 mmol) of trifluoroacetic acid were added to a solution of 20 mg (0.034 mmol) of the compound from Example 39A in 0.5 ml of dichloromethane. The mixture was stirred at RT for four days. The volatile components were then removed on a rotary evaporator and the residue was purified by preparative HPLC (Method 13). The combined product fractions were concentrated to a small residual volume of aqueous phase and adjusted to pH 8 with saturated aqueous sodium bicarbonate solution. After two extractions with in each case 20 ml of dichloromethane, the combined organic phases were dried over sodium sulphate, filtered and concentrated. Drying under high vacuum gave 15 mg (94% of theory, purity 96%) of the title compound.

¹H NMR (400 MHz, CDCl₃, δ/ppm): 7.99 (d, 1H), 7.73 (d, 1H), 7.49 (dd, 1H), 7.32 (dd, 1H), 7.30-7.27 (m, 2H), 6.38-6.25 (m, 4H), 5.16 (s, 2H), 4.62 (br. s, 1H), 2.91 (d, 3H), 2.26 (s, 3H).

LC/MS (Method 5, ESIpos): Rₜ=1.01 min, m/z=441/443 [M+H]⁺.

### Example 82

### 5-({3-[(Z)-1-Fluoro-2-{4-[(trifluoromethyl)sulphanyl]phenyl}vinyl]-5-methyl-1H-pyrazol-1-yl}-methyl)-N-methylpyridine-2-amine

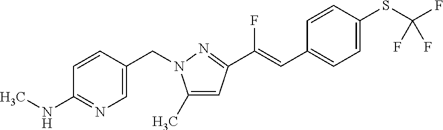 [SMILES: CNc1ccc(Cn2nc(/C(F)=C/c3ccc(SC(F)(F)F)cc3)cc2C)cn1]

Analogously to the process described under Example 79, 100 mg (0.215 mmol, purity 92%) of the compound from Example 68 and 2.7 ml (21.5 mmol) of a 33% strength methylamine solution in ethanol gave 16 mg (18% of theory) of the title compound. In this case, the reaction time in the microwave oven was 5 h at 150° C. For the preparative HPLC purification of the crude product, Method 20 was used.

¹H NMR (400 MHz, CDCl₃, δ/ppm): 7.99 (d, 1H), 7.63 (dd, 4H), 7.32 (dd, 1H), 6.37 (d, 1H), 6.36 (d, 1H), 6.28 (s, 1H), 5.16 (s, 2H), 4.55 (br. s, 1H), 2.91 (d, 3H), 2.25 (s, 3H).

LC/MS (Method 5, ESIpos): Rₜ=0.96 min, m/z=423 [M+H]⁺.

### Example 83

### 5-[(3-{(Z)-1-Fluoro-2-[4-(1,1,1-trifluoro-2-methylpropan-2-yl)phenyl]vinyl}-5-methyl-1H-pyrazol-1-yl)methyl]-N-methylpyridine-2-amine

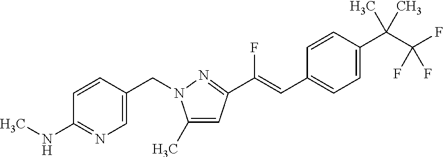 [SMILES: CNc1ccc(Cn2nc(/C(F)=C/c3ccc(C(C)(C)C(F)(F)F)cc3)cc2C)cn1]

Analogously to the process described under Example 79, 182 mg (0.414 mmol, purity 94%) of the compound from Example 69 and 5.2 ml (41.4 mmol) of a 33% strength methylamine solution in ethanol gave 97 mg (57% of theory) of the title compound. In this case, the reaction time in the microwave oven was 6 h at 135° C.

¹H NMR (400 MHz, CDCl₃, δ/ppm): 7.98 (d, 1H), 7.60 (d, 2H), 7.47 (d, 2H), 7.33 (dd, 1H), 6.37 (d, 1H), 6.35 (d, 1H), 6.26 (s, 1H), 5.16 (s, 2H), 4.72 (br. s, 1H), 2.91 (d, 3H), 2.24 (s, 3H), 1.58 (s, 6H).

LC/MS (Method 5, ESIpos): Rₜ=1.01 min, m/z=433 [M+H]⁺.

### Example 84

### 5-({3-[(Z)-1-Fluoro-2-{4-[1-(trifluoromethyl)cyclopropyl]phenyl}vinyl]-5-methyl-1H-pyrazol-1-yl}methyl)-N-methylpyridine-2-amine

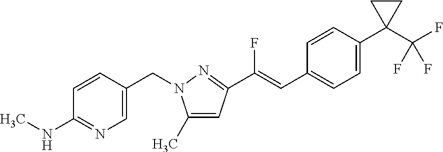 [SMILES: CNc1ccc(Cn2nc(/C(F)=C/c3ccc(C4(C(F)(F)F)CC4)cc3)cc2C)cn1]

Analogously to the process described under Example 79, 31 mg (0.072 mmol) of the compound from Example 70 and 888 μl (7.16 mmol) of a 33% strength methylamine solution in ethanol gave 17 mg (53% of theory, purity 97%) of the title compound. In this case, the reaction time in the microwave oven was 6.5 h at 150° C. For the preparative HPLC purification of the crude product, Method 27 was used.

¹H NMR (400 MHz, CDCl₃, δ/ppm): 7.97 (d, 1H), 7.57 (d, 2H), 7.46-7.40 (m, 2H), 7.34 (dd, 1H), 6.36 (m, 2H), 6.26 (s, 1H), 5.16 (s, 2H), 4.97 (br. s, 1H), 2.91 (d, 3H), 2.24 (s, 3H), 1.35 (m, 2H), 1.03 (s, 2H).

LC/MS (Method 6, ESIpos): Rₜ=2.00 min, m/z=431 [M+H]⁺.

### Example 85

### 5-[(3-{(Z)-1-Fluoro-2-[4-(trifluoromethyl)phenyl]vinyl}-5-methyl-1H-pyrazol-1-yl)methyl]-N-methylpyridine-2-amine

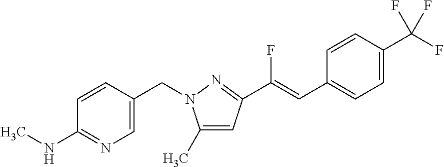 [SMILES: CNc1ccc(Cn2nc(/C(F)=C/c3ccc(C(F)(F)F)cc3)cc2C)cn1]

Analogously to the process described under Example 79, 60 mg (0.152 mmol) of the compound from Example 71 and 1.9 ml (15.2 mmol) of a 33% strength methylamine solution in ethanol gave 16 mg (59% of theory, purity 94%) of the title compound. In this case, the reaction time in the microwave oven was 5 h at 150° C. For the preparative HPLC purification of the crude product, Method 20 was used. The substance obtained in this manner was finally triturated with pentane, filtered off and dried under high vacuum.

¹H NMR (400 MHz, CDCl₃, δ/ppm): 10.21 (br. s, 1H), 7.78 (d, 1H), 7.70 (d, 2H), 7.60 (d, 2H), 7.56 (s, 1H), 6.76 (d, 1H), 6.40 (d, 1H), 6.34 (s, 1H), 5.15 (s, 2H), 2.98 (s, 3H), 2.29 (s, 3H).

LC/MS (Method 2, ESIpos): Rₜ=1.08 min, m/z=391 [M+H]⁺.

### Example 86

### 5-[(3-{(Z)-1-Fluoro-2-[4-(trimethylsilyl)phenyl]vinyl}-5-methyl-1H-pyrazol-1-yl)methyl]-N-methylpyridine-2-amine

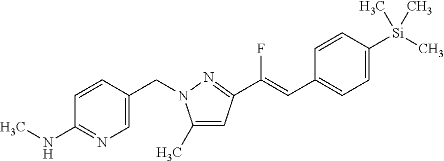

Analogously to the process described under Example 79, 107 mg (0.269 mmol) of the compound from Example 72 and 3.3 ml (26.8 mmol) of a 33% strength methylamine solution in ethanol gave 72 mg (68% of theory) of the title compound. In this case, the reaction time in the microwave oven was 3 h at 135° C., followed by 2 h at 150° C. For the preparative HPLC purification of the crude product, Method 27 was used.

¹H NMR (400 MHz, CDCl₃, δ/ppm): 7.98 (d, 1H), 7.59 (d, 2H), 7.51 (d, 2H), 7.33 (dd, 1H), 6.36 (d, 1H), 6.35 (d, 1H), 6.26 (s, 1H), 5.16 (s, 2H), 4.67 (br. s, 1H), 2.91 (d, 3H), 2.24 (s, 3H), 0.26 (m, 9H).

LC/MS (Method 6, ESIpos): Rₜ=2.14 min, m/z=395 [M+H]⁺.

### Example 87

### 5-({3-[(Z)-2-(4-tert-Butylphenyl)-1-fluorovinyl]-5-methyl-1H-pyrazol-1-yl}methyl)-N-methylpyridine-2-amine

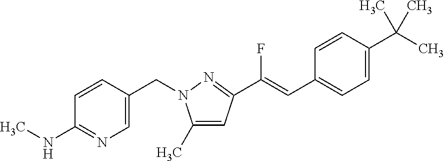 [SMILES: CNc1ccc(Cn2nc(/C(F)=C/c3ccc(C(C)(C)C)cc3)cc2C)cn1]

Analogously to the process described under Example 79, 146 mg (0.380 mmol) of the compound from Example 73 and 4.7 ml (38.0 mmol) of a 33% strength methylamine solution in ethanol gave 103 mg (72% of theory) of the title compound. In this case, the reaction time in the microwave oven was 5 h at 150° C. For the preparative HPLC purification of the crude product, Method 20 was used.

¹H NMR (400 MHz, CDCl₃, δ/ppm): 7.98 (d, 1H), 7.56 (d, 2H), 7.38 (d, 2H), 7.33 (dd, 1H), 6.36 (d, 1H), 6.35 (d, 1H), 6.25 (s, 1H), 5.16 (s, 2H), 4.67 (br. s, 1H), 2.91 (d, 3H), 2.23 (s, 3H), 1.33 (s, 9H).

LC/MS (Method 5, ESIpos): Rₜ=0.98 min, m/z=397 [M+H]⁺.

### Example 88

### 5-({3-[(Z)-2-(4-Cyclohexylphenyl)-1-fluorovinyl]-5-methyl-1H-pyrazol-1-yl}methyl)-N-methylpyridine-2-amine

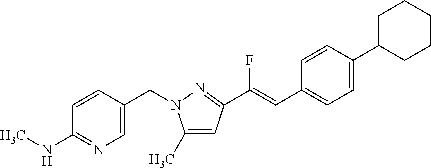 [SMILES: CNc1ccc(Cn2nc(/C(F)=C/c3ccc(C4CCCCC4)cc3)cc2C)cn1]

At RT, 0.55 ml (7.14 mmol) of trifluoroacetic acid was added to a solution of 70 mg (0.123 mmol, purity 98%) of the compound from Example 40A in 0.55 ml of dichloromethane. The mixture was stirred at RT for 40 h. The mixture was then diluted with dichloromethane and the mixture was neutralized with saturated aqueous sodium bicarbonate solution. After phase separation, and extraction of the aqueous phase with dichloromethane, the combined organic phases were dried over magnesium sulphate, filtered and concentrated. The residue was purified by thick-layer chromatography (silica gel, cyclohexane/ethyl acetate 4:6). The product zone was extracted with dichloromethane/methanol 95:5. After concentration, the residue was triturated with pentane, filtered off and dried under high vacuum. This gave 18 mg (35% of theory) of the title compound.

¹H NMR (400 MHz, CDCl₃, δ/ppm): 7.99 (d, 1H), 7.54 (d, 2H), 7.32 (dd, 1H), 7.19 (d, 2H), 6.35 (d, 1H), 6.34 (d, 1H), 6.24 (s, 1H), 5.16 (s, 2H), 4.57-4.51 (m, 1H), 2.90 (d, 3H), 2.54-2.43 (m, 1H), 2.23 (s, 3H), 1.92-1.79 (m, 4H), 1.75 (d, 1H), 1.50-1.32 (m, 4H), 1.32-1.20 (m, 1H).

LC/MS (Method 2, ESIpos): Rₜ=1.31 min, m/z=405 [M+H]⁺.

### Example 89

### 5-({3-[(Z)-1-Fluoro-2-(4-isopropylphenyl)vinyl]-5-methyl-1H-pyrazol-1-yl}methyl)-N-methyl-pyridine-2-amine

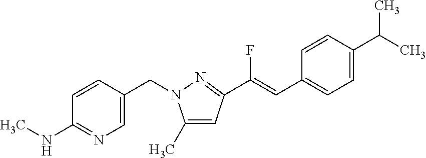 [SMILES: CNc1ccc(Cn2nc(/C(F)=C/c3ccc(C(C)C)cc3)cc2C)cn1]

Analogously to the process described under Example 79, 266 mg (0.720 mmol) of the compound from Example 74 and 8.9 ml (71.9 mmol) of a 33% strength methylamine solution in ethanol gave 179 mg (68% of theory) of the title compound. In this case, the reaction time in the microwave oven was 5 h at 150° C.

¹H NMR (400 MHz, CDCl₃, δ/ppm): 7.99 (d, 1H), 7.55 (d, 2H), 7.32 (dd, 1H), 7.22 (d, 2H), 6.36 (d, 1H), 6.34 (d, 1H), 6.25 (s, 1H), 5.16 (s, 2H), 4.59 (br. s, 1H), 2.95-2.86 (m, 4H), 2.23 (s, 3H), 1.26 (d, 6H).

LC/MS (Method 5, ESIpos): Rₜ=0.95 min, m/z=365 [M+H]⁺.

### Example 90

### 5-({3-[(Z)-1-Fluoro-2-(4-isobutylphenyl)vinyl]-5-methyl-1H-pyrazol-1-yl}methyl)-N-methyl-pyridine-2-amine

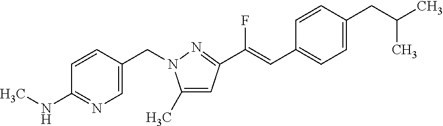 [SMILES: CNc1ccc(Cn2nc(/C(F)=C/c3ccc(CC(C)C)cc3)cc2C)cn1]

Analogously to the process described under Example 79, 388 mg (1.01 mmol) of the compound from Example 75 and 12.5 ml (101 mmol) of a 33% strength methylamine solution in ethanol gave 230 mg (60% of theory) of the title compound. In this case, the reaction time in the microwave oven was 5 h at 150° C.

¹H NMR (400 MHz, CDCl₃, δ/ppm): 7.99 (d, 1H), 7.52 (d, 2H), 7.32 (dd, 1H), 7.13 (d, 2H), 6.35 (d, 1H), 6.34 (d, 1H), 6.25 (s, 1H), 5.16 (s, 2H), 4.60 (br. s, 1H), 2.91 (d, 3H), 2.47 (d, 2H), 2.24 (s, 3H), 1.93-1.81 (m, 1H), 0.91 (d, 6H).

LC/MS (Method 5, ESIpos): Rₜ=0.99 min, m/z=379 [M+H]⁺.

### Example 91

### 5-[(3-{(Z)-1-Fluoro-2-[4-(pentafluoro-λ⁶-sulphanyl)phenyl]vinyl}-5-methyl-1H-pyrazol-1-yl)-methyl]-N-methylpyridine-2-amine

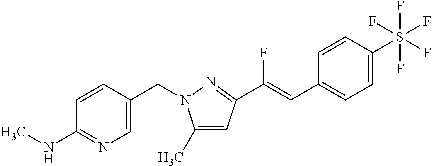 [SMILES: CNc1ccc(Cn2nc(/C(F)=C/c3ccc(S(F)(F)(F)(F)F)cc3)cc2C)cn1]

Analogously to the process described under Example 81, 90 mg (0.150 mmol) of the compound from Example 41A and 700 μl (9.09 mmol) of trifluoroacetic acid in 700 μl dichloromethane gave 32 mg (47% of theory) of the title compound. In this case, the reaction time was 45 h at RT. For the extraction that followed after the preparative HPLC, ethyl acetate (instead of dichloromethane) was used.

¹H NMR (400 MHz, CDCl₃, δ/ppm): 7.97 (d, 1H), 7.72 (d, 2H), 7.66 (d, 2H), 7.34 (dd, 1H), 6.40 (d, 1H), 6.38 (d, 1H), 6.30 (s, 1H), 5.16 (s, 2H), 4.91 (br. s, 1H), 2.91 (d, 3H), 2.26 (s, 3H).

LC/MS (Method 2, ESIpos): Rₜ=1.14 min, m/z=449 [M+H]⁺.

### Example 92

### N-Ethyl-2-({4-[(Z)-2-fluoro-2-(5-methyl-1-{[6-(methylamino)pyridin-3-yl]methyl}-1H-pyrazol-3-yl)vinyl]phenyl}sulphanyl)-2-methylpropanamide

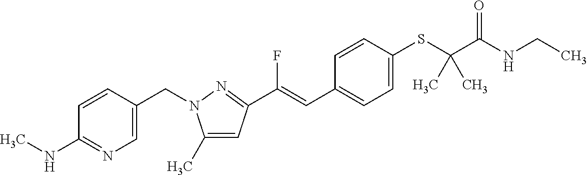 [SMILES: CCNC(=O)C(C)(C)Sc1ccc(/C=C(\F)c2cc(C)n(Cc3ccc(NC)nc3)n2)cc1]

Analogously to the process described under Example 79, 50 mg (0.106 mmol) of the compound from Example 76 and 1.3 ml (10.6 mmol) of a 33% strength methylamine solution in ethanol gave 36 mg (66% of theory, purity 92%) of the title compound. In this case, the reaction time in the microwave oven was 5 h at 150° C. Here, purification of the crude product was carried out by thick-layer chromatography (silica gel, dichloromethane/methanol 95:5). The product zone was extracted with dichloromethane/methanol 9:1, the extract was concentrated and the residue obtained was dried under high vacuum.

¹H NMR (400 MHz, CDCl₃, δ/ppm): 7.99 (d, 1H), 7.53 (d, 2H), 7.35 (d, 1H), 7.31 (dd, 1H), 7.29-7.27 (m, 1H), 6.90-6.85 (m, 1H), 6.36 (d, 1H), 6.33 (d, 1H), 6.26 (s, 1H), 5.16 (s, 2H), 4.60 (m, 1H), 3.32 (m, 2H), 2.91 (d, 3H), 2.24 (s, 3H), 1.52 (s, 6H), 1.16 (t, 3H).

LC/MS (Method 2, ESIpos): Rₜ=0.97 min, m/z=468 [M+H]⁺.

### Example 93

### 2-({4-[(Z)-2-Fluoro-2-(5-methyl-1-{[6-(methylamino)pyridin-3-yl]methyl}-1H-pyrazol-3-yl)-vinyl]phenyl}sulphanyl)-2-methyl-1-(pyrrolidin-1-yl)propan-1-one

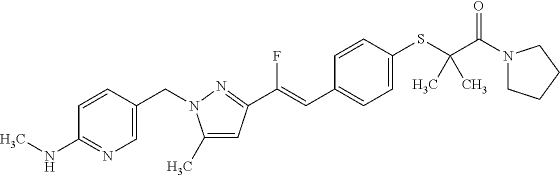 [SMILES: CNc1ccc(Cn2nc(/C(F)=C/c3ccc(SC(C)(C)C(=O)N4CCCC4)cc3)cc2C)cn1]

Analogously to the process described under Example 79, 90 mg (0.180 mmol) of the compound from Example 77 and 2.2 ml (18.0 mmol) of a 33% strength methylamine solution in ethanol gave 43 mg (49% of theory) of the title compound. In this case, the reaction time in the microwave oven was 5 h at 150° C. For the preparative HPLC purification of the crude product, Method 20 was used.

¹H NMR (400 MHz, CDCl₃, δ/ppm): 7.99 (d, 1H), 7.52 (d, 2H), 7.34-7.28 (m, 3H), 6.37 (s, 1H), 6.30 (d, 1H), 6.26 (s, 1H), 5.16 (s, 2H), 4.61-4.55 (m, 1H), 4.02 (br. s, 1H), 3.52 (br. s, 1H), 2.91 (d, 3H), 2.24 (s, 3H), 2.20-1.91 (m, 2H), 1.88-1.79 (m, 2H), 1.56 (s, 6H).

LC/MS (Method 2, ESIpos): Rₜ=1.08 min, m/z=494 [M+H]⁺.

### Example 94

### 5-({3-[(Z)-2-{4-[(Diisopropylamino)methyl]phenyl}-1-fluorovinyl]-5-methyl-1H-pyrazol-1-yl}-methyl)-N-methylpyridine-2-amine

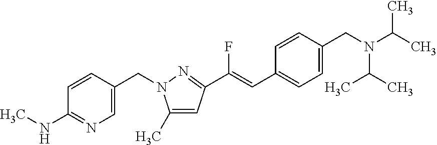 [SMILES: CNc1ccc(Cn2nc(/C(F)=C/c3ccc(CN(C(C)C)C(C)C)cc3)cc2C)cn1]

Analogously to the process described under Example 81, 95 mg (0.162 mmol) of the compound from Example 42A and 800 μl (10.4 mmol) of trifluoroacetic acid in 800 μl dichloromethane gave 48 mg (68% of theory) of the title compound. For the preparative HPLC purification of the crude product, Method 37 was used.

¹H NMR (400 MHz, CDCl₃, δ/ppm): 7.99 (d, 1H), 7.54 (d, 2H), 7.37 (d, 2H), 7.33 (dd, 1H), 6.36 (d, 1H), 6.34 (d, 1H), 6.25 (s, 1H), 5.16 (s, 2H), 4.62 (br. s, 1H), 3.65 (br. s, 2H), 3.04 (br. s, 2H), 2.91 (d, 3H), 2.23 (s, 3H), 1.04 (d, 12H).

LC/MS (Method 7, ESIpos): Rₜ=1.25 min, m/z=436 [M+H]⁺.

### Example 95

### 5-[(3-{(Z)-2-[6-(2,6-Dimethylmorpholin-4-yl)pyridin-3-yl]-1-fluorovinyl}-5-methyl-1H-pyrazol-1-yl)methyl]-N-methylpyridine-2-amine

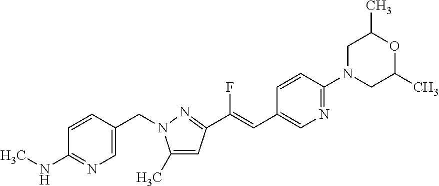

Analogously to the process described under Example 79, 120 mg (0.265 mmol, purity 94%) of the compound from Example 65 and 3.3 ml (26.5 mmol) of a 33% strength methylamine solution in ethanol gave 99 mg (86% of theory) of the title compound. In this case, the reaction time in the microwave oven was 1.5 h at 100° C. For the preparative HPLC purification of the crude product, Method 37 was used. The substance obtained after neutralization with sodium bicarbonate and extraction was finally triturated with pentane, filtered off and dried under high vacuum.

¹H NMR (400 MHz, CDCl₃, δ/ppm): 8.32 (d, 1H), 7.98 (d, 1H), 7.89 (dd, 1H), 7.32 (dd, 1H), 6.64 (d, 1H), 6.35 (d, 1H), 6.24 (d, 1H), 6.23 (s, 1H), 5.15 (s, 2H), 4.62 (br. s, 1H), 4.08 (d, 2H), 3.79-3.68 (m, 2H), 2.90 (d, 3H), 2.55 (dd, 2H), 2.24 (s, 3H), 1.27 (d, 6H).

LC/MS (Method 5, ESIpos): Rₜ=0.68 min, m/z=437 [M+H]⁺.

### Example 96

### N-Ethyl-5-[(3-{(Z)-1-fluoro-2-[4-(1,1,1-trifluoro-2-methylpropan-2-yl)phenyl]vinyl}-5-methyl-1H-pyrazol-1-yl)methyl]pyridine-2-amine

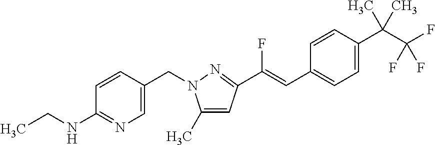 [SMILES: CCNc1ccc(Cn2nc(/C(F)=C/c3ccc(C(C)(C)C(F)(F)F)cc3)cc2C)cn1]

Analogously to the process described under Example 79, 120 mg (0.274 mmol) of the compound from Example 69 were reacted in a microwave oven with ethylamine. Here, the compound was initially heated at 135° C. with 2.7 ml (5.48 mmol) of a 2 M solution of ethylamine in ethanol for 3 h. After addition of another 2.7 ml (5.48 mmol) of the 2 Methylamine solution in ethanol, the mixture was heated at 145° C. for a further 6 h. Finally, 1 ml (11 mmol) of a 70% strength aqueous ethylamine solution was added, and the mixture was heated at 145° C. for another 8 h. This gave 99 mg (86% of theory) of the title compound.

¹H NMR (400 MHz, CDCl₃, δ/ppm): 7.97 (d, 1H), 7.60 (d, 2H), 7.47 (d, 2H), 7.31 (dd, 1H), 6.35 (d, 1H), 6.34 (d, 1H), 6.26 (s, 1H), 5.15 (s, 2H), 4.56 (br. s, 1H), 3.33-3.24 (m, 2H), 2.24 (s, 3H), 1.58 (s, 6H), 1.24 (t, 3H).

LC/MS (Method 5, ESIpos): Rₜ=1.03 min, m/z=447 [M+H]⁺.

### Example 97

### 2-{4-[(Z)-2-(1-{[6-(Ethylamino)pyridin-3-yl]methyl}-5-methyl-1H-pyrazol-3-yl)-2-fluorovinyl]-phenyl}-1,1,1,3,3,3-hexafluoropropan-2-ol

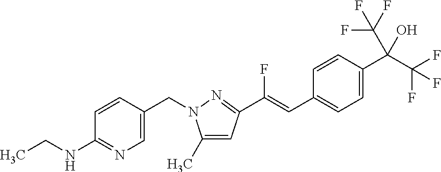 [SMILES: CCNc1ccc(Cn2nc(/C(F)=C/c3ccc(C(O)(C(F)(F)F)C(F)(F)F)cc3)cc2C)cn1]

Analogously to the process described under Example 79, 150 mg (0.304 mmol) of the compound from Example 78 and 2.6 ml (30.4 mmol) of a 70% strength aqueous ethylamine solution gave 44 mg (29% of theory) of the title compound. In this case, the reaction time in the microwave oven was 12 h at 145° C. The crude product was purified by two preparative HPLCs (first according to Method 20, then according to Method 38).

¹H NMR (400 MHz, CDCl₃, δ/ppm): 7.95 (d, 1H), 7.73-7.64 (m, 4H), 7.32 (dd, 1H), 6.38 (d, 1H), 6.35 (d, 1H), 6.28 (s, 1H), 5.16 (s, 2H), 4.56 (br. s, 1H), 3.32-3.22 (m, 2H), 2.25 (s, 3H), 1.24 (t, 3H).

LC/MS (Method 5, ESIpos): Rₜ=0.92 min, m/z=503 [M+H]⁺.

### Example 98

### 2-Chloro-4-[(3-{(Z)-1-fluoro-2-[4-(trimethylsilyl)phenyl]vinyl}-5-methyl-1H-pyrazol-1-yl)-methyl]pyridine

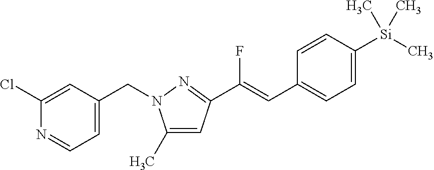 [SMILES: Cc1cc(/C(F)=C/c2ccc([Si](C)(C)C)cc2)nn1Cc1ccnc(Cl)c1]

41 mg (0.364 mmol) of potassium tert-butoxide were added to a solution of 400 mg (1.46 mmol) of the compound from Example 11A and 307 mg (1.89 mmol) of 2-chloro-4-(chloromethyl)pyridine in 13 ml of THF. The mixture was stirred under reflux for 4 h. A further 94 mg (0.342 mmol) of the compound from Example 11A and 41 mg (0.364 mmol) of potassium tert-butoxide were then added, and the mixture was stirred under reflux for another 18 h. After cooling to RT, 50 ml of ethyl acetate and 50 ml of water were added, and the phases were separated. The aqueous phase was extracted twice with 50 ml of ethyl acetate. The combined organic phases were dried over sodium sulphate, filtered and concentrated. The residue was purified initially by column chromatography (silica gel, mobile phase cyclohexane/ethyl acetate 85:15) and then by preparative HPLC (Method 27). The combined product fractions were concentrated to a small residual volume of aqueous phase, adjusted to pH 8 with saturated aqueous sodium bicarbonate solution and extracted three times with ethyl acetate. The combined ethyl acetate phases were dried over sodium sulphate, filtered and concentrated. The residue was dried under high vacuum. This gave 275 mg (47% of theory) of the title compound.

¹H NMR (400 MHz, CDCl₃, δ/ppm): 8.35 (d, 1H), 7.60 (d, 2H), 7.52 (d, 2H), 7.02 (s, 1H), 6.91 (d, 1H), 6.38 (d, 1H), 6.36 (s, 1H), 5.31 (s, 2H), 2.23 (s, 3H), 0.27 (s, 9H).

LC/MS (Method 2, ESIpos): Rₜ=1.64 min, m/z=400/402 [M+H]⁺.

### Example 99

### 4-({3-[(Z)-2-(4-tert-Butylphenyl)-1-fluorovinyl]-5-methyl-1H-pyrazol-1-yl}methyl)-2-chloro-pyridine

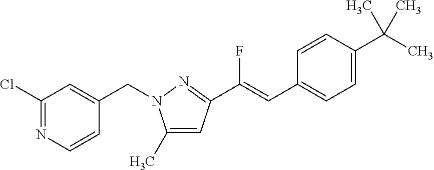 [SMILES: Cc1cc(/C(F)=C/c2ccc(C(C)(C)C)cc2)nn1Cc1ccnc(Cl)c1]

200 mg (0.774 mmol) of the compound from Example 12A and 257 mg (1.16 mmol) of (2-chloropyridin-4-yl)methyl methanesulphonate [for the preparation, see, for example, U.S. Pat. No. 6,759,428-B2, Example 37, Step 1] were initially charged in 100 ml of THF, and 130 mg (1.16 mmol) of solid potassium tert-butoxide were added at a temperature of 0° C. The reaction mixture was then stirred at RT for 16 h. About 250 ml of water were then added, and the mixture was extracted three times with in each case about 100 ml of ethyl acetate. The combined organic extracts were washed successively with water and saturated aqueous sodium chloride solution. After drying over anhydrous magnesium sulphate, the mixture was filtered and the filtrate was freed from the solvent on a rotary evaporator. The residue obtained was separated into its components by preparative HPLC (Method 34). After evaporation of the product fractions, it was found that the product was a mixture of the title compound and the regioisomeric alkylation product (“benzylation” at the other pyrazole nitrogen atom). This regioisomer mixture was then separated by a second preparative HPLC (Method 39). This gave 163 mg (55% of theory) of the title compound and 45 mg of the regioisomeric alkylation product.

¹H NMR (400 MHz, CDCl₃, δ/ppm): 8.35 (d, 1H), 7.56 (d, 2H), 7.39 (d, 2H), 7.02 (s, 2H), 6.91 (d, 1H), 6.36 (d, 1H), 6.35 (s, 1H), 5.31 (s, 2H), 2.23 (s, 3H), 1.33 (s, 9H).

LC/MS (Method 5, ESIpos): Rₜ=1.41 min, m/z=384/386 [M+H]⁺.

### Example 100

### 1-{4-[(3-{(Z)-1-Fluoro-2-[4-(trimethylsilyl)phenyl]vinyl}-5-methyl-1H-pyrazol-1-yl)methyl]-pyridin-2-yl}piperazine

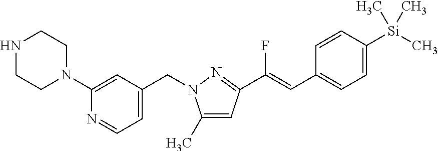 [SMILES: Cc1cc(/C(F)=C/c2ccc([Si](C)(C)C)cc2)nn1Cc1ccnc(N2CCNCC2)c1]

Under argon, a mixture of 200 mg (0.50 mmol) of the compound from Example 98 and 861 mg (10.0 mmol) of piperazine was stirred at 150° C. overnight. After cooling to RT, the piperazine which had sublimed in the reflux condenser was removed, and 30 ml of water and 30 ml of ethyl acetate were added to the content of the flask. After phase separation, the aqueous phase was extracted twice with in each case 30 ml of ethyl acetate. The combined organic phases were washed once with 50 ml of saturated sodium chloride solution, dried over sodium sulphate, filtered and concentrated. Drying of the residue under high vacuum gave 208 mg (89% of theory, purity 96% of theory) of the title compound.

¹H NMR (400 MHz, CDCl₃, δ/ppm): 8.12 (d, 1H), 7.60 (d, 2H), 7.52 (d, 2H), 6.44-6.30 (m, 3H), 6.28 (s, 1H), 5.22 (s, 2H), 3.48-3.42 (m, 4H), 2.98-2.92 (m, 4H), 2.21 (s, 3H), 0.27 (m, 9H).

LC/MS (Method 5, ESIpos): Rₜ=1.08 min, m/z=450 [M+H]⁺.

### Example 101

### 1-[4-({3-[(Z)-2-(4-tert-Butylphenyl)-1-fluorovinyl]-5-methyl-1H-pyrazol-1-yl}methyl)pyridin-2-yl]piperazine

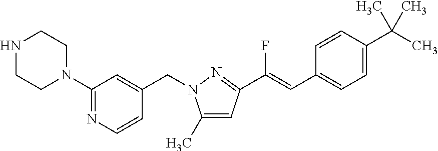 [SMILES: Cc1cc(/C(F)=C/c2ccc(C(C)(C)C)cc2)nn1Cc1ccnc(N2CCNCC2)c1]

A solution of 153 mg (0.399 mmol) of the compound from Example 99 and 687 mg (7.91 mmol) of piperazine in 6 ml of ethanol in a closed vessel was heated in a microwave oven (Biotage Initiator with dynamic irradiation power control) at 180° C. for 2 h. After cooling to RT, about 50 ml of water were added and the mixture was extracted three times with in each case about 50 ml of ethyl acetate. The combined organic extracts were washed successively with water and saturated aqueous sodium chloride solution. After drying over anhydrous magnesium sulphate, the mixture was filtered and the solvent was removed on a rotary evaporator. The crude product obtained was purified by preparative HPLC (Method 14). After evaporation of the product fractions, the product obtained was dissolved in about 10 ml of methanol and passed through an ion exchanger column (Polymerlabs, Stratospheres SPE, PL-HCO₃ MP SPE, capacity 0.9 mmol) to convert the formic acid salt (from the HPLC) into the free acid. Evaporation and drying under high vacuum gave 142 mg (81% of theory) of the title compound.

¹H NMR (400 MHz, CDCl₃, δ/ppm): 8.12 (d, 1H), 7.56 (d, 2H), 7.38 (d, 2H), 6.36 (d, 1H), 6.33 (d, 1H), 6.32 (s, 1H), 6.27 (s, 1H), 5.22 (s, 2H), 3.47-3.43 (m, 4H), 2.97-2.93 (m, 4H), 2.21 (s, 3H), 1.33 (s, 9H).

LC/MS (Method 5, ESIpos): Rₜ=1.03 min, m/z=434 [M+H]⁺.

### Example 102

### 1-Cyclopropyl-4-{4-[(3-{(Z)-1-fluoro-2-[4-(trifluoromethoxy)phenyl]vinyl}-5-methyl-1H-pyrazol-1-yl)methyl]pyridin-2-yl}piperazine

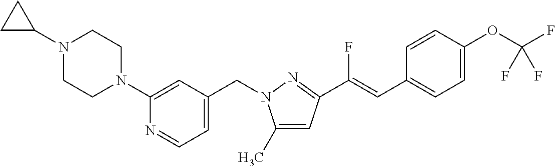 [SMILES: Cc1cc(/C(F)=C/c2ccc(OC(F)(F)F)cc2)nn1Cc1ccnc(N2CCN(C3CC3)CC2)c1]

64 mg (0.573 mmol) of potassium tert-butoxide were added to a solution of 126 mg (0.441 mmol) of the compound from Example 3A and 144 mg (0.573 mmol) of the compound from Example 32A in 3.8 ml of THF. The mixture was initially stirred under reflux for 18 h. A further 25 mg (0.220 mmol) of potassium tert-butoxide were then added, after a few hours followed by another 25 mg (0.220 mmol) of potassium tert-butoxide and a further 72 mg (0.287 mmol) of the compound from Example 32A. The mixture was then stirred under reflux for a further 6 h. After cooling to RT, 50 ml of ethyl acetate and 50 ml of dilute aqueous sodium chloride solution were added. After phase separation, the aqueous phase was extracted twice with in each case 50 ml of ethyl acetate. The combined organic phases were dried over sodium sulphate, filtered and concentrated. The residue was treated with methanol, and the solid formed was filtered off, washed twice with in each case 0.5 ml of methanol and dried under high vacuum. This gave 51 mg (23% of theory) of the title compound.

¹H NMR (400 MHz, CDCl₃, δ/ppm): 8.11 (d, 1H), 7.63 (d, 2H), 7.19 (d, 2H), 6.43-6.26 (m, 4H), 5.22 (s, 2H), 3.49-3.44 (m, 4H), 2.71-2.66 (m, 4H), 2.22 (s, 3H), 1.66-1.59 (m, 1H), 0.50-0.42 (m, 4H).

LC/MS (Method 5, ESIpos): Rₜ=1.01 min, m/z=502 [M+H]⁺.

### Example 103

### 1-{4-[(3-{(Z)-2-[3-Chloro-4-(trifluoromethoxy)phenyl]-1-fluorovinyl}-5-methyl-1H-pyrazol-1-yl)-methyl]pyridin-2-yl}-4-cyclopropylpiperazine

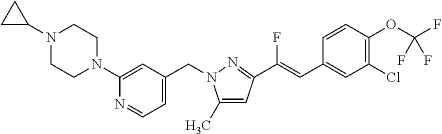 [SMILES: Cc1cc(/C(F)=C/c2ccc(OC(F)(F)F)c(Cl)c2)nn1Cc1ccnc(N2CCN(C3CC3)CC2)c1]

64 mg (0.573 mmol) of potassium tert-butoxide were added to a solution of 141 mg (0.441 mmol) of the compound from Example 5A and 144 mg (0.573 mmol) of the compound from Example 32A in 3.8 ml of THF. The mixture was stirred under reflux for 18 h. After cooling to RT, 30 ml of water were added, and the precipitate formed was filtered off and washed twice with water. The solid was then taken up in methanol and purified by preparative HPLC (Method 13). Two separate batches of product fractions were collected and in each case neutralized with aqueous sodium bicarbonate solution and concentrated to a small residual volume of aqueous phase. After two extractions with in each case 30 ml of ethyl acetate, the combined organic phases of each of the batches were dried over sodium sulphate, filtered and concentrated. Drying under high vacuum gave 66 mg (27% of theory, purity 95%) of the title compound from the first batch. The second batch was re-purified again by preparative HPLC (Method 40) and gave, after drying under high vacuum, a further 85 mg (36% of theory) of the title compound.

¹H NMR (400 MHz, CDCl₃, δ/ppm): 8.12 (d, 1H), 7.73 (d, 1H), 7.49 (dd, 1H), 7.29 (dd, 1H), 6.39-6.26 (m, 4H), 5.22 (s, 2H), 3.50-3.43 (m, 4H), 2.73-2.66 (m, 4H), 2.22 (s, 3H), 0.49-0.43 (m, 4H).

LC/MS (Method 5, ESIpos): Rₜ=1.05 min, m/z=536/538 [M+H]⁺.

### Example 104

### 1-Cyclopropyl-4-{4-[(3-{(Z)-1-fluoro-2-[4-(1,1,1-trifluoro-2-methylpropan-2-yl)phenyl]vinyl}-5-methyl-1H-pyrazol-1-yl)methyl]pyridin-2-yl}piperazine

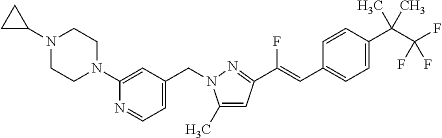 [SMILES: Cc1cc(/C(F)=C/c2ccc(C(C)(C)C(F)(F)F)cc2)nn1Cc1ccnc(N2CCN(C3CC3)CC2)c1]

64 mg (0.573 mmol) potassium tert-butoxide were added to a solution of 138 mg (0.441 mmol) of the compound from Example 7A and 144 mg (0.573 mmol) of the compound from Example 32A in 3.8 ml of THF. The mixture was stirred under reflux for 18 h. After cooling to RT, 30 ml of water were added, and the precipitate formed was filtered off and washed twice with water. The solid was then triturated with methanol, filtered off and dried under high vacuum. This gave 179 mg (73% of theory, purity 95%) of the title compound.

¹H NMR (400 MHz, CDCl₃, δ/ppm): 8.11 (d, 1H), 7.61 (d, 2H), 7.48 (d, 2H), 6.44-6.26 (m, 4H), 5.22 (s, 2H), 3.49-3.43 (m, 4H), 2.71-2.66 (m, 4H), 2.21 (s, 3H), 1.66-1.59 (m, 1H), 1.58 (s, 6H), 0.50-0.42 (m, 4H).

LC/MS (Method 6, ESIpos): Rₜ=2.06 min, m/z=428 [M+H]⁺.

### Example 105

### 1-Cyclopropyl-4-[4-({3-[(Z)-1-fluoro-2-{4-[1-(trifluoromethyl)cyclopropyl]phenyl}vinyl]-5-methyl-1H-pyrazol-1-yl}methyl)pyridin-2-yl]piperazine

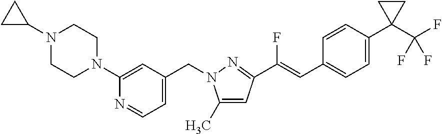 [SMILES: Cc1cc(/C(F)=C/c2ccc(C3(C(F)(F)F)CC3)cc2)nn1Cc1ccnc(N2CCN(C3CC3)CC2)c1]

84 mg (0.754 mmol) potassium tert-butoxide were added to a solution of 138 mg (0.441 mmol) of the compound from Example 9A and 144 mg (0.573 mmol) of the compound from Example 32A in 5 ml of THF. The mixture was stirred under reflux for 18 h. After cooling to RT, 50 ml of water and 50 ml of dilute aqueous sodium chloride solution were added, and after phase separation the aqueous phase was extracted twice with in each case 50 ml of ethyl acetate. The combined organic phases were dried over sodium sulphate, filtered and concentrated. The residue was triturated with warm methanol, filtered off and dried under high vacuum. This gave 187 mg (61% of theory) of the title compound.

¹H NMR (400 MHz, CDCl₃, δ/ppm): 8.11 (d, 1H), 7.58 (d, 2H), 7.44 (d, 2H), 6.34-6.26 (m, 4H), 5.22 (s, 2H), 3.48-3.44 (m, 4H), 2.71-2.66 (m, 4H), 2.21 (s, 3H), 1.66-1.60 (m, 1H), 1.37-1.32 (m, 2H), 1.06-1.01 (m, 2H), 0.49-0.43 (m, 4H).

LC/MS (Method 5, ESIpos): Rₜ=1.10 min, m/z=526 [M+H]⁺.

### Example 106

### 1-Cyclopropyl-4-{4-[(3-{(Z)-1-fluoro-2-[4-(trimethylsilyl)phenyl]vinyl}-5-methyl-1H-pyrazol-1-yl)methyl]pyridin-2-yl}piperazine

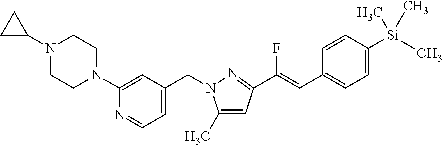 [SMILES: Cc1cc(/C(F)=C/c2ccc([Si](C)(C)C)cc2)nn1Cc1ccnc(N2CCN(C3CC3)CC2)c1]

Under argon, 3 Å molecular sieves and 490 μl (2.44 mmol) of [(1-ethoxy-1-cyclopropyl)oxy]-trimethylsilane were added to a solution of 183 mg (0.407 mmol) of the compound from Example 100 and 233 μl (4.06 mmol) of acetic acid in 4 ml of methanol. After 10 min of stirring at RT, 77 mg (1.22 mmol) of sodium cyanoborohydride were added and the mixture was heated to the boil for 2 h. After cooling to RT, the molecular sieve was filtered off and washed with methanol. The filtrate was concentrated. The solid obtained was triturated with 14.5 ml of a water/acetonitrile/DMSO mixture and then filtered off. Drying under high vacuum gave 51 mg (26% of theory) of the title compound as a first batch. The filtrate obtained was concentrated and the residue was purified by preparative HPLC (Method 27). The combined product fractions were concentrated to a small residual volume of aqueous phase and adjusted to pH 8 with saturated aqueous sodium bicarbonate solution. After three extractions with ethyl acetate, the combined ethyl acetate phases were dried over sodium sulphate, filtered and concentrated. The residue was dried under high vacuum. This gave a further 101 mg (51% of theory) of the title compound as a second batch.

¹H NMR (400 MHz, CDCl₃, δ/ppm): 8.11 (d, 1H), 7.60 (d, 2H), 7.51 (d, 2H), 6.44-6.26 (m, 4H), 5.22 (s, 2H), 3.49-3.44 (m, 4H), 2.72-2.66 (m, 4H), 2.21 (s, 3H), 1.67-1.55 (m, 1H), 0.50-0.40 (m, 4H), 0.27 (s, 9H).

LC/MS (Method 2, ESIpos): Rₜ=1.30 min, m/z=490 [M+H]⁺.

### Example 107

### 1-Cyclopropyl-4-{4-[(3-{(Z)-1-fluoro-2-[4-(pentafluoro-λ⁶-sulphanyl)phenyl]vinyl}-5-methyl-1H-pyrazol-1-yl)methyl]pyridin-2-yl}piperazine

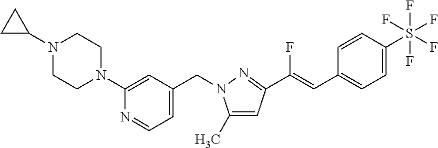 [SMILES: Cc1cc(/C(F)=C/c2ccc(S(F)(F)(F)(F)F)cc2)nn1Cc1ccnc(N2CCN(C3CC3)CC2)c1]

Analogously to the process described in Example 105, 145 mg (0.441 mmol) of the compound from Example 18A and 144 mg (0.573 mmol) of the compound from Example 32A gave 128 mg (53% of theory) of the title compound.

¹H NMR (400 MHz, CDCl₃, δ/ppm): 8.12 (d, 1H), 7.72 (d, 2H), 7.67 (d, 2H), 6.48-6.26 (m, 4H), 5.23 (s, 2H), 3.49-3.44 (m, 4H), 2.71-2.66 (m, 4H), 2.23 (s, 3H), 1.66-1.59 (m, 1H), 0.51-0.41 (m, 4H).

LC/MS (Method 5, ESIpos): Rₜ=1.04 min, m/z=543 [M+H]⁺.

### Example 108

### 2-{4-[(Z)-2-(1-{[2-(4-Cyclopropylpiperazin-1-yl)pyridin-4-yl]methyl}-5-methyl-1H-pyrazol-3-yl)-2-fluorovinyl]phenyl}-1,1,1,3,3,3-hexafluoropropan-2-ol

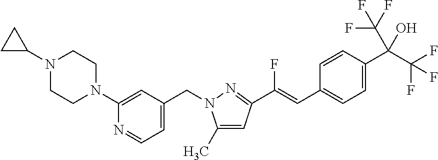 [SMILES: Cc1cc(/C(F)=C/c2ccc(C(O)(C(F)(F)F)C(F)(F)F)cc2)nn1Cc1ccnc(N2CCN(C3CC3)CC2)c1]

48 mg (0.424 mmol) of potassium tert-butoxide were added to a solution of 120 mg (0.326 mmol) of the compound from Example 16A and 107 mg (0.424 mmol) of the compound from Example 32A in 2.8 ml of THF. The mixture was initially stirred under reflux for 18 h. A further 48 mg (0.424 mmol) of potassium tert-butoxide were then added, and the mixture was stirred under reflux for another 8 h. After cooling to RT, 30 ml of water were added, and the precipitate formed was filtered off and washed twice with in each case 2 ml of water. This gave 93 mg (49% of theory) of the title compound as a first batch. Form the filtrate obtained, which had been combined with the wash solutions, a solid was filtered off which for its part was washed twice with in each case 2 ml of water. This solid was subsequently recrystallized from 3 ml of methanol and washed twice with in each case 0.5 ml of methanol. This gave 56 mg (30% of theory) of the title compound as a second batch.

¹H NMR (400 MHz, CDCl₃, δ/ppm): 8.11 (d, 1H), 7.73-7.66 (m, 4H), 6.47-6.25 (m, 4H), 5.23 (s, 2H), 4.48 (s, 1H), 3.51-3.43 (m, 4H), 2.72-2.67 (m, 4H), 2.22 (s, 3H), 1.66-1.60 (m, 1H), 0.49-0.42 (m, 4H).

LC/MS (Method 5, ESIpos): Rₜ=0.95 min, m/z=584 [M+H]⁺.

### Example 109

### N-{4-[(Z)-2-(1-{[2-(4-Cyclopropylpiperazin-1-yl)pyridin-4-yl]methyl}-5-methyl-1H-pyrazol-3-yl)-2-fluorovinyl]benzyl}-N-isopropylpropane-2-amine

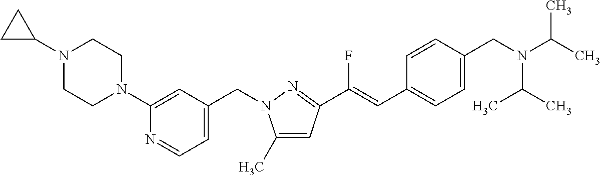 [SMILES: Cc1cc(/C(F)=C/c2ccc(CN(C(C)C)C(C)C)cc2)nn1Cc1ccnc(N2CCN(C3CC3)CC2)c1]

At 0° C., 92 mg (0.824 mmol) of potassium tert-butoxide were added to a solution of 200 mg (0.634 mmol) of the compound from Example 20A and 208 mg (0.824 mmol) of the compound from Example 32A in 6 ml of THF. The mixture was initially stirred at RT for 1 h and then under reflux for 18 h. After cooling to RT, the mixture was diluted with ethyl acetate and washed once with water. The aqueous phase was re-extracted once with ethyl acetate. The combined organic phases were dried over magnesium sulphate, filtered and concentrated. The residue was purified by preparative HPLC (Method 37). The combined product fractions were concentrated to a small residual volume of aqueous phase, and saturated aqueous sodium bicarbonate solution was added. After two extractions with ethyl acetate, the combined ethyl acetate phases were dried over magnesium sulphate, filtered and concentrated. The residue obtained was triturated with pentane, filtered off and washed with pentane. Drying under high vacuum gave 277 mg (82% of theory) of the title compound.

¹H NMR (400 MHz, CDCl₃, δ/ppm): 8.11 (d, 1H), 7.55 (d, 2H), 7.37 (d, 2H), 6.42-6.26 (m, 4H), 5.22 (s, 2H), 3.63 (s, 2H), 3.49-3.43 (m, 4H), 3.08-2.96 (m, 2H), 2.71-2.66 (m, 4H), 2.20 (s, 3H), 1.66-1.58 (m, 1H), 1.02 (d, 12H), 0.50-0.41 (m, 4H).

LC/MS (Method 5, ESIpos): Rₜ=0.66 min, m/z=531 [M+H]⁺.

### Example 110

### 1-[4-({3-[(Z)-2-(4-tert-Butylphenyl)-1-fluorovinyl]-5-methyl-1H-pyrazol-1-yl}methyl)pyridin-2-yl]-4-(2,2,2-trifluoroethyl)piperazine

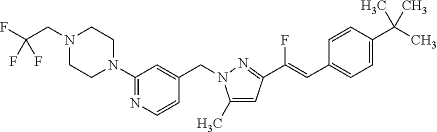 [SMILES: Cc1cc(/C(F)=C/c2ccc(C(C)(C)C)cc2)nn1Cc1ccnc(N2CCN(CC(F)(F)F)CC2)c1]

At a temperature of 0° C., 104 μl (0.616 mmol) of trifluoromethanesulphonic anhydride were added to a solution of 45 μl (0.616 mmol) of 2,2,2-trifluoroethanol and 107 μl (0.770 mmol) of triethylamine in 5 ml of dichloromethane. After 2 h of stirring at 0° C., 133 mg (0.308 mmol) of the compound from Example 101, dissolved in 1 ml of dichloromethane, were added. The cooling bath was removed, and stirring was continued at RT for 40 h. About 20 ml of water were then added, and the mixture was extracted three times with in each case about 20 ml of ethyl acetate. The combined organic extracts were washed with saturated aqueous sodium chloride solution, dried over anhydrous magnesium sulphate, filtered and freed from the solvent on a rotary evaporator. The crude product obtained was purified by preparative HPLC (Method 14). After evaporation of the product fractions, the product obtained was dissolved in about 5 ml of methanol and passed through an ion exchanger column (Polymerlabs, Stratospheres SPE, PL-HCO₃ MP SPE, capacity 0.9 mmol) to convert the formic acid salt (from the HPLC) into the free base. Evaporation and drying under high vacuum gave 122 mg (77% of theory) of the title compound.

¹H NMR (400 MHz, CDCl₃, δ/ppm): 8.12 (d, 1H), 7.56 (d, 2H), 7.39 (d, 2H), 6.35 (d, 1H), 6.34 (d, 1H), 6.32 (s, 1H), 6.26 (s, 1H), 5.22 (s, 2H), 3.52 (dd, 4H), 3.00 (quart, 2H), 2.75 (dd, 4H), 2.21 (s, 3H), 1.33 (s, 9H).

LC/MS (Method 5, ESIpos): Rₜ=1.40 min, m/z=516 [M+H]⁺.

### Example 111

### 3-{(Z)-2-Fluoro-2-[4-(trifluoromethoxy)phenyl]vinyl}-5-methyl-1-(4-methylbenzyl)-1H-pyrazole

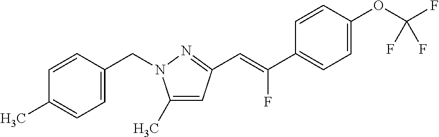 [SMILES: Cc1ccc(Cn2nc(/C=C(\F)c3ccc(OC(F)(F)F)cc3)cc2C)cc1]

At 0° C., 7.3 μl (0.094 mmol) of methanesulphonyl chloride were added to a solution of 32 mg (0.078 mmol) of the compound from Example 43A and 14 μl (0.101 mmol) of triethylamine in 0.5 ml of dichloromethane. The mixture was stirred initially at 0° C. for a few minutes and then at RT for 18 h. A further 140 μl (1.01 mmol) of triethylamine and 73 μl (0.940 mmol) of methanesulphonyl chloride were added, and the mixture was stirred at RT for another 2 h. Two portions of in each case 100 μl of 1,8-diazabicyclo[5.4.0]undec-7-ene (DBU) were then added in succession, and the mixture was stirred at RT for three days. A further 500 μl of 1,8-diazabicyclo[5.4.0]undec-7-ene (DBU) were added, and the mixture was stirred at RT for another four days. The mixture was then concentrated on a rotary evaporator and the residue was purified by preparative HPLC (Method 16). After removal of the solvent, the solid that remained was triturated with water, and the mixture was extracted three times with dichloromethane. The combined dichloromethane phases were dried over magnesium sulphate, filtered and concentrated. The residue was dried under high vacuum. This gave 8 mg (26% of theory, purity 95%) of the title compound.

¹H NMR (400 MHz, CDCl₃, δ/ppm): 7.63 (d, 2H), 7.24 (d, 2H), 7.12 (d, 2H), 7.01 (d, 2H), 6.52 (d, 1H), 6.51 (d, 1H), 5.25 (s, 2H), 2.32 (s, 3H), 2.23 (s, 3H).

LC/MS (Method 5, ESIpos): Rₜ=1.46 min, m/z=391 [M+H]⁺.

### Example 112

### 1-(3-Bromobenzyl)-3-{(Z)-2-fluoro-2-[4-(trifluoromethoxy)phenyl]vinyl}-5-methyl-1H-pyrazole

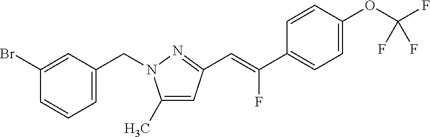 [SMILES: Cc1cc(/C=C(\F)c2ccc(OC(F)(F)F)cc2)nn1Cc1cccc(Br)c1]

Analogously to the process described under Example 6, 120 mg (0.419 mmol) of the compound from Example 45A and 126 mg (0.503 mmol) 1-bromo-3-(bromomethyl)benzene gave 65 mg (33% of theory) of the title compound. Here, the pre-purification of the crude products by silica gel chromatography was dispensed with; purification was by preparative HPLC according to Method 41.

¹H NMR (400 MHz, CDCl₃, δ/ppm): 7.64 (d, 2H), 7.41 (d, 1H), 7.26 (s, 1H, obscured by the CHCl₃ signal), 7.24 (d, 2H, partially obscured by the CHCl₃ signal), 7.19 (t, 1H), 7.01 (d, 1H), 6.55 (s, 1H), 6.50 (d, 1H), 5.26 (s, 2H), 2.25 (s, 3H).

LC/MS (Method 5, ESIpos): Rₜ=1.46 min, m/z=455/457 [M+H]⁺.

### Example 113

### 1-{3-[(3-{(Z)-2-Fluoro-2-[4-(trifluoromethoxy)phenyl]vinyl}-5-methyl-1H-pyrazol-1-yl)methyl]-phenyl}azetidin-3-ol

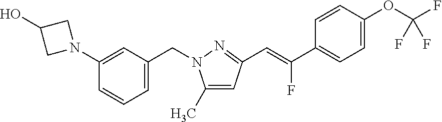 [SMILES: Cc1cc(/C=C(\F)c2ccc(OC(F)(F)F)cc2)nn1Cc1cccc(N2CC(O)C2)c1]

Under argon, a mixture of 60 mg (0.132 mmol) of the compound from Example 112, 66 mg (0.198 mmol) of the compound from Example 23A, 8 mg (0.009 mmol) of tris(dibenzylideneacetone)dipalladium, 13 mg (0.026 mmol) of 2-dicyclohexylphosphino-2′,4′,6′-triisopropylbiphenyl (X-Phos) and 25 mg (0.264 mmol) of sodium tert-butylate in 1.3 ml of toluene was heated in a microwave oven (Biotage Initiator with dynamic irradiation power control) at 80° C. for 1.5 h. After cooling to RT, about 50 ml of dichloromethane were added, and the mixture was washed successively with in each case about 50 ml of water and saturated aqueous sodium chloride solution. After drying of the organic phase over anhydrous magnesium sulphate, the mixture was filtered and the filtrate was concentrated on a rotary evaporator. The residue obtained was purified by MPLC (silica gel, mobile phase cyclohexane/ethyl acetate 9:1). After concentration of the product fractions, 140 mg of the tert-butyldiphenylsilyl-protected intermediate were obtained. This intermediate was dissolved in 5 ml of THF, and 132 μl (0.132 mmol) of a 1 M solution of tetra-n-butylammonium fluoride in THF were added at 0° C. After the reaction mixture had been stirred at RT for 10 min, it was diluted with a little methanol and then completely separated into its components by preparative HPLC (Method 14). After concentration of the product fractions, the solid obtained was triturated with about 5 ml of pentane, filtered off with suction and then dried under high vacuum. This gave 37 mg (60% of theory) of the title compound.

¹H NMR (400 MHz, CDCl₃, δ/ppm): 7.63 (d, 2H), 7.24 (d, 2H), 7.14 (t, 1H), 6.52 (d, 1H, J=40 Hz), 6.52 (d, 1H, J=4 Hz), 6.46 (d, 1H), 6.37 (d, 1H), 6.20 (s, 1H), 5.22 (s, 2H), 4.76-4.69 (m, 1H), 4.13 (t, 2H), 3.63 (dd, 2H), 2.32 (d, 1H), 2.23 (s, 3H).

LC/MS (Method 5, ESIpos): Rₜ=1.23 min, m/z=448 [M+H]⁺.

### Example 114

### {3-[(3-{(Z)-2-Fluoro-2-[4-(trifluoromethoxy)phenyl]vinyl}-5-methyl-1H-pyrazol-1-yl)methyl]-phenyl}(pyrrolidin-1-yl)methanone

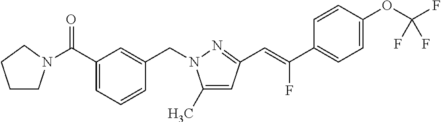 [SMILES: Cc1cc(/C=C(\F)c2ccc(OC(F)(F)F)cc2)nn1Cc1cccc(C(=O)N2CCCC2)c1]

At a temperature of 0° C., 33 mg (0.293 mmol) of solid potassium tert-butoxide were added to a solution of 70 mg (0.245 mmol) of the compound from Example 45A and 83 mg (0.293 mmol) of the compound from Example 46A in 3 ml of anhydrous dioxane. After removal of the ice/water bath, the reaction mixture was stirred at RT for 30 min. About 30 ml of water were then added, and the mixture was extracted three times with in each case about 30 ml of ethyl acetate. The combined organic extracts were washed with saturated aqueous sodium chloride solution, dried over anhydrous magnesium sulphate, filtered and freed from the solvent on a rotary evaporator. The crude product obtained in this manner was purified by preparative HPLC (Method 14). This gave 55 mg of a mixture of the title compound and the regioisomeric alkylation product (benzylation at the other pyrazole nitrogen atom). The regioisomer mixture was then separated by another preparative HPLC (Method 42). This gave 17 mg (15% of theory) of the title compound and 19 mg of the regioisomeric benzylation product.

¹H NMR (400 MHz, CDCl₃, δ/ppm): 7.64 (d, 2H), 7.42 (d, 1H), 7.35 (t, 1H), 7.26 (s, 1H, obscured by the CHCl₃ signal), 7.24 (d, 2H, partially obscured by the CHCl₃ signal), 7.12 (d, 1H), 6.55 (s, 1H), 6.50 (d, 1H), 5.31 (s, 2H), 3.62 (t, 2H), 3.35 (t, 2H), 2.24 (s, 3H), 1.94 (quint, 2H), 1.85 (quint, 2H).

LC/MS (Method 5, ESIpos): Rₜ=1.27 min, m/z=474 [M+H]⁺, 947 [2M+H]⁺.

### Example 115

### Methyl 3-[(3-{(Z)-2-fluoro-2-[4-(trifluoromethoxy)phenyl]vinyl}-5-methyl-1H-pyrazol-1-yl)methyl]benzoate

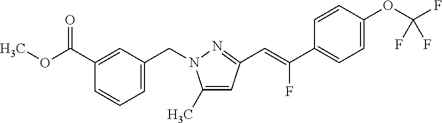 [SMILES: COC(=O)c1cccc(Cn2nc(/C=C(\F)c3ccc(OC(F)(F)F)cc3)cc2C)c1]

At a temperature of 0° C., 118 mg (1.05 mmol) of solid potassium tert-butoxide were added to a solution of 200 mg (0.699 mmol) of the compound from Example 45A and 240 mg (1.05 mmol) of methyl 3-(bromomethyl)benzoate in 8.7 ml of anhydrous dioxane. After removal of the ice/water bath, the reaction mixture was stirred at RT for 18 h. 100 ml of water were then added, and the mixture was extracted three times with in each case about 100 ml of ethyl acetate. The combined organic extracts were dried over anhydrous magnesium sulphate, filtered and then freed from the solvent on a rotary evaporator. The crude product obtained in this manner was purified by preparative HPLC (Method 46). This gave 89 mg (30% of theory) of the title compound. In addition, 95 mg (31% of theory) of a second fraction consisting of the regioisomeric alkylation product (benzylation at the other pyrazole nitrogen atom) were obtained.

¹H NMR (400 MHz, CDCl₃, δ/ppm): 7.96 (d, 1H), 7.86 (s, 1H), 7.64 (d, 2H), 7.40 (t, 1H), 7.25 (d, 1H and d, 2H; both partially obscured by the CHCl₃ signal), 6.55 (s, 1H), 6.51 (d, 1H), 5.33 (s, 2H), 3.91 (s, 3H), 2.25 (s, 3H).

LC/MS (Method 8, ESIpos): Rₜ=1.39 min, m/z=435 [M+H]⁺.

### Example 116

### 2-Chloro-5-[(3-{(Z)-2-fluoro-2-[4-(trifluoromethoxy)phenyl]vinyl}-5-methyl-1H-pyrazol-1-yl)-methyl]pyridine

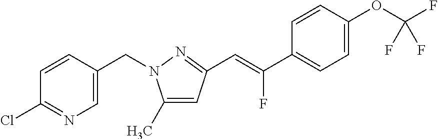 [SMILES: Cc1cc(/C=C(\F)c2ccc(OC(F)(F)F)cc2)nn1Cc1ccc(Cl)nc1]

175 mg (0.611 mmol) of the compound from Example 45A and 203 mg (0.917 mmol) of (6-chloropyridin-3-yl)methyl methanesulphonate [lit.: K. C. Iee et al., J. Org. Chem. 1999, 64 (23), 8576-8581] were initially charged in 7.3 ml of 1,4-dioxane, and 103 mg (0.917 mmol) of solid potassium tert-butoxide were added at a temperature of 0° C. The reaction mixture was then stirred at RT for 16 h. About 100 ml of water were then added, and the mixture was extracted three times with in each case about 100 ml of ethyl acetate. The combined organic extracts were washed successively with water and saturated aqueous sodium chloride solution. After drying over anhydrous magnesium sulphate, the mixture was filtered and the filtrate was freed from the solvent on a rotary evaporator. The residue obtained was separated into its components by preparative HPLC (Method 44). Concentration of the product fractions gave 80 mg (30% of theory) of the title compound.

¹H NMR (400 MHz, CDCl₃, δ/ppm): 8.26 (d, 1H), 7.64 (d, 2H), 7.40 (dd, 1H), 7.29 (d, 1H), 7.25 (d, 2H, partially obscured by the CHCl₃ signal), 6.54 (d, 1H), 6.47 (d, 1H), 5.27 (s, 2H), 2.27 (s, 3H).

LC/MS (Method 8, ESIpos): Rₜ=1.35 min, m/z=412/414 [M+H]⁺.

### Example 117

### 5-[(3-{(Z)-2-Fluoro-2-[4-(trifluoromethoxy)phenyl]vinyl}-5-methyl-1H-pyrazol-1-yl)methyl]-N-methylpyridine-2-amine

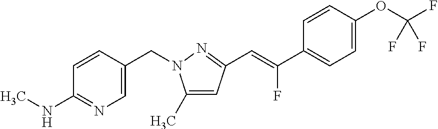 [SMILES: CNc1ccc(Cn2nc(/C=C(\F)c3ccc(OC(F)(F)F)cc3)cc2C)cn1]

A mixture of 77 mg (0.187 mmol) of the compound from Example 116 and 2.3 ml (18.5 mmol) of a 8 M solution of methylamine in ethanol was heated in a microwave oven (Biotage Initiator with dynamic irradiation power control) at 145° C. for 7 h. After cooling to RT, the volatile components were substantially removed on a rotary evaporator, and the residue was purified by preparative HPLC (Method 45). After evaporation of the product fractions, the residue was re-dissolved in about 5 ml of methanol and the solution was passed through an ion exchanger column (Polymerlabs, Stratospheres SPE, PL-HCO₃ MP SPE, capacity 0.9 mmol) to convert the formic acid salt (from the HPLC) into the free acid. Concentration and drying of the residue under high vacuum gave 45 mg (60% of theory) of the title compound.

¹H NMR (400 MHz, CDCl₃, δ/ppm): 7.98 (d, 1H), 7.63 (d, 2H), 7.29 (dd, 1H), 7.24 (d, 2H), 6.50 (d, 1H), 6.49 (d, 1H), 6.35 (d, 1H), 5.13 (s, 2H), 4.63 (broad, 1H), 2.90 (s, broad, 3H), 2.27 (s, 3H).

LC/MS (Method 5, ESIpos): Rₜ=0.93 min, m/z=407 [M+H]⁺.

### Example 118

### 2-Chloro-4-[(3-{(Z)-2-fluoro-2-[4-(trifluoromethoxy)phenyl]vinyl}-5-methyl-1H-pyrazol-1-yl)methyl]pyridine

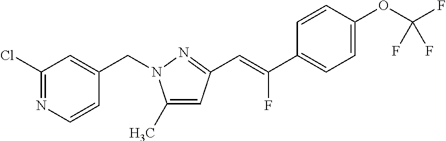 [SMILES: Cc1cc(/C=C(\F)c2ccc(OC(F)(F)F)cc2)nn1Cc1ccnc(Cl)c1]

Analogously to the process described under Example 99, 120 mg (0.419 mmol) of the compound from Example 45A and 112 mg (0.503 mmol) of (2-chloropyridin-4-yl)methyl methanesulphonate [for the preparation see, for example, U.S. Pat. No. 6,759,428-B2, Example 37, Step 1] gave, after the first HPLC purification of the crude product (Method 14), 115 mg of a mixture of the title compound and the regioisomeric alkylation product (“benzylation” at the other pyrazole nitrogen atom). This regioisomer mixture was then separated by another preparative HPLC (Method 43). This gave 45 mg (26% of theory) of the title compound and 18 mg of the regioisomeric alkylation product.

¹H NMR (400 MHz, CDCl₃, δ/ppm): 8.34 (d, 1H), 7.65 (d, 2H), 7.25 (d, 2H, partially obscured by the CHCl₃ signal), 7.00 (s, 1H), 6.90 (d, 1H), 6.59 (d, 1H), 6.48 (d, 1H), 5.28 (s, 2H), 2.26 (s, 3H).

LC/MS (Method 5, ESIpos): Rₜ=1.32 min, m/z=412/414 [M+H]⁺.

### Example 119

### 1-{4-[(3-{(Z)-2-Fluoro-2-[4-(trifluoromethoxy)phenyl]vinyl}-5-methyl-1H-pyrazol-1-yl)methyl]pyridin-2-yl}piperazine

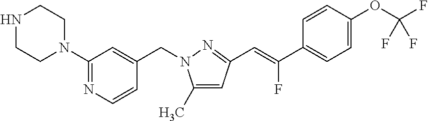 [SMILES: Cc1cc(/C=C(\F)c2ccc(OC(F)(F)F)cc2)nn1Cc1ccnc(N2CCNCC2)c1]

Analogously to the process described under Example 101, 45 mg (0.109 mmol) of the compound from Example 118 and 188 mg (2.19 mmol) of piperazine gave 52 mg (50% of theory) of the title compound. In this case, the reaction time was 1.75 h, and prior to the aqueous work-up the solvent was substantially removed on a rotary evaporator.

¹H NMR (400 MHz, CDCl₃, δ/ppm): 8.11 (d, 1H), 7.64 (d, 2H), 7.24 (d, 2H, partially obscured by the CHCl₃ signal), 6.55 (s, 1H), 6.51 (d, 1H), 6.31 (d, 1H), 6.28 (s, 1H), 5.19 (s, 2H), 3.46 (dd, 4H), 2.96 (dd, 4H), 2.24 (s, 3H).

LC/MS (Method 5, ESIpos): Rₜ=0.86 min, m/z=462 [M+H]⁺.

### Example 120

### 1-Cyclopropyl-4-{4-[(3-{(Z)-2-fluoro-2-[4-(trifluoromethoxy)phenyl]vinyl}-5-methyl-1H-pyrazol-1-yl)methyl]pyridin-2-yl}piperazine

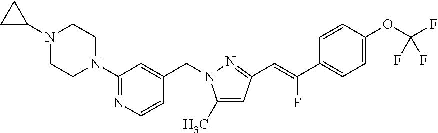 [SMILES: Cc1cc(/C=C(\F)c2ccc(OC(F)(F)F)cc2)nn1Cc1ccnc(N2CCN(C3CC3)CC2)c1]

Under argon, 21 mg of 3 Å molecular sieve and 116 mg (0.663 mmol) of [(1-ethoxy-1-cyclopropyl)oxy]trimethylsilane were added to a solution of 51 mg (0.111 mmol) of the compound from Example 119 and 63 μl (1.11 mmol) of acetic acid in 2 ml of methanol. After 10 min of stirring at RT, 21 mg (0.332 mmol) of sodium cyanoborohydride were added and the mixture was heated at the boil for 2 h. After cooling to RT, the molecular sieve was filtered off and washed with methanol, and the filtrate was concentrated. The residue obtained was taken up in about 50 ml of ethyl acetate and washed successively with in each case about 50 ml of saturated aqueous sodium bicarbonate solution (twice) and saturated aqueous sodium chloride solution (once). After drying over anhydrous magnesium sulphate, the mixture was filtered and the filtrate was freed from the solvent on a rotary evaporator. The crude product was then initially pre-purified by MPLC (silica gel, dichloromethane/methanol 20:1), and the product was then isolated by HPLC (Method 14). After evaporation, the product fractions were once more dissolved in about 5 ml of methanol and the solution was passed through an ion exchanger column (Polymerlabs, Stratospheres SPE, PL-HCO₃ MP SPE, capacity 0.9 mmol) to convert the formic acid salt (from the HPLC) into the free acid. Concentration and drying under high vacuum gave 16 mg (30% of theory) of the title compound.

¹H NMR (400 MHz, CDCl₃, δ/ppm): 8.11 (d, 1H), 7.64 (d, 2H), 7.24 (d, 2H, partially obscured by the CHCl₃ signal), 6.55 (s, 1H), 6.50 (d, 1H), 6.29 (d, 1H), 6.28 (s, 1H), 5.19 (s, 2H), 3.47 (dd, 4H), 2.69 (dd, 4H), 2.24 (s, 3H), 1.67-1.60 (m, 1H), 0.49-0.45 (m, 4H).

LC/MS (Method 8, ESIpos): Rₜ=0.99 min, m/z=502 [M+H]⁺.

### Example 121

### 2-{3-[(3-{(Z)-2-Fluoro-2-[4-(trifluoromethoxy)phenyl]vinyl}-5-methyl-1H-pyrazol-1-yl)methyl]phenyl}propan-2-ol

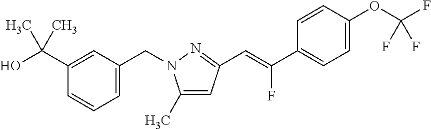 [SMILES: Cc1cc(/C=C(\F)c2ccc(OC(F)(F)F)cc2)nn1Cc1cccc(C(C)(C)O)c1]

Analogously to the process described under Example 53, 80 mg (0.184 mmol) of the compound from Example 115 and 405 μl (0.405 mmol) of a 1 M solution of methylmagnesium bromide in THF gave 48 mg (60% of theory) of the title compound. Here, the reaction time at RT was about 18 h. The crude product was purified by preparative HPLC according to Method 45, and the product obtained in this manner was finally triturated with pentane.

¹H NMR (400 MHz, CDCl₃, δ/ppm): 7.63 (d, 2H), 7.38 (d, 1H), 7.32 (s, 1H), 7.28 (t, 1H), 7.24 (d, 2H), 6.92 (d, 1H), 6.54 (d, 1H), 6.51 (s, 1H), 5.30 (s, 2H), 2.25 (s, 3H), 1.81 (s, broad, 1H), 1.55 (s, 6H).

LC/MS (Method 8, ESIpos): Rₜ=1.33 min, m/z=435 [M+H]⁺.

### B. EVALUATION OF THE PHARMACOLOGICAL ACTIVITY

The pharmacological activity of the compounds according to the invention can be demonstrated by in vitro and in vivo studies such as are known to the person skilled in the art. The usefulness of the substances according to the invention can be illustrated by way of example by in vitro (tumour) cell experiments and in vivo tumour models such as are described below. The connection between an inhibition of the HIF transcription activity and the inhibition of tumour growth is demonstrated by numerous studies described in the literature (cf. e.g. Warburg, 1956; Semenza, 2007).

### B-1. HIF-Luciferase Assay:

HCT 116 cells were transfected in a stable manner with a plasmid which contained a luciferase reporter under the control of an HIF-responsive sequence. These cells were sown in microtitre plates [20 000 cells/cavity in RPMI 1640 medium with 10% foetal calf serum (FCS) and 100 μg/ml of hygromycin]. Incubation was carried out overnight under standard conditions (5% CO₂, 21% O₂, 37° C., moistened). The following morning the cells were incubated with various concentrations of the test substances (0-10 μmol/l) in a hypoxia chamber (1% O₂). After 24 h, Bright Glo reagent (Promega, Wisconsin, USA) was added in accordance with the manufacturer's instructions, and after 5 min the luminescence was measured. Cells which were incubated under normoxia served as background controls.

The IC₅₀ values from this assay for representative working examples are listed in the following table (in same cases as means of up to four individual determinations):

| Example No. | IC₅₀ [nmol/L] |
|---|---|
| 2 | 30 |
| 4 | 2 |
| 7 | 40 |
| 9 | 0.4 |
| 10 | 0.4 |
| 11 | 0.2 |
| 12 | 0.3 |
| 13 | 0.5 |
| 14 | 0.2 |
| 15 | 2 |
| 16 | 0.5 |
| 17 | 1 |
| 18 | 20 |
| 23 | 1 |
| 25 | 6 |
| 26 | 1.5 |
| 27 | 4 |
| 28 | 5 |
| 29 | 5 |
| 30 | 6 |
| 31 | 3 |
| 32 | 4 |
| 33 | 4 |
| 34 | 5 |
| 35 | 5 |
| 36 | 4 |
| 37 | 3 |
| 38 | 4 |
| 39 | 2 |
| 40 | 2 |
| 41 | 3 |
| 42 | 4 |
| 43 | 2 |
| 44 | 0.5 |
| 46 | 0.7 |
| 48 | 2 |
| 49 | 0.8 |
| 50 | 1 |
| 51 | 1 |
| 52 | 1 |
| 53 | 4 |
| 54 | 3 |
| 55 | 4 |
| 56 | 2 |
| 57 | 1 |
| 58 | 2 |
| 59 | 2 |
| 61 | 4 |
| 62 | 2.5 |
| 63 | 3 |
| 64 | 2 |
| 79 | 1 |
| 80 | 2.8 |
| 81 | 3 |
| 82 | 1 |
| 83 | 0.4 |
| 84 | 2 |
| 85 | 4 |
| 86 | 0.3 |
| 87 | 0.7 |
| 88 | 2 |
| 89 | 2 |
| 90 | 2 |
| 91 | 0.3 |
| 92 | 5 |
| 93 | 5 |
| 94 | 1 |
| 95 | 0.6 |
| 96 | 1 |
| 97 | 20 |
| 102 | 0.5 |
| 103 | 2.5 |
| 104 | 0.4 |
| 105 | 0.5 |
| 106 | 0.5 |
| 107 | 0.3 |
| 108 | 3 |
| 109 | 1.5 |
| 110 | 1 |
| 111 | 40 |
| 113 | 4 |
| 114 | 40 |
| 117 | 30 |
| 120 | 20 |
| 121 | 40 |

### B-2. Suppression of HIF Target Genes In Vitro

Human bronchial carcinoma cells (H460 and A549) were incubated for 16 h with variable concentrations of the test substances (1 nM to 10 μM) under normoxic conditions and under a 1% oxygen partial pressure (see HIF-luciferase assay). The total RNA was isolated from the cells and transcribed into cDNA and the mRNA expression of HIF target genes was analysed in real time PCR. Active test substances already lower the mRNA expression of the HIF target genes compared with untreated cells under normoxic conditions, but above all under hypoxic conditions.

### B-3. Human Xenograft Tumour Models

Human tumour xenograft models in immunodeficient mice were used for evaluation of the substances. For this, tumour cells were cultured in vitro and implanted subcutaneously, or tumour xenotransplant pieces were transplanted further subcutaneously. The animals were treated by oral, subcutaneous or intraperitoneal therapy after the tumour was established. The activity of the test substances was analysed in monotherapy and in combination therapy with other pharmacological active substances. The tumour inhibitory potency of the test substances on tumours of advanced size (approx. 100 mm²) was moreover characterized. The state of health of the animals was checked daily, and the treatments were performed in accordance with animal protection regulations. The tumour area was measured with slide gauges (length L, breadth B=shorter dimension). The tumour volume was calculated from the formula (L×B²)/2. The inhibition in tumour growth was determined at the end of the study as the T/C ratio of the tumour areas and tumour weights and as the TGI value (tumour growth inhibition, calculated from the formula [1−(T/C)]×100) (T=tumour size in the treated group; C=tumour size in the untreated control group).

The influence of the test substances on the tumour vessel architecture and the blood flow within the tumour was identified with the aid of computer microtomography and ultrasound microstudies on treated and untreated tumour-carrying mice.

### B-4. Determination of Pharmacokinetic Parameters Following Intravenous and Oral Administration:

The substance to be investigated was administered to animals (e.g. mice or rats) intravenously as a solution (e.g. in corresponding plasma with a small addition of DMSO or in a PEG/ethanol/water mixture), and oral administration took place as a solution (e.g. in a Solutol/ethanol/water or PEG/ethanol/water mixture) or as a suspension (e.g. in tylose), in each case via a stomach tube. After administration of the substance, blood was taken from the animals at specified points in time. This was heparinized, and plasma was then obtained therefrom by centrifugation. The substance was quantified analytically in the plasma via LC-MS/MS. From the plasma concentration/time plots determined in this way, the pharmacokinetic parameters, such as AUC (area under the concentration/time curve), Cₘₐₓ (maximum plasma concentration), T_{1/2} (half life), V_{SS} (distribution volume) and CL (clearance), and the absolute and the relative bioavailability F and Fᵣₑₜ (i.v./p.o. comparison or comparison of suspension to solution after p.o. administration), were calculated using an internal standard and with the aid of a validated computer program.

### C. WORKING EXAMPLES OF PHARMACEUTICAL COMPOSITIONS

The compounds according to the invention can be converted into pharmaceutical formulations as follows.

### Tablet:

### Composition:

100 mg of the compound according to the invention, 50 mg of lactose (monohydrate), 50 mg of maize starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Preparation:

The mixture of compound according to the invention, lactose and starch is granulated with a 5% strength solution (w/w) of the PVP in water. After drying, the granules are mixed with the magnesium stearate for 5 minutes. This mixture is pressed with a conventional tablet press (for tablet format see above). A pressing force of 15 kN is used as the recommended value for the pressing.

### Suspension for Oral Administration:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to an individual dose of 100 mg of the compound according to the invention.

### Preparation:

The Rhodigel is suspended in ethanol and the compound according to the invention is added to the suspension. The water is added with stirring. The mixture is stirred for approx. 6 h until swelling of the Rhodigel has ended.

### Solution for Oral Administration:

### Composition:

500 mg of the compound according to the invention, 2.5 g of polysorbate and 97 g of polyethylene glycol 400.20 g of oral solution correspond to an individual dose of 100 mg of the compound according to the invention.

### Preparation:

The compound according to the invention is suspended in the mixture of polyethylene glycol and polysorbate, while stirring. The stirring operation is continued until dissolution of the compound according to the invention is complete.

i.v. Solution:

The compound according to the invention is dissolved in a concentration below the saturation solubility in a physiologically acceptable solvent (e.g. isotonic saline solution, glucose solution 5% and/or PEG 400 solution 30%). The solution is subjected to sterile filtration and is transferred into sterile and pyrogen-free injection containers.

### D. LITERATURE REFERENCES

Globocan 2002 Report
IARC International Agency for Research on Cancer: Globocan 2002, http://www-dep.iarc.fr/globocan/downloads.htm
American Cancer Society, Cancer Facts and Figures 2005
American Cancer Society: Cancer Facts and Figures 2007, http://www.cancer.org/docroot/STT/content/STT_{—}1x_Cancer_Facts_Figures_{—}2007.asp
Gibbs J B, 2000
Gibbs J B: Mechanism-based target identification and drug discovery in cancer research, Science 2000, 287 (5460), 1969-1973.
Semenza and Wang, 1992
Semenza G L, Wang G L: A nuclear factor induced by hypoxia via de novo protein synthesis binds to the human erythropoietin gene enhancer at a site required for transcriptional activation, Mol. Cell. Biol. 1992, 12 (12), 5447-5454.
Wang and Semenza, 1995
Wang G L, Semenza G L: Purification and characterization of hypoxia-inducible factor 1, J. Biol. Chem. 1995, 270 (3), 1230-1237.
Wang, Jiang et al., 1995
Wang G L, Jiang B H, Rue E A, Semenza G L: Hypoxia-inducible factor 1 is a basic-helix-loop-helix-PAS heterodimer regulated by cellular O₂ tension, PNAS 1995, 92 (12), 5510-5514.
Jiang, Rue et al., 1996
Jiang B H, Rue E, Wang G L, Roe R, Semenza G L: Dimerization, DNA binding, and transactivation properties of hypoxia-inducible factor 1, J. Biol. Chem. 1996, 271 (30), 17771-17778.
Makino, Cao et al., 2001
Makino Y, Cao R, Svensson K, Bertilsson G, Asman M, Tanaka H, Cao Y, Poellinger L: Nature 2001, 414 (6863), 550-554.
Jiang, Semenza et al., 1996
Jiang B H, Semenza G L, Bauer C, Marti H H: Hypoxia-inducible factor 1 levels vary exponentially over a physiologically relevant range of O₂ tension, Am. J. Physiol. 1996, 271, 1172-1180.
Maxwell, Wiesener et al., 1999
Maxwell P H, Wiesener M S, Chang G W, Clifford S C, Vaux E C, Cockman M E, Wykoff C C, Ratcliffe P J: The tumour suppressor protein VHL targets hypoxia-inducible factors for oxygen-dependent proteolysis, Nature 1999, 399 (6733), 271-275.
Hirota and Semenza, 2006
Hirota K, Semenza G L: Regulation of angiogenesis by hypoxia-inducible factor 1, Crit. Rev. Oncol. Hematol. 2006, 59 (1), 15-26.
Chen, Zhao et al., 2003
Chen J, Zhao S, Nakada K, Kuge Y, Tamaki N, Okada F, Wang J, Shindo M, Higashino F, Takeda K, Asaka M, Katoh H, Sugiyama T, Hosokawa M, Kobayashi M: Dominant-negative hypoxia-inducible factor-1alpha reduces tumorigenicity of pancreatic cancer cells through the suppression of glucose metabolism, Am. J. Pathol. 2003, 162 (4), 1283-1291.
Stoeltzing, McCarty et al., 2004
Stoeltzing O, McCarty M F, Wey J S, Fan F, Liu W, Belcheva A, Bucana C D, Semenza G L, Ellis L M: Role of hypoxia-inducible factor-1alpha in gastric cancer cell growth, angiogenesis, and vessel maturation, J. Natl. Cancer Inst. 2004, 96 (12), 946-956.
Li, Lin et al., 2005
Li L, Lin X, Stayer M, Shoemaker A, Semizarov D, Fesik S W, Shen Y: Evaluating hypoxia-inducible factor-1alpha as a cancer therapeutic target via inducible RNA interference in vivo, Cancer Res. 2005, 65 (16), 7249-7258.
Mizukami, Jo et al., 2005
Mizukami Y, Jo W S, Duerr E M, Gala M, Li J, Zhang X, Zimmer M A, Iliopoulos O, Zukerberg L R, Kohgo Y, Lynch M P, Rueda B R, Chung D C: Induction of interleukin-8 preserves the angiogenic response in HIF-1alpha-deficient colon cancer cells, Nat. Med. 2005, 11 (9), 992-997.
Li, Shi et al., 2006
Li J, Shi M, Cao Y, Yuan W, Pang T, Li B, Sun Z, Chen L, Zhao R C: Knockdown of hypoxia-inducible factor-1 alpha in breast carcinoma MCF-7 cells results in reduced tumor growth and increased sensitivity to methotrexate, Biochem. Biophys. Res. Commun. 2006, 342, 1341-1351.
Semenza, 2007
Semenza G L: Drug Discov. Today 2007, 12 (19-20), 853-859.
Weidemann and Johnson, 2008
Weidemann A, Johnson R S: Cell Death and Differentiation 2008, 15, 621-627.
Aiello et al., 1994
Aiello et al.: New Engl. J. Med. 1994, 331, 1480.
Peer et al., 1995
Peer et al.: Lab. Invest. 1995, 72, 638.
Lopez et al., 1996
Lopez et al.: Invest. Ophthalmol. Vis. Sci. 1996, 37, 855.
Warburg, 1956
Warburg O: Science 1956, 123 (3191), 309-314.

1. A compound of the formula (I) 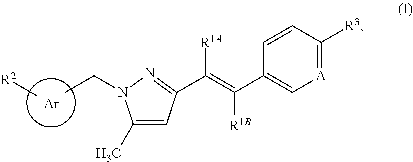
in which
one of the two radicals R^{1A} and R^{1B} represents fluorine and the other represents hydrogen,
Ar with the substituent R² represents a phenyl or pyridyl ring of the formula 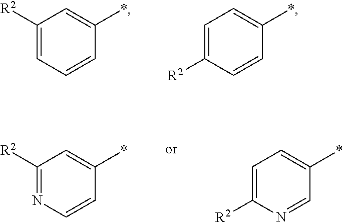
in which * denotes the point of attachment to the neighbouring CH₂ group,
R² represents hydrogen or a substituent selected from the group consisting of halogen, cyano, (C₁-C₆)-alkyl, (C₂-C₆)-alkenyl, (C₃-C₆)-cycloalkyl, (C₁-C₆)-alkoxy, (C₃-C₆)-cycloalkoxy, (C₁-C₄)alkoxycarbonyl, (C₁-C₄)-alkylsulphonyl, —NR⁵R⁶ and —C(═O)—NR⁵R⁶,
where (C₁-C₆)-alkyl for its part may be substituted up to three times by fluorine and up to two times by identical or different radicals selected from the group consisting of hydroxyl, (C₁-C₄)-alkoxy, (C₁-C₄)-alkylcarbonyloxy and (C₃-C₆)-cycloalkyl
and
the cycloalkyl groups mentioned for their part may be substituted up to two times by identical or different radicals selected from the group consisting of fluorine, (C₁-C₄)-alkyl, trifluoromethyl, hydroxyl, hydroxymethyl, (C₁-C₄)-alkoxy and (C₁-C₄)-alkylcarbonyloxy,
and in which
R⁵ and R⁶ independently of one another represent hydrogen, (C₁-C₆)-alkyl or (C₃-C₆)-cycloalkyl
or
R⁵ and R⁶ are attached to one another and together with the nitrogen atom to which they are attached form a 4- to 6-membered heterocycle which may contain a further heteroatom from the group consisting of N, O, S and S(O)₂ and which may be substituted up to two times by identical or different substituents selected from the group consisting of fluorine, cyano, hydroxyl, (C₁-C₄)-alkoxy, oxo, (C₁-C₄)-alkyl and (C₃-C₆)-cycloalkyl,
where (C₁-C₄)-alkyl for its part may be substituted up to three times by fluorine,
R³ represents a substituent selected from the group consisting of halogen, cyano, pentafluorothio, tri-(C₁-C₄)-alkylsilyl, (C₁-C₆)-alkyl, —NR⁷R⁸, —OR⁸, —SR⁸, —S(O)₂—R⁸, (C₃-C₆)-cycloalkyl and 4- to 6-membered heterocyclyl,
where (C₁-C₆)-alkyl for its part may be substituted by a radical selected from the group consisting of amino, —NR⁷R⁸, hydroxyl, —OR⁸, (C₃-C₆)-cycloalkyl and 4- to 6-membered heterocyclyl and also up to six times by fluorine
and
the cycloalkyl and heterocyclyl groups mentioned for their part may be substituted up to two times by identical or different radicals selected from the group consisting of fluorine, (C₁-C₄)-alkyl, trifluoromethyl, hydroxyl and (C₁-C₄)-alkoxy,
and in which
R⁷ represents hydrogen or (C₁-C₄)-alkyl
and
R⁸ represents (C₁-C₆)-alkyl or (C₃-C₆)-cycloalkyl,
where (C₁-C₆)-alkyl for its part may be substituted by a radical selected from the group consisting of hydroxyl, (C₁-C₄)-alkoxy, —NR⁹R¹⁰ and —C(═O)—NR⁹R¹⁰ and also up to three times by fluorine, in which
R⁹ and R¹⁰ independently of one another represent hydrogen or (C₁-C₄)-alkyl or are attached to one another and together with the nitrogen atom to which they are attached form a pyrrolidine, piperidine or morpholine ring,
and
A represents N or C—R⁴, in which
R⁴ represents hydrogen, fluorine, chlorine, cyano, methyl, trifluoromethyl or methoxy,
and salts, solvates and solvates of the salts thereof.

2. A compound of the formula (I) according to claim 1, in which one of the two radicals R^{1A} and R^{1B} represents fluorine and the other represents hydrogen,
Ar with the substituent R² represents a phenyl or pyridyl ring of the formula 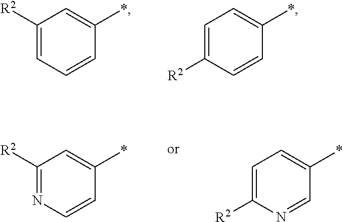
in which * denotes the point of attachment to the neighbouring CH₂ group,
R² represents a substituent selected from the group consisting of chlorine, (C₁-C₄)-alkyl, (C₃-C₆)-cycloalkyl, methoxy, ethoxy, methoxycarbonyl, ethoxycarbonyl, —NR⁵R⁶ and —C(═O)—NR⁵R⁶,
where (C₁-C₄)-alkyl for its part may be substituted by a radical selected from the group consisting of hydroxyl, acetoxy, cyclopropyl and cyclobutyl and up to three times by fluorine
and
(C₃-C₆)-cycloalkyl and cyclopropyl and cyclobutyl for their part may be substituted up to two times by identical or different radicals selected from the group consisting of fluorine, methyl, trifluoromethyl, hydroxyl, hydroxymethyl, methoxy and acetoxy,
and in which
R⁵ represents hydrogen or methyl,
R⁶ represents hydrogen or (C₁-C₄)-alkyl,
or
R⁵ and R⁶ are attached to one another and together with the nitrogen atom to which they are attached form a 4- to 6-membered heterocycle which may contain a further heteroatom from the group consisting of N, O and S and which may be substituted by a radical selected from the group consisting of cyano, hydroxyl, methoxy, ethoxy, (C₁-C₄)-alkyl, cyclopropyl and cyclobutyl,
where (C₁-C₄)-alkyl for its part may be substituted up to three times by fluorine,
R³ represents a substituent selected from the group consisting of pentafluorothio, trimethylsilyl, (C₁-C₆)-alkyl, —OR⁸, —SR⁸, (C₃-C₆)-cycloalkyl and 4- to 6-membered heterocyclyl,
where (C₁-C₆)-alkyl for its part may be substituted by hydroxyl or —OR⁸ and also up to six times by fluorine
and
(C₃-C₆)-cycloalkyl and 4- to 6-membered heterocyclyl for their part may be substituted up to two times by identical or different radicals selected from the group consisting of fluorine, methyl, trifluoromethyl, hydroxyl, methoxy and ethoxy,
and in which
R⁸ represents (C₁-C₄)-alkyl which may be substituted by a radical selected from the group consisting of hydroxyl, methoxy and ethoxy and also up to three times by fluorine,
and
A represents N or C—R⁴, in which
R⁴ represents hydrogen, fluorine or chlorine,
and salts, solvates and solvates of the salts thereof.

3. A compound of the formula (I) according to claim 1, in which one of the two radicals R^{1A} and R^{1B} represents fluorine and the other represents hydrogen,
Ar with the substituent R² represents a phenyl or pyridyl ring of the formula 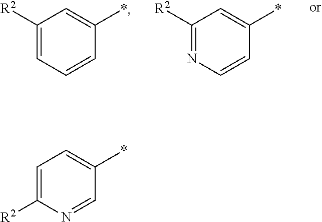
in which * denotes the point of attachment to the neighbouring CH₂ group,
R² represents a substituent selected from the group consisting of (C₁-C₄)-alkyl, cyclopropyl, cyclobutyl, —NR⁵R⁶ and —C(═O)—NR⁵R⁶,
where (C₁-C₄)-alkyl for its part may be substituted by a radical selected from the group consisting of hydroxyl, acetoxy, cyclopropyl and cyclobutyl and also up to three times by fluorine
and
the cyclopropyl and cyclobutyl groups mentioned for their part may be substituted by a radical selected from the group consisting of hydroxyl, hydroxymethyl and acetoxy,
and in which
R⁵ represents hydrogen,
R⁶ represents (C₁-C₄)-alkyl,
or
R⁵ and R⁶ are attached to one another and together with the nitrogen atom to which they are attached form a 4- to 6-membered heterocycle which may contain a further heteroatom from the group consisting of N and O and which may be substituted by a radical selected from the group consisting of cyano, hydroxyl, (C₁-C₄)-alkyl and cyclopropyl,
where (C₁-C₄)-alkyl for its part may be substituted up to three times by fluorine,
R³ represents a substituent selected from the group consisting of trifluoromethoxy, trifluoromethylsulphanyl, pentafluorothio, trimethylsilyl, (C₁-C₄)-alkyl, cyclopropyl, cyclobutyl, cyclohexyl, oxetan-3-yl and tetrahydro-2H-pyran-4-yl,
where (C₁-C₄)-alkyl for its part may be substituted by hydroxyl and also up to six times by fluorine
and
cyclopropyl, cyclobutyl, cyclohexyl, oxetanyl and tetrahydropyranyl for their part may be substituted by fluorine or trifluoromethyl,
and
A represents C—R⁴, in which
R⁴ represents hydrogen or fluorine,
and salts, solvates and solvates of the salts thereof.

4. A compound of the formula (I) according to claim 1, in which
R^{1A} represents fluorine,
R^{1B} represents hydrogen,
Ar with the substituent R² represents a phenyl or pyridyl ring of the formula 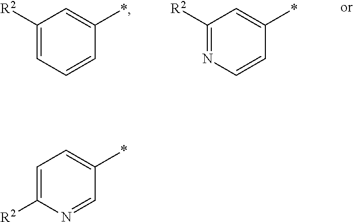
in which * denotes the point of attachment to the neighbouring CH₂ group,
R² represents the group —NR⁵R⁶, in which
R⁵ represents hydrogen,
R⁶ represents methyl or ethyl,
or
R⁵ and R⁶ are attached to one another and together with the nitrogen atom to which they are attached form a substituted heterocycle of the formula 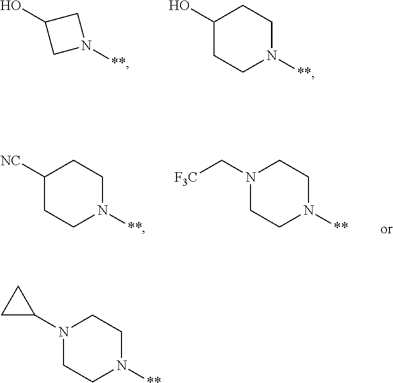
in which ** denotes the point of attachment to the ring Ar,
or
R² represents a substituted isopropyl, isobutyl or cyclopropyl group of the formula 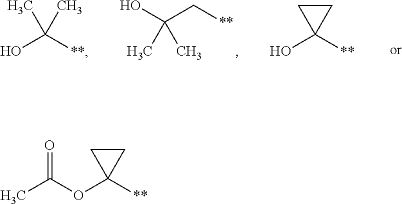
in which ** denotes the point of attachment to the ring Ar,
R³ represents trifluoromethyl, trifluoromethoxy, trifluoromethylsulphanyl, pentafluorothio, trimethylsilyl, tert-butyl or a group of the formula 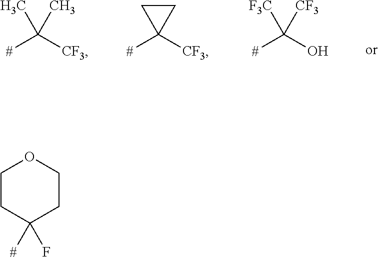
in which # denotes the point of attachment to the neighbouring ring,
and
A represents C—R⁴, in which
R⁴ represents hydrogen or fluorine,
and salts, solvates and solvates of the salts thereof.

5. A process for preparing compounds of the formula (I) as defined in claim 1, wherein either
[A-1] a fluorinated pyrazolylmethylbenzothiazolylsulphone of the formula (II) 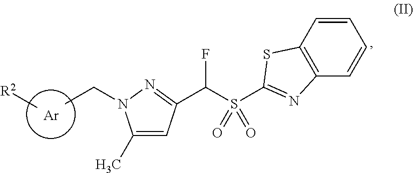
in which Ar and R² have the meanings given in claim 1,
is reacted in an inert solvent in the presence of a base with an aldehyde of the formula (III) 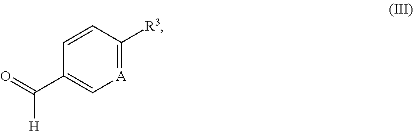
in which A and R³ have the meanings given in claim 1,
to give a compound of the formula (I-A) according to the invention 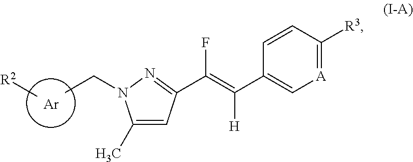
in which A, Ar, R² and R³ have the meanings given in claim 1,
or
[A-2] initially a fluorinated pyrazolylmethylbenzothiazolylsulphone of the formula (IV) 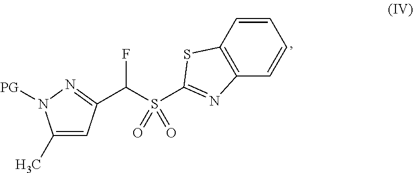
in which
PG represents a protective group,
is reacted in an inert solvent in the presence of a base with an aldehyde of the formula (III) 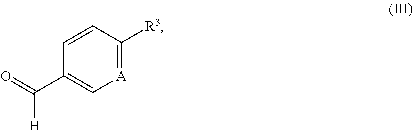
in which A and R³ have the meanings given in claim 1,
to give a compound of the formula (V) 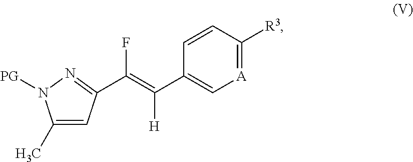
in which A, PG and R³ have the meanings given above,
the protective group PG is then removed by customary methods and the resulting pyrazole derivative of the formula (VI) 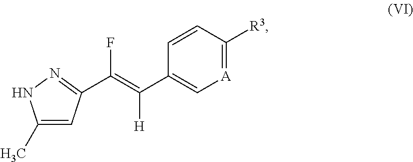
in which A and R³ have the meanings given in claim 1,
is then alkylated in an inert solvent in the presence of a base with a compound of the formula (VII) 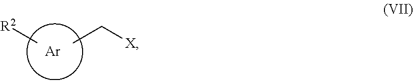
in which Ar and R² have the meanings given in claim 1
and
X represents a leaving group,
to give a compound of the formula (I-A) according to the invention 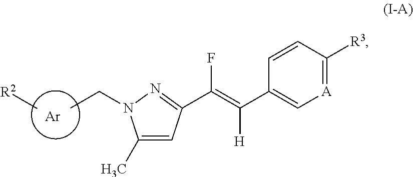
in which A, Ar, R² and R³ have the meanings given in claim 1,
or
[B-1] a fluorinated arylmethylbenzothiazolylsulphone of the formula (VIII) 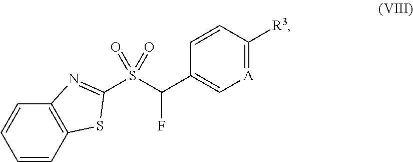
in which A and R³ have the meanings given in claim 1,
is reacted in an inert solvent in the presence of a base with a pyrazolecarbaldehyde of the formula (IX) 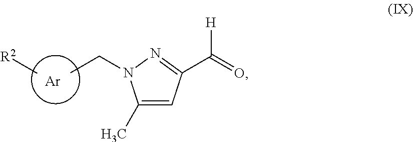
in which Ar and R² have the meanings given in claim 1,
to give a compound of the formula (I-B) according to the invention 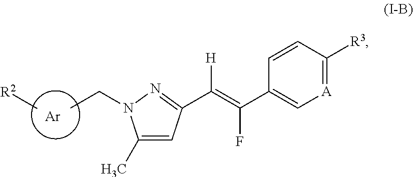
in which A, Ar, R² and R³ have the meanings given in claim 1,
or
[B-2] a fluorinated arylmethylbenzothiazolylsulphone of the formula (VIII) 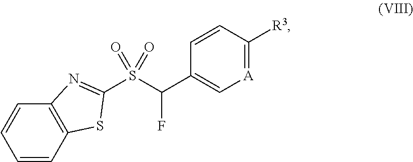
in which A and R³ have the meanings given in claim 1,
is reacted in an inert solvent in the presence of a base first with a protected pyrazolecarbaldehyde of the formula (X) 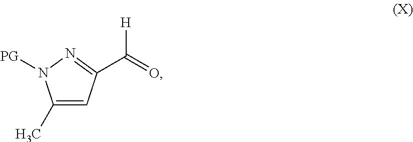
in which
PG represents a protective group,
to give a compound of the formula (XI) 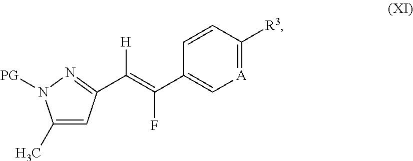
in which A, PG and R³ have the meanings given above,
the protective group PG is then removed by customary methods and the resulting pyrazole derivative of the formula (XII) 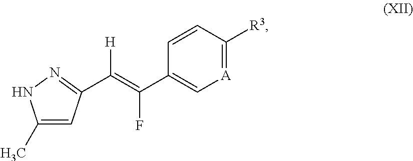
in which A and R³ have the meanings given in claim 1,
is then alkylated in an inert solvent in the presence of a base with a compound of the formula (VII) 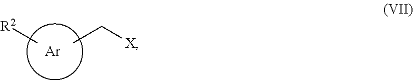
in which Ar and R² have the meanings given in claim 1
and
X represents a leaving group,
to give a compound of the formula (I-B) according to the invention 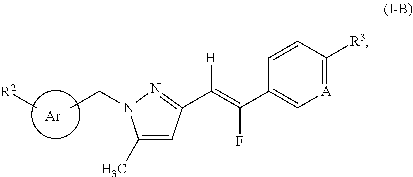
in which A, Ar, R² and R³ have the meanings given in claim 1,
and the compound of the formula (I-A) or (I-B) is optionally separated into its enantiomers and/or diastereomers and/or converted with the appropriate (i) solvents and/or (ii) bases or acids into a solvate, salt and/or solvate of the salt.

6. (canceled)

7. (canceled)

8. (canceled)

9. (canceled)

10. (canceled)

11. A pharmaceutical composition comprising a compound as defined in claim 1 in combination with one or more inert, non-toxic, pharmaceutically suitable auxiliary substances.

12. A pharmaceutical composition comprising a compound as defined in claim 1 in combination with one or more other active compounds.

13. (canceled)

14. (canceled)

15. A method for the treatment and/or prevention of a cancer disease or tumour disease comprising administering to a human or animal in need thereof an effective amount of at least one compound as defined in claim 1.

16. A method for the treatment and/or prevention of ischaemic cardiovascular diseases, cardiac insufficiency, cardiac infarction, arrhythmia, stroke, pulmonary hypertension, fibrotic diseases of the kidney and lung, psoriasis, diabetic retinopathy, macular degeneration, rheumatic arthritis, or Chugwash polycythaemia comprising administering to a human or animal in need thereof an effective amount of at least one compound as defined in claim 1.
